SEQUENCE LISTING (39 sequences)

SEQ ID NO: 1 - protein, 6 aa, Artificial Sequence
  Description of Artificial Sequence Synthetic peptide
SGGGGS

SEQ ID NO: 2 - protein, 7 aa, Artificial Sequence
  Description of Artificial Sequence Synthetic peptide
KASGGPE

SEQ ID NO: 3 - DNA, 21 bp, Artificial Sequence
  Description of Artificial Sequence Synthetic primer
gatcttgcaactccatttcgg

SEQ ID NO: 4 - DNA, 21 bp, Artificial Sequence
  Description of Artificial Sequence Synthetic primer
ggctatgtcaacactgaacac

SEQ ID NO: 5 - DNA, 20 bp, Artificial Sequence
  Description of Artificial Sequence Synthetic primer
ccagacagagacgtgtacag

SEQ ID NO: 6 - DNA, 24 bp, Artificial Sequence
  Description of Artificial Sequence Synthetic primer
tcgatggtattaggatagaagcac

SEQ ID NO: 7 - DNA, 21 bp, Artificial Sequence
  Description of Artificial Sequence Synthetic primer
gcagtacatcaacaacctcag

SEQ ID NO: 8 - DNA, 22 bp, Artificial Sequence
  Description of Artificial Sequence Synthetic primer
cagcatctacatccttcttcag

SEQ ID NO: 9 - DNA, 20 bp, Artificial Sequence
  Description of Artificial Sequence Synthetic primer
tcaagaaggatgtggatggt

SEQ ID NO: 10 - DNA, 20 bp, Artificial Sequence
  Description of Artificial Sequence Synthetic primer
ttggtagagtgctgtaagga

SEQ ID NO: 11 - DNA, 21 bp, Artificial Sequence
  Description of Artificial Sequence Synthetic primer
ggacttagtccagaaatacct

SEQ ID NO: 12 - DNA, 20 bp, Artificial Sequence
  Description of Artificial Sequence Synthetic primer
ctttcagcccaaagaattcc

SEQ ID NO: 13 - DNA, 21 bp, Artificial Sequence
  Description of Artificial Sequence Synthetic primer
tctacaaagaggtggacagag

SEQ ID NO: 14 - DNA, 20 bp, Artificial Sequence
  Description of Artificial Sequence Synthetic primer
gttagaagtgaggctgtgag

SEQ ID NO: 15 - DNA, 22 bp, Artificial Sequence
  Description of Artificial Sequence Synthetic primer
ttgagtctatctggtaacactg

SEQ ID NO: 16 - DNA, 20 bp, Artificial Sequence
  Description of Artificial Sequence Synthetic primer
acataacacgcattcattgg

SEQ ID NO: 17 - DNA, 22 bp, Artificial Sequence
  Description of Artificial Sequence Synthetic primer
tcttctggcgtatttcattctg

SEQ ID NO: 18 - DNA, 20 bp, Artificial Sequence
  Description of Artificial Sequence Synthetic primer
ctggtcatcattgtttggag

SEQ ID NO: 19 - DNA, 20 bp, Artificial Sequence
  Description of Artificial Sequence Synthetic primer
agacaaagatggagaaggac

SEQ ID NO: 20 - DNA, 22 bp, Artificial Sequence
  Description of Artificial Sequence Synthetic primer
aatacgtgctgaatactgagag

SEQ ID NO: 21 - DNA, 21 bp, Artificial Sequence
  Description of Artificial Sequence Synthetic primer
gcagttaaagaacatggagac

SEQ ID NO: 22 - DNA, 22 bp, Artificial Sequence
  Description of Artificial Sequence Synthetic primer
tctgtgaatgagttgtagagtg

SEQ ID NO: 23 - DNA, 22 bp, Artificial Sequence
  Description of Artificial Sequence Synthetic primer
aaagtcaagagcaccataacct

SEQ ID NO: 24 - DNA, 21 bp, Artificial Sequence
  Description of Artificial Sequence Synthetic primer
caccaccagtttatcatcctc

SEQ ID NO: 25 - DNA, 20 bp, Artificial Sequence
  Description of Artificial Sequence Synthetic primer
ataagccagactttgttgga

SEQ ID NO: 26 - DNA, 22 bp, Artificial Sequence
  Description of Artificial Sequence Synthetic primer
caacttgaactctcatcttagg

SEQ ID NO: 27 - DNA, 21 bp, Artificial Sequence
  Description of Artificial Sequence Synthetic primer
gacaggatgaaagacatggag

SEQ ID NO: 28 - DNA, 21 bp, Artificial Sequence
  Description of Artificial Sequence Synthetic primer
aaagcattccaacccaatctg

SEQ ID NO: 29 - DNA, 22 bp, Artificial Sequence
  Description of Artificial Sequence Synthetic primer
tgaattatacaagagccgagga

SEQ ID NO: 30 - DNA, 21 bp, Artificial Sequence
  Description of Artificial Sequence Synthetic primer
gacacagcaccaatactacac

SEQ ID NO: 31 - DNA, 20 bp, Artificial Sequence
  Description of Artificial Sequence Synthetic primer
acaaagtgcaagattctgcc

SEQ ID NO: 32 - DNA, 20 bp, Artificial Sequence
  Description of Artificial Sequence Synthetic primer
attcacatggcataaagccc

SEQ ID NO: 33 - DNA, 20 bp, Artificial Sequence
  Description of Artificial Sequence Synthetic primer
agactcttaactgtagcgtg

SEQ ID NO: 34 - DNA, 21 bp, Artificial Sequence
  Description of Artificial Sequence Synthetic primer
aatgtgctgttccataacctc

SEQ ID NO: 35 - DNA, 23 bp, Artificial Sequence
  Description of Artificial Sequence Synthetic primer
caaggagaaatacgagaagttgg

SEQ ID NO: 36 - DNA, 20 bp, Artificial Sequence
  Description of Artificial Sequence Synthetic primer
ctggtaagtacacgatgctg

SEQ ID NO: 37 - DNA, 21 bp, Artificial Sequence
  Description of Artificial Sequence Synthetic primer
gcgaggactttaatcttggtg

SEQ ID NO: 38 - DNA, 22 bp, Artificial Sequence
  Description of Artificial Sequence Synthetic primer
ctcagaactttggtgtcattgg

SEQ ID NO: 39 - protein, 4 aa, Artificial Sequence
  Description of Artificial Sequence Synthetic peptide
DEAD

(19) United States
(12) Patent Application Publication
(10) Pub. No.: US 2011/0020221 A1
(43) Pub. Date: Jan. 27, 2011

(54) CANCER STEM CELL EXPRESSION PATTERNS AND COMPOUNDS TO TARGET CANCER STEM CELLS

(75) Inventors: David M. Berman, Towson, MD (US); Xiaobing He, Ann Arbor, MI (US); William Matsui, Baltimore, MD (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/757,649

(22) Filed: Apr. 9, 2010

Related U.S. Application Data
(60) Provisional application No. 61/168,178, filed on Apr. 9, 2009.

(51) Int. Cl. A61K 39/395; A61P 35/00; A61K 51/00; A61P 35/02

(52) U.S. Cl. 424/1.49; 424/130.1; 424/178.1; 424/138.1; 424/158.1

(57) ABSTRACT

Described herein are therapeutic targets expressed in cancer stem cells and methods for treating and diagnosing cancer by targeting such cells with antibodies, compounds, nucleic acid, or other therapeutic agent. In one embodiment described herein, therapeutic agents for the treatment of cancer are provided based on the identification of cancer stem cell targets. The present invention also includes therapeutic targets for cancer therapy and cancer stem cell-targeted therapy. The invention includes the treatment of cancer by the administration of compounds or agents that target cancer stem cells.

This application claims and is entitled to priority benefit of U.S. provisional application Ser. No. 61/168,178 filed Apr. 9, 2009 which is incorporated herein by reference in its entirety.

### FIELD OF THE INVENTION

Described herein are therapeutic targets expressed in cancer stem cells and methods for treating and diagnosing cancer by targeting such cells with antibodies, compounds, nucleic acid, or other therapeutic agent. In one embodiment described herein, therapeutic agents for the treatment of cancer are provided based on the identification of cancer stem cell targets. The present invention also includes therapeutic targets for cancer therapy and cancer stem cell-targeted therapy. The invention includes the treatment of cancer by the administration of compounds or agents that target cancer stem cells. In one embodiment the cancer this is treated is urothelial cancer, and the cancer stem cells that are targeted are urothelial cancer stem cells. The invention includes the administration of the agents of the invention in novel dosing regimens. The invention also includes the monitoring of patients who are being treated with the agents of the invention via the detection of cancer stem cells before, during and after treatment. The detection can be done in the patient using imaging techniques, or the detection can be done with a specimen from the patient, such as a blood sample, bone marrow sample or biopsy. The invention includes the diagnosis, monitoring and staging of cancer by the detection of one or more of the cancer stem cell genes described herein.

### BACKGROUND OF THE INVENTION

Cancer is one of the most significant health conditions. The American Cancer Society's Cancer Facts and Figures, 2003, predicts over 1.3 million Americans will receive a cancer diagnosis this year. In the United States, cancer is second only to heart disease in mortality accounting for one of four deaths. In 2002, the National Institutes of Health estimated total costs of cancer totaled $171.6 billion, with $61 billion in direct expenditures. The incidence of cancer is widely expected to increase as the US population ages, further augmenting the impact of this condition. The current treatment regimens for cancer, established in the 1970s and 1980s, have not changed dramatically. These treatments, which include chemotherapy, radiation and other modalities including newer targeted therapies, have shown limited overall survival benefit when utilized in most advanced stage common cancers since, among other things, these therapies primarily target tumor bulk rather than cancer stem cells.

More specifically, conventional cancer diagnosis and therapies to date have attempted to selectively detect and eradicate neoplastic cells that are largely fast-growing (i.e., cells that form the tumor bulk). Standard oncology regimens have often been largely designed to administer the highest dose of irradiation or a chemotherapeutic agent without undue toxicity, i.e., often referred to as the “maximum tolerated dose” (MTD) or “no observed adverse effect level” (NOAEL). Many conventional cancer chemotherapies (e.g., alkylating agents such as cyclophosphamide, antimetabolites such as 5-Fluorouracil, plant alkaloids such as vincristine) and conventional irradiation therapies exert their toxic effects on cancer cells largely by interfering with cellular mechanisms involved in cell growth and DNA replication. Chemotherapy protocols also often involve administration of a combination of chemotherapeutic agents in an attempt to increase the efficacy of treatment. Despite the availability of a large variety of chemotherapeutic agents, these therapies have many drawbacks (see, e.g., Stockdale, 1998, “Principles Of Cancer Patient Management” in Scientific American Medicine, vol. 3, Rubenstein and Federman, eds., ch. 12, sect. X). For example, chemotherapeutic agents are notoriously toxic due to non-specific side effects on fast-growing cells whether normal or malignant; e.g. chemotherapeutic agents cause significant, and often dangerous, side effects, including bone marrow depression, immunosuppression, gastrointestinal distress, etc.

Other types of traditional cancer therapies include surgery, hormonal therapy, immunotherapy, epigenetic therapy, anti-angiogenesis therapy, targeted therapy (e.g. therapy directed to a cancer target such as Gleevec® and other tyrosine kinase inhibitors, Velcade®, Sutent®, et al.), and radiation treatment to eradicate neoplastic cells in a patient (see, e.g., Stockdale, 1998, “Principles of Cancer Patient Management,” in Scientific American: Medicine, vol. 3, Rubenstein and Federman, eds., ch. 12, sect. IV). All of these approaches can pose significant drawbacks for the patient including a lack of efficacy (in terms of long-term outcome (e.g. due to failure to target cancer stem cells) and toxicity (e.g. due to non-specific effects on normal tissues)). Accordingly, new therapies and/or regimens for improving the long-term prospect of cancer patients are needed.

### Cancer Stem Cells

Cancer stem cells comprise a unique subpopulation (often 0.1-10% or so) of a tumor that, relative to the remaining 90% or so of the tumor (i.e., the tumor bulk), are more tumorigenic, relatively more slow-growing or quiescent, and often relatively more chemoresistant than the tumor bulk. Given that conventional therapies and regimens have, in large part, been designed to attack rapidly proliferating cells (i.e. those cancer cells that comprise the tumor bulk), cancer stem cells which are often slow-growing may be relatively more resistant than faster growing tumor bulk to conventional therapies and regimens. Cancer stem cells can express other features which make them relatively chemoresistant such as multi-drug resistance and anti-apoptotic pathways. The aforementioned would constitute a key reason for the failure of standard oncology treatment regimens to ensure long-term benefit in most patients with advanced stage cancers—i.e. the failure to adequately target and eradicate cancer stem cells. In some instances, a cancer stem cell(s) is the founder cell of a tumor (i.e., it is the progenitor of the cancer cells that comprise the tumor bulk).

Cancer stem cells have been identified in a large variety of cancer types. For instance, Bonnet et al., using flow cytometry were able to isolate the leukemia cells bearing the specific phenotype CD34+CD38−, and subsequently demonstrate that it is these cells (comprising <1% of a given leukemia), unlike the remaining 99+% of the leukemia bulk, that are able to recapitulate the leukemia from whenst it was derived when transferred into immunodeficient mice. See, e.g., “Human acute myeloid leukemia is organized as a hierarchy that originates from a primitive hematopoietic cell,” Nat Med 3:730-737 (1997). That is, these cancer stem cells were found as <1 in 10,000 leukemia cells yet this low frequency population was able to initiate and serially transfer a human leukemia into severe combined immunodeficiency/non-obese diabetic (NOD/SCID) mice with the same histologic phenotype as in the original tumor.

Cox et al. identified small subfractions of human acute lymphoblastic leukemia (ALL) cells which had the phenotypes CD34⁺/CD10⁻ and CD34⁺/CD19⁻, and were capable of engrafting ALL tumors in immunocompromised mice—i.e. the cancer stem cells. In contrast, no engraftment of the mice was observed using the ALL bulk, despite, in some cases, injecting 10-fold more cells. See Cox et al., “Characterization of acute lymphoblastic leukemia progenitor cells,” Blood 104(19): 2919-2925 (2004).

Multiple myeloma was found to contain small subpopulations of cells that were CD138− and, relative to the large bulk population of CD138+ myeloma cells, had greater clonogenic and tumorigenic potential. See Matsui et al., “Characterization of clonogenic multiple myeloma cells,” Blood 103(6): 2332. The authors concluded that the CD138-subpopulation of multiple myeloma was the cancer stem cell population.

Kondo et al. isolated a small population of cells from a C6-glioma cell line, which was identified as the cancer stem cell population by virtue of its ability to self-renew and recapitulate gliomas in immunocompromised mice. See Kondo et al., “Persistence of a small population of cancer stem-like cells in the C6 glioma cell line,” Proc. Natl. Acad. Sci. USA 101:781-786 (2004). In this study, Kondo et al. determined that cancer cell lines contain a population of cancer stem cells that confer the ability of the line to engraft immunodeficient mice.

Breast cancers were shown to contain a small population of cells with stem cell characteristics (bearing surface markers CD44+CD24^{low lin-}). See Al-Hajj et al., “Prospective identification of tumorigenic breast cancer cells,” Proc. Natl. Acad. Sci. USA 100:3983-3988 (2003). As few as 200 of these cells, corresponding to 1-10% of the total tumor cell population, are able to form tumors in NOD/SCID mice. In contrast, implantation of 20,000 cells that lacked this phenotype (i.e. the tumor bulk) was unable to re-grow the tumor.

A subpopulation of cells derived from human prostate tumors was found to self-renew and to recapitulate the phenotype of the prostate tumor from which they were derived thereby constituting the prostate cancer stem cell population. See Collins et al., “Prospective Identification of Tumorigenic Prostate Cancer Stem Cells,” Cancer Res 65(23):10946-10951 (2005).

Fang et al. isolated a subpopulation of cells from melanoma with cancer stem cell properties. In particular, this subpopulation of cells could differentiate and self-renew. In culture, the subpopulation formed spheres whereas the more differentiated cell fraction from the lesions were more adherent. Moreover, the subpopulation containing sphere-like cells were more tumorigenic than the adherent cells when grafted into mice. See Fang et al., “A Tumorigenic Subpopulation with Stem Cell Properties in Melanomas,” Cancer Res 65(20): 9328-9337 (2005).

Singh et al. identified brain tumor stem cells. When isolated and transplanted into nude mice, the CD133+ cancer stem cells, unlike the CD133− tumor bulk cells, form tumors that can then be serially transplanted. See Singh et al., “Identification of human brain tumor initiating cells,” Nature 432:396-401 (2004); Singh et al., “Cancer stem cells in nervous system tumors,” Oncogene 23:7267-7273 (2004); Singh et al., “Identification of a cancer stem cell in human brain tumors,” Cancer Res. 63:5821-5828 (2003).

He et al. identified urothelial cancer stem cells. The cells co-express 67-kDa laminin receptor and the basal cell-specific cytokeratin CK17 and lack the carcinoembryonic antigen family member CEACAM6 (CD66c). When isolated and transplanted into nude mice, the 67-LR bright cancer stem cells, unlike the 67-LR dim tumor bulk cells, form tumors that can then be serially transplanted. See He et al., “Differentiation of a highly tumorigenic basal cell compartment in urothelial carcinoma.” Stem Cells 27:1487-1495 (2009).

Since conventional cancer therapies target rapidly proliferating cells (i.e., cells that form the tumor bulk) these treatments are believed to be relatively ineffective at targeting and impairing cancer stem cells. In fact, cancer stem cells, including leukemia stem cells, have indeed been shown to be relatively resistant to conventional chemotherapeutic therapies (e.g. Ara-C, daunorubicin) as well as newer targeted therapies (e.g. Gleevec®, Velcade®). Examples of cancer stem cells from various tumors that are resistant to chemotherapy, and the mechanism by which they are resistant, are described in Table 1.

For example, leukemic stem cells are relatively slow-growing or quiescent, express multi-drug resistance genes, and utilize other anti-apoptotic mechanisms—features which contribute to their chemoresistance. See Jordan et al., “Targeting the most critical cells: approaching leukemia therapy as a problem in stem cell biology”, Nat Clin Pract Oncol. 2: 224-225 (2005). Further, cancer stem cells by virtue of their chemoresistance may contribute to treatment failure, and may also persist in a patient after clinical remission and these remaining cancer stem cells may therefore contribute to relapse at a later date. See Behbood et al., “Will cancer stem cells provide new therapeutic targets?” Carcinogenesis 26(4): 703-711 (2004). Therefore, targeting cancer stem cells is expected to provide for improved long-term outcomes for cancer patients. Accordingly, new therapeutic agents and/or regimens designed to target cancer stem cells are needed to reach this goal.

### SUMMARY OF THE INVENTION

The present invention is directed to agents used for the treatment of cancer. The invention also relates to agents that target cancer stem cells. The agents of the invention target genes, gene products, and/or proteins that are expressed in cancer stem cells. In one embodiment, the cancer being treated is bladder cancer. In another embodiment, the cancer stem cells being targeted are bladder cancer stem cells. The present invention also relates to methods for treating and/or managing cancer comprising administration of a therapeutically effective regimen of a agent that targets cancer stem cells. The present invention also relates to methods for the prevention, of cancer comprising administration of a prophylactically effective regimen of an agent that targets cancer stem cells. The invention includes agents that target proteins that are expressed by cancer stem cells. In one embodiment, the cancer being treated with these agents is bladder cancer. In some embodiments of the methods, the therapeutically effective regimen stabilizes, reduces or eliminates cancer stem cells. The therapeutically effective regimen of the invention, in some embodiments, includes monitoring the cancer stem cell population in a patient receiving the agents of the invention, and possibly altering the therapeutic regimen based on the results of such monitoring. In some embodiments of the methods, the prophylactically effective regimen stabilizes, reduces or eliminates cancer stem cells. The prophylactically effective regimen of the invention, in some embodiments, includes monitoring the cancer stem cell population in a patient receiving the agents of the invention, and possibly altering the prophylactic regimen based on the results of such monitoring. Monitoring cancer stem cell populations can be preformed, for example, as described in US Patent Publication 2008/01185518.

The invention includes a method for monitoring the cancer stem cell population in a patient prior to, during, and/or following treatment for cancer comprising determining the amount of cancer stem cells i) in a sample obtained from the patient and/or ii) within a patient via in vivo imaging. In certain embodiment, the patients being monitored are being treated with a agent (or combination of agents) of the invention that target a protein or pathway that is upregulated in cancer stem cells. In certain aspects of this embodiment, the method can further comprise comparing the amount of cancer stem cells within the patient or in the sample obtained from the patient to the amount of cancer stem cells in a reference sample, or to a predetermined reference range, wherein a stabilization or a decrease in the amount of cancer stem cells in the patient or patient sample relative to the reference sample, or to a predetermined reference range, indicates that the cancer therapy is effective; whereas, an increase in the amount of cancer stem cells in the patient or patient sample relative to the reference sample, or to a predetermined reference range, indicates that the cancer therapy is ineffective. In different aspects of this embodiment, the reference sample is a sample obtained from the patient from an earlier time (e.g. prior to undergoing cancer therapy or prior to the last treatment) or the reference sample is a sample obtained from, or within, a second patient having the same type of cancer that is in remission, or the reference sample is a sample obtained from, or within, a healthy person with no detectable cancer. In certain embodiments, the cancer stem cells of the patient are monitored by measuring a the expression of a gene, protein or pathway, that is active in the cancer stem cell. Representative genes, gene products, and pathways that are expressed or active in cancer stem cells are provided by the invention in the Tables. The expression pattern of the cancer stem cells, as utilized in the invention, can also be molecular gene signatures that are useful as a diagnostic and/or prognostic indicator. The gene signature can also be utilized to monitoring the response of the patient prior to, during, or after treatment.

The present invention relates to methods for monitoring the amount of cancer stem cells prior to, during, and/or following cancer treatment of a patient with an agent of the invention. In particular, the methods provide measuring the amount of cancer stem cells i) in a sample obtained from a patient and/or ii) in a patient via in vivo imaging, at different time points before, during and/or after a treatment regimen for cancer with an agent of the invention. The change in amount of cancer stem cells over time allows the physician to judge the effectiveness of the treatment regimen and then to decide to continue, alter, or halt the treatment regimen if need be. The present invention also provides kits for monitoring cancer stem cells prior to, during, and/or following cancer treatment of a patient.

An agent of the invention includes a nucleotide-based molecules (including but not limited to microRNA, siRNA, piRNA, antisense, ribozymes, protein-nucleic acids, aptamers, double stranded RNA (dsRNA), linked nucleic acids (LNA), 2-5A antisense) that target mRNA that are expressed in cancer stem cells. The agent or compound of the invention can also be small molecules that target the proteins and metabolic pathways expressed in cancer stem cells, including the proteins and gene products described in the Tables. The compounds or agents may include inhibitors of proteases, kinases, G-protein coupled receptors, transcription factors, ion channels. The pathways that can be inhibited by the agents of compounds of the invention are described in the Tables. The agents of the invention also include biologics, including antibodies to the proteins described in the Table. Biologics of the invention also include immunotoxins, protein toxins, protein inhibitors, and receptor decoys that target gene products described in the Tables.

The present invention is directed to a method for monitoring the cancer stem cell population in a patient prior to, during, and/or following treatment for cancer with an agent of the invention. The invention includes determining the amount of cancer stem cells i) in a sample obtained from the patient and/or ii) within a patient via in vivo imaging. In certain aspects of this embodiment, the method can further comprise comparing the amount of cancer stem cells within the patient or in the sample obtained from the patient to the amount of cancer stem cells in a reference sample, or to a predetermined reference range, wherein a stabilization or a decrease in the amount of cancer stem cells in the patient or patient sample relative to the reference sample, or to a predetermined reference range, indicates that the cancer therapy is effective; whereas, an increase in the amount of cancer stem cells in the patient or patient sample relative to the reference sample, or to a predetermined reference range, indicates that the cancer therapy is ineffective. In different aspects of this embodiment, the reference sample is a sample obtained from the patient from an earlier time (e.g. prior to undergoing cancer therapy or prior to the last treatment) or the reference sample is a sample obtained from, or within, a second patient having the same type of cancer that is in remission, or the reference sample is a sample obtained from, or within, a healthy person with no detectable cancer.

The invention also includes vaccines that generate immune response to cancer stem cell antigens, and in particular to the gene products described in the Tables. The gene products in the Tables (either in whole, or using peptides derived from those proteins, as well as modified peptides of these protein) that are expressed by cancer stem cells can be used as cancer vaccines that target generate immunity to cancer stem cells.

Agents of the invention can be used to treat patient in combination with other therapies, including but not limited to chemotherapy, immunotherapy, cancer vaccines, radiation, differentiation agents.

In a specific embodiment, the invention provides agents for the treatment of bladder cancer stem cells. Examples of proteins that are expressed in bladder cancer stem cells, and agents that target those proteins are provided herein. In another embodiment, cancer stem cells from tumors other than bladder cancer are also therapeutically targeted by the agents of the invention. Examples of tumors that are also targeted by the agents of the invention are solid tumors and hematologic tumors. As defined herein, cancer stem cells from different tumor types share similar properties, and as such, share similar gene expression patterns. Therefore, cancer stem cells from different tumors can be targeted by the same agents. As such, the methods and agents described herein apply to multiple tumor types. The examples described herein apply to all tumor types.

### DEFINITIONS

As used herein, the terms “about” or “approximately”, unless otherwise indicated, refer to a value that is no more than 10% above or below the value being modified by the term.

As used herein, the term “administer continuously,” in the context of administration of a therapy to a subject, refers to the administration of a therapy to a subject at a frequency that is expected to maintain a specific plasma concentration of the therapy. For instance, in some embodiments of the therapies that are administered continuously, the administration to the subject is at a frequency that is expected to maintain less than a 50% change in the plasma concentration of the therapy, e.g., a 20-50% change, a 10-30% change, a 5-25% change, or a 1-20% change in plasma concentration of the therapy.

As used herein, the term “agent” refers to any molecule, compound, nucleic acid, nucleic acid based moiety, antibody, antibody-based molecule, protein, protein-based molecule and/or substance for use in the prevention, treatment, management and/or diagnosis of cancer.

As used herein, the term “amount,” as used in the context of the amount of a particular cell population or cells, refers to the frequency, quantity, percentage, relative amount, or number of the particular cell population or cells.

As used herein, the term “cancer cells” refer to cells that acquire a characteristic set of functional capabilities during their development, including the ability to evade apoptosis, self-sufficiency in growth signals, insensitivity to anti-growth signals, tissue invasion/metastasis, significant growth potential, and/or sustained angiogenesis. The term “cancer cell” is meant to encompass both pre-malignant and malignant cancer cells.

As used herein, the term “cancer stem cell(s)” refers to a cell that can be a progenitor of a highly proliferative cancer cell. A cancer stem cell has the ability to re-grow a tumor as demonstrated by its ability to form tumors in immunocompromised mice, and typically to form tumors upon subsequent serial transplantation in immunocompromised mice. Cancer stem cells are also typically slow-growing relative to the bulk of a tumor; that is, cancer stem cells are generally quiescent. In certain embodiments, but not all, the cancer stem cell may represent approximately 0.1 to 10% of a tumor. As used herein, the definition of cancer stem cell includes the terms highly tumorigenic cell, cancer stem-like cell, HTC, and CSC.

As used herein, the term “compound” refers to small molecules. Examples of such small molecules would include low molecular weight molecules. Other examples of compounds include molecules that are generated by organic synthesis, and low molecular weight molecules that are metabolites or anti-metabolites. In one embodiment, compounds can be administered directly to patients, or can be conjugated to antibodies or protein-based agents. In another embodiment, compounds can be administered in combination with other agents. In a specific embodiment, the agent administered in combination with a compound is an antibody or antibody-based therapeutic.

As used herein, the phrase “diagnostic agent” refers to any agent, molecule, compound, and/or substance that is used for the purpose of diagnosing cancer. Non-limiting examples of diagnostic agents include antibodies, antibody fragments, or other proteins, including those conjugated to a detectable agent. As used herein, the term “detectable agents” refer to any agent, molecule, compound and/or substance that is detectable by any methodology available to one of skill in the art. Non-limiting examples of detectable agents include dyes, gases, metals, or radioisotopes. As used herein, diagnostic agent and “imaging agent” are equivalent terms.

As used herein, the term “effective amount” refers to the amount of a therapy that is sufficient to result in therapeutic benefit to a patient with cancer, In one embodiment, the cancer patient has been diagnosed with bladder cancer. In one embodiment, the effective amount is administered to a patient that has been diagnosed with cancer. The effective amount can result in the prevention of the development, recurrence, or onset of cancer and one or more symptoms thereof, to enhance or improve the efficacy of another therapy, reduce the severity, the duration of cancer, ameliorate one or more symptoms of cancer, prevent the advancement of cancer, cause regression of cancer, and/or enhance or improve the therapeutic effect(s) of another therapy. “Effective amount” also refers to the amount of a therapy that is sufficient to result in the prevention of the development, recurrence, or onset of cancer and one or more symptoms thereof, to enhance or improve the prophylactic effect(s) of another therapy, reduce the severity, the duration of cancer, ameliorate one or more symptoms of cancer, prevent the advancement of cancer, cause regression of cancer, and/or enhance or improve the therapeutic effect(s) of another therapy. In an embodiment of the invention, the amount of a therapy is effective to achieve one, two, three, or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, (12) an increase in the number of patients in remission, (13) an increase in the length or duration of remission, (14) a decrease in the recurrence rate of cancer, (15) an increase in the time to recurrence of cancer, and (16) an amelioration of cancer-related symptoms and/or quality of life.

As used herein, the phrase “elderly human” refers to a human between 65 years old or older, preferably 70 years old or older.

As used herein, the phrase “human adult” refers to a human 18 years of age or older.

As used herein, the phrase “human child” refers to a human between 24 months of age and 18 years of age.

As used herein, the phrase “human infant” refers to a human less than 24 months of age, preferably less than 12 months of age, less than 6 months of age, less than 3 months of age, less than 2 months of age, or less than 1 month of age.

As used herein, the phrase “human patient” refers to any human, whether elderly, an adult, child or infant.

As used herein, the term “specifically binds to an antigen” and analogous terms refer to peptides, polypeptides, proteins, fusion proteins and antibodies or fragments thereof that specifically bind to an antigen or a fragment and do not specifically bind to other antigens. A peptide, polypeptide, protein, or antibody that specifically binds to an antigen may bind to other peptides, polypeptides, or proteins with lower affinity as determined by, e.g., immunoassays, BIAcore, or other assays known in the art. Antibodies or fragments that specifically bind to an antigen may be cross-reactive with related antigens. Preferably, antibodies or fragments that specifically bind to an antigen do not cross-react with other antigens. An antibody binds specifically to an antigen when it binds to the antigen with higher affinity than to any cross-reactive antigen as determined using experimental techniques, such as radioimmunoassays (RIAs) and enzyme-linked immunosorbent assays (ELISAs). See, e.g., Paul, ed., 1989, Fundamental Immunology, 2^{nd} ed., Raven Press, New York at pages 332-336 for a discussion regarding antibody specificity.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, the terms “manage,” “managing,” and “management” in the context of the administration of a therapy to a subject refer to the beneficial effects that a subject derives from a therapy or a combination of therapies, while not resulting in a cure of cancer. In certain embodiments, a subject is administered one or more therapies to “manage” cancer so as to prevent the progression or worsening of the condition. In one embodiment, the beneficial effect of the therapy is prophylactic, and in another embodiment, the beneficial effect of the therapy is therapeutic.

As used herein, the term “marker” in the context of a cell or tissue (e.g. a normal or cancer cell or tumor) means any antigen, molecule or other chemical or biological entity that is specifically found in or on a tissue that it is desired to identified or identified in or on a particular tissue affected by a disease or disorder. In specific embodiments, the marker is a cell surface antigen that is differentially or preferentially expressed by specific cell types. For example, a leukemia cancer stem cell differentially expresses CD123 relative to a normal hematopoietic stem cell.

As used herein, the term “marker phenotype” in the context of a tissue (e.g., a normal or cancer cell or a tumor cell) means any combination of antigens (e.g., receptors, ligands, and other cell surface markers), molecules, or other chemical or biological entities that are specifically found in or on a tissue that it is desired to identify a particular tissue affected by a disease or disorder. In specific embodiments, the marker phenotype is a cell surface phenotype. In accordance with this embodiment, the cell surface phenotype may be determined by detecting the expression of a combination of cell surface antigens. Non-limiting examples of cell surface phenotypes of cancer stem cells of certain tumor types include CD34⁺/CD38⁻, CD123+, CD44⁺/CD24⁻, CD133⁺, CD34⁺/CD10⁻/CD19⁻, CD138⁻/CD34⁻/CD19⁺, CD133⁺/RC2⁺, CD44⁺/alpha₂beta₁^{hi}/CD133⁺, CLL-1, SLAMs, and other cancer stem cell surface phenotypes mentioned herein, as well as those that are known in the art.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “predetermined reference range” refers to a reference range for the particular biological entity e.g., cancer stem cell, for a subject or a population of subjects. Each laboratory may establish its own reference range for each particular assay, or a standard reference range for each assay may be made available and used locally, regionally, nationally, or worldwide or may be patient-specific. In one specific embodiment, the term refers to a reference range for the amount of cancer stem cells in a patient (e.g., as determined by in vivo imaging) or a specimen from a patient. In another specific embodiment, the term refers to a reference range for the amount of cancer cells in a patient (e.g. as described by in vivo imaging) or a specimen from a patient.

As used herein, the terms “prevent,” “preventing” and “prevention” in the context of the administration of a therapy to a subject refer to the prevention or inhibition of the recurrence, onset, and/or development of a cancer or a symptom thereof in a subject resulting from the administration of a therapy (e.g., a prophylactic agent), or a combination of therapies (e.g., a combination of prophylactic agents). In some embodiments, such terms refer to one, two, three, or more results following the administration of one or more therapies: (1) a stabilization, reduction or elimination of the cancer stem cell population, (2) a stabilization, reduction or elimination in the cancer cell population, (3) an increase in response rate, (4) an increase in the length or duration of remission, (5) a decrease in the recurrence rate of cancer, (6) an increase in the time to recurrence of cancer, (7) an increase in the disease-free, relapse-free, progression-free, and/or overall survival of the patient, and (8) an amelioration of cancer-related symptoms and/or quality of life. In specific embodiments, such terms refer to a stabilization, reduction or elimination of the cancer stem cell population.

As used herein, the term “proliferation based therapy” refers to any agent, molecule, compound, substance, and/or method that differentially impairs, inhibits or kills rapidly proliferating cell populations (e.g., cancer cells) in comparison with cell populations that divide more slowly. Proliferation based therapies may include, but are not limited to those chemotherapeutic and radiation therapies that are typically used in oncology. A proliferation based agent may differentially impair, inhibit or kill rapidly proliferating cells by any mechanism known to one skilled in the art including, but not limited to, disrupting DNA function (including DNA replication), interfering with enzymes involved in DNA repair, intercalating DNA, interfering with RNA transcription or translation, interfering with enzymes involved with DNA replication, interfering with a topoisomerase, such as topoisomerase II, interfering with mitosis, and inhibiting enzymes necessary for the synthesis of proteins needed for cellular replication. Specific examples of proliferation based therapies include, but are not limited to, alkylating agents, nitrosoureas, antimetabolites, antibiotics, procarbazine, hydroxyurea, platinum-based agents, anthracyclines, topoisomerase II inhibitors, spindle poisons, and mitotic inhibitors.

As used herein, the phrase “prophylactic agent” refers to any agent, molecule, compound, and/or substance that is used for the purpose of preventing cancer. Examples of prophylactic agents include, but are not limited to, proteins, immunoglobulins (e.g., multi-specific Igs, single chain Igs, Ig fragments, polyclonal antibodies and their fragments, monoclonal antibodies and their fragments), antibody conjugates or antibody fragment conjugates, peptides (e.g., peptide receptors, selectins), binding proteins, chemospecific agents, chemotoxic agents (e.g., anti-cancer agents), proliferation based therapy, and small molecule drugs.

As used herein, the term “prophylactically effective regimen” refers to an effective regimen for dosing, timing, frequency and duration of the administration of one or more therapies for the prevention of cancer or a symptom thereof. In a specific embodiment, the regimen achieves one, two, three, or more of the following results: (1) a stabilization, reduction or elimination of the cancer stem cell population, (2) a stabilization, reduction or elimination in the cancer cell population, (3) an increase in response rate, (4) an increase in the length or duration of remission, (5) a decrease in the recurrence rate of cancer, (6) an increase in the time to recurrence of cancer, (7) an increase in the disease-free, relapse-free, progression-free, and/or overall survival of the patient, and (8) an amelioration of cancer-related symptoms and/or quality of life.

As used herein, the term “refractory” is most often determined by failure to reach a clinical endpoint, e.g., response, extended duration of response, extended disease free, survival, relapse free survival, progression free survival and overall survival. Another way to define being refractory to a therapy is that a patient has failed to achieve a response to a therapy such that the therapy is determined to not be therapeutically effective.

As used herein, the term “small reduction,” in the context of a particular cell population (e.g., circulating endothelial cells and/or circulating endothelial progenitors) refers to less than a 30% reduction in the cell population (e.g., the circulating endothelial cell population and/or the circulating endothelial progenitor population).

As used herein, the term “stabilizing” and analogous terms, when used in the context of a cancer stem cell population or cancer cell population, refer to the prevention of an increase in the cancer stem cell population or cancer cell population, respectively. In other words, the amount of cancer stem cells or the amount of cancer cells that a cancer is composed of is maintained, and does not increase, or increases by less than 10%, preferably less than 0.5%.

As used herein, the terms “subject” and “patient” are used interchangeably. As used herein, the term “subject” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the term “therapeutic agent” refers to any molecule, agent, nucleic acid, nucleic acid-based moiety, antibody, or antibody based agent and/or substance that is used for the purpose of treating and/or managing a disease or disorder. Examples of therapeutic agents include, but are not limited to, proteins, immunoglobulins (e.g., multi-specific Igs, single chain Igs, Ig fragments, polyclonal antibodies and their fragments, monoclonal antibodies and their fragments), peptides (e.g., peptide receptors, selectins), binding proteins, biologics, chemospecific agents, chemotoxic agents (e.g., anti-cancer agents), proliferation-based therapy, radiation, chemotherapy, anti-angiogenic agents, and small molecule drugs.

As used herein, the term “therapeutically effective regimen” refers to a regimen for dosing, timing, frequency, and duration of the administration of one or more therapies for the treatment and/or management of cancer or a symptom thereof. In a specific embodiment, the regimen achieves one, two, three, or more of the following results: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, and (12) a increase in the number of patients in remission.

As used herein, the terms “therapies” and “therapy” can refer to any method(s), composition(s), and/or agent(s) that can be used in the prevention, treatment and/or management of a cancer or one or more symptoms thereof. In certain embodiments, the terms “therapy” and “therapies” refer to chemotherapy, small molecule therapy, radioimmunotherapy, toxin therapy, prodrug-activating enzyme therapy, biologic therapy, antibody therapy, surgical therapy, hormone therapy, immunotherapy, anti-angiogenic therapy, targeted therapy, epigenetic therapy, demethylation therapy, histone deacetylase inhibitor therapy, differentiation therapy, radiation therapy, or a combination of the foregoing and/or other therapies useful in the prevention, management and/or treatment of a cancer or one or more symptoms thereof.

As used herein, the terms “treat,” “treatment,” and “treating” in the context of the administration of a therapy to a subject refer to the reduction or inhibition of the progression and/or duration of cancer, the reduction or amelioration of the severity of cancer, and/or the amelioration of one or more symptoms thereof resulting from the administration of one or more therapies. In specific embodiments, such terms refer to one, two or three or more results following the administration of one, two, three or more therapies: (1) a stabilization, reduction or elimination of the cancer stem cell population; (2) a stabilization, reduction or elimination in the cancer cell population; (3) a stabilization or reduction in the growth of a tumor or neoplasm; (4) an impairment in the formation of a tumor; (5) eradication, removal, or control of primary, regional and/or metastatic cancer; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the tumor is maintained and does not increase or increases by less than 10%, preferably less than 5%, preferably less than 4%, preferably less than 2%, and (12) an increase in the number of patients in remission. In certain embodiments, such terms refer to a stabilization or reduction in the cancer stem cell population. In some embodiments, such terms refer to a stabilization or reduction in the growth of cancer cells. In some embodiments, such terms refer to a stabilization or reduction in the cancer stem cell population and a reduction in the cancer cell population. In some embodiments, such terms refer to a stabilization or reduction in the growth and/or formation of a tumor. In some embodiments, such terms refer to the eradication, removal, or control of primary, regional, or metastatic cancer (e.g., the minimization or delay of the spread of cancer). In some embodiments, such terms refer to a reduction in mortality and/or an increase in survival rate of a patient population. In further embodiments, such terms refer to an increase in the response rate, the durability of response, or number of patients who respond or are in remission. In some embodiments, such terms refer to a decrease in hospitalization rate of a patient population and/or a decrease in hospitalization length for a patient population.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### DESCRIPTION OF THE DRAWINGS

FIG. 1: Immunohistochemical staining for cytokeratins (CKs) demonstrates aspects of urothelial differentiation in urothelial carcinomas. A. Prevalence and patterns of CK17 and 18 expression in primary urothelial carcinomas. B. Representative photomicrograph showing peripheral staining for the basal cell marker cytokeratin 17 that is most intense at the tumor-stroma interface and C. staining for the intermediate cell marker cytokeratin 18 that shows equivalent intensity at the peripheral and interior aspects of tumor nodules.

FIG. 2: Urothelial differentiation of human SW780 urothelial carcinoma xenografts. A. Basal-like urothelial cells at the tumor (T)—stroma (S) interface showing intense immunoperoxidase staining of CK17, similar to the CK17 staining pattern of benign urothelial basal cells (Inset). Staining for CK18 (B) and CK20 in interior of tumor nodules corresponding to their superficial expression in benign urothelium (insets). D. Double immunoperoxidase staining for the proliferation-associated protein Ki67 (brown) and the basal cell marker CK17 (blue/green) indicates approximately equal proliferation rates in basal-like and non-basal like cells.

FIG. 3: CK17-positive basal cancer cells and CK17-negative differentiated cancer cells express the surface markers 67LR and CEACAM6, respectively. A. Photomicrograph of SW780 human UC xenograft showing basal localization of CK17 (TOP) and 67LR (bottom) on adjacent sections. Insets show higher magnification view of tumor-stromal interface. B. Photomicrograph showing CK17 and CEACAM6 expression. Note mutually exclusive expression of the two proteins, with basal-like cells at tumor-stroma interface showing evidence of CK17 expression but not CEACAM6. CEACAM6-positive cells are in the internal aspect of tumor nodules.

FIG. 4: Basal-like cancer cells are enriched for tumor propagating ability in xenograft assays. A. CK17 and 67LR fail to co-localize in vitro. B. In contrast, the two markers strictly co-localize in single cells cultured briefly after dissociation from xenografts grown subcutaneously in athymic mice. C. Number of tumors identified at different starting doses of 67LR bright, 67LR dim, and control sorted cells from SW780 xenografts. Note that all of the tumor forming ability is associated with 67LR bright (basal) cells, which are ˜10-fold more potent in initiating tumor xenografts than the parental tumor (control sorted cells). D. Number of tumors identified at different starting doses of CD66c (CEACAM6) dim (basal) or bright (central) cells isolated from passage 2 of the XBL8 human UC xenograft.

FIG. 5: Gene expression in SW780 human UC xenograft cell subpopulations: 67LR bright cancer HTC genes drive aspects of human UC progression. A. Venn diagram of differentially expressed genes (Log Odds >3.0) indicates that 67LR bright cells are very distinct from 67LR dim and control (bulk)-sorted cells, whereas bulk and 67-LR dim cells have no significant differences in expression (even though 67LR bright cells are a component of the bulk population). B. Color values represent log 10 transformed p-values for association of gene expression in panel C. Positive values (in the white-red scale) are used for concordant upregulation, while Negative values (in the white-blue scale) are used for concordant down-regulation C. Concordant variation of gene expression between different stages of UC progression (obtained from publicly available data sets) (Cancer contrasts) and either 67LR bright basal HTCs (heatmap on the right) or 67LR dim non-tumorigenic cells (heatmap on the left). Orange/red boxes show concordant upregulation in more advanced clinical stages and in cancer cells. Blue boxes show concordant down-regulation in UC progression and in cancer cells. The gene expression program of 67LR bright HTCs proved to be concordantly enriched in many more contrasts than the gene expression program of 67LR nontumorigenic dim cells.

FIG. 6: Fluorescence-Activated-Cell-Sorting human SW780 UC xenograft cells by 67LR expression levels. (A). SW780 xenograft single cells (R1) were gated based on forward scatter (FSC) and side scatter (SSC). (B). Live cells (R2) were gated based on negative 7AAD staining. Mouse IgG and IgM (C), mouse IgG alone (D), or IgM alone (E) isotype controls show no staining. (F) Live SW780 xenograft tumor cells were sorted into two populations based on 67LR staining: 67LR-Bright cells (R4) and 67LR Dim tumor cells (R3). Mouse cells were identified and excluded via staining with antibodies against mouse major histocompatibility antigen (mMHC1). (G) and (H) show post-sort analysis demonstrated high purity of sorted R4 and R3 cells respectively.

FIG. 7: Fluorescence-Activated-Cell-Sorting of human XBL8 UC xenograft cells by CD66c (CEACAM6) expression levels. (A). XBL8 xenograft single cells (P1) were gated based on FSC and SSC. (B) Live cells (P2) were gated based on negative 7-AAD staining. Mouse IgG and rat IgG (C), mouse IgG alone (D), or rat IgG alone (E) isotype controls showed no staining. (F). Live XBL8 xenograft tumor cells were sorted into two populations based on CD66c and CD44 staining: CD66c-Dim/CD44-Bright (P4) and CD66c-Bright/CD44-ALL (Bright and Dim) (P3).

FIG. 8: Photomicrograph showing immunohistochemical staining of normal human urothelium with antibodies against CEACAM6. Note: Basal urothelial cells (arrow) lack detectable CEACAM6.

FIG. 9: Basal cell gene expression does not correspond to CD44 expression. Photomicrographs show immunohistochemical staining (brown) of tissue sections from low passage tissue derived PT2 and SW780 cell line derived xenografts. Note: The basal compartment at the tumor-stroma interface preferentially expresses the basal cell marker CK17 and preferentially downregulates the more differentiated markers CK18 and CEACAM6. CD44, in contrast is expressed at similar levels in both compartments in these two tumors and in XBL8 xenografts (see FIG. 7). Insets are higher magnification photomicrographs showing basal compartment (arrows).

FIG. 10: Multipotency of basal-like HTCs from SW780 xenograft cells. Photomicrographs show hematoxylin and eosin stained (H&E) or immoperoxidase stained sections of tumors inoculated from purified 67LR− Bright cells. These tumors recapitulated the spatially organized differentiation profile of parental SW780 xenografts as demonstrated by immunoperoxidase staining with antibodies against cytokeratins (CK) 17, 18, and 20.

FIG. 11: Multipotency of basal-like HTCs from XBL8 xenograft cells. Photomicrographs show immoperoxidase stained sections of the parental XBL8 tumor xenograft (left) or xenografts inoculated from purified CD66c (CEACAM6)-Dim XBL8 cells (right). Inoculating CD66c-Dim cells recapitulated the spatially organized differentiation profile of parental XBL8 xenografts as demonstrated by immunoperoxidase staining with antibodies against cytokeratin (CK) 17, CD44, and CD66c.

FIG. 12: Confirmation of microarray expression data by real time reverse transcriptase-polymerase chain reaction analysis. Data were obtained by analysis of total RNA isolated from flow-sorted 67LR bright and 67LR dim cells. The same RNA samples were used in both types of analysis. Note that for 17 of 17 transcripts, direction and scale of differential expression are similar.

FIG. 13: Enhanced expression of beta-catenin in basal compartment SW780 xenograft. Photomicrograph showing indirect immunofluourescent detection (green) of membrane and cytoplasmic beta-catenin in the periphery of tumor nodules at the tumor-stroma interface. Nuclei (blue) are counterstained with DAPI.

FIG. 14: The experimental design used to obtain gene expression profiles of urothelial cancer stem cells. The arrow direction represents the labeling scheme used (the arrow point indicates the use of one dye, while the arrow tail indicates the use of the other dye).

FIG. 15: Venn diagram showing the number of features (left) and of genes (right) found to be differentially expressed in the three possible contrasts involving BRIGHT, DIM and BULK samples. The total number of features or genes which were not differentially expressed is shown outside the circles on the bottom right of each box. Six genes measured by distinct features were differentially expressed in all comparisons performed.

FIG. 16: Cat-plot showing the number of overlapping genes between BRIGHT, DIM and BULK samples. For this purpose the gene lists for each group of samples were ordered by increasing (top panel) and decreasing (bottom panel) expression mean intensity. Only DIM and BULK show overlap at the top of the lists.

FIG. 17: List of antibodies used in immunodection studies

FIG. 18 depicts Table 1: Examples of cancer stem cells from various tumors that are resistant to chemotherapy, and the mechanism by which they are resistant.

FIG. 19 depicts Table 2: Gene expression was comprehensively profiled in 67LR bright HTCs and 67LR dim cells. The table lists selected functional pathways that were significantly enriched (adjusted P <0.05) among differentially expressed genes between 67LR bright and dim cells, along with selected genes in each pathway showing statistically significant (adjusted P <0.001) differential expression. Genes are listed according to official symbols in descending order of statistical significance. *Kremen2 was added to the Wnt pathway category, but this addition was not considered in statistical analysis.

FIG. 20 depicts Table 3: Bright vs. Dim.

FIG. 21 depicts Table 4: KEGG.

FIG. 22 depicts Table 5: Cancer Stem Cell integral membrane proteins for targeting with antibodies.

FIG. 23 depicts Table 6: Cancer integral membrane proteins for targeting with antibodies.

FIG. 24 depicts Table 7: Cancer stem cell plasma membrane proteins for targeting with antibodies.

FIG. 25 depicts Table 8: Cancer plasma membrane proteins for targeting with antibodies.

FIG. 26 depicts Table 9: Cancer stem cell surface proteins for targeting with antibodies.

FIG. 27 depicts Table 10: Cancer surface proteins for targeting with antibodies.

FIG. 28 depicts Table 11: Cancer stem cell anchor proteins for targeting with antibodies.

FIG. 29 depicts Table 12: Cancer anchor proteins for targeting with antibodies.

### DETAILED DESCRIPTION OF THE INVENTION

### Monitoring Cancer Stem Cells

Production of Therapeutic Antibodies

In a specific embodiment, a method of treating cancer comprising administering to a human patient diagnosed with cancer an antibody that binds to a cancer stem cell surface antigen in an amount sufficient to inhibit the proliferation of cancer cells in the patient.

In a specific embodiment, a method of treating cancer comprising administering to a human patient diagnosed with cancer an antibody fragment attached to a therapeutic moiety, wherein said antibody fragment binds to a cancer stem cell surface antigen, in an amount sufficient to inhibit the proliferation of cancer cells in the patient.

In a specific embodiment, a method of treating cancer comprising administering to a human patient diagnosed with cancer an antibody that binds to a cancer stem cell surface antigen in an amount sufficient to inhibit the proliferation of cancer cells in the patient.

In a specific embodiment, a method of treating cancer comprising administering to a human patient diagnosed with cancer an antibody fragment attached to a therapeutic moiety, wherein said antibody fragment binds to a cancer stem cell surface antigen, in an amount sufficient to inhibit the proliferation of cancer cells in the patient.

In a specific embodiment, a method of treating cancer comprising administering to a human patient diagnosed with a solid tumor an antibody that binds to a cancer stem cell surface antigen in an amount sufficient to inhibit the proliferation of cancer cells in the patient.

In a specific embodiment, a method of treating cancer comprising administering to a human patient diagnosed with a solid tumor an antibody fragment attached to a therapeutic moiety, wherein said antibody fragment binds to a cancer stem cell surface antigen, in an amount sufficient to inhibit the proliferation of cancer cells in the patient.

In certain embodiments, antibodies or fragments thereof that bind to a marker on cancer stem cells are substantially non-immunogenic in the treated subject. Methods for obtaining non-immunogenic antibodies include, but are not limited to, chimerizing the antibody, humanizing the antibody, and isolating antibodies from the same species as the subject receiving the therapy. Antibodies or fragments thereof that bind to markers in cancer stem cells can be produced using techniques known in the art. See, for example, paragraphs 539-573 of U.S. Publication No. 2005/0002934 A1, which is incorporated by reference in its entirety.

In some embodiments, the therapy comprises the use of X-rays, gamma rays and other sources of radiation to destroy cancer stem cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer stem cells, cancer cells and/or a tumor mass.

In some embodiments, the therapy used is a proliferation based therapy. Non-limiting examples of such therapies include a chemotherapy and radiation therapy as described herein.

Antibodies for use in the methods of the invention include, but are not limited to, synthetic antibodies, monoclonal antibodies (mAbs), recombinantly produced antibodies, multispecific antibodies (including bi-specific antibodies), human antibodies, humanized antibodies, chimeric antibodies, intrabodies, diabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In particular, antibodies to be used in the methods of the invention include immunoglobulin molecules and immunologically active portions of immunoglobulin molecules, i.e., molecules that contain an antigen binding site that binds to a CD22 or CD40 antigen, or bispecifically to the CD1d and CD5 antigens. The immunoglobulin molecules can be of any type (e.g., IgG, IgE, IgM, IgD, IgA and IgY), class (e.g., IgG1, IgG2, IgG3, IgG4, IgA1 and IgA2) or subclass of immunoglobulin molecule.

Variants and derivatives of antibodies include antibody fragments that retain the ability to specifically bind to an epitope. In certain embodiments, fragments include Fab fragments; Fab′; F(ab′)2; a bispecific Fab; a single chain Fab chain comprising a variable region, also known as, a sFv; a disulfide-linked Fv, or dsFv; a camelized VH; a bispecific sFv; a diabody; and a triabody. Derivatives of antibodies also include one or more CDR sequences of an antibody combining site. In certain embodiments, the antibody to be used with the invention comprises a single-chain Fv (“scFv”).

The antibodies used in the methods of the invention may be from any animal origin including birds and mammals (e.g., human, murine, donkey, sheep, rabbit, goat, guinea pig, camel, horse, or chicken).

In certain embodiments, the antibodies of the invention are monoclonal antibodies (mAbs). Monoclonal antibodies can be prepared using a wide variety of techniques known in the art including the use of hybridoma, recombinant, and phage display technologies, or a combination thereof. For example, mAbs can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981) (each of which is herein incorporated by reference in their entireties).

Antibodies can also be generated using various phage display methods. Examples of phage display methods that can be used to make the antibodies include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al, 1997, Gene 187:9-18; Burton et al., 1994, Advances in Immunology 57:191-280; PCT Application No. PCT/GB91/O1 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated by reference herein in its entirety.

In certain embodiments, the antibodies of the invention are chimeric antibodies or single chain antibodies. Techniques developed for the production of “chimeric antibodies” (Morrison et al., 1984, Proc Natl Acad Sci 81:851; Neuberger et al., 1984 Nature 312:604; Takeda et al., 1985, Nature 314:452, each incorporated by reference herein in its entirety) and single chain antibodies (U.S. Pat. No. 4,946,778; Bird, 1988, Science 242:423; Huston et al, 1988, Proc Natl Acad Sci USA 85:5879; and Ward et al, 1989, Nature 334:544, each incorporated by reference herein in its entirety) are well known in the art.

In a certain embodiment, antibodies used in the methods of the invention are humanized antibodies. Humanized antibodies can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089, each of which is herein incorporated by reference in its entirety), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al, 1994, PNAS 91:969-973, each of which is incorporated by reference herein in its entirety), chain shuffling (U.S. Pat. No. 5,565,332, herein incorporated by reference in its entirety), and techniques disclosed in, e.g., U.S. Pat. No. 6,407,213, U.S. Pat. No. 5,766,886, WO 9317105, Tan et al., 2002, J. Immunol. 169:1119 25, Caldas et al., 2000 Protein Eng. 13(5):353-60, Morea et al., 2000, Methods 20(3):267 79, Baca et al., 1997, J. Biol. Chem. 272(16):10678-84, Roguska et al., 1996, Protein Eng. 9(10):895 904, Couto et al., 1995 Cancer Res. 55 (23 Supp):5973s-5977s, Couto et al., 1995, Cancer Res. 55(8):1717-22, Sandhu JS, 1994, Gene 150(2):409-10, and Pedersen et al., 1994, J. Mol. Biol. 235(3):959-73 U.S. Patent Pub. No. US 2005/0042664 A1 (Feb. 24, 2005), each of which is incorporated by reference herein in its entirety. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Reichmann et al., 1988, Nature 332:323, each of which is incorporated by reference herein in its entirety).

Single domain antibodies can be produced by methods well-known in the art. (See, e.g., Riechmann et al., 1999, J. Immunol. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety).

Further, antibodies that bind to a desired antigen can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438, herein incorporated by reference in their entireties).

Bispecific antibodies can be prepared using techniques that are known in the art. (See, e.g., U.S. Pat. Nos. 5,534,254, 5,837,242, 6,492,123; U.S. patent application publication Nos. 20040058400 and 20030162709, which are all herein incorporated by reference in their entireties.

The present invention contemplates the use of antibodies recombinantly fused or chemically conjugated (including both covalently and non-covalently conjugations) to a polypeptide. Fused or conjugated antibodies of the present invention may be used for ease in purification. For example, the antibodies or fragments thereof for use in present invention can be fused to marker sequences, such as a peptide to facilitate purification. See e.g., PCT publication WO 93/21232; EP 439,095; Naramura et al., 1994, Immunol Lett 39:91; U.S. Pat. No. 5,474,981; Gillies et al., 1992, Proc Natl Acad Sci USA 89:1428; Fell et al., 1991, J Immunol 146:2446, which are herein incorporated by reference in their entireties.

In certain aspects, the antibodies used in the present invention can be produced intracellularly, in the periplasmic space, or directly secreted into the medium. If the antibodies are produced intracellularly, as a first step, the particulate debris, either host cells or lysed fragments, may be removed, for example, by centrifugation or ultrafiltration.

Exemplary methods for the use of host cells and vectors in the production of antibody can be found in U.S. Pat. Nos. 4,816,567 and 6,331,415 to Cabilly et al., each of which is incorporated by reference herein in its entirety.

In one embodiment, comprising administering an antibody that binds to a cancer stem cell surface antigen (e.g. described in CSC SA table).

Antibodies that target, activate, inhibit or kill the regulatory B cell CD1d^{hi}CD5+ subset and which can be used in the therapeutic regimens described herein can be made using techniques well known in the art. The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Sambrook et al, 2001, Molecular Cloning: A Laboratory Manual, Cold Spring Harbor Laboratory Press, Cold Spring Harbor, N.Y.; Ausubel et al, Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al (eds.) (1999) Genome Analysis: A Laboratory Manual, Cold Spring Harbor Laboratory Press, each of which is incorporated by reference herein in its entirety.

Currently available therapies and their dosages, routes of administration and recommended usage are known in the art and have been described in such literature as the Physician's Desk Reference (60^{th} ed., 2006). Routes of administration include parenterally, intravenously, subcutaneously, intracranially, intrahepatically, intranodally, intraureterally, subureterally, subcutaneously, and intraperitoneally. Agents of the invention can also be administered into the bladder via a catheter, or injected directly into the bladder.

In a specific embodiment, cycling therapy involves the administration of a first cancer therapeutic for a period of time, followed by the administration of a second cancer therapeutic for a period of time, optionally, followed by the administration of a third cancer therapeutic for a period of time and so forth, and repeating this sequential administration, i.e., the cycle in order to reduce the development of resistance to one of the cancer therapeutics, to avoid or reduce the side effects of one of the cancer therapeutics, and/or to improve the efficacy of the cancer therapeutics.

When two therapeutically effective regimens are administered to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). When two prophylactically effective regimens are administered to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the cancer therapeutics may be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the components of the combination cancer therapeutics are not administered in the same pharmaceutical composition, it is understood that they can be administered in any order to a subject in need thereof. For example, a first therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof. In another embodiment, a first prophylactically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof. In various embodiments, the cancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the cancer therapeutics are administered within the same office visit. In another embodiment, the combination cancer therapeutics are administered at 1 minute to 24 hours apart.

In a specific embodiment, the combination therapies have the same mechanism of action. In another specific embodiment, the combination therapies each have a different mechanism of action.

Antibody Conjugates

The present invention provides antibody conjugates that bind to a surface antigen described herein. In some embodiments, the antibody conjugates of the present invention comprise an antibody that binds to a surface antigen described herein expressed on the surface of cells conjugated to a cytotoxic agent or other moiety (e.g., an anticellular moiety). In some embodiments, the antibody conjugates of the present invention comprise an antibody that binds to the extracellular domain of a surface antigen described herein conjugated to a cytotoxic agent or other moiety (e.g., an anticellular moiety). In a specific embodiment, the antibody conjugate of the invention is an immunotoxin. In one embodiment, the antibody is conjugated to a cytotoxic agent or otherwise anticellular agent, either directly or through a chemical linker. In another embodiment, the antibody is linked to the cytotoxic agent or otherwise anticellular moiety through a chemical (covalent) bond, such as a peptide bond (with or without a peptide linker), disulfide bond, or sterically hindered disulfide bond. The antibody can be linked at its amino terminus or its carboxyl terminus to the cytotoxic agent or otherwise anticellular moiety. Alternatively, the antibody can replace a domain of the cytotoxic agent or otherwise anticellular moiety that is not required for cytotoxicity so long as antibody retains its specificity for a surface antigen described herein.

In certain embodiments, an antibody conjugate comprises an antibody to a surface antigen described herein linked via a peptide linker (also referred to as a spacer) to a cytotoxic agent or an otherwise anticellular moiety. The linker for the conjugate may be 5, 8, 10, 12, 15, 25, 50, or 75 amino acids in length, but the length may otherwise vary to provide optimal binding of the conjugate to the surface antigen. In a specific embodiment, the peptide linker is 6 or 7 amino acids long. In another embodiment, the amino terminus of an antibody to a surface antigen descried herein is attached to a cytotoxic agent or an otherwise anticellular moiety through the peptide linker Ser-(Gly)₄-Ser. In another embodiment, the carboxyl terminus of an antibody to a surface target described herein is linked to a cytotoxic agent or an otherwise anticellular moiety through a Lys-Ala-Ser-Gly-Gly-Pro-Glu linker. The constituent amino acids of a spacer may be selected to influence some property of the conjugate, such as its folding, net charge, or hydrophobicity. Linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

An antibody conjugate of the invention may be composed of one or two polypeptide chains, together comprising the antibody moiety and the cytotoxic agent or anticellular moiety. In one embodiment, the cytotoxic agent is attached to the variable heavy (V_{H}) region of an Fv antibody fragment against a surface antigen described herein, where the V_{H} region is bound by an amino acid linker to the variable light (V_{L}) chain region. In another embodiment, the cytotoxic agent is attached to the V_{H} region of an Fv antibody against a surface antigen described herein where the V_{H} region is bound to a V_{L} chain region through at least one disulfide linkage (e.g., formed between respective cysteines in each chain). Disulfide linked Fv chains may have a reduced tendency to aggregate, show a generally longer serum half-life, and are said to be “stabilized.” Thus, a disulfide-stabilized antibody-cytotoxin conjugate comprises at least two polypeptides linked by at least one disulfide linkage. The two polypeptides can be separated by a termination codon and downstream initiation codon and ribosome binding site, so that the chains are encoded as separate open reading frames, or they can be joined by a peptide linker. In another embodiment, the cytotoxic agent is attached to the V_{H} region of an Fv antibody against a surface antigen described herein, where the V_{H} region is bound through a peptide linker and at least one disulfide bond to a V_{L} chain region. In a specific embodiment, the cytotoxic agent is attached to V_{L} region of an Fv antibody against a surface antigen described herein, where the V_{L} region is bound by a linker to the V_{H} chain region. In another embodiment, the cytotoxic agent is attached to the V_{L} region of an Fv antibody against a surface antigen described herein, where the V_{L} region is bound through at least one disulfide bond to a V_{H} chain region. In another embodiment, the cytotoxic agent is attached to the V_{L} region of an Fv antibody against a surface antigen described herein, where the V_{L} region is bound through a peptide linker and at least one disulfide bond to a V_{H} chain region. In yet another embodiment of the invention, the V_{L} and V_{H} sequences will be followed respectively by part or all of the light and heavy chain constant regions, e.g., the whole kappa light chain constant region and the C_{H1} domain of the heavy chain constant region, with or without the heavy chain hinge domain. Thus, the antibody segments and genes encoding the cytotoxic agent may occur in any order on a single plasmid, or may be expressed separately from separate plasmids. For example, in another embodiment of the invention, the V_{L} gene and any light chain constant region will be on one plasmid, while the V_{H} gene, any heavy chain constant region, and the gene for a proteinaceous cytotoxin will be on a second plasmid. In either case, the V_{L} and/or V_{H} genes may be preceded by a signal sequence that directs the secretion of the recombinant fusion protein from the cell. See, e.g., U.S. Pat. Nos. 6,147,203, 6,074,644 and 6,051,405 which are referenced herein in their entirety.

In another embodiment, the polypeptide chains expressed by the plasmid may be sequestered in inclusion bodies that are retained within the cell. The polypeptides may be expressed in a variety of expression systems that are routinely available to one skilled in the art, including bacterial expression systems such as E. coli and yeast expression systems, such as Pichia. See, e.g., Vrieto et al., 2004, Protein Expression and Purification 33: 1123-133, which is incorporated herein by reference in its entirety, for methods of expressing polypeptides sequestered in inclusion bodies.

In another embodiment, this invention provides for single chain antibody conjugates, in which the antibody comprises the V_{L} or V_{H} regions alone, rather than as components of Fv fragments. The amino terminus or carboxyl terminus of the variable chain is then conjugated to a selected cytotoxic agent, such conjugation may be through a peptide linker. See, e.g., U.S. Pat. No. 6,074,644, which is referenced herein in its entirety.

Antibodies, or the encoded antibodies, cytotoxic agents, or antibody conjugates may be altered by being subjected to random mutagenesis by error-prone PCR, random nucleotide insertion or other methods prior to recombination. A polynucleotide encoding an antibody to a surface antigen described herein may be recombined with one or more components, motifs, sections, parts, domains, fragments, etc. of one or more cytotoxin molecules.

The invention provides nucleic acids comprising nucleotide sequences encoding an antibody conjugate of the invention. The invention also provides nucleic acids comprising a nucleotide sequence that encode the antibody moiety and/or the proteinaceous cytotoxic agent of an antibody conjugate of the invention. Thus, for example, in one embodiment, the invention provides for a nucleic acid comprising a nucleotide sequence encoding an antibody conjugate comprising a V_{H} region of an Fv fragment attached to a proteinaceous cytotoxic agent (e.g., Pseudomonas exotoxin). The nucleotides that encode the V_{H} region are linked to the nucleotides that encode the V_{L} region through nucleotides that encode a peptide linker. Alternatively, or in combination, the encoded V_{H} region contains cysteine residues that form disulfide linkages with a V_{L} region of an Fv fragment. In another embodiment, the invention provides for nucleic acids comprising nucleotide sequence encoding an antibody conjugate of the invention in which the V_{L} chain is substituted for the V_{H} chain and vice versa. In another embodiment, the invention provides for nucleic acids comprising a nucleotide sequence encoding an antibody conjugate, in which the antibody comprises the V_{L} or V_{H} region alone. See, e.g., U.S. Pat. No. 6,074,644, which is referenced herein in its entirety.

In some embodiments, an antibody conjugate of the invention binds to an epitope on a surface antigen described herein that results in selective and potent cell killing, as assessed by a cytotoxicity assay. In certain embodiments, binding of an antibody conjugate to its epitope on a surface antigen described herein is followed by internalization of the antibody conjugate-surface antigen complex. In a particular embodiment, the cytotoxicity of a particular antibody-cytotoxin conjugate is measured by a cell viability assay, using cell lines that express a surface antigen described herein. In a particular embodiment, cell viability is tested using an assay in which cells are seeded into 96-well plates at a concentration of 2×10⁴ of cells/well. Serial dilutions of antibody conjugates in 0.2% human serum albumin (HSA) are added to the cells, resulting in final concentrations ranging from 0.1 to 1000 ng/ml in 150 μl. After incubation for 48 hours, 10 μl of WST-8 (Dojindo Molecular Technologies) is added to each well, and the incubation is carried out for 4 hours at 37° C. The absorbance of the sample at 450 nm is measured with a reference wavelength of 650 nm. Cytotoxicity is defined by IC₅₀, 50% inhibition of cell viability, which is midway between the level of viability in the absence of antibody conjugate and that in the presence of 10 μg/ml cycloheximide.

The present invention also provides for panels of antibody conjugates that bind to a surface antigen described herein. In specific embodiments, the invention provides for panels of antibody conjugates having different affinities for a surface antigen described herein, different specificities for a surface antigen described herein, and/or different dissociation rates. The invention provides panels of at least 10, preferably at least 25, at least 50, at least 75, at least 100, at least 125, at least 150, at least 175, at least 200, at least 250, at least 300, at least 350, at least 400, at least 450, at least 500, at least 550, at least 600, at least 650, at least 700, at least 750, at least 800, at least 850, at least 900, at least 950, or at least 1000 antibodies. Panels of antibody conjugates can be used, for example, in 96 well plates for assays such as ELISAs and cytotoxicity assays.

Set forth herein is a more detailed description of antibodies to surface antigens described herein encompassed within the various aspects of the invention. Such antibodies can be conjugated to cytotoxic agents or other moieties (e.g., an anticellular moiety) and used as potent and specific immunotoxins. Alternatively, the unconjugated antibodies (i.e., naked antibodies) can be used as therapeutic agents. Moreover, the unconjugated antibodies (i.e., naked antibodies) can be used as prophylactic agents.

The present invention provides methods for assessing specificity, affinity, and cytotoxicity of antibodies and antibody conjugates, such that the antibodies and antibody conjugates of the invention may be useful to specifically target and impair cells expressing a surface antigen described herein. The present invention provides antibodies that may differentially or preferentially bind to one or more epitopes on a surface antigen described herein. The present invention provides methods for comparing various antibodies to surface antigens described herein with respect to their differential epitope binding. The present invention provides methods for assessing the affinity of various antibodies to surface antigens described herein for their specific antigen on cells.

Antibodies of the invention include, but are not limited to, monoclonal antibodies, monospecific antibodies, polyclonal antibodies, multispecific antibodies, diabolizes, triabodies, tetrabodies, human antibodies, humanized antibodies, camelized antibodies, chimeric antibodies, single chain antibodies, single domain antibodies, Fab fragments, F(ab′) fragments, F(ab′)₂ fragments, Fv fragments (i.e., the smallest functional module of an antibody), single chain Fvs (scFv), disulfide-stabilized Fvs (dsFv), Fd, V_{H}, V_{L}, Vₐₗₚₕₐ, V_{beta}, and anti-idiotypic (anti-Id) antibodies (including, e.g., anti-Id antibodies to antibodies of the invention), intrabodies, and epitope-binding fragments of any of the above. In some embodiments, the antibodies are monoclonal antibodies. In other embodiments, the antibodies are Fv fragments, including V_{H}, and V_{L} regions.

In particular, antibodies of the present invention include immunoglobulin molecules and immunologically active portions of immunoglobulin molecules, i.e., molecules that contain an antigen binding site that binds to a surface antigen described herein. The immunoglobulin molecules of the invention can be of any type (e.g., IgG, IgE, IgM, IgD, IgA, and IgY), class (e.g., IgG₁, IgG₂, IgG₃, IgG₄, IgA₁, and IgA₂), or subclass of immunoglobulin molecule. In a specific embodiment, an antibody of the invention is an IgG antibody. In another specific embodiment, an antibody of the invention is not an IgA antibody.

The antibodies of the invention may be from any animal origin including birds and mammals (e.g., human, mouse, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies of the present invention may be monospecific, bispecific, trispecific, or of greater multispecificity. Multispecific antibodies may be specific for different epitopes of a polypeptide of a surface antigen described herein or may be specific for both a polypeptide of a surface antigen described herein, as well as for a heterologous epitope, such as a heterologous polypeptide or solid support material, as long as such epitopes are not found in human tissues. See, e.g., PCT publications WO 93/17715, WO 92/08802, WO 91/00360, and WO 92/05793; Tutt, et al., J. Immunol. 147:60-69 (1991); U.S. Pat. Nos. 4,474,893, 4,714,681, 4,925,648, 5,573,920, and 5,601,819; and Kostelny et al., J. Immunol. 148:1547-1553 (1992).

In one embodiment, antibodies of the invention bind to cells that express a surface antigen described herein (preferably, human surface antigen). In a specific embodiment, antibodies of the invention bind to a fragment of a surface antigen described herein comprising 8 to 285, 8 to 275, 8 to 250, 8 to 200, 8 to 175, 8 to 150, 8 to 100, 8 to 50, 8 to 25, 15 to 50, 15 to 75, 15 to 100 or 15 to 150 amino acids of the extracellular domain of a mature surface antigen described herein (preferably, the mature human form of a surface antigen described herein). In another embodiment, the antibodies of the invention bind to the extracellular domain of a human surface antigen described herein or a fragment thereof.

An antibody of the invention may be composed of one or two polypeptide chains. In one embodiment, an Fv antibody that binds to a surface antigen described herein is composed of a single polypeptide chain, where the V_{H} region is bound by an amino acid linker to the V_{L} chain region. In a preferred embodiment, such scFvs are stable at 37° C. for about 2 minutes, 5 minutes, 10 minutes, 15 minutes, 20 minutes, 25 minutes, 30 minutes, 1 hour, 1.5 hours, 2 hours, 3 hours, 4 hours, 5 hours, 6 hours, 7 hours, 12 hours, 14 hours, 24 hours, 48 hours, 1 week, 2 weeks, 1 month, 3 months, 6 months, 1 year or longer as assessed by a technique known to one of skill in the art or described herein. In another embodiment, the V_{H} region of an Fv antibody that binds to a surface target described herein is bound to a V_{L} chain region through at least one disulfide linkage (e.g., formed between respective cysteines in each chain). In certain embodiments, the disulfide linked Fv chains have a reduced tendency to aggregate as measured by, e.g., HPLC and have a longer serum half-life. Thus, in a specific embodiment, a disulfide-stabilized Fv (dsFv) antibody comprises at least two polypeptides linked by at least one disulfide linkage. The two polypeptides can be separated by a termination codon and downstream initiation codon and ribosome binding site, so that the chains are encoded as separate open reading frames, or they can be additionally joined by a peptide linker. In order to provide disulfide covalent bonds between the V_{H} and V_{L} chains of dsFv fragments, cysteine residues are necessary. Cysteine residues can be introduced in the proper position of each V_{H} and V_{L}, determined by alignment to reference sequences, by standard molecular biology techniques (e.g., site directed mutagenesis). See Pastan et al., U.S. Pat. No. 6,147,203, which is incorporate by reference herein in its entirety, especially columns 5-7.

In another embodiment, the V_{H} and V_{L} sequences will be followed respectively by part or all of the light and heavy chain constant regions, e.g., the whole kappa light chain constant region and the C_{H1} domain of the heavy chain constant region, with or without the heavy chain hinge domain. Thus, the genes encoding the antibody segments may occur in any order on a single plasmid, or may be expressed separately from separate plasmids. For example, in another embodiment of the invention, the V_{L} gene and any light chain constant region will be on one plasmid, while the V_{H} gene and any heavy chain constant region will be on a second plasmid. In either case, the V_{L} and/or V_{H} genes may be preceded by a signal sequence that directs the secretion of the recombinant fusion protein from the cell. See, e.g., U.S. Pat. Nos. 6,147,203, 6,074,644, 6,051,405 which are incorporated herein by reference in their entirety.

In another embodiment, the invention provides for single chain antibodies, in which the antibody comprises the V_{L} or V_{H} regions alone, rather than as components of Fv fragments. See, e.g., U.S. Pat. No. 6,074,644, which is incorporated herein by reference in its entirety.

An antibody can also be conjugated to a second antibody to form an antibody heteroconjugate as described by Segal in U.S. Pat. No. 4,676,980, which is incorporated herein by reference in its entirety. In a specific embodiment, two or more antibodies are cross-linked to each to produce a bispecific or multispecific antibody.

In one embodiment, an antibody of the invention is not only found to be specific for a surface antigen described herein but also binds with very high affinity, based on binding assays using a cell lines that expresses a surface antigen described herein.

The present invention is based, in part, upon the discovery that even though a particular antibody to a surface antigen described herein might bind to a surface antigen described herein specifically and with high affinity, that does not guarantee that it will constitute an effective immunotoxin when linked to a cytotoxic agent. In one embodiment, antibodies with high affinity and specificity are chosen for conjugation to a cytotoxic agent, and they are then assessed for their ability to selectively and potently kill cells that express a surface antigen described herein. In a specific embodiment, the antibody to a surface antigen described herein binds an epitope that overlaps that of an antibody known to cause specific cytotoxicity to cells expressing an antigen described herein.

The present invention provides antibodies that exhibit a high association rate (kₒₙ) value in an assay known to one of skill in the art or described herein, e.g., a plasmon resonance assay. See, e.g., U.S. Application Publication No. 20020098189 (which is incorporated herein by reference in its entirety) for methods for producing and identifying antibodies with a high k_{on value. In a specific embodiment, an antibody of the present invention has an association rate constant or k}ₒₙ rate (antibody (Ab)+antigen (Ag)^{kon}.−> Ab-Ag) of at least 2×10⁵ M⁻¹s⁻¹, at least 5×10⁵ M⁻¹s⁻¹, at least 10⁶M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

The present invention that have a low dissociation rate (k_{off}) in an assay known to one of skill in the art or described herein, e.g., a plasmon resonance assay. In a specific embodiment, an antibody of the invention has a k_{off} rate (antibody (Ab)+antigen (Ag)^{k off}−> Ab-Ag) of less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

The present invention provides antibodies that bind to a surface antigen described herein with high affinity. In a specific embodiment, an antibody of the invention has an affinity constant or K^{a} (kₒₙk_{off}) of at least 5×10⁶ M⁻¹, at least 10⁷ M⁻¹, at least 5×10⁷ M⁻¹, at least 10⁸ M⁻¹, at least 5×10⁸ M−1, at least 10⁹ M⁻¹, at least 5×10⁹ M⁻¹, at least 10¹⁰ M⁻¹, at least 5×10¹⁰° M⁻¹, at least 10¹¹ M⁻¹, at least 5×10¹¹M⁻¹, at least 10¹²M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In another embodiment, the antibodies of the invention has a Kₐ of between 5×10⁶ M⁻¹ to 5×10¹⁵ M⁻¹, 1×10⁷ M⁻¹ to 5×10¹²M⁻¹, or 5×10⁷ M⁻¹ to 5×10¹⁰ M⁻¹.

In another embodiment, an antibody has a dissociation constant or K_{d} (k_{off}kₒₙ) of less than 5×10⁻³ M, less than 10⁻⁴ M, less than 5×10⁻⁴ M, less than 10⁻⁵ M, less than 5×10⁻⁵ M, less than 10⁻⁶M, less than 5×10⁻⁶M, less than 10⁻⁷ M, less than 5×10⁻⁷ M, less than 10⁻⁸M, less than 5×10⁻⁸M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹²M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

In another embodiment, the antibodies of the invention have a dissociation constant (K_{d}) of less than 4000 pM, less than 3700 pM, less than 3500 pM, less than 3000 pM, less than 2750 pM, less than 2500 pM, less than 2000 pM, less than 1500 pM, less than 1000 pM, less than 750 pM, less than 500 pM, less than 250 pM, less than 200 pM, less than 150 pM, less than 100 pM, or less than 75 pM as assessed using an assay described herein or known to one of skill in the art (e.g., a BIAcore assay).

In a specific embodiment, the antibodies used in accordance with the methods of the invention bind to a surface antigen described herein and have a dissociation constant (K_{D}) of between 25 to 4000 pM, 25 to 3750 pM, 25 to 3500 pM, or 25 to 3000 pM as assessed using an assay described herein or known to one of skill in the art (e.g., a BIAcore assay).

The present invention provides antibodies that have a half-maximal inhibitory concentration (IC₅₀) of less than 50 ng/ml, less than 45 ng/ml, less than 40 nM, less than 35 nM, or less than 30 nM as assessed by an assay known to one of skill in the art or described herein. In a specific embodiment, the antibodies of the invention have an IC₅₀ of between 25 ng/ml to 75 ng/ml, 25 ng/ml to 50 ng/ml, or 25 ng/ml to 40 ng/ml.

In one embodiment, the invention provides for a nucleic acid comprising a nucleotide sequence encoding an antibody comprising a V_{H} region of an Fv fragment linked to the nucleotides that encode the V_{L} region through nucleotides that encode a peptide linker. Alternatively, or in combination, the encoded V_{H} region contains cysteine residues that form disulfide linkages with a cysteine-containing V_{L} region of an Fv fragment. In another embodiment, this invention provides for nucleic acids comprising nucleotide sequences encoding any of the antibodies described herein, in which the antibody comprises the V_{L} or V_{H} region alone. See, e.g., U.S. Pat. No. 6,074,644, which is referenced herein in its entirety.

Those skilled in the art will realize that additional modifications, deletions, insertions and the like may be made to the antibody to the surface antigen described herein. Especially, deletions or other changes may be made to the antibody in order to increase stability, affinity, specificity, or, when combined with the cytotoxic agent, cytotoxicity or other impairment to cells expressing a surface antigen described herein and/or to decrease non-specific cytotoxicity or impairment toward cells that lack a surface antigen described herein. Typical modifications include, but are not limited to, introduction of an upstream methionine for transcription initiation, mutation of residues to cysteine in V_{H} or V_{L} regions for the creation of disulfide linkages, etc. All such constructions may be made by methods of genetic engineering well known to those skilled in the art. Fragments, analogs, and derivatives of antibodies to surface targets described herein can be useful in the present invention provided that when fused to the cytotoxic agent portion of the conjugate, such fragments, analogs, and derivatives maintain the ability to bind the native surface antigen described herein expressed on the surface of a cell. Preferably, the binding kinetics of the fragments, analogs, or derivatives remain the same or vary by no more than 25% (preferably, no more than 15%, 10% or 5%) as determined by an assays described herein.

To improve or alter the characteristics of antibodies to a surface target described herein, protein engineering may be employed. Recombinant DNA technology known to those skilled in the art can be used to create novel mutant proteins or “muteins” including single or multiple amino acid substitutions, deletions, additions, or fusion proteins. Such modified polypeptides can show, e.g., enhanced activity or increased stability. In addition, they may be purified in higher yields and show better solubility than the corresponding natural polypeptide, at least under certain purification and storage conditions. For instance, for many proteins, it is known in the art that one or more amino acids may be deleted from the amino terminus or carboxyl terminus without substantial loss of biological function.

Antibodies may be altered by random mutagenesis by error-prone PCR, random nucleotide insertion or other methods prior to recombination.

The present invention also provides antibodies that bind to a surface antigen described herein, the antibodies comprising derivatives of the V_{H} domains, V_{H} CDRs, V_{L} domains, and V_{L} CDRs described herein. Standard techniques known to those of skill in the art can be used to introduce mutations in the nucleotide sequence encoding a molecule of the invention, including, for example, site directed mutagenesis and PCR mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

The antibodies of the invention include derivatives that are modified, i.e., by the covalent attachment of any type of molecule to the antibody such that covalent attachment does not adversely affect binding to the antigen or, in the case of antibody conjugates of the invention, adversely affect its ability to bind to a surface antigen described herein and adversely affect its ability to specifically kill or impair cells that express a surface antigen described herein. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The present invention also provides antibodies that bind to a surface antigen described herein that comprise a framework region known to those of skill in the art (e.g., a human or non-human fragment). The framework region may be naturally occurring or consensus framework regions. In one embodiment, the framework region of an antibody of the invention is human (see, e.g., Clothia et al., 1998, J. Mol. Biol. 278:457-479 for a listing of human framework regions, which is incorporated by reference herein in its entirety).

In another embodiment, the present invention provides for antibodies that bind to a surface antigen described herein, said antibodies comprising the amino acid sequence of the human or murine framework regions with one or more amino acid substitutions at one, two, three, or more of the following residues: (a) rare framework residues that differ between the murine antibody framework (i.e., donor antibody framework) and the human antibody framework (i.e., acceptor antibody framework); (b) Venier zone residues when differing between donor antibody framework and acceptor antibody framework; (c) interchain packing residues at the V_{H}/V_{L} interface that differ between the donor antibody framework and the acceptor antibody framework; (d) canonical residues that differ between the donor antibody framework and the acceptor antibody framework sequences, particularly the framework regions crucial for the definition of the canonical class of the murine antibody CDR loops; (e) residues that are adjacent to a CDR; (g) residues capable of interacting with the antigen; (h) residues capable of interacting with the CDR; and (i) contact residues between the V_{H} domain and the V_{L} domain.

The present invention also provides antibodies of the invention that bind to a surface antigen described herein that comprise constant regions known to those of skill in the art. In one embodiment, the constant regions of an antibody of the invention are human. In another embodiment, the constant regions are derived from murine.

The present invention provides for antibodies that bind to a surface antigen described herein, the antibodies having an extended half-life in vivo and the use of such antibodies to produce an antibody conjugate of the invention. In a specific embodiment, the present invention provides antibodies that bind to a surface antigen described herein, which have a half-life in a subject, preferably a mammal and most preferably a human, of greater than about 2 minutes, 4 minutes, 5 minutes, 10 minutes, 12 minutes, 15 minutes, 20 minutes, 25 minutes, 30 minutes, 45 minutes, 1 hour, 1.5 hours, 2 hours, 3 hours, 4 hours, 5 hours, 6 hours, 7 hours, 12 hours, 14 hours, 24 hours, 48 hours, 1 week, 2 weeks, 1 month, 3 months, 6 months, 1 year 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months. In another embodiment, the present invention provides antibodies that bind to a surface antigen described herein, which have a half-life in a subject, preferably a mammal and most preferably a human, of 2 minutes, 5 minutes, 10 minutes, 15 minutes, 30 minutes, 1 hour, 1.5 hours, 2 hours, 3 hours, 4 hours, 5 hours, 6 hours, 7 hours, 12 hours, 14 hours, 24 hours, 48 hours, 1 week, 2 weeks, 1 month, 3 months, 6 months, 1 year 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibody conjugates (e.g., monoclonal antibodies, single chain antibodies, Fv fragments, and Fab fragments) in vivo, for example, inert polymer molecules such as high molecular weight polyethylene glycol (PEG) can be attached to the antibodies with or without a multifunctional linker either through site-specific conjugation of the PEG to the amino or carboxyl terminus of the antibodies (whichever end is not conjugated to the cytotoxic agent) or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or ion-exchange chromatography. Polyethylene glycol-derivatized antibodies can be tested for binding activity as well as for in vivo efficacy using methods well-known to those of skill in the art, for example, by immunoassays described herein.

Antibodies having an increased half-life in vivo can also be generated by introducing one or more amino acid modifications (i.e., substitutions, insertions, or deletions) into an IgG constant domain, or FcRn binding fragment thereof (preferably a Fc or hinge Fc domain fragment). See, e.g., International Publication No. WO 98/23289; International Publication No. WO 97/34631; International Publication No. WO 02/060919; and U.S. Pat. No. 6,277,375, each of which is incorporated herein by reference in its entirety.

Furthermore, antibodies can be conjugated to albumin in order to make the antibody more stable in vivo or have a longer half-life in vivo. The techniques are well-known in the art; see, e.g., International Publication Nos. WO 93/15199, WO 93/15200, and WO 01/77137; and European Patent No. EP 413,622, all of which are incorporated herein by reference.

Monitoring Cancer Stem Cells

In accordance with the invention, cancer stem cells comprise a unique subpopulation (often 0.1-10% or so) of a tumor that, in contrast to the remaining 90% or so of the tumor (i.e., the tumor bulk), are relatively more tumorigenic and relatively more slow-growing or quiescent. Given that conventional therapies and regimens have, in large part, been designed to attack rapidly proliferating cells (i.e., those cancer cells that comprise the tumor bulk), slower growing cancer stem cells may be relatively more resistant than faster growing tumor bulk to conventional therapies and regimens. This would explain another reason for the failure of standard oncology treatment regimens to ensure long-term benefit in most patients with advanced stage cancers. In a specific embodiment, a cancer stem cell(s) is the founder cell of a tumor (i.e., it is the progenitor of cancer cells). In some embodiments, a cancer stem cell(s) has one, two, three, or more or all of the following characteristics or properties: (i) can harbor the ability to initiate a tumor and/or to perpetuate tumor growth, (ii) can be generally relatively less mutated than the bulk of a tumor (e.g. due to slower growth and thus fewer DNA replication-dependent errors, improved DNA repair, and/or epigenetic/non-mutagenic changes contributing to their malignancy), (iii) can have many features of a normal stem cell(s) (e.g., similar cell surface antigen and/or intracellular expression profile, self-renewal programs, multi-drug resistance, an immature phenotype, etc., characteristic of normal stem cells) and may be derived from a normal stem cell(s), (iv) can be potentially responsive to its microenvironment (e.g., the cancer stem cells may be capable of being induced to differentiate and/or divide asymmetrically), (v) can be the source of metastases, (vi) can be slow-growing or quiescent, (vii) can be symmetrically-dividing, (viii) can be tumorigenic (e.g. as determined by NOD/SCID implantation experiments), (ix) can be relatively resistant to traditional therapies (i.e. chemoresistant), and (x) can comprise a subpopulation of a tumor (e.g. relative to the tumor bulk).

The samples for use in the methods of this invention may be taken from any animal subject, preferably mammal, most preferably a human. The subject from which a sample is obtained and utilized in accordance with the methods of this invention includes, without limitation, an asymptomatic subject, a subject manifesting or exhibiting 1, 2, 3, 4 or more symptoms of cancer, a subject clinically diagnosed as having cancer, a subject predisposed to cancer, a subject suspected of having cancer, a subject undergoing therapy for cancer, a subject that has been medically determined to be free of cancer (e.g., following therapy for the cancer), a subject that is managing cancer, or a subject that has not been diagnosed with cancer. In certain embodiments, the term “has no detectable cancer,” as used herein, refers to a subject or subjects in which there is no detectable cancer by conventional methods, e.g., MRI. In other embodiments, the term refers to a subject or subjects free from any disorder.

In certain embodiments, the amount of cancer stem cells in a subject or a sample from a subject is/are assessed prior to therapy or regimen (e.g. at baseline) or at least 1, 2, 4, 6, 7, 8, 10, 12, 14, 15, 16, 18, 20, 30, 60, 90 days, 6 months, 9 months, 12 months, or >12 months after the subject begins receiving the therapy or regimen. In certain embodiments, the amount of cancer stem cells is assessed after a certain number of doses (e.g., after 2, 5, 10, 20, 30 or more doses of a therapy). In other embodiments, the amount of cancer stem cells is assessed after 1 week, 2 weeks, 1 month, 2 months, I year, 2 years, 3 years, 4 years or more after receiving one or more therapies.

In certain embodiments, a positive or negative control sample is a sample that is obtained or derived from a corresponding tissue or biological fluid or tumor as the sample to be analyzed in accordance with the methods of the invention. This sample may come from the same patient or different persons and at the same or different time points.

For clarity of disclosure, and not by way of limitation, the following pertains to analysis of a blood sample from a patient. However, as one skilled in the art will appreciate, the assays and techniques described herein can be applied to other types of patient samples, including a body fluid (e.g. blood, bone marrow, plasma, urine, bile, ascitic fluid), a tissue sample suspected of containing material derived from a cancer (e.g. a biopsy) or homogenate thereof. The amount of sample to be collected will vary with the particular type of sample and method of determining the amount of cancer stem cells used and will be an amount sufficient to detect the cancer stem cells in the sample.

A sample of blood may be obtained from a patient having different developmental or disease stages. Blood may be drawn from a subject from any part of the body (e.g., a finger, a hand, a wrist, an arm, a leg, a foot, an ankle, a stomach, and a neck) using techniques known to one of skill in the art, in particular methods of phlebotomy known in the art. In a specific embodiment, venous blood is obtained from a subject and utilized in accordance with the methods of the invention. In another embodiment, arterial blood is obtained and utilized in accordance with the methods of the invention. The composition of venous blood varies according to the metabolic needs of the area of the body it is servicing. In contrast, the composition of arterial blood is consistent throughout the body. For routine blood tests, venous blood is generally used.

The amount of blood collected will vary depending upon the site of collection, the amount required for a method of the invention, and the comfort of the subject. In some embodiments, any amount of blood is collected that is sufficient to detect the amount of cancer stem cells. In a specific embodiment, 1 cc or more of blood is collected from a subject.

The amount of cancer stem cells in a sample can be expressed as the percentage of, e.g., overall cells, overall cancer cells or overall stem cells in the sample, or quantitated relative to area (e.g. cells per high power field), or volume (e.g. cells per ml), or architecture (e.g. cells per bone spicule in a bone marrow specimen).

In some embodiments, the sample may be a blood sample, bone marrow sample, or a tissue/tumor biopsy sample, wherein the amount of cancer stem cells per unit of volume (e.g., 1 mL) or other measured unit (e.g., per unit field in the case of a histological analysis) is quantitated. In certain embodiments, the cancer stem cell population is determined as a portion (e.g., a percentage) of the cancerous cells present in the blood or bone marrow or tissue/tumor biopsy sample or as a subset of the cancerous cells present in the blood or bone marrow or tissue/tumor biopsy sample. The cancer stem cell population, in other embodiments, can be determined as a portion (e.g., percentage) of the total cells. In yet other embodiments, the cancer stem cell population is determined as a portion (e.g., a percentage) of the total stem cells present in the blood sample.

In other embodiments, the sample from the patient is a tissue sample (e.g., a biopsy from a subject with or suspected of having cancerous tissue), where the amount of cancer stem cells can be measured, for example, by immunohistochemistry or flow cytometry, or on the basis of the amount of cancer stem cells per unit area, volume, or weight of the tissue. In certain embodiments, the cancer stem cell population (the amount of cancer stem cells) is determined as a portion (e.g., a percentage) of the cancerous cells present in the tissue sample or as a subset of the cancerous cells present in the tissue sample. In yet other embodiments, the cancerous stem cell population (the amount of cancer stem cells) is determined as a portion (e.g., a percentage) of the overall cells or stem cell cells in the tissue sample.

The amount of cancer stem cells in a test sample can be compared with the amount of cancer stem cells in reference sample(s) to assess the efficacy of the regimen. In one embodiment, the reference sample is a sample obtained from the subject undergoing therapy at an earlier time point (e.g., prior to receiving the regimen as a baseline reference sample, or at an earlier time point while receiving the therapy). In this embodiment, the therapy desirably results in a decrease in the amount of cancer stem cells in the test sample as compared with the reference sample. In another embodiment, the reference sample is obtained from a healthy subject who has no detectable cancer, or from a patient that is in remission for the same type of cancer. In this embodiment, the therapy desirably results in the test sample having an equal amount of cancer stem cells, or less than the amount of cancer stem cells than are detected in the reference sample.

In other embodiments, the cancer stem cell population in a test sample can be compared with a predetermined reference range and/or a previously detected amount of cancer stem cells determined for the subject to gauge the subject's response to the regimens described herein. In a specific embodiment, a stabilization or reduction in the amount of cancer stem cells relative to a predetermined reference range and/or earlier (previously detected) cancer stem cell amount determined for the subject indicates an improvement in the subject's prognosis or a positive response to the regimen, whereas an increase relative to the predetermined reference range and/or earlier cancer stem cell amount indicates the same or worse prognosis, and/or a failure to respond to the regimen. The cancer stem cell amount can be used in conjunction with other measures to assess the prognosis of the subject and/or the efficacy of the regimen. In a specific embodiment, the predetermined reference range is based on the amount of cancer stem cells obtained from a patient or population(s) of patients suffering from the same type of cancer as the patient undergoing the therapy. Exemplary cancer stem cell therapy is described in, for example, US Patent publication 2008/0118518A1.

Generally, since stem cell antigens can be present on both cancer stem cells and normal stem cells, a sample from the cancer-afflicted patient will have a higher stem cell count than a sample from a healthy subject who has no detectable cancer, due to the presence of the cancer stem cells. The therapy will desirably result in a cancer stem cell count for the test sample (e.g., the sample from the patient undergoing therapy) that decreases and becomes increasingly closer to the stem cell count in a reference sample that is sample from a healthy subject who has no detectable cancer.

If the reduction in amount of cancer stem cells is determined to be inadequate upon comparing the amount of cancer stem cells in the sample from the subject undergoing the regimen with the reference sample, then the medical practitioner has a number of possible options to adjust the regimen. For instance, the medical practitioner can then increase either the dosage or intensity of the therapy administered, the frequency of the administration, the duration of administration, combine the therapy with another therapy(ies), change the management altogether including halting therapy, or any combination thereof.

In certain embodiments, the dosage, frequency and/or duration of administration of a therapy is modified as a result of the change in the amount of cancer stem cells detected in or from the treated patient. For example, if a subject receiving therapy for leukemia has a cancer stem cell measurement of 2.5% of his tumor prior to therapy and 5% after 6 weeks of therapy, then the therapy or regimen may be altered or stopped because the increase in the percentage of cancer stem cells indicates that the therapy or regimen is not optimal. Alternatively, if another subject with leukemia has a cancer stem cell measurement of 2.5% of his tumor prior to therapy and 1% after 6 weeks of therapy, then the therapy or regimen may be continued because the decrease in the percentage of cancer stem cells indicates that the therapy or regimen is effective.

The amount of cancer stem cells can be monitored/assessed using standard techniques known to one of skill in the art. Cancer stem cells can be monitored by, e.g., obtaining a sample, such as a tissue/tumor sample, blood sample or a bone marrow sample, from a subject and detecting cancer stem cells in the sample. The amount of cancer stem cells in a sample (which may be expressed as percentages of, e.g., overall cells or overall cancer cells) can be assessed by detecting the expression of antigens on cancer stem cells. Techniques known to those skilled in the art can be used for measuring these activities. Antigen expression can be assayed, for example, by immunoassays including, but not limited to, western blots, immunohistochemistry, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, immunofluorescence, protein A immunoassays, flow cytometry, and FACS analysis. In such circumstances, the amount of cancer stem cells in a test sample from a subject may be determined by comparing the results to the amount of stem cells in a reference sample (e.g., a sample from a subject who has no detectable cancer) or to a predetermined reference range, or to the patient him/herself at an earlier time point (e.g. prior to, or during therapy).

In a specific embodiment, the cancer stem cell population in a sample from a patient is determined by flow cytometry. This method exploits the differential expression of certain surface markers on cancer stem cells relative to the bulk of the tumor. Labeled antibodies (e.g., fluorescent antibodies) can be used to react with the cells in the sample, and the cells are subsequently sorted by FACS methods. In some embodiments, a combination of cell surface markers are utilized in order to determine the amount of cancer stem cells in the sample. For example, both positive and negative cell sorting may be used to assess the amount of cancer stem cells in the sample. Cancer stem cells for specific tumor types can be determined by assessing the expression of markers on cancer stem cells. In certain embodiments, the tumors harbor cancer stem cells and their associated markers as set forth in Table 2, which provides a non-limiting list of cancer stem cell phenotypes associated with various types of cancer.

In certain in vivo techniques, an imaging agent or diagnostic agent is used which binds to biological molecules on cancer cells or cancer stem cells, e.g., cancer cell or cancer stem cell surface antigens. For instance, a fluorescent tag, radionuclide, heavy metal, or photon-emitter is attached to an antibody (including an antibody fragment) that binds to a cancer stem cell surface antigen. Exemplary cancer stem cell surface antigens are listed in Table 2. The medical practitioner can infuse the labeled antibody into the patient either prior to, during, or following treatment, and then the practitioner can place the patient into a total body scanner/developer which can detect the attached label (e.g., fluorescent tag, radionuclide, heavy metal, photon-emitter). The scanner/developer (e.g., CT, MRI, or other scanner, e.g. detector of fluorescent label, that can detect the label) records the presence, amount/quantity, and bodily location of the bound antibody. In this manner, the mapping and quantitation of tag (e.g. fluorescence, radioactivity, etc.) in patterns (i.e., different from patterns of normal stem cells within a tissue) within a tissue or tissues indicates the treatment efficacy within the patient's body when compared to a reference control such as the same patient at an earlier time point or a patient or healthy individual who has no detectable cancer. For example, a large signal (relative to a reference range or a prior treatment date, or prior to treatment) at a particular location indicates the presence of cancer stem cells. If this signal is increased relative to a prior date it suggests a worsening of the disease and failure of therapy or regimen. Alternatively, a signal decrease indicates that the therapy or regimen has been effective.

Any in vitro or in vivo (ex vivo) assays known to those skilled in the art that can detect and/or quantify cancer stem cells can be used to monitor cancer stem cells in order to evaluate the therapeutic utility of a cancer therapy or regimen disclosed herein for cancer or one or more symptoms thereof; or these assays can be used to assess the prognosis of a patient. The results of these assays then may be used to possibly maintain or alter the cancer therapy or regimen. Moreover, any in vitro or in vivo (ex vivo) assays known to those skilled in the art that can detect and/or quantify cancer stem cells can be used to monitor cancer stem cells in order to evaluate the prophylactic utility of a cancer therapy or regimen disclosed herein for cancer or one or more symptoms thereof; or these assays can be used to assess the prognosis of a patient. The results of these assays then may be used to possibly maintain or alter the cancer therapy or regimen.

The amount of cancer stem cells in a specimen can be compared to a predetermined reference range and/or an earlier amount of cancer stem cells previously determined for the subject (either prior to, or during therapy) in order to gauge the subject's response to the treatment regimens described herein. In a specific embodiment, a stabilization or reduction in the amount of cancer stem cells relative to a predetermined reference range and/or earlier cancer stem cell amount previously determined for the subject (either prior to, or during therapy) indicates that the therapy or regimen was effective and thus possibly an improvement in the subject's prognosis, whereas an increase relative to the predetermined reference range and/or cancer stem cell amount detected at an earlier time point indicates that the therapy or regimen was ineffective and thus possibly the same or a worsening in the subject's prognosis. The cancer stem cell amount can be used with other standard measures of cancer to assess the prognosis of the subject and/or efficacy of the therapy or regimen: such as response rate, durability of response, relapse-free survival, disease-free survival, progression-free survival, and overall survival. In certain embodiments, the dosage, frequency and/or duration of administration of a therapy is modified as a result of the determination of the amount or change in the amount of cancer stem cells at various time points which may include prior to, during, and/or following therapy.

The present invention also relates to methods for determining that a cancer therapy or regimen is effective at targeting and/or impairing cancer stem cells by virtue of monitoring cancer stem cells over time and detecting a stabilization or decrease in the amount of cancer stem cells during and/or following the course of the cancer therapy or regimen.

In a certain embodiment, a therapy or regimen may be described or marketed as an anti-cancer stem cell therapy or regimen based on the determination that a therapy or regimen is effective at targeting and/or impairing cancer stem cells by virtue of having monitored or detected a stabilization or decrease in the amount of cancer stem cells during therapy.

The present invention is also directed to methods to treat cancer involving i) determining that a cancer therapy is effective by virtue of its ability to decrease cancer stem cells as determined by the monitoring of cancer stem cells, and ii) administering the therapy to a human(s) with cancer. The present invention is also directed to methods to treat cancer involving i) administering to a human with cancer a cancer therapy, ii) determining the amount of cancer stem cells prior to, during, and/or following therapy through the monitoring of cancer stem cells, and iii) continuing, altering, or halting therapy based on such monitoring. The present invention is also directed toward the assaying for/screening of a therapy(s) for anti-cancer stem cell activity involving i) administration of the therapy to a human with cancer, ii) monitoring cancer stem cells in or from the human prior to, during, and/or following therapy, and iii) determining whether the therapy resulted in a decrease in the amount of cancer stem cells.

### In Vivo Assays

In certain in vivo techniques, an imaging agent, or diagnostic moiety, is used which binds to molecules on cancer cells or cancer stem cells, e.g., cancer cell or cancer stem cell surface antigens. For instance, a fluorescent tag, radionuclide, heavy metal, or photon-emitter is attached to an antibody (including an antibody fragment) that binds to a cancer stem cell surface antigen. Exemplary cancer stem cell surface antigens are listed in Table 2. The medical practitioner can infuse the labeled antibody into the patient either prior to, during, or following treatment, and then the practitioner can place the patient into a total body scanner/developer which can detect the attached label (e.g., fluorescent tag, radionuclide, heavy metal, photon-emitter). The scanner/developer (e.g., CT, MRI, or other scanner, e.g. detector of fluorescent label, that can detect the label) records the presence, amount/quantity, and bodily location of the bound antibody. In this manner, the mapping and quantitation of tag (e.g. fluorescence, radioactivity, etc.) in patterns (i.e., different from patterns of normal stem cells within a tissue) within a tissue or tissues indicates the treatment efficacy within the patient's body when compared to a reference control such as the same patient at an earlier time point or a patient who has no detectable cancer. For example, a large signal (relative to a reference range or a prior treatment date, or prior to treatment) at a particular location indicates the presence of cancer stem cells. If this signal is increased relative to a prior date it suggests a worsening of the disease and failure of therapy or regimen. Alternatively, a signal decrease indicates that therapy or regimen has been effective.

Similarly, in some embodiments of the invention, the efficacy of the therapeutic regimen in reducing the amount of cancer cells in animals (including humans) undergoing treatment can be evaluated using in vivo techniques. In one embodiment, the medical practitioner performs the imaging technique with labeled molecule that specifically binds the surface of a cancer cell, e.g., a cancer cell surface antigen. See, e.g., Table 2, for a list of certain cancer cell surface antigens. In this manner, the mapping and quantitation of tag (e.g., fluorescence, radioactivity) in patterns within a tissue or tissues indicates the treatment efficacy within the body of the patient undergoing treatment.

In a specific embodiment, the amount of cancer stem cells is detected in vivo in a subject according to a method comprising the steps of: (a) administering to the subject an effective amount of a labeled cancer stem cell marker binding agent that specifically binds to a cell surface marker found on the cancer stem cells, and (b) detecting the labeled agent in the subject following a time interval sufficient to allow the labeled agent to concentrate at sites in the subject where the cancer stem cell surface marker is expressed. In accordance with this embodiment, the cancer stem cell surface marker-binding agent is administered to the subject according to any suitable method in the art, for example, parenterally (e.g. intravenously), intraureterally, subureterally, into the bladder via a catheter, injected directly into the bladder, subcutaneously or intraperitoneally. In accordance with this embodiment, the effective amount of the agent is the amount which permits the detection of the agent in the subject. This amount will vary according to the particular subject, the label used, and the detection method employed. For example, it is understood in the art that the size of the subject and the imaging system used will determine the amount of labeled agent needed to detect the agent in a subject using imaging. In the case of a radiolabeled agent for a human subject, the amount of labeled agent administered is measured in terms of radioactivity, for example from about 5 to 20 millicuries of ⁹⁹Tc. The time interval following the administration of the labeled agent which is sufficient to allow the labeled agent to concentrate at sites in the subject where the cancer stem cell surface marker is expressed will vary depending on several factors, for example, the type of label used, the mode of administration, and the part of the subject's body that is imaged. In a particular embodiment, the time interval that is sufficient is 6 to 48 hours, 6 to 24 hours, or 6 to 12 hours. In another embodiment the time interval is 5 to 20 days or 5 to 10 days. The presence of the labeled cancer stem cell surface marker-binding agent can be detected in the subject using imaging means known in the art. In general, the imaging means employed depend upon the type of label used. Skilled artisans will be able to determine the appropriate means for detecting a particular label. Methods and devices that may be used include, but are not limited to, computed tomography (CT), whole body scan such as position emission tomography (PET), magnetic resonance imaging (MRI), fluorescence, chemiluminescence, an imager which can detect and localize fluorescent label and sonography. In a specific embodiment, the cancer stem cell surface marker-binding agent is labeled with a radioisotope and is detected in the patient using a radiation responsive surgical instrument (Thurston et al., U.S. Pat. No. 5,441,050). In another embodiment, the cancer stem cell surface marker-binding agent is labeled with a fluorescent compound and is detected in the patient using a fluorescence responsive scanning instrument. In another embodiment, the cancer stem cell surface marker-binding agent is labeled with a positron emitting metal and is detected in the patient using positron emission-tomography. In yet another embodiment, the cancer stem cell surface marker-binding agent is labeled with a paramagnetic label and is detected in a patient using magnetic resonance imaging (MRI).

Any in vitro or in vivo (ex vivo) assays known to those skilled in the art that can detect and/or quantify cancer stem cells can be used to monitor cancer stem cells in order to evaluate the therapeutic utility of a cancer therapy or regimen disclosed herein for cancer or one or more symptoms thereof; or these assays can be used to assess the prognosis of a patient. Any in vitro or in vivo (ex vivo) assays known to those skilled in the art that can detect and/or quantify cancer stem cells can be used to monitor cancer stem cells in order to evaluate the prophylactic utility of a cancer therapy or regimen disclosed herein for cancer or one or more symptoms thereof; or these assays can be used to assess the prognosis of a patient. The results of these assays then may be used to possibly maintain or alter the cancer therapy or regimen.

Compounds and agents of the invention are listed herein according to their target. Compounds and agents are listed herein in the following manner (target symbol, target, compound or agent): MMP1, matrix metallopeptidase 1 (interstitial collagenase), doxycycline, illomastat, minocycline, marimastat, cipemastat; FABP4, fatty acid binding protein 4, sc-202606 (aka: ((2′-(5-Ethyl-3,4-diphenyl-1H-pyrazol-1-yl)(1,1′-biphenyl)-3-yl)oxy)-acetic acid); DLC1, deleted in liver cancer 1, SAHA (aka: suberoylanilide hydroxamic acid); NUAK1, NUAK family SNF1-like kinase 1, BX795 (Mol formula: C23H26N₇O2S); GALT, galectin-7,1,5-Bis(b-D-galactopyranosylthio)-2,4-dinitrobenzene, 5-Benzylsulfanyl-2,4-dinitrophenyl 1-thio-b-D-galactopyrano-side; MMP13, matrix metallopeptidase 13 (collagenase 3), N,N′-bisaryl-pyrimidine-4,6-dicarboxamide derivatives; CXCR4, chemokine (C—X—C motif) receptor 4, msx-122 (name), mozobil, T134; HMOX1, heme oxygenase (decycling) 1, Tin protoporphyrin IX dichloride, Zinc protoporphyrin IX; TGM1, transglutaminase 1,1-dimethyl, 2-[(oxopropyl)thio]imidazolium, N-benzyloxycarbonyl-1-glutaminyl-6-dimethylsulfonium-5-oxo-1-norleucine. In all tables and discussion herein, gene names may also include the names of proteins expressed by such genes. For the avoidance of doubt, proteins that are targeted by agents of the invention are identified from the information in the tables using known methods available to one of skill in the art.

Antibodies of the invention target cancer stem cells and bind to target proteins expressed by the genes listed in Table 2 (genes that are up in 67LR bright cells), Table 3, Table 5, Table 7, Table 9, Table 11. Antibodies of the invention that target cancer cells bind targets expressed by the genes listed in Table 2 (both genes that are up and genes that are down in 67LR bright cells), Table 3, Table 5, Table 6, Table 7, Table 8, Table 9, Table 10, Table 11, Table 12. In all tables and discussion herein, gene names may also include the names of proteins expressed by such genes. For the avoidance of doubt, proteins that are targeted by agents of the invention are identified from the information in the tables using known methods available to one of skill in the art. For the avoidance of doubt, antibodies of the invention to the targets and target proteins listed in the tables includes all variations, fragments, conjugates, modifications, and other derivates as described herein.

Cytotoxic Agents

Any cytotoxic agent or otherwise anticellular agent known to one of skill in the art can be used to produce the antibody conjugates of the invention. A cytotoxic agent includes any agent that is detrimental to cells. Exemplary cytotoxic agents include chemotherapeutic agents, radioisotopes, cytotoxins such as cytostatic or cytocidal agents, or other anticellular agents, including known therapeutic agents.

Non-limiting examples of cytotoxic agents include antimetabolites (e.g., cytosine arabinoside, aminopterin, methotrexate, 6-mercaptopurine, 6-thioguanine, cytarabine, and 5-fluorouracil decarbazine); alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, cis-dichlorodiammine-platinum (II) (CDDP), and cisplatin); vinca alkaloid; anthracyclines (e.g., daunorubicin (formerly daunomycin) and doxorubicin); antibiotics (e.g., dactinomycin (formerly actinomycin), bleomycin, mithramycin, and anthramycin (AMC)); calicheamicin; CC-1065 and derivatives thereof; auristatin molecules (e.g., auristatin PHE, bryostatin-1, and dolastatin-10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); DNA-repair enzyme inhibitors (e.g., etoposide or topotecan); kinase inhibitors (e.g., compound ST1571, imatinib mesylate (Kantarjian et al., Clin Cancer Res. 8(7):2167-76 (2002)); demecolcine; and other cytotoxic agents (e.g., paclitaxel, cytochalasin B, gramicidin D, ethidium bromide, emetine, mitomycin, etoposide, tenoposide, vincristine, vinblastine, colchicine, doxorubicin, daunorubicin, dihydroxy anthracenedione, mitoxantrone, mithramycin, actinomycin D, 1-dehydrotestosterone, glucorticoids, procaine, tetracaine, lidocaine, propranolol, and puromycin and analogs or homologues thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305); topoisomerase inhibitors (e.g., camptothecin, irinotecan, SN-38, topotecan, 9-aminocamptothecin, GG211 (GI147211), DX-8951f, IST-622, rubitecan, pyrazoloacridine, XR5000, saintopin, UCE6, UCE1022, TAN-1518A, TAN 1518B, KT6006, KT6528, ED-110, NB-506, ED-110, NB-506, and rebeccamycin); bulgarein; DNA minor groove binders such as Hoechst dye 33342 and Hoechst dye 33258; nitidine; fagaronine; epiberberine; coralyne; beta-lapachone; BC-4-1; antisense oligonucleotides (e.g., those disclosed in the U.S. Pat. Nos. 6,277,832, 5,998,596, 5,885,834, 5,734,033, and 5,618,709); adenosine deaminase inhibitors (e.g., fludarabine phosphate and 2-chlorodeoxyadenosine); and pharmaceutically acceptable salts, solvates, clathrates, and prodrugs thereof.

Other examples of cytotoxic agents which can be used to produce the antibody conjugates of the invention include antimitotic drugs, such as auristatin, derivatives of auristatin, monomethylauristatin, and derivatives of monomethylauristatin, such as monomethylauristatin F and monomethylauristatin E.

Other examples of cytotoxic agents which can be used to produce the antibody conjugates of the invention include maytansine (Cassady et al., 2004, Chem. Pharm. Bull. 52(1):1-26), and maytansine derivatives such as DM1 (Tassone et al., 2008, Blood 104(12): 3688-3696; Erickson et al., 2006, Cancer Res 66(8) 4426-4433) and DM4 (Erickson et al., 2006, Cancer Res 66(8) 4426-4433)

Other examples of cytotoxic agents which can be used to produce the antibody conjugates of the invention are single-walled carbon nanotubes (Gannon et al., 2007 Cancer, 110:2654-2665).

In one embodiment, the antibody to a surface antigen described herein is conjugated to a radioactive metal ion, such as the alpha-emitters ²¹¹astatine, ²¹²bismuth, ²¹³bismuth; the beta-emitters ¹³¹iodine, ⁹⁰yttrium, ¹⁷⁷lutetium, ¹⁵³samarium, and ¹⁰⁹palladium; or macrocyclic chelators useful for conjugating radiometal ions, including but not limited to, ¹³¹indium, ¹³¹L, ¹³¹yttrium, ¹³¹holmium, ¹³¹samarium, to polypeptides or any of those listed herein. In certain embodiments, the macrocyclic chelator is 1,4,7,10-tetraazacyclododecane-N,N′,N″,N′″-tetraacetic acid (DOTA), which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

In a specific embodiment, the antibody to a surface antigen described herein is conjugated to a proteinaceous agent that modifies a given biological response and leads to cytotoxicity. In one embodiment, the antibody to a surface antigen described herein is conjugated to a plant-, fungus-, or bacteria-derived toxin. Non-limiting examples of such toxins include A chain toxins, ribosome inactivating proteins, ricin A, deglycosylated ricin A chain, abrin, alpha sarcin, aspergillin, restrictocin, ribonucleases, diphtheria toxin, bacterial endotoxin, saporin toxin, Granzyme B or the lipid A moiety of bacterial endotoxin, cholera toxin, or Pseudomonas exotoxin and derivatives and variants thereof.

Fragments, analogs, and derivatives of proteinaceous cytotoxins can be useful in the present invention provided that when fused to the antibody portion of the conjugate, of an antibody to a surface antigen described herein, such fragments, analogs, and derivatives allow the conjugate to bind a native surface antigen described herein expressed on the surface of a cell. Preferably, the binding kinetics of the fragments, analogs, or derivatives remain the same or vary only by not more than 25%. The cytotoxin may be from any species. The nucleotide and/or amino acid sequences of cytotoxins can be found in the literature or public databases, or the nucleotide and/or amino acid sequences can be determined using cloning and sequencing techniques known to one of skill in the art. In some embodiments, the cytotoxin is derived from mammals. In other embodiments, the cytotoxin is derived from bacteria. In yet other embodiments, the cytotoxin is derived from fungi. In a preferred embodiment, the cytotoxin is Pseudomonas exotoxin A (PE), or an analog, derivative, or a fragment thereof.

In one embodiment of the invention, the proteinaceous cytotoxin comprises an amino acid sequence which contains at least one conservative amino acid substitution, but not more than 40 conservative amino acid substitutions, even more preferably, not more than 30 conservative amino acid substitutions, still more preferably, not more than 20 conservative amino acid substitutions, and still even more preferably, not more than 10 conservative amino acid substitutions relative to the native amino acid sequence of the chosen fragment (e.g., the native PE or diphtheria toxin amino acid sequence), which result in a silent change, i.e., no change in an activity necessary for cytotoxicity in the context of the conjugate. In another embodiment of the invention, a cytotoxin comprises an amino acid sequence that contains at least one conservative amino acid substitution in the chosen fragment; but not more than 10, 9, 8, 7, 6, 5, 4, 3, 2 or 1 conservative amino acid substitutions relative to the native amino acid sequence (e.g., the native PE or diphtheria toxin amino acid sequence), which result in a silent change. In yet another embodiment, a cytotoxic polypeptide comprises an amino acid sequence that contains one or more conservative substitutions or a combination of non-conservative and conservative amino acid substitutions relative to the native amino acid sequence of the chosen fragment, which results in a silent change.

To improve or alter the characteristics of cytotoxic polypeptides, protein engineering may be employed. Recombinant DNA technology known to those skilled in the art can be used to create novel mutant proteins or “muteins” including single or multiple amino acid substitutions, deletions, additions, or fusion proteins. Such modified polypeptides can show, e.g., enhanced activity or increased stability. In addition, they may be purified in higher yields and show better solubility than the corresponding natural polypeptide, at least under certain purification and storage conditions. For instance, for many proteins, it is known in the art that one or more amino acids may be deleted from the amino terminus or carboxyl terminus without substantial loss of biological function. Exemplary cytotoxin variants suitable for forming the conjugates of the invention are found herein.

In a specific embodiment, a cytotoxic polypeptide is at least 50%, at least 65%, at least 70%, at least 75%, at least 80%, at least 85%, at least 90%, or at least 95% identical to the amino acid sequence of chosen fragment of the native cytotoxin (e.g., the native PE or diphtheria toxin amino acid sequence).

Therapeutic Agents

Any therapeutic agent known to one of skill in the art can be used to produce the antibody conjugates of the invention. A therapeutic agent includes any agent that, when conjugated to an antibody or fragment thereof of the invention, can be used to treat cancer. In certain embodiments, a cytotoxic agent as exemplified in section 5.3 can be a therapeutic agent.

An antibody of the invention can be conjugated to therapeutic agents such as macrocyclic chelators useful for conjugating radiometal ions. In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo, et al., 1998, Clin Cancer Res 4:2483-90; Peterson, et al., 1999, Bioconjug Chem 10:553; and Zimmerman, et al., 1999, Nucl Med Biol 26:943-50 each incorporated by reference in their entireties.

Examples of useful therapeutic radioisotopes (ordered by atomic number) include 47Sc, 67Cu, 90Y, 109Pd, 125I, 131I, 186Re, 188Re, 199Au, 211At, 212Pb and 217Bi. These atoms can be conjugated to the peptide directly, indirectly as part of a chelate, or, in the case of iodine, indirectly as part of an iodinated Bolton-Hunter group. The radioiodine can be introduced either before or after this group and is coupled to the peptide or protein.

Examples of therapeutic agents which can be used to produce the antibody conjugates of the invention include, Bc1-2 family inhibitors and Bc1-2 inhibitors, including ABT-737.

Other examples of therapeutic agents which can be used to produce the antibody conjugates of the invention include nanoparticles, such as perfluorocarbon nanoparticles, (Tran et al., 2007, Int. J. Nanomedicine 2(4):515-526) and paramagnetic nanoparticles (Cyrus et al., 2008, Arterioscler Thromb Vasc Biol (on-line publication)).

An antibody or fragment thereof may be conjugated to a therapeutic agent that modifies a given biological response. Therapeutic agents are not to be construed as limited to classical chemical therapeutic agents. For example, the therapeutic agent may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, .alpha.-interferon, nerve growth factor, platelet derived growth factor, tissue plasminogen activator, an apoptotic agent, e.g., TNF-.alpha., AIM I (see, International Publication No. WO 97/33899 herein incorporated by reference in its entirety), AIM II (see, International Publication No. WO 97/34911 herein incorporated by reference in its entirety), Fas Ligand (Takahashi, et al., 1994, J Immunol, 6:1567 herein incorporated by reference in its entirety), and VEGI (see, International Publication No. WO 99/23105 herein incorporated by reference in its entirety), a thrombotic agent or an anti-angiogenic agent, e.g., angiostatin or endostatin; or, a biological response modifier such as, for example, a lymphokine (e.g., interleukin-1 (“IL-1”), interleukin-2 (“IL-2”), interleukin-6 (“IL-6”), granulocyte macrophage colony stimulating factor (“GM-CSF”), and granulocyte colony stimulating factor (“G-CSF”)), or a growth factor (e.g., growth hormone (“GH”)).

Antibodies of the invention or fragments thereof can also be conjugated to Lectins. Lectins are proteins, commonly derived from plants, that bind to carbohydrates. Among other activities, some lectins are toxic. Some of the most cytotoxic substances known are protein toxins of bacterial and plant origin (Frankel et al., Ann Rev Med 37:125-142 (1986) herein incorporated by reference in its entirety). These molecules binding the cell surface and inhibit cellular protein synthesis. The most commonly used plant toxins are ricin and abrin; the most commonly used bacterial toxins are diphtheria toxin and Pseudomonas exotoxin A. In ricin and abrin, the binding and toxic functions are contained in two separate protein subunits, the A and B chains. The ricin B chain binds to the cell surface carbohydrates and promotes the uptake of the A chain into the cell. Once inside the cell, the ricin A chain inhibits protein synthesis by inactivating the 60S subunit of the eukaryotic ribosome Endo, et al., J Biol Chem 262: 5908-5912 (1987) herein incorporated by reference in its entirety). Other plant derived toxins, which are single chain ribosomal inhibitory proteins, include pokeweed antiviral protein, wheat germ protein, gelonin, dianthins, momorcharins, trichosanthin, and many others (Strip, et al., FEBS Lett 195:1-8 (1986) herein incorporated by reference in its entirety). Diphtheria toxin and Pseudomonas exotoxin A are also single chain proteins, and their binding and toxicity functions reside in separate domains of the same protein Pseudomonas exotoxin A has the same catalytic activity as diphtheria toxin. Ricin has been used therapeutically by binding its toxic a-chain, to targeting molecules such as Abs to enable site-specific delivery of the toxic effect. Bacterial toxins have also been used as anti-tumor conjugates. As intended herein, a toxic peptide chain or domain is conjugated to a agent of this invention and delivered in a site-specific manner to a target site where the toxic activity is desired, such as a metastatic focus.

Methods for Producing Antibody Conjugates to a Surface Antigen Described Herein

Techniques for conjugating cytotoxic agents or otherwise anticellular moieties to antibodies are well known, e.g., Amon et al., “Monoclonal Antibodies For Immunotargeting Of Drugs In Cancer Therapy,” in Monoclonal Antibodies And Cancer Therapy, Reisfeld et al. (eds.), pp. 243-56 (Alan R. Liss, Inc. 1985); Hellstrom et al., “Antibodies For Drug Delivery,” in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review,” in Monoclonal Antibodies 84: Biological And Clinical Applications, Pinchera et al. (eds.), pp. 475-506 (1985); “Analysis, Results, And Future Prospective Of The Therapeutic Use Of Radiolabelled Antibody In Cancer Therapy,” in Monoclonal Antibodies For Cancer Detection And Therapy, Baldwin et al. (eds.), pp. 303-16 (Academic Press 1985), and Thorpe et al., 1982, Immunol. Rev. 62:119-58; each of which is incorporated herein by reference in its entirety.

Methods for fusing or conjugating proteins, polypeptides, or peptides to an antibody are known in the art, and can be used to conjugate proteinaceous cytotoxins to antibodies, or for connecting cytotoxins to antibodies through a peptide linker. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references are incorporated herein by reference in their entireties).

The conjugates of the present invention can be made by standard recombinant DNA techniques or by protein synthetic techniques, e.g., by use of a peptide synthesizer. For example, a nucleic acid molecule encoding a conjugate of the invention can be synthesized by conventional techniques including automated DNA synthesizers. Alternatively, PCR amplification of gene fragments can be carried out using anchor primers that give rise to complementary overhangs between two consecutive gene fragments, which can subsequently be annealed and re-amplified to generate a chimeric gene sequence (see, e.g., Current Protocols in Molecular Biology, Ausubel et al., eds., John Wiley & Sons, 1992).

The nucleotide sequences encoding a conjugate or portion thereof of the invention (antibody to a surface antigen described herein and/or proteinaceous cytotoxin sequences, such as but not limited to Pseudomonas exotoxin or diphtheria toxin) may be obtained from any information available to those of skill in the art (i.e., from GenBank, the literature, or by routine cloning). The nucleotide sequence coding for a conjugate, or for the antibody or cytotoxin moiety of the conjugate, can be inserted into an appropriate expression vector, i.e., a vector that contains the necessary elements for the transcription and translation of the inserted protein-coding sequence. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Methods for Producing Antibodies

Antibodies that bind to an antigen can be produced by any method known in the art for the synthesis of antibodies, in particular, by chemical synthesis or preferably, by recombinant expression techniques. In some embodiments, an immunogen that comprises an epitope unique to a protein component is used to generate an antibody specific for the component.

Polyclonal antibodies specific for an antigen can be produced by various procedures well-known in the art. As a non-limiting example, the antigen (i.e., a surface antigen described herein) can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Kohler & Milstein, 1975, Nature 256:495-497; Pasqualini & Arap, 2004, PNAS USA 101:257-259; Steinitz et al., 1977, Nature 269:420-422; Vollmers et al., 1989, Cancer Res. 49:2471-2476; Vollmers & Brandlein, 2002 Hum. Antibodies 11(4):131-142; Harlow et al., Antibodies: A Laboratory Manual, (Cold Spring Harbor Laboratory Press, 2nd ed. 1988); Hammerling, et al., in: Monoclonal Antibodies and T Cell Hybridomas 563 681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a surface antigen described herein. Ascites fluid, which generally contains high levels of antibodies, can be generated by immunizing mice with positive hybridoma clones.

Further, the antibodies of the present invention can also be generated using various phage display methods known in the art. In phage display methods, functional antibody domains are displayed on the surface of phage particles that carry the polynucleotide sequences encoding them. In particular, DNA sequences encoding V_{H} and V_{L} domains are amplified from animal cDNA libraries (e.g., human or murine cDNA libraries of tissues expressing a surface antigen described herein). The DNA encoding the V_{H} and V_{L} domains are recombined together with an scFv linker by PCR and cloned into a phagemid vector. The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/01134; Griffiths et al., 1994, EMBO J. 13:3245-3260; Winter et al., 1994, Annu. Rev. Immunol. 12:433-455; Liv et al., 2004, Cancer Res. 64:704-710; International publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the herein references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described herein. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including V_{H} or V_{L} nucleotide sequences, a restriction site, and a flanking sequence to protect the restriction site can be used to amplify the V_{H} or V_{L} sequences in scFv clones. Utilizing cloning techniques known to those of skill in the art, the PCR amplified V_{H} domains can be cloned into vectors expressing a V_{H} constant region, e.g., the human gamma 4 constant region, and the PCR amplified V_{L} domains can be cloned into vectors expressing a V_{L} constant region, e.g., human kappa or lambda constant regions. Preferably, the vectors for expressing the V_{H} or V_{L} domains comprise an EF—1.alpha. promoter, a secretion signal, a cloning site for the variable domain, constant domains, and a selection marker such as neomycin. The V_{H} and V_{L} domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use humanized antibodies or chimeric antibodies. Completely human antibodies and humanized antibodies are particularly desirable for therapeutic treatment of human subjects. Human antibodies can be made by a variety of methods known in the art including phage display methods described herein using antibody libraries derived from human immunoglobulin sequences. See also U.S. Pat. Nos. 4,444,887 and 4,716,111; and International publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice that are incapable of expressing functional endogenous immunoglobulins, but that can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM, and IgE antibodies. For an overview of this technology for producing human antibodies, see Lonberg and Huszar, 1995, Int. Rev. Immunol. 13:65 93.

For a detailed discussion of methods for producing human antibodies and human monoclonal antibodies and protocols for producing such antibodies, see, e.g., Tomizuka et al., 2000 PNAS USA 97:722-727; Davis et al., 2004, Methods Mol. Biol. 248:191-200; Lagerkvist et al., 1995, Biotechniques 18:862-869; Babcook et al., 1996 PNAS USA 93:7843-7848; International publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described herein.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,311,415, which are incorporated herein by reference in their entirety.

A humanized antibody is an antibody that is capable of binding to a predetermined antigen and that comprises a framework region having substantially the amino acid sequence of a human immunoglobulin and a CDR having substantially the amino acid sequence of a non-human immunoglobulin. A humanized antibody comprises substantially all of at least one, and typically two, variable domains (Fab, Fab′, F(ab′)₂, Fabc, Fv), in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA, and IgE, and any isotype, including IgG1, IgG2, IgG3, and IgG4. Usually the constant domain is a complement fixing constant domain where it is desired that the humanized antibody exhibit cytotoxic activity, and the class is typically IgG1. Where such cytotoxic activity is not desirable, the constant domain may be of the IgG2 class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and most preferably greater than 95%. A humanized antibody can be produced using variety of techniques known in the art, including but not limited to, CDR-grafting (see e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see e.g., European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973, each of which is incorporated herein by its entirety by reference), chain shuffling (see e.g., U.S. Pat. No. 5,565,332, which is incorporated herein in its entirety by reference), and techniques disclosed in, e.g., U.S. Pat. No. 6,407,213, U.S. Pat. No. 5,766,886, International Publication No. WO 9317105, Tan et al., J. Immunol. 169:1119 25 (2002), Caldas et al., Protein Eng. 13(5):353 60 (2000), Morea et al., Methods 20(3):267 79 (2000), Baca et al., J. Biol. Chem. 272(16):10678 84 (1997), Roguska et al., Protein Eng. 9(10):895 904 (1996), Couto et al., Cancer Res. 55 (23 Supp):5973s-5977s (1995), Couto et al., Cancer Res. 55(8):1717 22 (1995), Sandhu J S, Gene 150(2):409 10 (1994), and Pedersen et al., J. Mol. Biol. 235(3):959 73 (1994), each of which is incorporated herein in its entirety by reference. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Diabodies, triabodies, and tetrabodies can be produced by techniques known to one of skill in the art. See, e.g., Kipriyanov, 2002, Methods Mol. Biol. 178:317-331; Todorovska et al., 2001J. Immunol. Methods 248:47-66; and Poljak et al., 1994, Structure 2:1121-1123, each of which are incorporated herein by reference in their entirety, for methods for producing diabodies, triabodies, and tetrabodies. Single domain antibodies can also be produced by techniques known to one of skill in the art. For a description of techniques to produce single domain antibodies, see, e.g., Holliger & Hudson, 2005 Nat. Biotechnol. 23:1126-1136, Riechmann et al., 1999, J. Immunol. Methods 231:25-38; and Dick, 1990, BMJ 300:659-600, each of which is incorporated herein by reference in its entirety.

Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies may also be used in the generation of intrabodies.

Pharmaceutical Compositions and Routes of Administration

The present invention provides compositions comprising an antibody or antibody conjugate of the invention and carrier. In one embodiment, the invention provides compositions comprising an antibody of the invention. In another embodiment, the invention provides compositions comprising an antibody conjugate, such as an immunotoxin.

The invention provides a pharmaceutical composition comprising an effective amount of an antibody or antibody conjugate of the invention and a pharmaceutically acceptable carrier or vehicle. In a specific embodiment, a pharmaceutical composition comprises an effective amount of an antibody of the invention and a pharmaceutical acceptable carrier or vehicle. In another embodiment, a pharmaceutical composition comprises an effective amount of an antibody conjugate of the invention and a pharmaceutically acceptable carrier or vehicle. The pharmaceutical compositions are suitable for veterinary and/or human administration.

The pharmaceutical compositions of the present invention can be in any form that allows for the composition to be administered to a subject, said subject preferably being an animal, including, but not limited to a human, mammal, or non-human animal, such as a cow, horse, sheep, pig, fowl, cat, dog, mouse, rat, rabbit, guinea pig, etc., and is more preferably a mammal, and most preferably a human.

The compositions of the invention can be in the form of a solid, liquid or gas (aerosol). Typical routes of administration may include, without limitation, oral, topical, parenteral, sublingual, rectal, vaginal, ocular, intradermal, intratumoral, intracerebral, intrathecal, and intranasal. Parenteral administration includes subcutaneous injections, intravenous, intramuscular, intraperitoneal, intrapleural, intrasternal injection or infusion techniques. In a specific embodiment, the compositions are administered parenterally. In a more specific embodiment, the compositions are administered intravenously. Pharmaceutical compositions of the invention can be formulated so as to allow an antibody or antibody conjugate of the invention to be bioavailable upon administration of the composition to a subject. Compositions can take the form of one or more dosage units, where, for example, a tablet can be a single dosage unit, and a container of an antibody or antibody conjugate of the invention in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of cancer the subject is in need of treatment of, the use of the composition as part of a multi-drug regimen, the particular form of the antibody or antibody conjugate of the invention, the manner of administration, and the composition employed.

The pharmaceutically acceptable carrier or vehicle may be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

The term “carrier” refers to a diluent, adjuvant or excipient, with which a conjugate of the invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to a subject, the conjugates of the invention and pharmaceutically acceptable carriers are sterile. Water is a preferred carrier when the conjugate of the invention is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The composition may be intended for oral administration, and if so, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as ethyl cellulose, carboxymethylcellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the pharmaceutical composition is in the form of a capsule, e.g., a gelatin capsule, it can contain, in addition to materials of the above type, a liquid carrier such as polyethylene glycol, cyclodextrin or a fatty oil.

The pharmaceutical composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates and agents for the adjustment of tonicity such as sodium chloride or dextrose. A parenteral composition can be enclosed in an ampoule, a disposable syringe or a multiple-dose vial made of glass, plastic or other material. Physiological saline is a preferred adjuvant. An injectable composition is preferably sterile.

The pharmaceutical compositions comprise an effective amount of an antibody or antibody conjugate of the invention such that a suitable dosage will be obtained (see infra, for suitable dosages). Typically, this amount is at least 0.01% of an antibody or antibody conjugate of the invention by weight of the composition. When intended for oral administration, this amount can be varied to be between 0.1% and 80% by weight of the composition. Preferred oral compositions can comprise from between 4% and 50% of the antibody or antibody conjugate of the invention by weight of the composition. Preferred compositions of the present invention are prepared so that a parenteral dosage unit contains from between 0.01% and 2% by weight of the antibody or antibody conjugate of the invention.

The compositions of the invention can be administered by any convenient route, for example, by infusion or bolus injection, by absorption through epithelial or mucocutaneous linings (e.g., oral mucosa, rectal and intestinal mucosa, etc.). Administration can be systemic or local. Various delivery systems are known, e.g., microparticles, microcapsules, capsules, etc., and may be useful for administering an antibody or antibody conjugate of the invention. In certain embodiments, more than one antibody or antibody conjugate of the invention is administered to a subject. Methods of administration may include, but are not limited to, oral administration and parenteral administration; parenteral administration including, but not limited to, intradermal, intramuscular, intraperitoneal, intravenous, subcutaneous; intranasal, epidural, sublingual, intranasal, intracerebral, intraventricular, intrathecal, intravaginal, transdermal, rectally, by inhalation, or topically to the ears, nose, eyes, or skin. The preferred mode of administration is left to the discretion of the practitioner, and will depend in-part upon the site of the medical condition (such as the site of cancer, a cancerous tumor or a pre-cancerous condition).

In one embodiment, the antibodies or antibody conjugates of the invention are administered parenterally. In a specific embodiment, the antibodies or antibody conjugates of the invention are administered intravenously. In another embodiment, the antibodies or antibody conjugates of the invention are administered by continuous infusion. In a particular embodiment, the antibodies or antibody conjugates of the invention are administered by an infusion that lasts for about 15 minutes, about 20 minutes, about 30 minutes, about 45 minutes, about 1 hour, or about 2 hours.

In specific embodiments, it can be desirable to administer one or more antibodies or antibody conjugates of the invention locally to the area in need of treatment. This can be achieved, for example, and not by way of limitation, by local infusion during surgery; topical application, e.g., in conjunction with a wound dressing after surgery; by injection; by means of a catheter; by means of a suppository; or by means of an implant, the implant being of a porous, non-porous, or gelatinous material, including membranes, such as silastic membranes, or fibers. In one embodiment, administration can be by direct injection at the site (or former site) of a cancer, tumor, or precancerous tissue. In certain embodiments, it can be desirable to introduce one or more antibodies or antibody conjugates of the invention into the central nervous system by any suitable route, including intraventricular and intrathecal injection. Intraventricular injection can be facilitated by an intraventricular catheter, for example, attached to a reservoir, such as an Ommaya reservoir. In certain embodiments, one or more agents of the invention can be injected intraperitoneally.

Pulmonary administration can also be employed, e.g., by use of an inhaler or nebulizer, and formulation with an aerosolizing agent, or via perfusion in a fluorocarbon or synthetic pulmonary surfactant. In certain embodiments, the antibodies or antibody conjugates of the invention can be formulated as a suppository, with traditional binders and carriers such as triglycerides.

In yet another embodiment, the antibody or antibody conjugates of the invention can be delivered in a controlled release system. In one embodiment, a pump can be used (see Sefton, CRC Crit. Ref Biomed. Eng. 1987, 14, 201; Buchwald et al., Surgery 1980, 88: 507; Saudek et al., N. Engl. J. Med. 1989, 321: 574). In another embodiment, polymeric materials can be used (see Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J. Macromol. Sci. Rev. Macromol. Chem. 1983, 23, 61; see also Levy et al., Science 1985, 228, 190; During et al., Ann. Neurol., 1989, 25, 351; Howard et al., J. Neurosurg., 1989, 71, 105). In yet another embodiment, a controlled-release system can be placed in proximity of the target of the antibodies or antibody conjugates of the invention, e.g., the brain, thus requiring only a fraction of the systemic dose (see, e.g., Goodson, in Medical Applications of Controlled Release, herein, vol. 2, 1984, pp. 115-138). Other controlled-release systems discussed in the review by Langer (Science 1990, 249, 1527-1533) can be used.

In another embodiment, polymeric materials can be used to achieve controlled or sustained release of the antibodies or antibody conjugates of the invention (see, e.g., U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in Remington's Pharmaceutical Sciences by E. W. Martin.

Sustained or directed release compositions that can be formulated include, but are not limited to, antibodies or antibody conjugates of the invention protected with differentially degradable coatings, e.g., by microencapsulation, multiple coatings, etc. It is also possible to freeze-dry the compositions and use the lyophilizates obtained, for example, for the preparation of products for injection.

In a preferred embodiment, the antibodies or antibody conjugates of the invention are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetics such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachet indicating the quantity of active agent. Where an antibody or antibody conjugate of the invention is to be administered by infusion, it can be dispensed, for example, with an infusion bottle containing sterile pharmaceutical grade water or saline. Where the antibody or antibody conjugate of the invention is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving complex are also suitable for orally administered compositions of the invention. In these later platforms, fluid from the environment surrounding the capsule is imbibed by the driving complex, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard carriers such as mannitol, lactose, starch, magnesium stearate, sodium saccharine, cellulose, magnesium carbonate, etc. Such carriers are preferably of pharmaceutical grade.

The pharmaceutical compositions of the invention can be intended for topical administration, in which case the carrier can be in the form of a solution, emulsion, ointment or gel base. The base, for example, can comprise one or more of the following: petrolatum, lanolin, polyethylene glycols, beeswax, mineral oil, diluents such as water and alcohol, and emulsifiers and stabilizers. Thickening agents can be present in a composition for topical administration. If intended for transdermal administration, the composition can be in the form of a transdermal patch or an iontophoresis device. Topical formulations can comprise a concentration of a conjugate of the invention of from between 0.01% and 10% w/v (weight per unit volume of composition).

The compositions can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The compositions can consist of gaseous dosage units, e.g., it can be in the form of an aerosol. The term aerosol is used to denote a variety of systems ranging from those of colloidal nature to systems consisting of pressurized packages. Delivery can be by a liquefied or compressed gas or by a suitable pump system that dispenses the active ingredients. Aerosols of the compositions can be delivered in single phase, bi-phasic, or tri-phasic systems in order to deliver the composition. Delivery of the aerosol includes the necessary container, activators, valves, subcontainers, spacers and the like, which together can form a kit. Preferred aerosols can be determined by one skilled in the art, without undue experimentation.

Whether in solid, liquid or gaseous form, the compositions of the present invention can comprise an additional active agent selected from among those including, but not limited to, an additional prophylactic agent, an additional therapeutic agent, an antiemetic agent, a hematopoietic colony stimulating factor, an adjuvant therapy, a vaccine or other immune stimulating agent, an antibody/antibody fragment-based agent, an anti-depressant and an analgesic agent. For instance in a particular embodiment, the pharmaceutical composition comprises a conjugate of the invention, an additional agent, and a pharmaceutically acceptable carrier or vehicle.

The pharmaceutical compositions can be prepared using methodology well known in the pharmaceutical art. For example, a composition intended to be administered by injection can be prepared by combining a conjugate of the invention with water so as to form a solution. A surfactant can be added to facilitate the formation of a homogeneous solution or suspension. Surfactants are complexes that can non-covalently interact with a conjugate of the invention so as to facilitate dissolution or homogeneous suspension of the conjugate of the invention in the aqueous delivery system.

In one embodiment, the pharmaceutical compositions of the present invention may comprise one or more other therapies.

The present invention provides pharmaceutical compositions comprising an antibody of the invention in an amount effective to reduce a cancer stem cell population and/or cancer cell population in an animal with or animal model for myeloid leukemia or another cancer associated with cells expressing a surface antigen described herein by about 25%, 30%, 40%, 50%, 75%, 80%, 85%, 90%, 95% or 98% relative to a negative control. In a specific embodiment, the reduction in the cancer stem cell population in the animal or animal model is at least 25% relative to a negative control. In some embodiments, the animal is a human. In other embodiments, the animal is a non-human animal.

The present invention provides pharmaceutical compositions comprising an antibody conjugate in an amount effective to reduce a cancer stem cell population and/or cancer cell population in an animal with or animal model for myeloid leukemia or another cancer associated with cells expressing a surface antigen described herein by about 25%, 30%, 40%, 50%, 75%, 80%, 85%, 90%, 95% or 98% relative to a negative control, wherein the conjugate comprises an antibody that binds to a surface antigen described herein and a cytotoxic agent or other anticellular moiety. In a specific embodiment, the reduction in the cancer stem cell population in the animal or animal model is at least 25% relative to a negative control. In some embodiments, the animal is human. In other embodiments, the animal is a non-human animal.

Therapeutic and Prophylactic Uses of Conjugates

The present invention provides methods of treating and/or managing a disorder characterized by cells expressing a surface antigen described herein, the methods comprising administering to a subject (preferably, a human) in need thereof a pharmaceutical composition comprising an effective amount of an antibody or antibody conjugate of the invention. The present invention provides methods of preventing a disorder characterized by cells expressing a surface antigen described herein, the methods comprising administering to a subject (preferably, a human) in need thereof a pharmaceutical composition comprising an prophylactically effective amount of an antibody or antibody conjugate of the invention. In one embodiment, the antibody or antibody conjugates of the invention are administered as monotherapy for the prevention, treatment, and/or management of a disorder characterized by cells expressing a surface antigen described herein. In other embodiments, the antibody or antibody conjugates are administered in combination with another therapy. In certain embodiments, the antibodies and antibody conjugates of the invention are administered in combination, and optionally with other therapies.

The present invention is directed to therapies which involve administering one or more of the antibody or antibody conjugates of the invention and compositions comprising the antibody or antibody conjugates to a subject, preferably a human subject, for preventing, treating, managing, and/or ameliorating disease or disorder that displays or is characterized by expression of a surface antigen described herein or one or more symptoms thereof. In one embodiment, the invention provides a method of preventing, treating, managing, and/or ameliorating a disease or disorder that displays or is characterized by expression of a surface antigen described herein or one or more symptoms thereof, said method comprising administering to a subject in need thereof an effective amount of one or more antibody or antibody conjugates of the invention. Such diseases and disorders include cancer, allergic diseases, inflammatory diseases, and autoimmune diseases.

The invention also provides methods comprising administering to a subject in need thereof an antibody or antibody conjugate of the invention and one or more therapies (e.g., one or more therapeutic agents) other than the antibody or antibody conjugate of the invention that are currently being used, have been used, are known to be useful, or may be useful in the treatment, management, and/or amelioration of a disease or disorder that displays or is characterized by expression of a surface antigen described herein or one or more symptoms thereof. The therapeutic agents of the combination therapies of the invention can be administered sequentially or concurrently. The invention also provides methods comprising administering to a subject in need thereof an antibody or antibody conjugate of the invention and one or more therapies (e.g., one or more prophylactic agents) other than the antibody or antibody conjugate of the invention that are currently being used, have been used, are known to be useful, or may be useful in the prevention of a disease or disorder that displays or is characterized by expression of a surface antigen described herein or one or more symptoms thereof. The prophylactic agents of the combination therapies of the invention can be administered sequentially or concurrently. In a specific embodiment, the combination therapies of the invention comprise an effective amount of an antibody or antibody conjugate of the invention and an effective amount of at least one other therapy which has the same mechanism of action as said an antibody or antibody conjugate. In a specific embodiment, the combination therapies of the invention comprise an effective amount of an antibody or antibody conjugate of the invention and an effective amount of at least one other therapy (e.g., prophylactic or therapeutic agent) which has a different mechanism of action than said antibody or antibody conjugate. In certain embodiments, the combination therapies of the present invention improve the prophylactic or therapeutic effect of an antibody or antibody conjugate of the invention by functioning together with an antibody or antibody conjugate to have an additive or synergistic effect. In certain embodiments, the combination therapies of the present invention reduce the side effects associated with the prophylactic or therapeutic agents. In certain embodiments, the combination therapies of the present invention improve the therapeutic effect of an antibody or antibody conjugate of the invention by functioning together with an antibody or antibody conjugate to have an additive or synergistic effect. In other embodiments, the combination therapies of the present invention reduce the side effects associated with the prophylactic agents. In other embodiments, the combination therapies are administered prior to, during, or after the administration of the compositions of the invention.

Cancer or a neoplastic disease, including, but not limited to, neoplasms, tumors, metastases, or any disease or disorder characterized by uncontrolled cell growth, can be treated, suppressed, delayed, managed, inhibited or prevented by administering to a subject in need thereof a prophylactically effective regimen or a therapeutically effective regimen, the regimen comprising administering to the patient an agent of the invention. In specific embodiments, the invention encompasses the treatment, suppression, delaying, management, inhibiting of growth and/or progression, and prevention of cancer or neoplastic disease as described herein.

In one embodiment, the antibody or antibody conjugates of the invention are administered as monotherapy for the prevention, treatment, and/or management of cancer.

One aspect of the invention relates to a method of preventing, treating, and/or managing cancer in a patient (e.g., a human patient), the method comprising administering to the patient a prophylactically effective regimen or a therapeutically effective regimen, the regimen comprising administering to the patient an antibody or antibody conjugate of the invention or a pharmaceutical composition of the invention, wherein the patient has been diagnosed with cancer.

One aspect of the invention relates to a method of treating and/or managing cancer in a patient (e.g., a human patient), the method comprising administering to the patient a therapeutically effective regimen, the regimen comprising administering to the patient an antibody or antibody conjugate of the invention or a pharmaceutical composition of the invention, wherein the patient has relapsed from cancer. Another aspect of the invention relates to a method of preventing cancer in a patient (e.g., a human patient), the method comprising administering to the patient a prophylactically effective regimen, the regimen comprising administering to the patient an antibody or antibody conjugate of the invention or a pharmaceutical composition of the invention, wherein the patient has relapsed from cancer.

One aspect of the invention relates to a method of treating and/or managing cancer in a patient (e.g., a human patient), the method comprising administering to the patient a therapeutically effective regimen, the regimen comprising administering to the patient an antibody or antibody conjugate of the invention or a pharmaceutical composition of the invention, wherein the patient has failed or is failing therapy. One aspect of the invention relates to a method of preventing cancer in a patient (e.g., a human patient), the method comprising administering to the patient a prophylactically effective regimen, the regimen comprising administering to the patient an antibody or antibody conjugate of the invention or a pharmaceutical composition of the invention, wherein the patient has failed or is failing therapy.

One aspect of the invention relates to a method of treating and/or managing cancer in a patient (e.g., a human patient), the method comprising administering to the patient a therapeutically effective regimen, the regimen comprising administering to the patient an antibody or antibody conjugate of the invention or a pharmaceutical composition of the invention, wherein the patient is in remission from cancer. One aspect of the invention relates to a method of preventing cancer in a patient (e.g., a human patient), the method comprising administering to the patient a prophylactically effective regimen, the regimen comprising administering to the patient an antibody or antibody conjugate of the invention or a pharmaceutical composition of the invention, wherein the patient is in remission from cancer.

One aspect of the invention relates to a method of treating and/or managing cancer in a patient (e.g., a human patient), the method comprising administering to the patient a therapeutically effective regimen, the regimen comprising administering to the patient an antibody or antibody conjugate of the invention or a pharmaceutical composition of the invention, wherein the patient is refractory to therapy. One aspect of the invention relates to a method of preventing cancer in a patient (e.g., a human patient), the method comprising administering to the patient a prophylactically effective regimen, the regimen comprising administering to the patient an antibody or antibody conjugate of the invention or a pharmaceutical composition of the invention, wherein the patient is refractory to therapy.

In one embodiment, the cancer is a hematologic cancer. For instance, the cancer can be leukemia, lymphoma, myelodysplastic syndrome (MDS), or myeloma. In another embodiment, the cancer is a solid tumor.

In one embodiment of this aspect, the patient has received or is receiving another therapy. In another embodiment of this aspect, the patient has not previously received a therapy for the treatment and/or management of the cancer. In another embodiment of this aspect, the patient has not previously received a therapy for the prevention of the cancer.

The medical practitioner can diagnose the patient using any of the conventional cancer screening methods including, but not limited to physical examination (e.g., prostate examination, rectal examination, breast examination, lymph nodes examination, abdominal examination, skin surveillance, testicular exam, general palpation), visual methods (e.g., colonoscopy, bronchoscopy, endoscopy), PAP smear analyses (cervical cancer), stool guaiac analyses, blood tests (e.g., complete blood count (CBC) test, prostate specific antigen (PSA) test, carcinoembryonic antigen (CEA) test, cancer antigen (CA)-125 test, alpha-fetoprotein (AFP), liver function tests), karyotyping analyses, bone marrow analyses (e.g., in cases of hematological malignancies), histology, cytology, flow cytometry, a sputum analysis, and imaging methods (e.g., computed tomography (CT), magnetic resonance imaging (MRI), ultrasound, X-ray imaging, mammography, PET scans, bone scans, radionuclide scans).

In some embodiments, the prior therapy has failed in the patient. In some embodiments, the therapeutically effective regimen comprising administration of an antibody or antibody conjugate of the invention is administered to the patient immediately after the patient has undergone the prior therapy. For instance, in certain embodiments, the outcome of the prior therapy may be unknown before the patient is administered the antibody or antibody conjugate.

Another aspect of the invention relates to a method of preventing relapse of cancer in a patient (e.g., a human patient), the method comprising administering to a patient in need thereof a therapeutically effective regimen, the regimen comprising administering to the patient an antibody or antibody conjugate of the invention, wherein the cancer in the patient has entered remission. Another aspect of the invention relates to a method of preventing relapse of cancer in a patient (e.g., a human patient), the method comprising administering to a patient in need thereof a prophylactically effective regimen, the regimen comprising administering to the patient an antibody or antibody conjugate of the invention, wherein the cancer in the patient has entered remission. In some embodiments of this aspect, through administration of a therapeutically effective regimen, the medical practitioner can effectively cure the cancer, or prevent its reoccurrence. In some embodiments of this aspect, through administration of a prophylactically effective regimen, the medical practitioner can effectively cure the cancer, or prevent its reoccurrence.

Another aspect of the invention relates to a method of treating cancer in a patient (e.g., a human patient), the method comprising administering to a patient in need thereof a therapeutically effective regimen, the regimen comprising administering to the patient an agent or composition of the invention, wherein the antibody or antibody conjugate is administered at a dose that is lower than the maximum tolerated dose (MTD) over a period of three months, four months, six months, nine months, 1 year, 2 years, 3 years, 4 years, or more. Another aspect of the invention relates to a method of preventing cancer in a patient (e.g., a human patient), the method comprising administering to a patient in need thereof a prophylactically effective regimen, the regimen comprising administering to the patient an agent or composition of the invention, wherein the antibody or antibody conjugate is administered at a dose that is lower than the maximum tolerated dose (MTD) over a period of three months, four months, six months, nine months, 1 year, 2 years, 3 years, 4 years, or more.

Another aspect of the invention relates to a method of treating and/or managing cancer in a patient (e.g., a human patient), the method comprising administering to a patient in need thereof a therapeutically effective regimen, the regimen comprising administering to the patient an antibody or antibody conjugate of the invention, wherein the antibody or antibody conjugate is administered at a dose that is lower than the human equivalent dosage (HED) of the no observed adverse effect level (NOAEL) over a period of three months, four months, six months, nine months, 1 year, 2 years, 3 years, 4 years, or more. The NOAEL, as determined in animal studies, is useful in determining the maximum recommended starting dose for human clinical trials. For instance, the NOAELs can be extrapolated to determine human equivalent dosages. Typically, such extrapolations between species are conducted based on the doses that are normalized to body surface area (i.e., mg/m²). In specific embodiments, the NOAELs are determined in mice, hamsters, rats, ferrets, guinea pigs, rabbits, dogs, primates (monkeys, marmosets, squirrel monkeys, baboons), micropigs, or minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005. Yet another aspect of the invention relates to a method of preventing cancer in a patient (e.g., a human patient), the method comprising administering to a patient in need thereof a prophylactically effective regimen, the regimen comprising administering to the patient an antibody or antibody conjugate of the invention, wherein the antibody or antibody conjugate is administered at a dose that is lower than the human equivalent dosage (HED) of the no observed adverse effect level (NOAEL).

While not being bound by any specific theory, Applicants believe that by the administration of the therapeutically effective regimens, the cancer stem cell population of a cancer/tumor is stabilized or reduced, so as to limit the potential repopulation of the tumor. In addition, while not being bound by any specific theory, Applicants believe that by the administration of the prophylactically effective regimens, the cancer stem cell population of a cancer/tumor is stabilized or reduced, so as to prevent the potential repopulation of the tumor.

In certain embodiments of these aspects, the regimens comprise administering a therapeutically effective regimen, wherein the regimen results in a reduction in the cancer stem cell population in the patient. In certain embodiments of these aspects, the regimens comprise administering a prophylactically effective regimen, wherein the regimen results in a reduction in the cancer stem cell population in the patient. In one embodiment, the patient undergoing the regimen is monitored to determine whether the regimen has resulted in a reduction in the cancer stem cell population in the patient.

Typically, the monitoring of the amount of cancer stem cells is conducted by detecting the amount of cancer stem cells in a specimen extracted from the patient. Methods of detecting the amount of cancer stem cells in a specimen are described infra in Section 5.9. This monitoring step is typically performed at least 1, 2, 4, 6, 7, 8, 10, 12, 14, 15, 16, 18, 20, or 30, 60, 90, 120 days, 6 months, 9 months, 12 months, or >12 months after the patient begins receiving the regimen.

In some embodiments, the specimen may be a blood specimen, wherein the amount of cancer stem cells per unit of volume (e.g., 1 ml) or other measured unit (e.g., per unit field in the case of a histological analysis) is quantitated. In certain embodiments, the amount of cancer stem cells is determined as a portion (e.g., a percentage) of the cancer cells present in the blood specimen, as a subset of the cancer cells present in the blood specimen, or as a subset of a subset of the cancer cells present in the blood specimen. The amount of cancer stem cells, in other embodiments, can be determined as a percentage of the total blood cells.

In other embodiments, the specimen extracted from the patient is a tissue specimen (e.g., a biopsy extracted from suspected cancerous tissue), where the amount of cancer stem cells can be measured, for example, on the basis of the amount of cancer stem cells per unit weight of the tissue. In certain embodiments, the amount of cancer stem cells is determined as a portion (e.g., a percentage) of the cancer cells present in the tissue, as a subset of the cancer cells present in the tissue, or as a subset of a subset of the cancer cells present in the tissue.

The amount of cancer stem cells in the extracted specimen can be compared with the amount of cancer stem cells measured in reference samples to assess the efficacy of the regimen, and the amelioration of the cancer under therapy. In one embodiment, the reference sample is a specimen extracted from the patient undergoing therapy, wherein the specimen is extracted from the patient at an earlier time point (e.g., prior to receiving the regimen, as a baseline reference sample, or at an earlier time point while receiving the therapy). In another embodiment, the reference sample is extracted from a healthy, noncancer-afflicted patient.

In other embodiments the amount of cancer stem cells in the extracted specimen can be compared with a predetermined reference range. In a specific embodiment, the predetermined reference range is based on i) the amount of cancer stem cells obtained from a population(s) of patients suffering from the same type of cancer as the patient undergoing the therapy, or ii) the amount of stem cells obtained from a population(s) of patients without cancer.

If the reduction in the amount of cancer stem cells is determined to be too small upon comparing the amount of cancer stem cells in the specimen extracted from the patient undergoing the regimen with the reference specimen, then the medical practitioner has a number of options to adjust the regimen. For instance, the medical practitioner can then increase either the dosage of the agent or composition of the invention administered, the frequency of the administration, the duration of administration, or any combination thereof. In a specific embodiment, after the determination is made, a second effective amount of an agent or composition of the invention can be administered to the patient.

In certain embodiments, if the reduction in the amount of cancer stem cells is determined to be acceptable upon comparing the amount of cancer stem cells in the sample obtained from the patient undergoing the therapeutic regimen with the reference sample, then the medical practitioner may elect not to adjust the regimen. In certain embodiments, if the reduction in the amount of cancer stem cells is determined to be acceptable upon comparing the amount of cancer stem cells in the sample obtained from the patient undergoing the prophylactic regimen with the reference sample, then the medical practitioner may elect not to adjust the regimen. For instance, the medical practitioner may elect not to increase either the dosage of the agent or composition of the invention being administered, the frequency of the administration, the duration of administration, or any combination thereof. Further, the medical practitioner may elect to add additional therapies or combine therapies.

In other embodiments, the regimens comprise administering a therapeutically effective regimen, wherein the regimen results in a reduction in the amount of cancer cells in the patient. In other embodiments, the regimens comprise administering a prophylactically effective regimen, wherein the regimen results in a reduction in the amount of cancer cells in the patient. In one embodiment, the patient undergoing the regimen is monitored to determine whether the regimen has resulted in a reduction in the amount of cancer cells in the patient.

Typically, the monitoring of the amount of cancer cells is conducted by detecting the amount of cancer cells in a specimen extracted from the patient. Methods of detecting the amount of cancer cells in a specimen are described infra in Section 5.10. This monitoring step is typically performed at least 1, 2, 4, 6, 7, 8, 10, 12, 14, 15, 16, 18, 20, or 30, 60, 90, 120 days, 6 months, 9 months, 12 months, or >12 months after the patient begins receiving the regimen.

In some embodiments, the specimen may be a blood specimen, wherein the amount of cancer cells per unit of volume (e.g., 1 ml) or other measured unit (e.g., per unit field in the case of a histological analysis) is quantitated. The cancer cell population, in certain embodiments, can be determined as a percentage of the total blood cells.

In some embodiments, the sample obtained from the patient may be a bone marrow specimen, wherein the amount of cancer cells per unit of volume (e.g., 1 ml) or other measured unit (e.g., per unit field in the case of a histological analysis) is quantitated. The cancer cell population, in certain embodiments, can be determined as a percentage of the total bone marrow cells.

In other embodiments, the specimen extracted from the patient is a tissue specimen (e.g., a biopsy extracted from suspected cancerous tissue), where the amount of cancer cells can be measured, for example, on the basis of the amount of cancer cells per unit weight of the tissue. The amount of cancer cells can also be measured using immunohistochemistry or flow cytometry.

The amount of cancer cells in the extracted specimen can be compared with the amount of cancer cells measured in reference samples to assess the efficacy of the regimen and amelioration of the cancer under therapy. In one embodiment, the reference sample is a specimen extracted from the patient undergoing therapy, wherein the specimen from the patient is extracted at an earlier time point (e.g., prior to receiving the regimen, as a baseline reference sample, or at an earlier time point while receiving the therapy). In another embodiment, the reference sample is extracted from a healthy, noncancer-afflicted patient.

In other embodiments the cancer cell population in the extracted specimen can be compared with a predetermined reference range. In a specific embodiment, the predetermined reference range is based on the amount of cancer cells obtained from a population(s) of patients suffering from the same type of cancer as the patient undergoing the therapy.

If the reduction in the cancer cell population is judged too small upon comparing the amount of cancer cells in the specimen extracted from the patients undergoing therapy with the reference specimen, then the medical practitioner has a number of options to adjust the therapeutic regimen. For instance, the medical practitioner can then either increase the dosage of the agent or composition of the invention administered, the frequency of the administration, the duration of administration, or any combination thereof. In a specific embodiment, after the determination is made, a second effective amount of a agent or composition of the invention can be administered to the patient.

Additional Therapies

Any therapy (e.g., therapeutic agent) which is useful, has been used, or is currently being used for the treatment and/or management of a disorder characterized by expression of a surface antigen described herein (e.g., cancer) can be used in compositions and methods of the invention. Any therapy (e.g., prophylactic agent) which is useful, has been used, or is currently being used for the prevention of a disorder characterized by expression of a surface antigen described herein (e.g., cancer) can be used in compositions and methods of the invention. Therapies include, but are not limited to, peptides, polypeptides, conjugates, nucleic acid molecules, small molecules, mimetic agents, synthetic drugs, inorganic molecules, and organic molecules. Non-limiting examples of cancer therapies include chemotherapies, radiation therapies, hormonal therapies, and/or biological therapies/immunotherapies and surgery. In certain embodiments, an effective regimen of the invention comprises the administration of a combination of therapies.

In some embodiments, the therapy(ies) used in combination with an antibody or antibody conjugate of the invention is an immunomodulatory agent.

In some embodiments, the therapy(ies) used in combination with an antibody or antibody conjugate of the invention is an anti-angiogenic agent.

In some embodiments, the therapy(ies) used in combination with an antibody or antibody conjugate of the invention is an inflammatory agent.

In certain embodiments, the therapy(ies) used is an alkylating agent, a nitrosourea, an antimetabolite, and anthracyclin, a topoisomerase II inhibitor, or a mitotic inhibitor.

The invention includes the use of agents that target cancer stem cells in combination with an antibody or antibody conjugate of the invention. In some embodiments, the agent used is an agent that binds to a marker, e.g., antigen on cancer stem cells. In a specific embodiment, the agent binds to an antigen that is expressed at a greater level on cancer stem cells than on normal stem cells. In a specific embodiment, the agent binds specifically to a cancer stem cell antigen. In other embodiments, the therapy(ies) used in accordance with the invention is an agent that binds to a marker on cancer stem cells. Non-limiting examples of antigens on cancer stem cells that can be used to target cancer stem cells include CD34+, CD44+, CD133+, CD34+, CD19+, CD20+, CD47+, CD96+, CD133+, and .alpha.2.beta.1hi. In one embodiment, the agent that binds to a marker on cancer stem cells is an antibody. In another embodiment, the agent that binds to a marker on cancer stem cells is a ligand. In certain embodiments, the antibody or ligand is attached directly or indirectly to a therapeutic moiety. Non-limiting examples of therapeutic moieties include, but are not limited to, therapeutic enzymes, chemotherapeutic agents, cytokines, radionuclides, antimetabolites, and toxins.

In certain embodiments, antibodies that bind to a marker on cancer stem cells are substantially non-immunogenic in the treated subject. Non-immunogenic antibodies include, but are not limited to, making the antibody chimeric, humanizing the antibody, and antibodies from the same species as subject receiving the therapy. Antibodies that bind to markers in cancer stem cells can be produced using techniques known in the art. See, for example, paragraphs 539-573 of U.S. Publ'n No. 2005/0002934 A1, which is incorporated by reference in its entirety.

In some embodiments, an antibody or antibody conjugate of the invention is used in combination with radiation therapy comprising the use of x-rays, gamma rays and other sources of radiation to destroy cancer stem cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer stem cells, cancer cells and/or a tumor mass.

Currently available cancer therapies and their dosages, routes of administration and recommended usage are known in the art and have been described in such literature as the Physician's Desk Reference (60th ed., 2006). In accordance with the present invention, the dosages and frequency of administration of chemotherapeutic agents are described herein.

In a specific embodiment, an improved method of treating cancer which involves the administration of a therapeutic agent which selectively kills, inhibits or modulates the growth of cancer cells wherein the improvement comprises administering a therapeutic agent which provides for the selective killing, inhibition or modulation of the growth of a cancer stem line (defined as symmetrically dividing stem cells) present within a cancerous tumor.

Target Cancers

Any type of cancer can be treated in accordance with the invention. Non-limiting examples of cancers that can be treated in accordance with the invention cancers include: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungaling (polyploid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy, et al., 1997, Informed Decisions. The Complete Book of Cancer Diagnosis, Treatment, and Recovery, Viking Penguin, Penguin Books U.S.A., Inc., United States of America).

The therapeutically effective regimens are also useful in the treatment of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T cell lymphoma, Burkitt's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoctanthoma, seminoma, thyroid follicular cancer and teratocarcinoma. In some embodiments, cancers associated with aberrations in apoptosis are treated in accordance with the methods of the invention. Such cancers may include, but are not limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders of the skin, lung, liver, bone, brain, stomach, colon, breast, prostate, bladder, kidney, pancreas, ovary, and/or uterus are treated in accordance with the methods of the invention. In other specific embodiments, a sarcoma, or melanoma is treated in accordance with the methods of the invention.

In a specific embodiment, the cancer being treated in accordance with the invention is leukemia, lymphoma, myeloma or myelodysplastic syndrome.

Non-limiting examples of leukemias and other blood-borne cancers that can be treated with the methods of the invention include acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocytic leukemia, acute undifferentiated leukemia, myelodysplastic syndrome (“MDS”), chronic myelocytic leukemia “CML”, chronic lymphocytic leukemia “CLL”, and hairy cell leukemia.

Non-limiting examples of lymphomas that can be treated in accordance with the methods of the invention include Hodgkin's disease, non-Hodgkin's Lymphoma, Multiple myeloma, Waldenstrom's macroglobulinemia, Heavy chain disease, and Polycythemia vera.

In another embodiment, the cancer being treated in accordance with the invention is a solid tumor. Examples of solid tumors that can be treated in accordance with the methods of the invention include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiform, astrocytoma, medulloblastoma, craniopharyngioma, ependymoma, pinealoma, hemangioblastoma, acoustic neuroma, oligodendroglioma, meningioma, skin cancer, melanoma, neuroblastoma, and retinoblastoma.

Methods of Treating Cancer

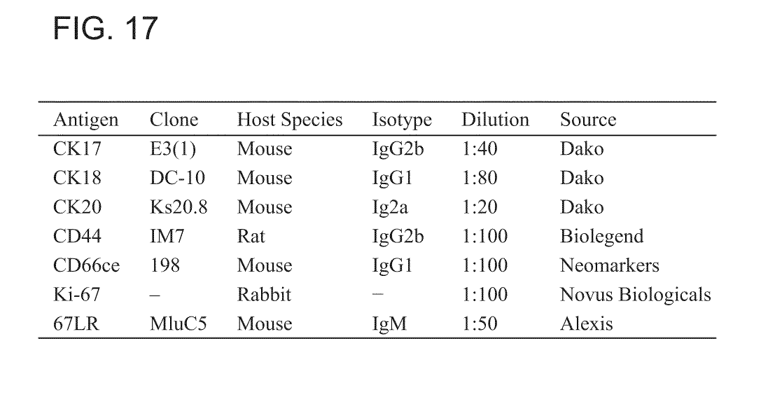

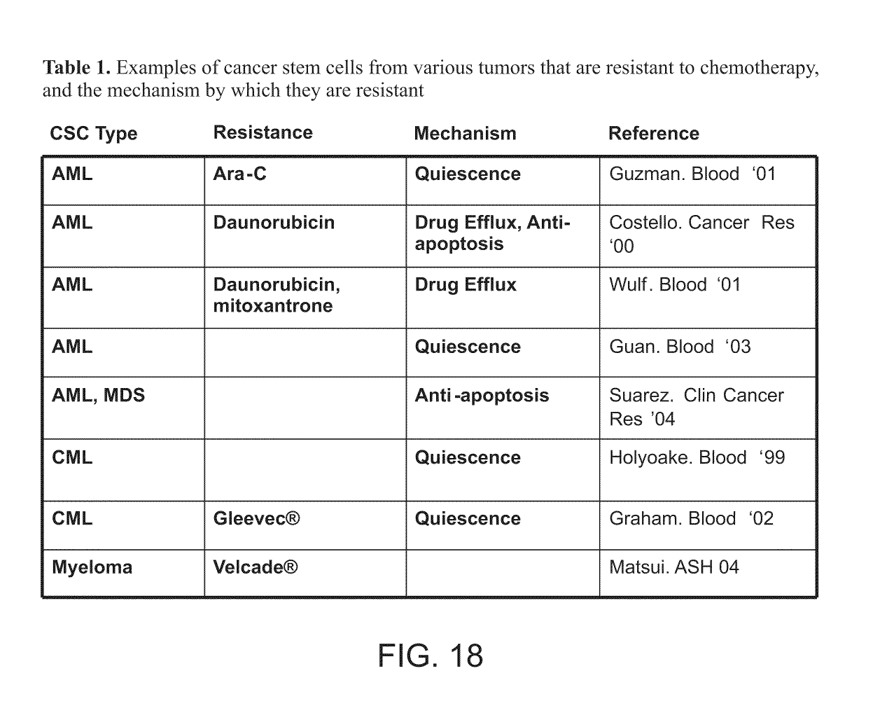

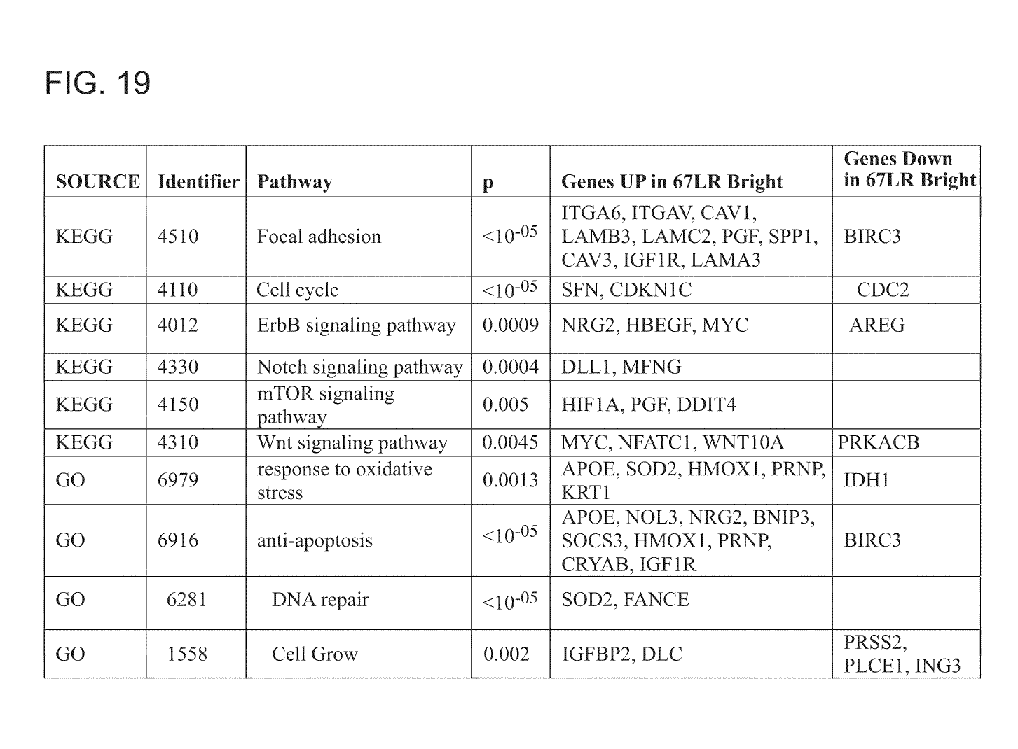

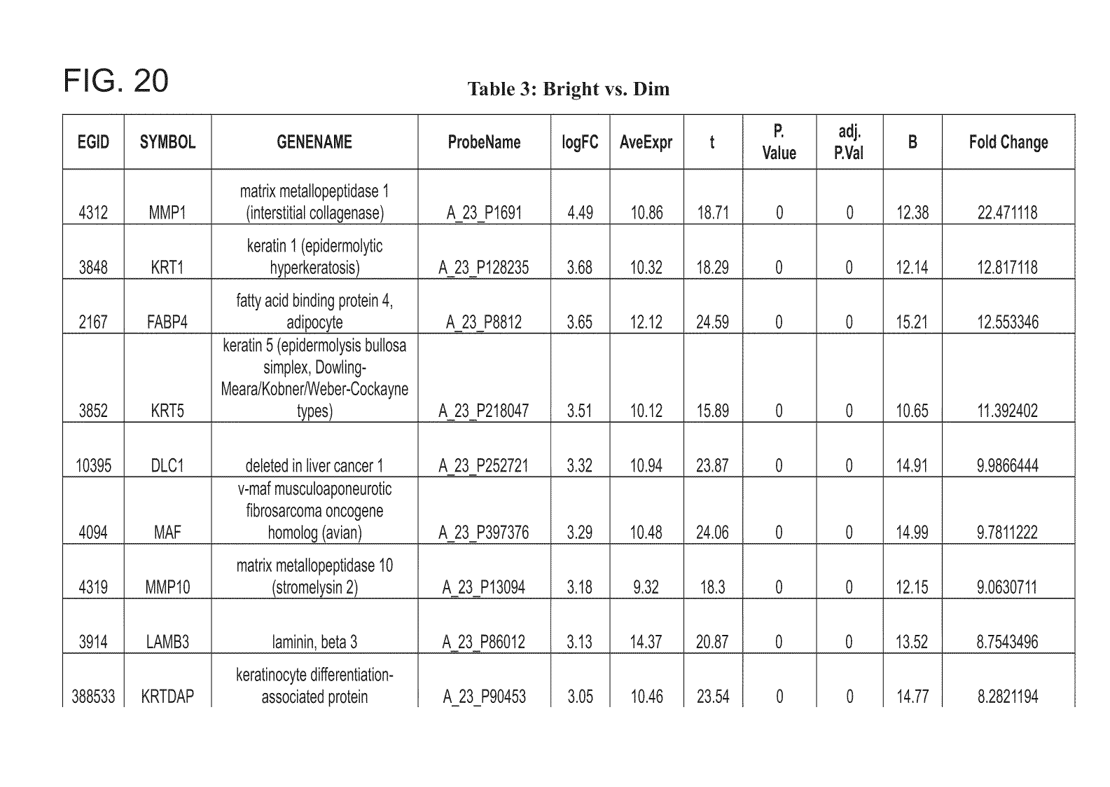

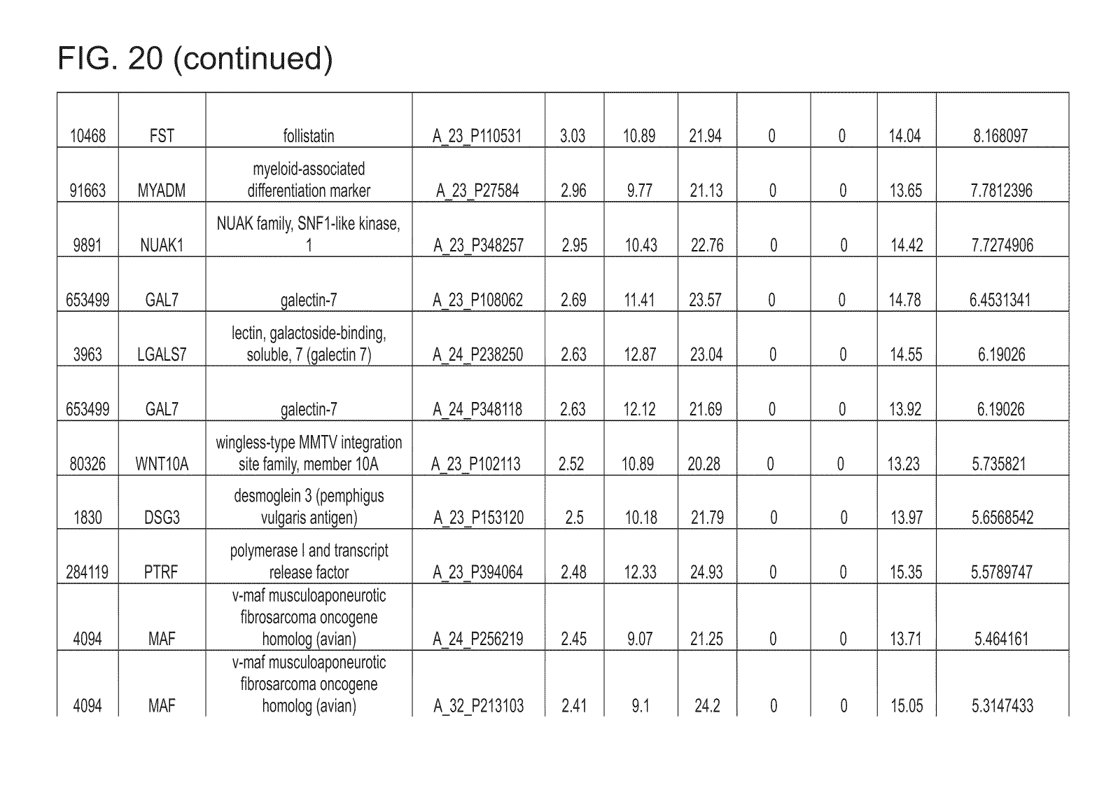

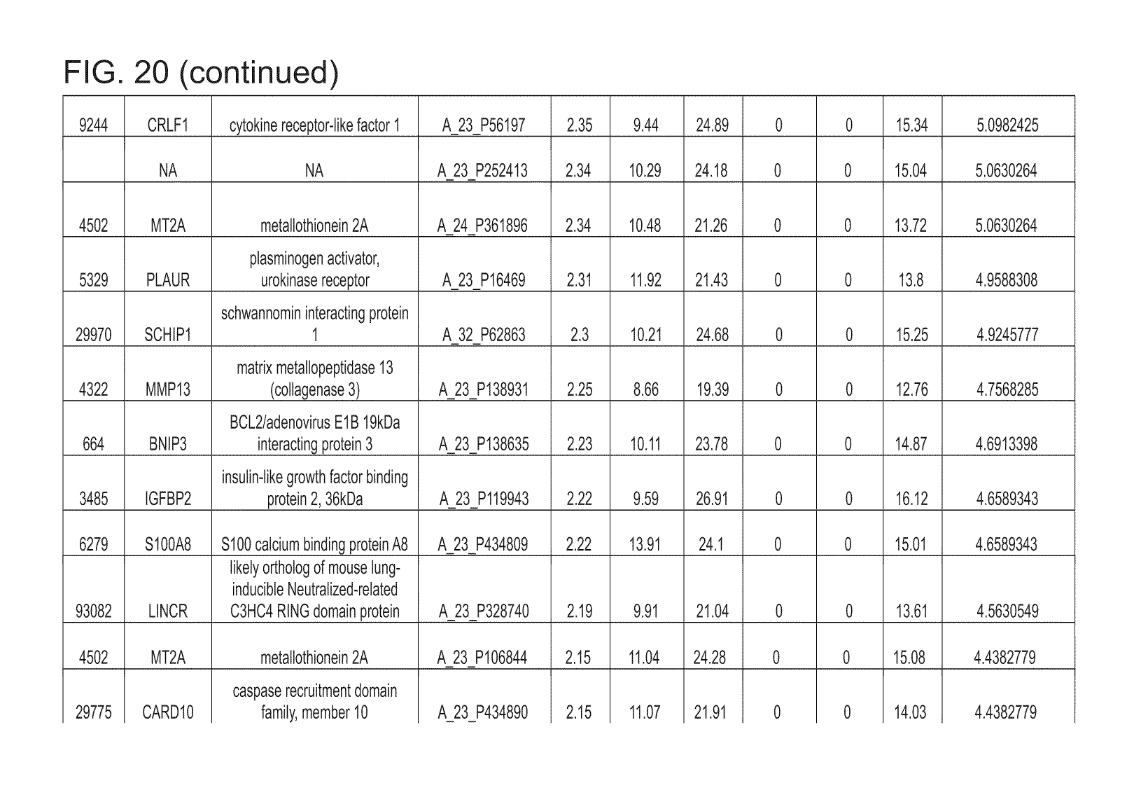

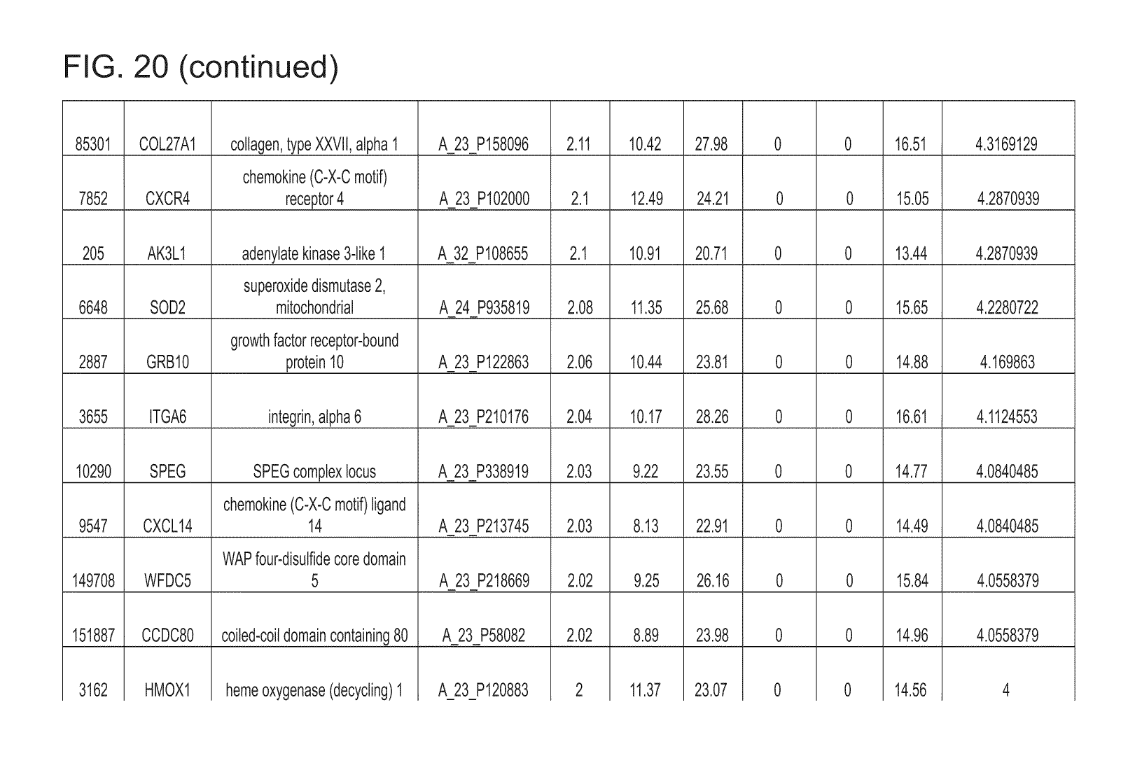

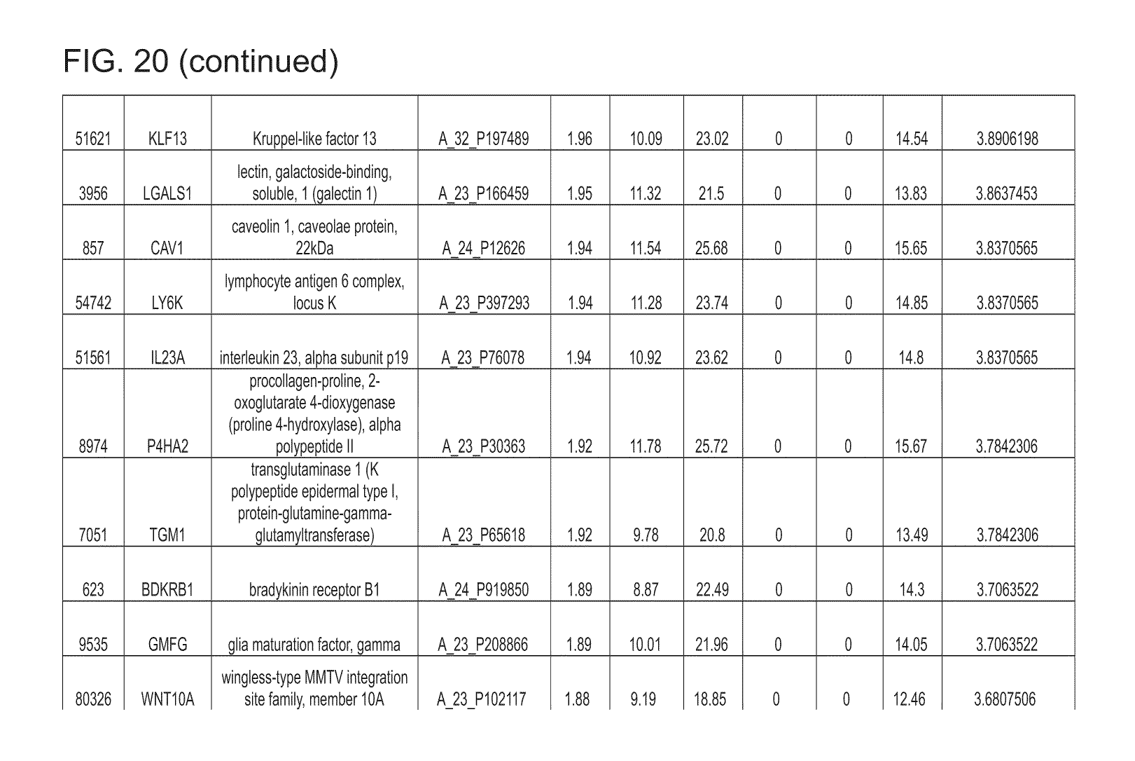

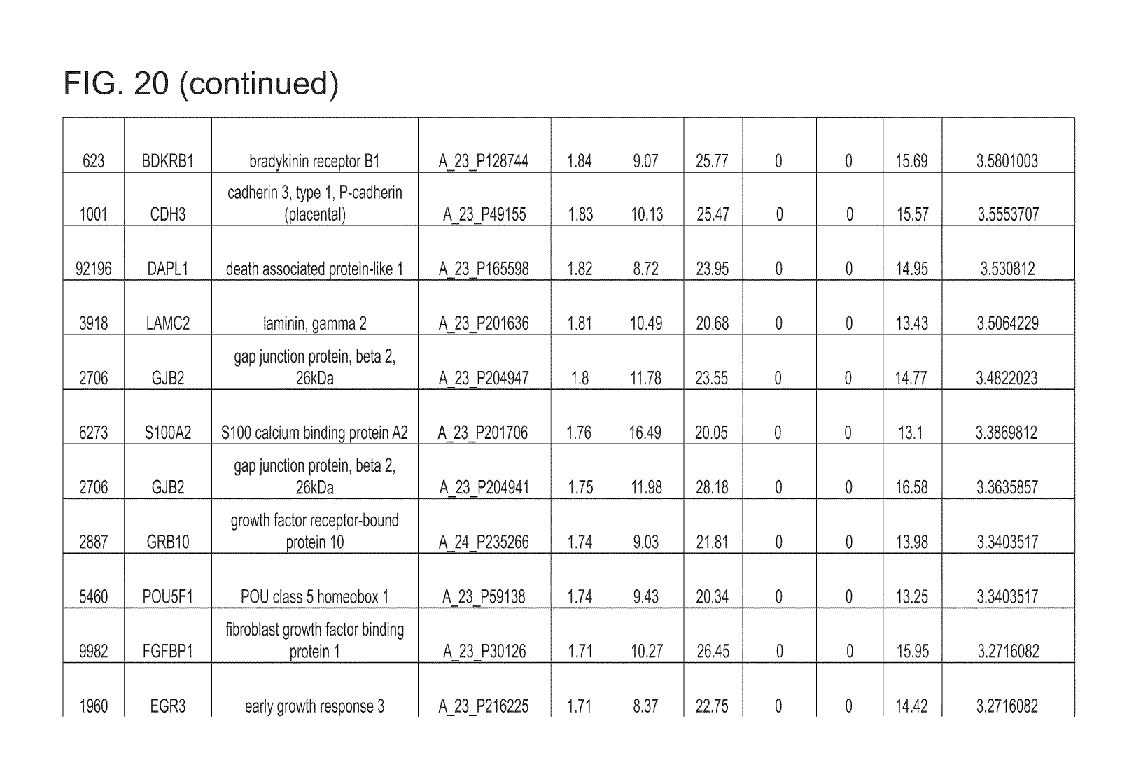

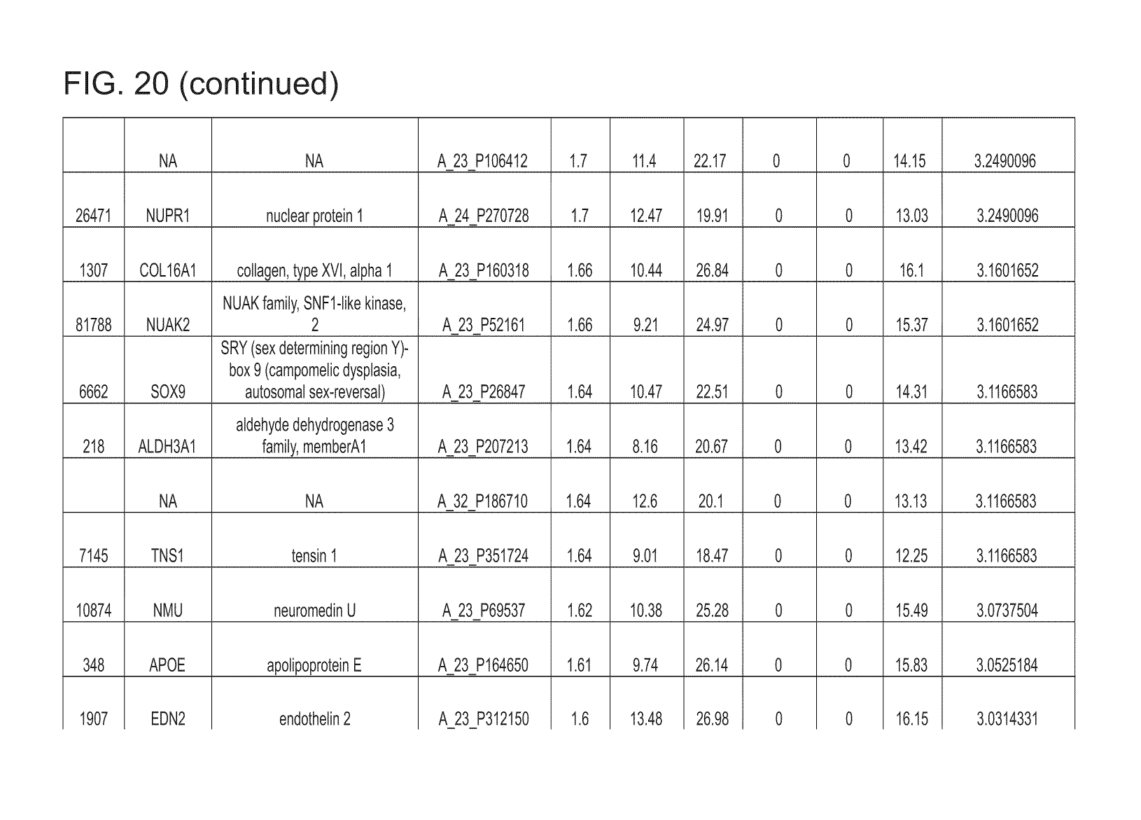

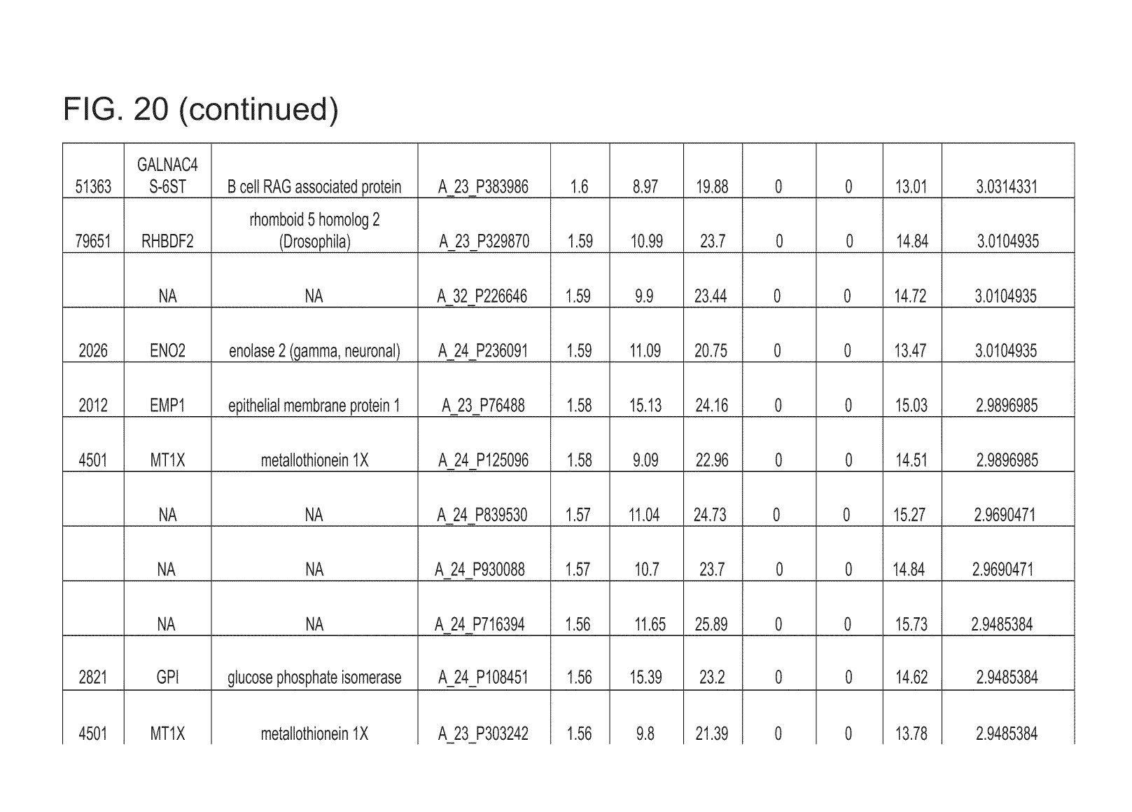

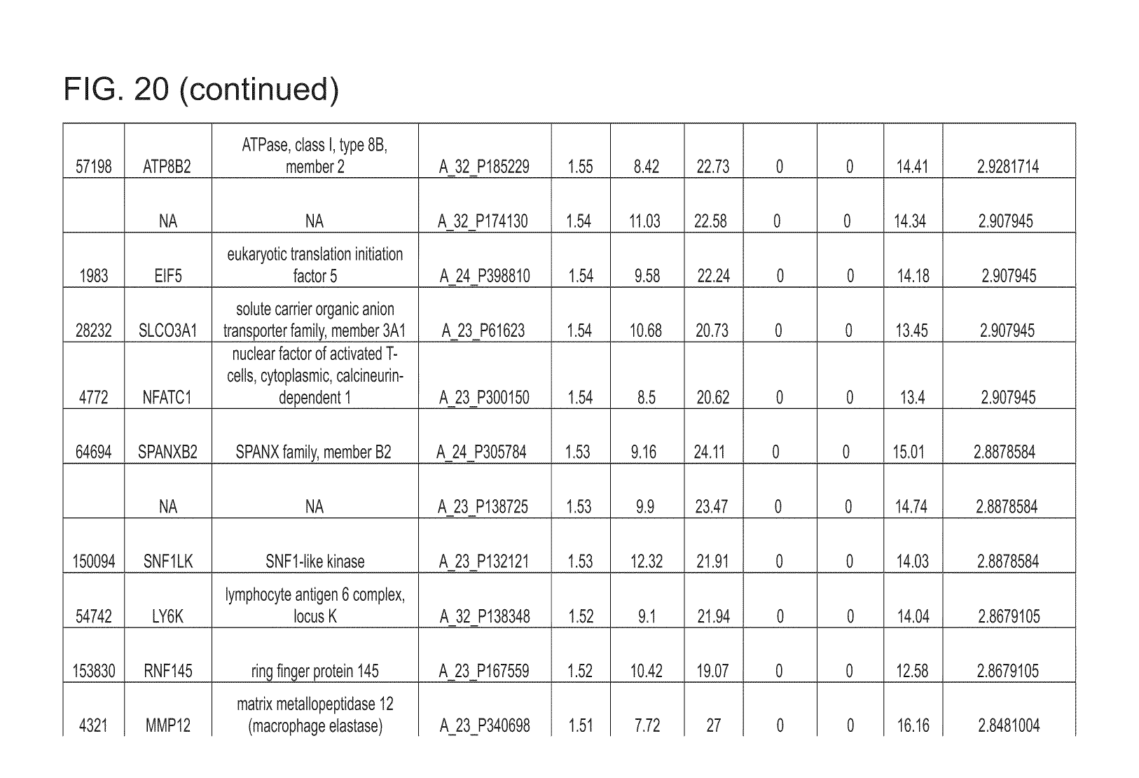

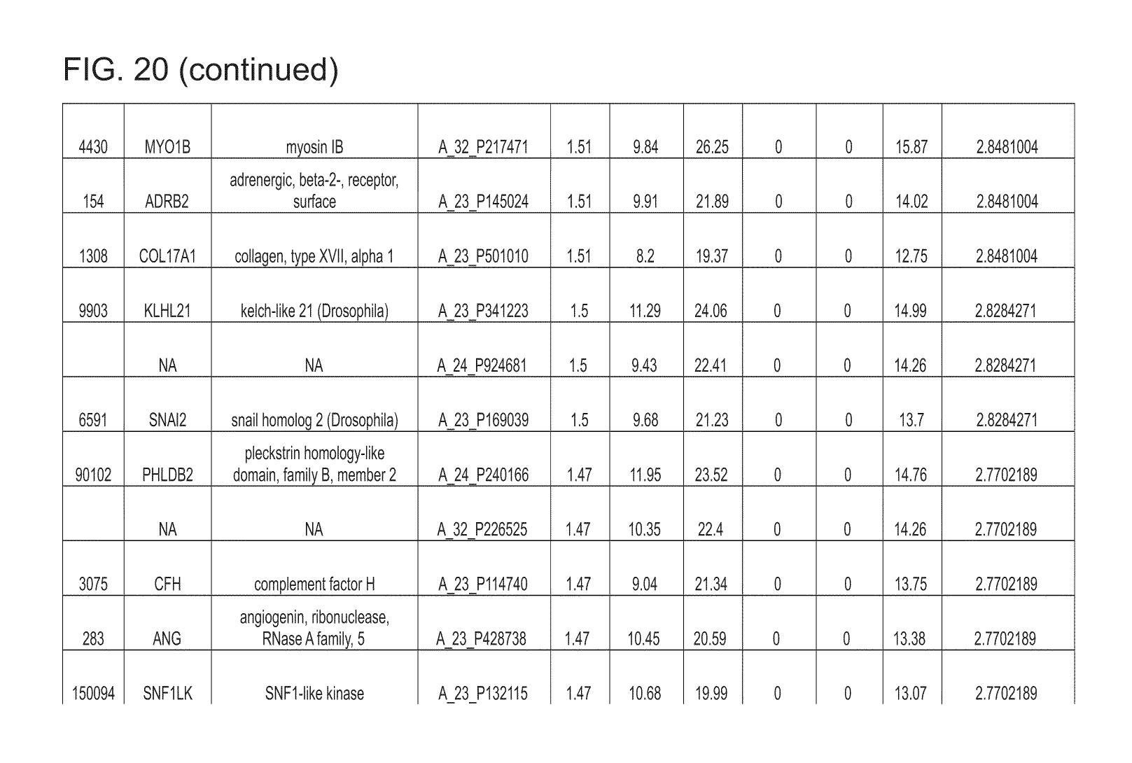

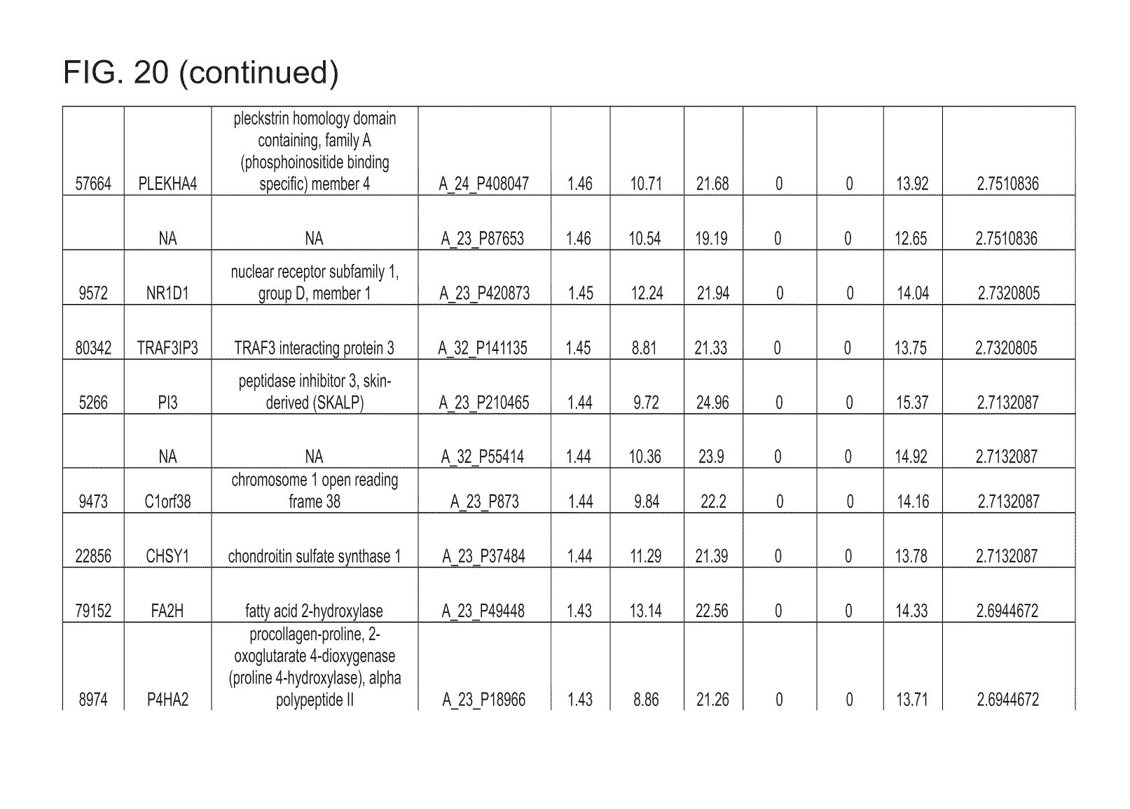

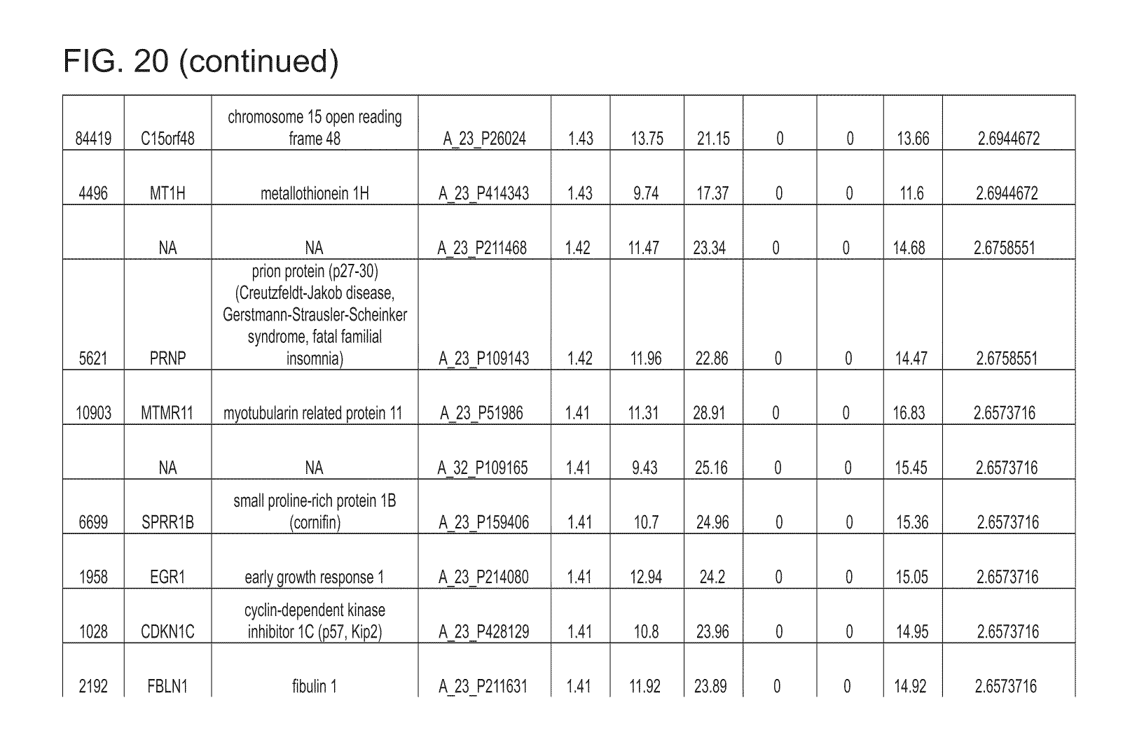

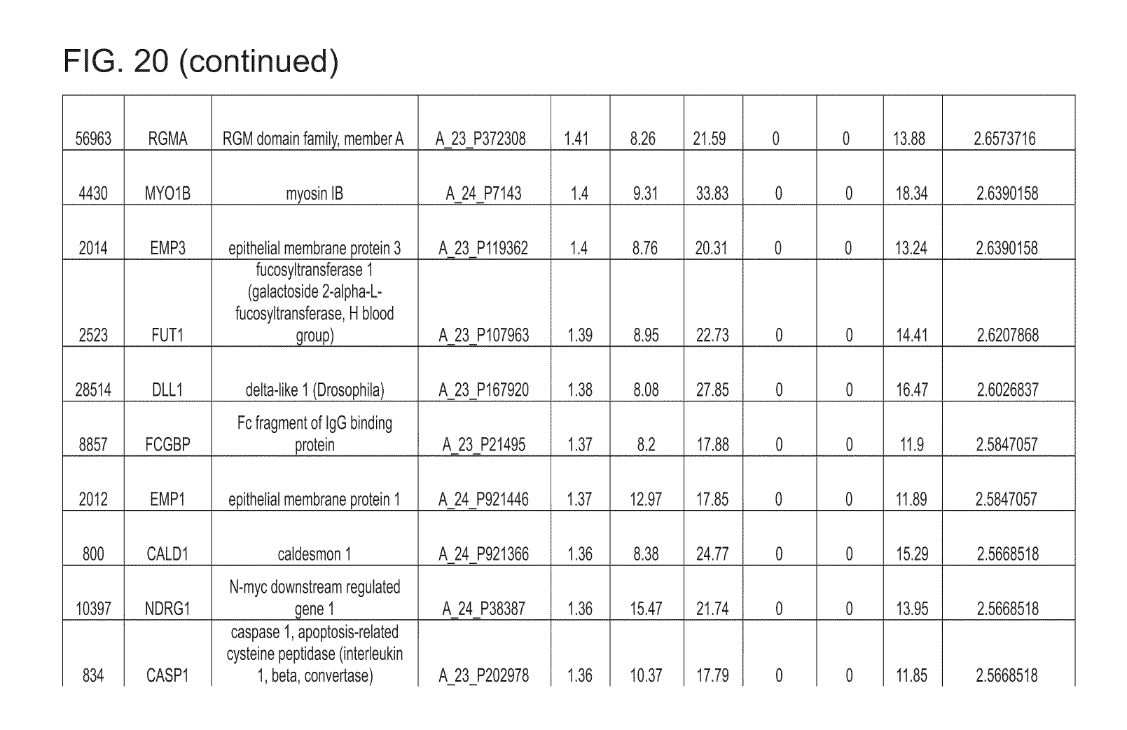

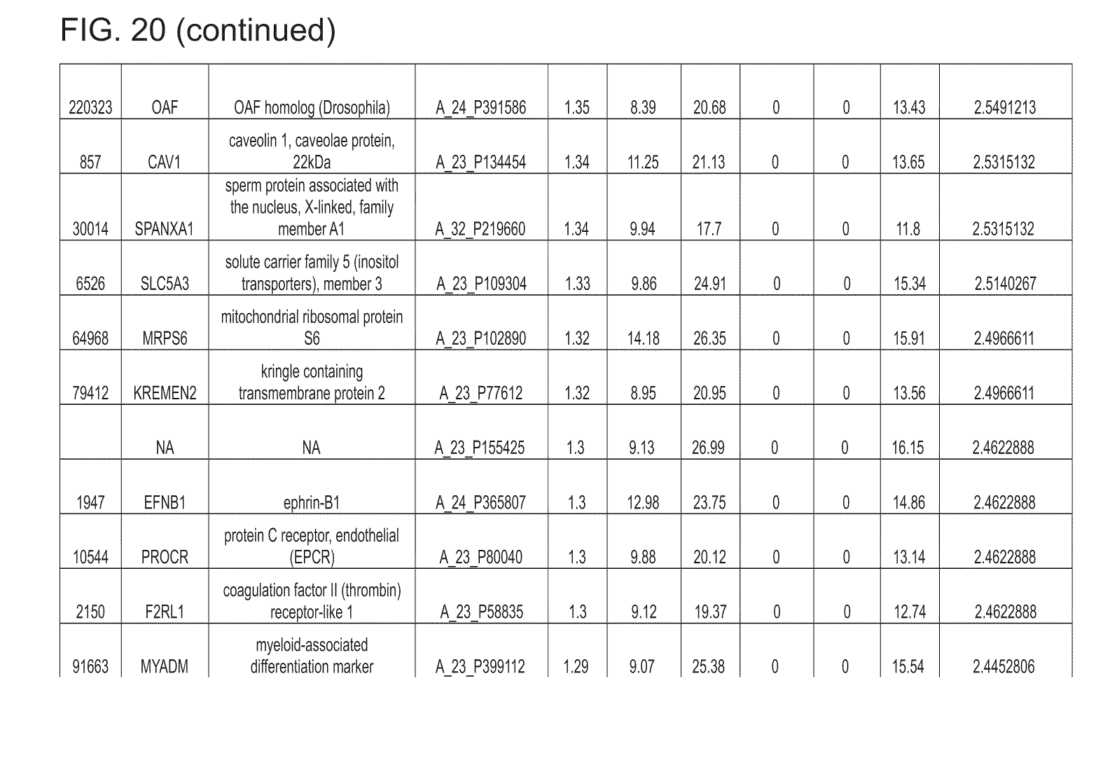

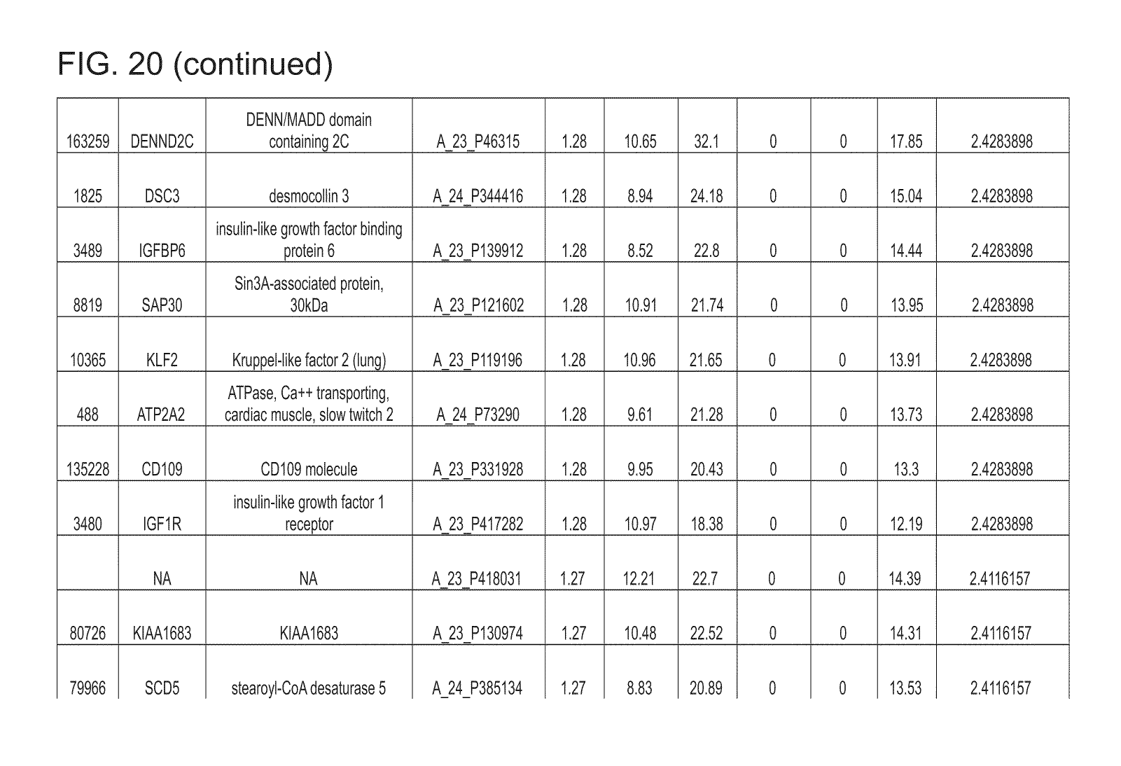

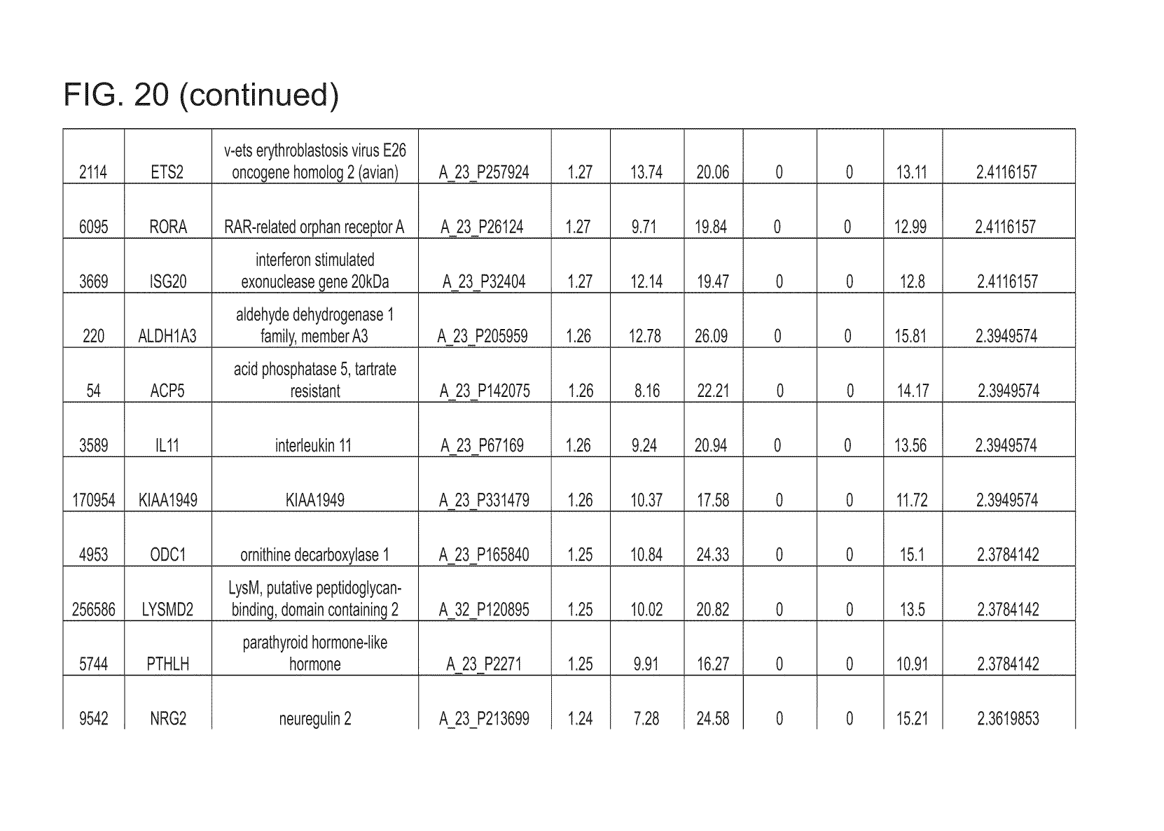

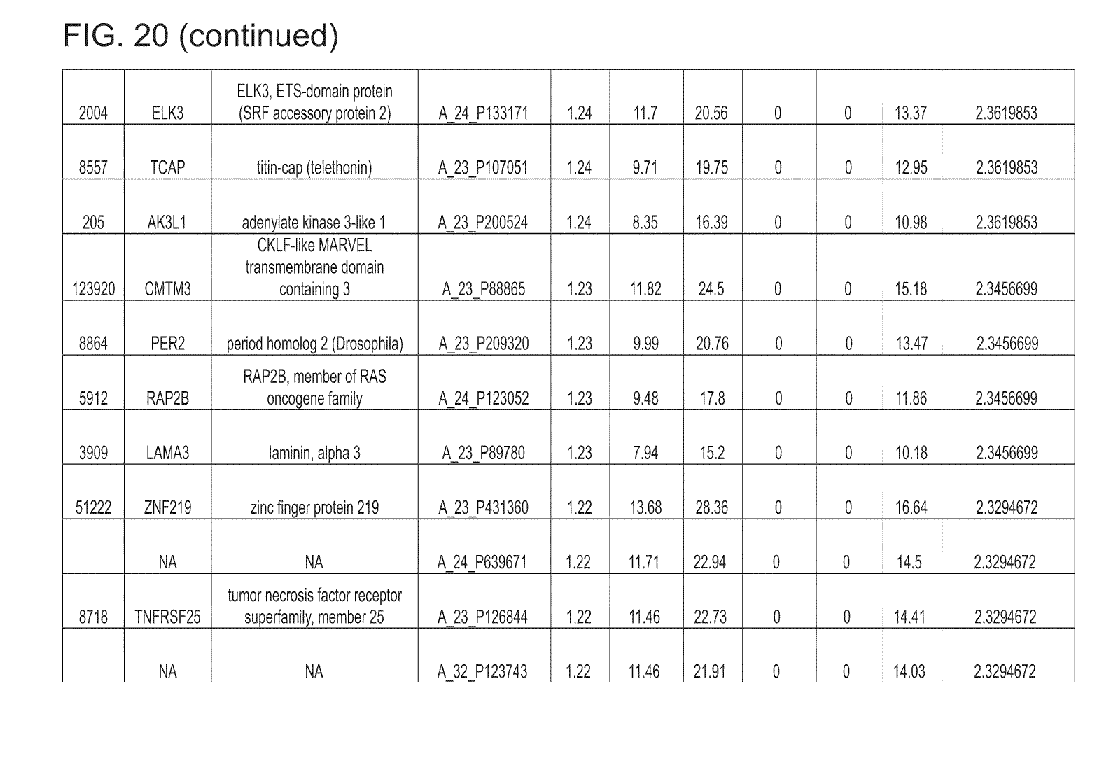

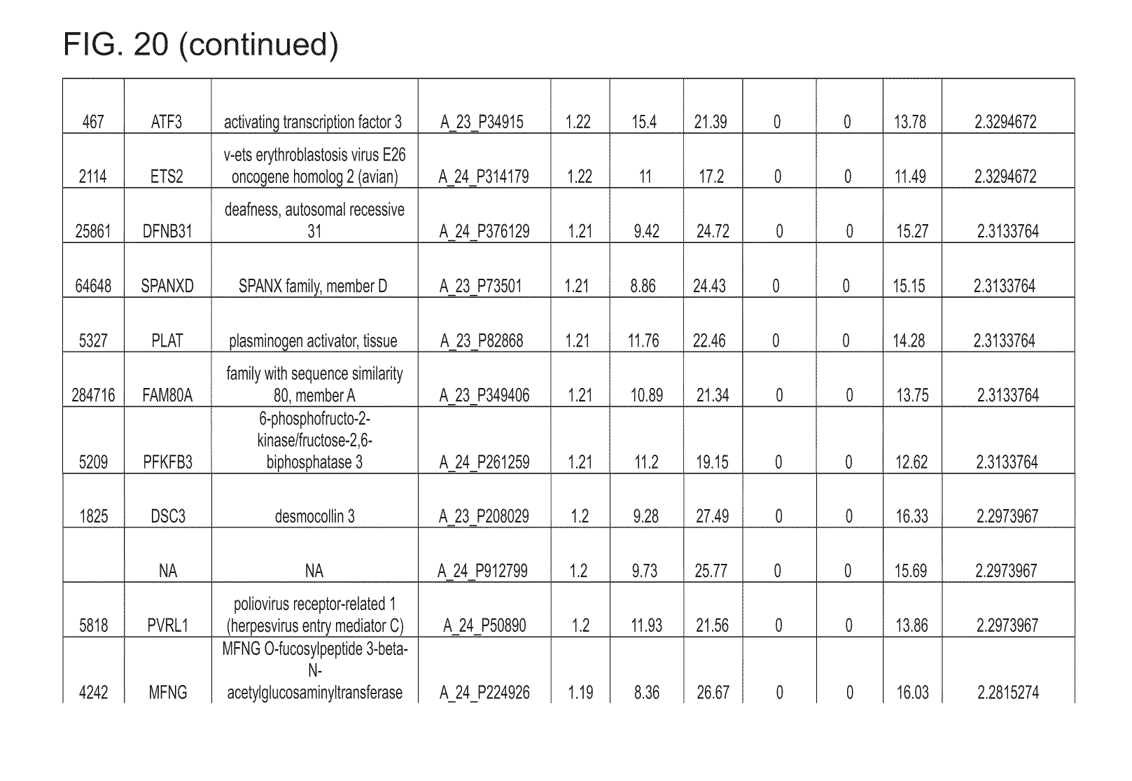

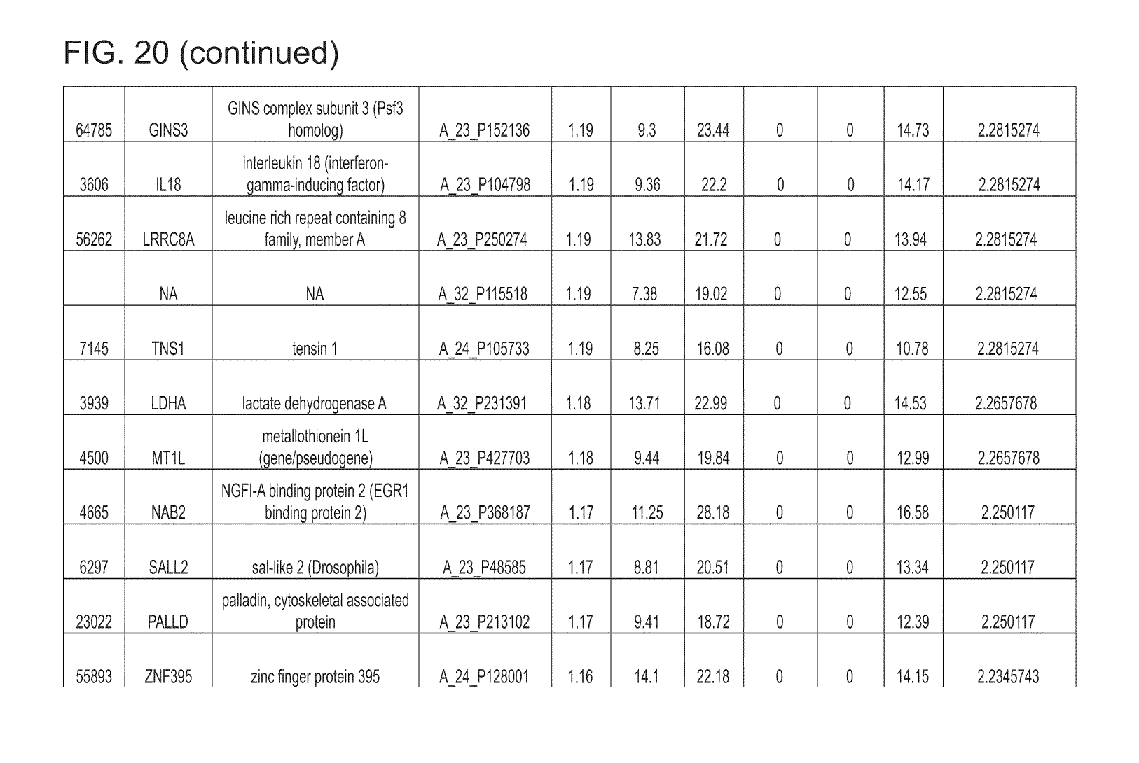

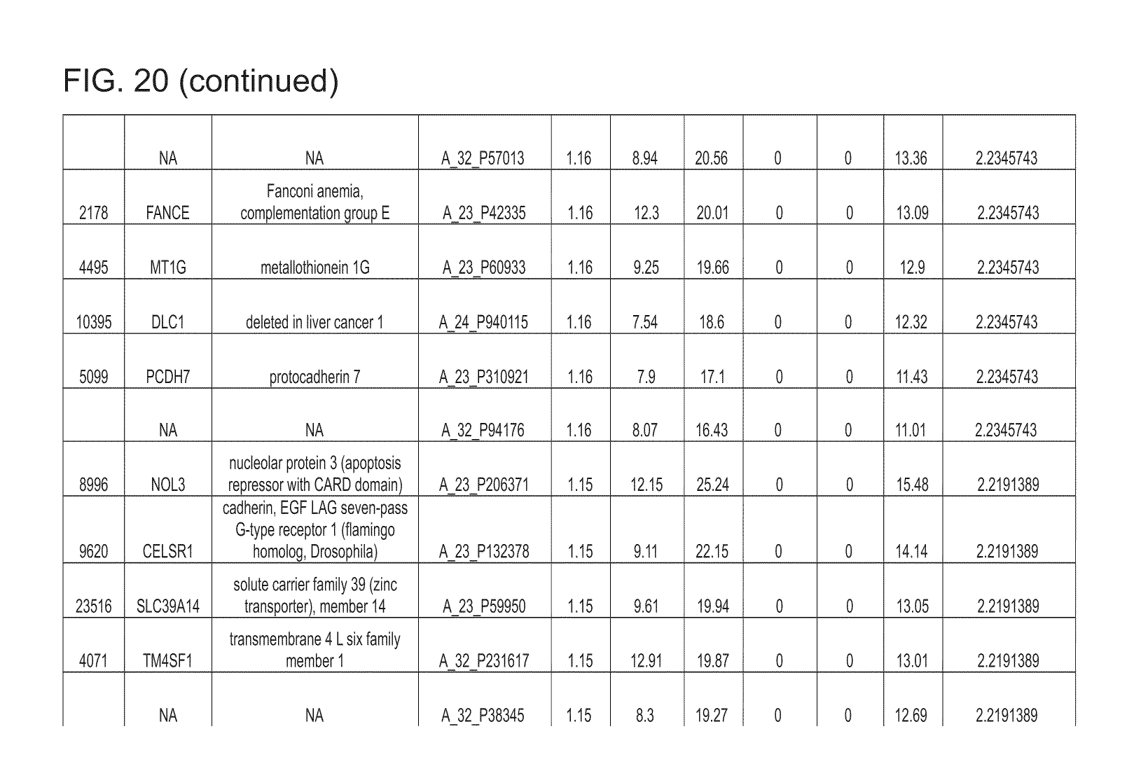

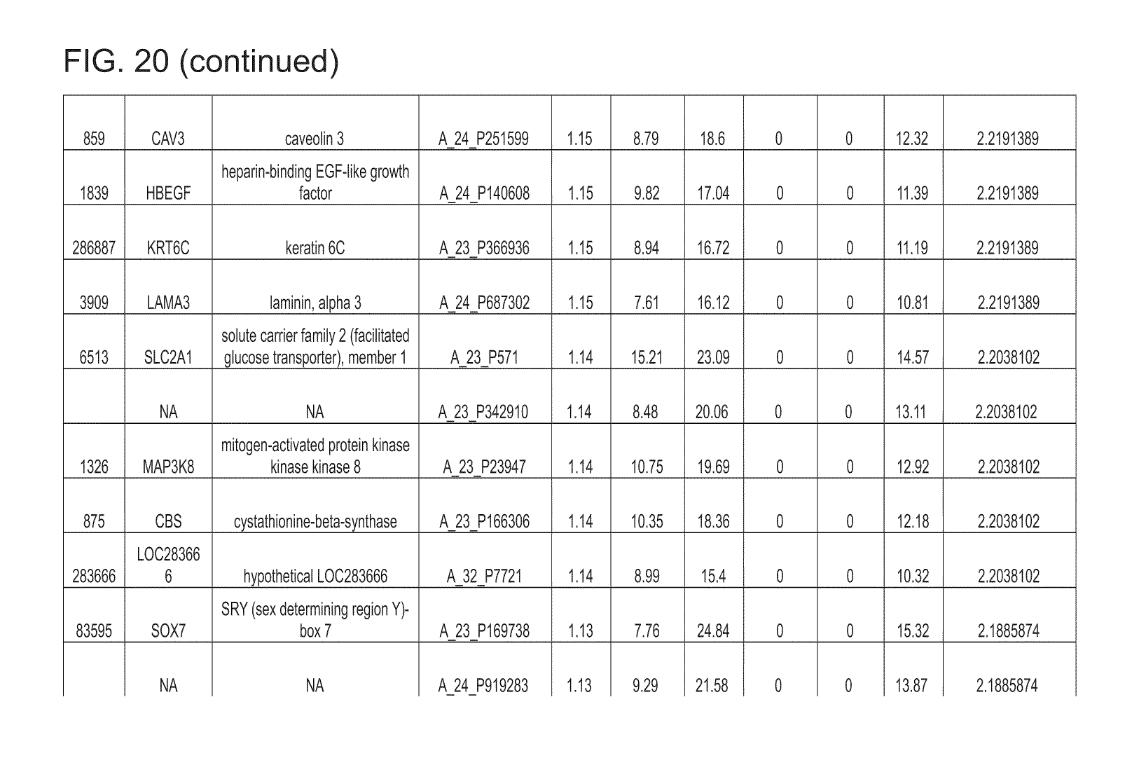

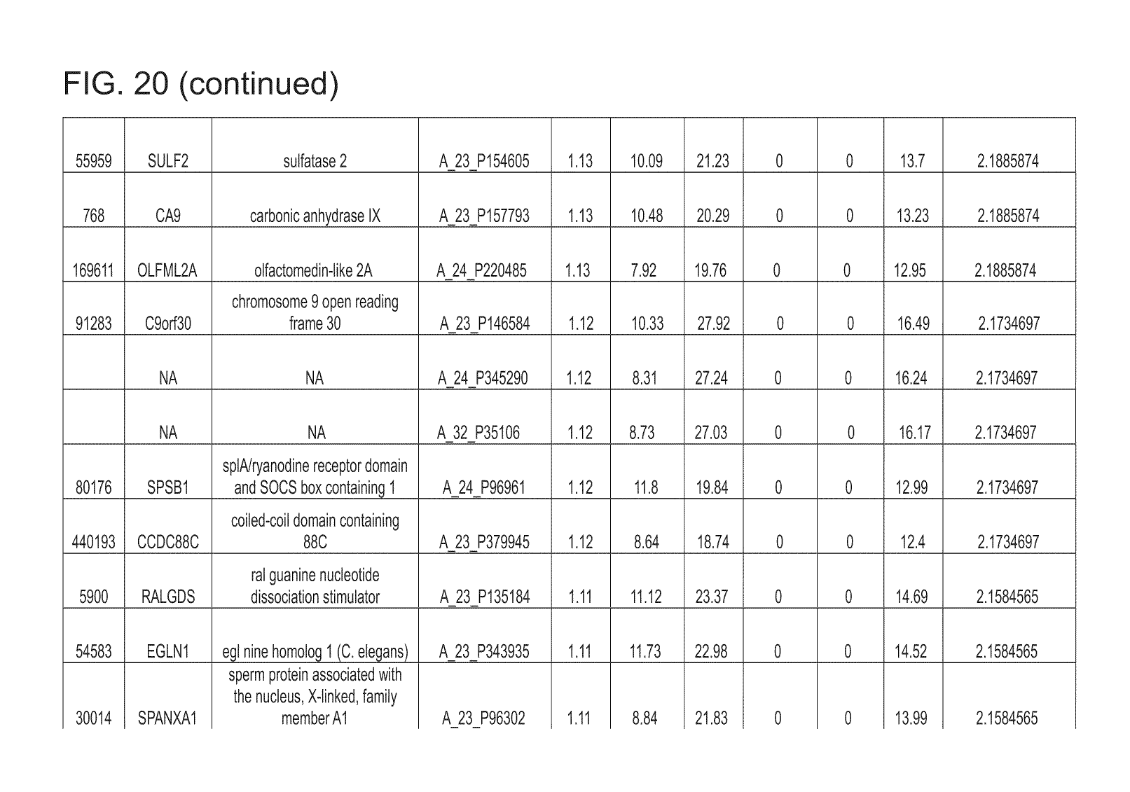

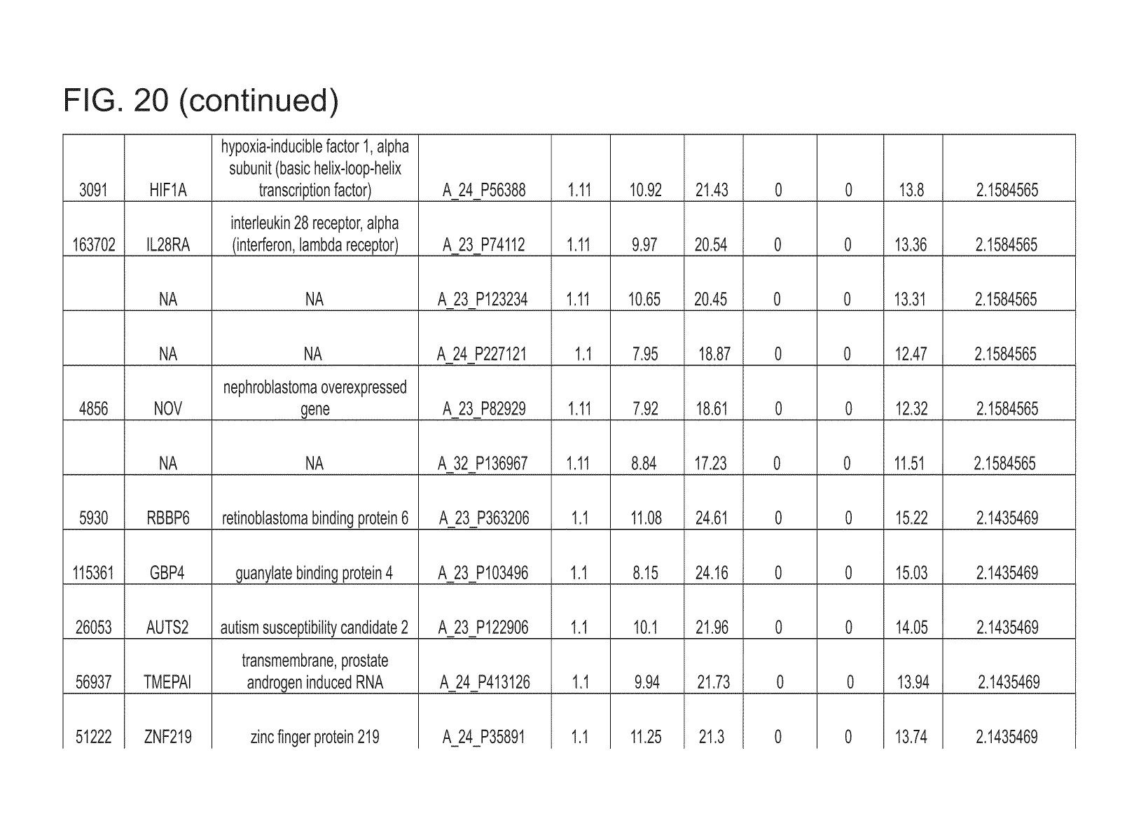

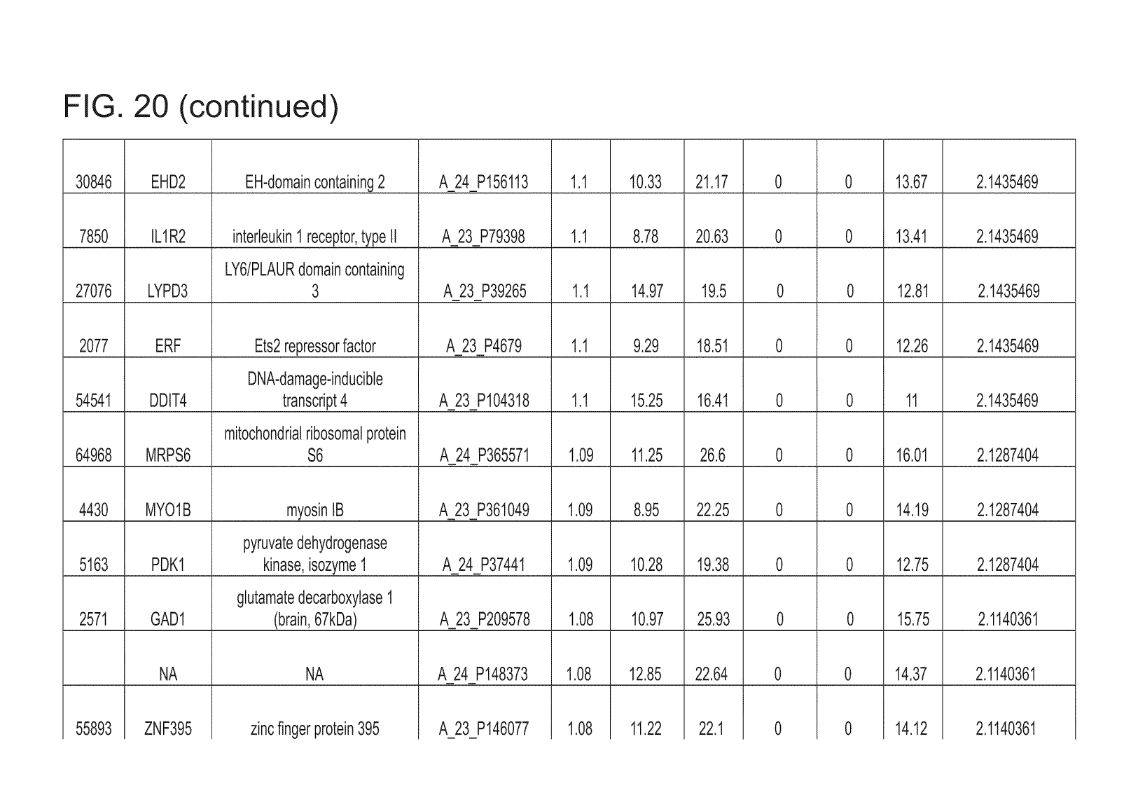

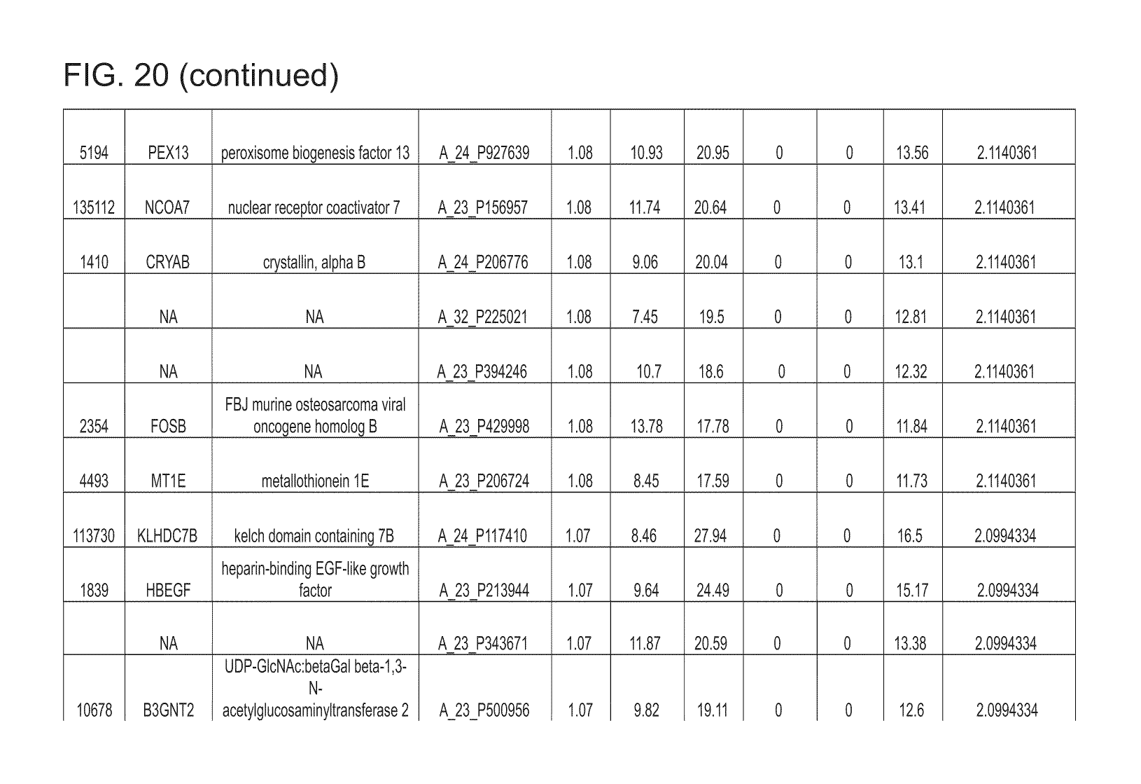

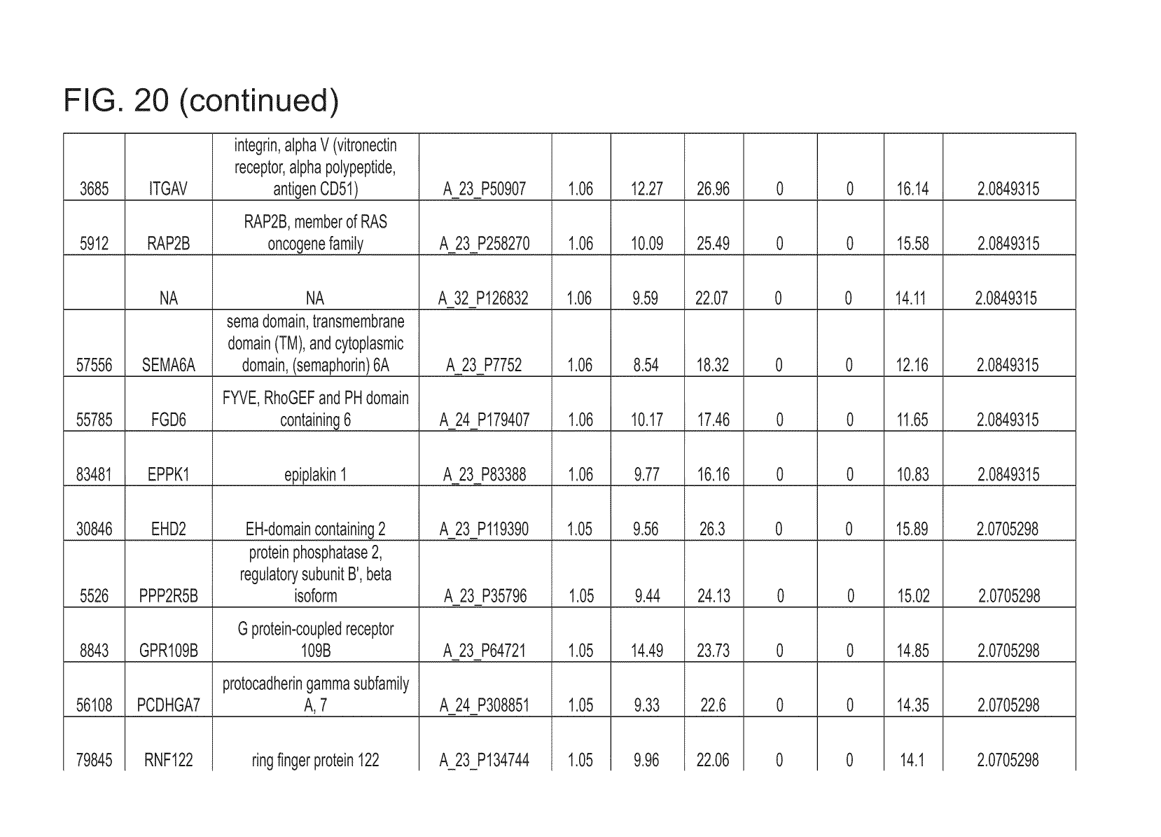

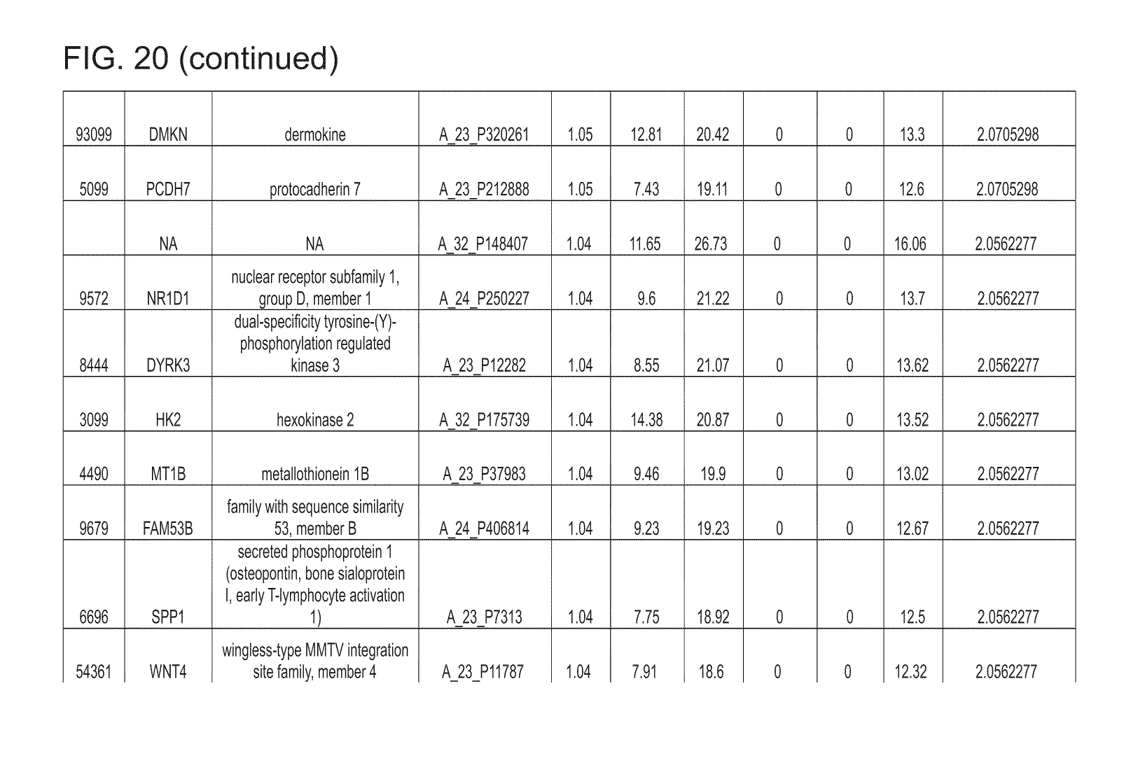

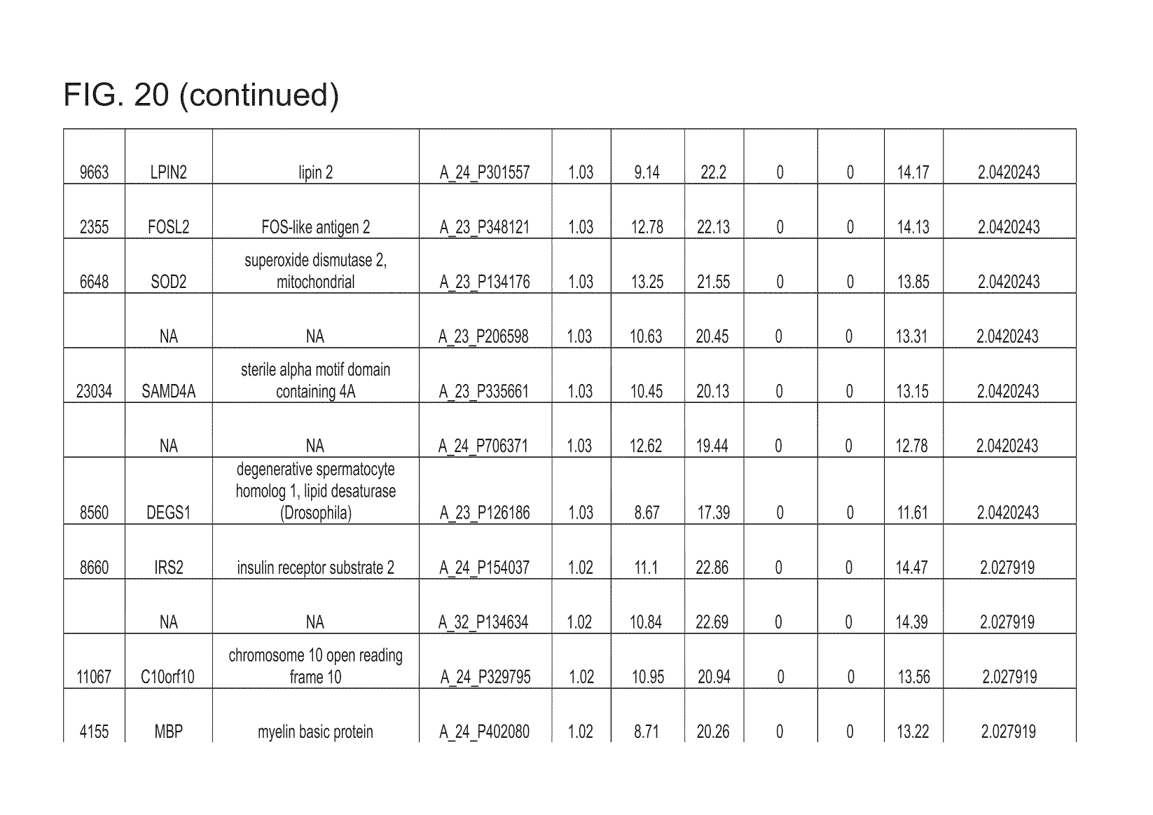

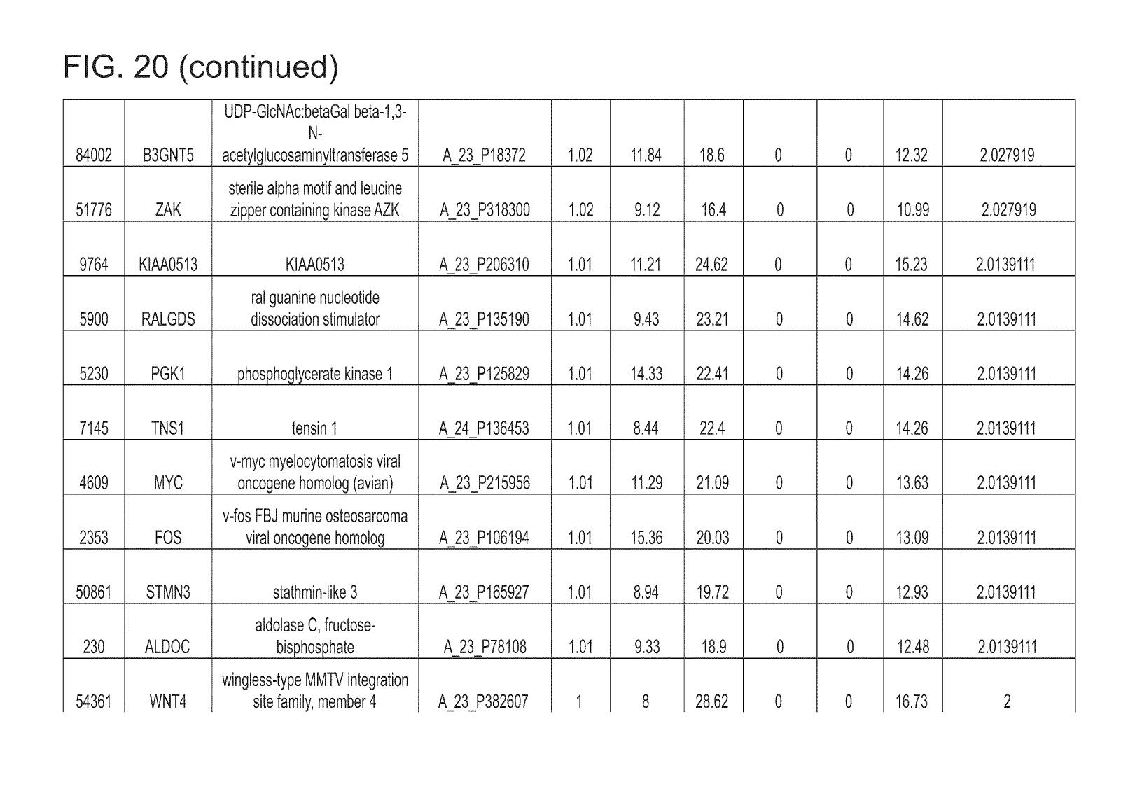

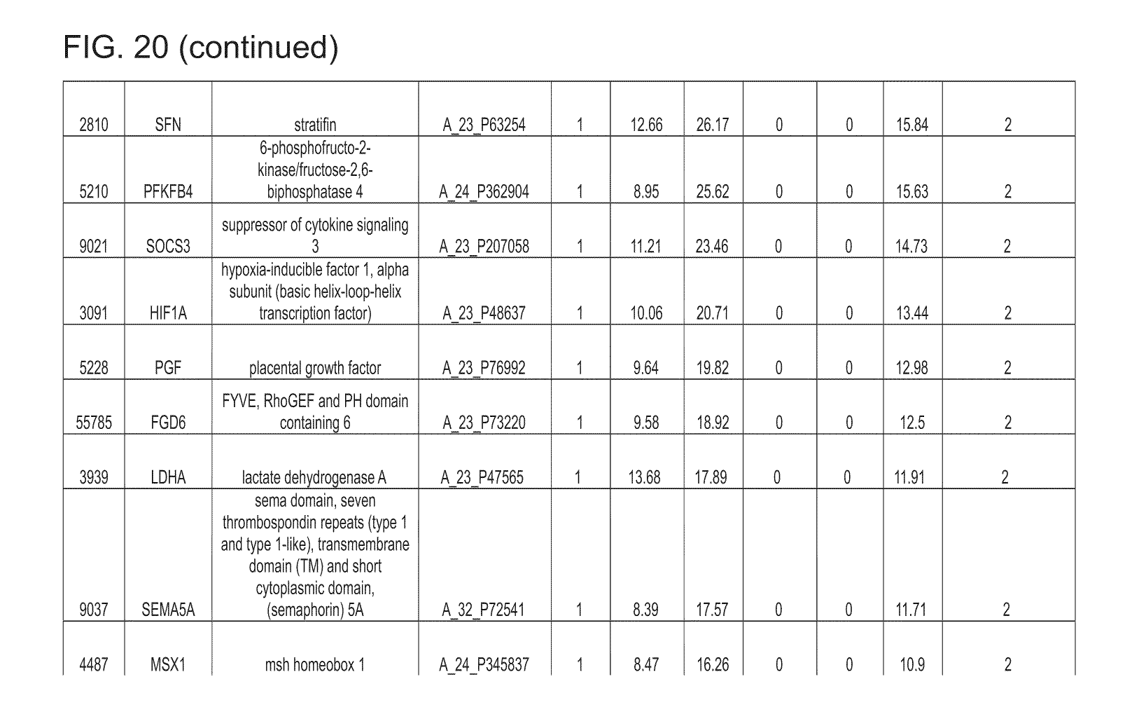

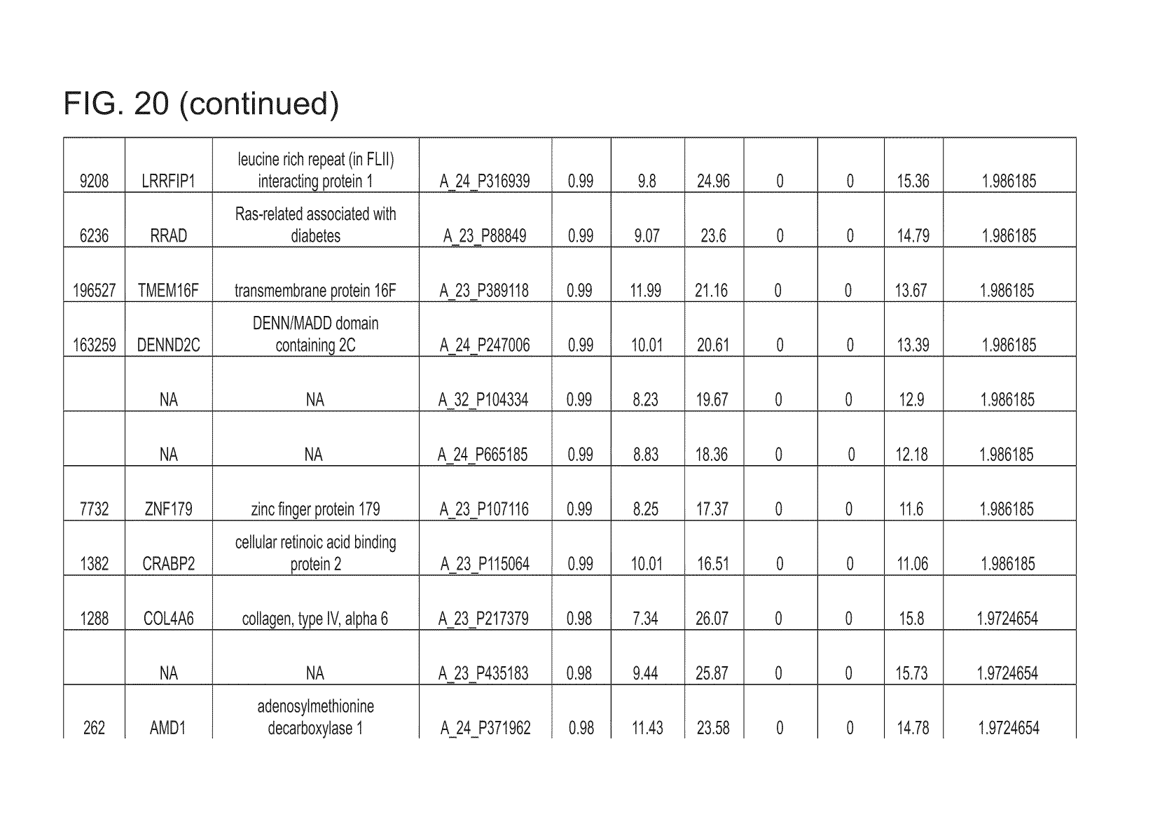

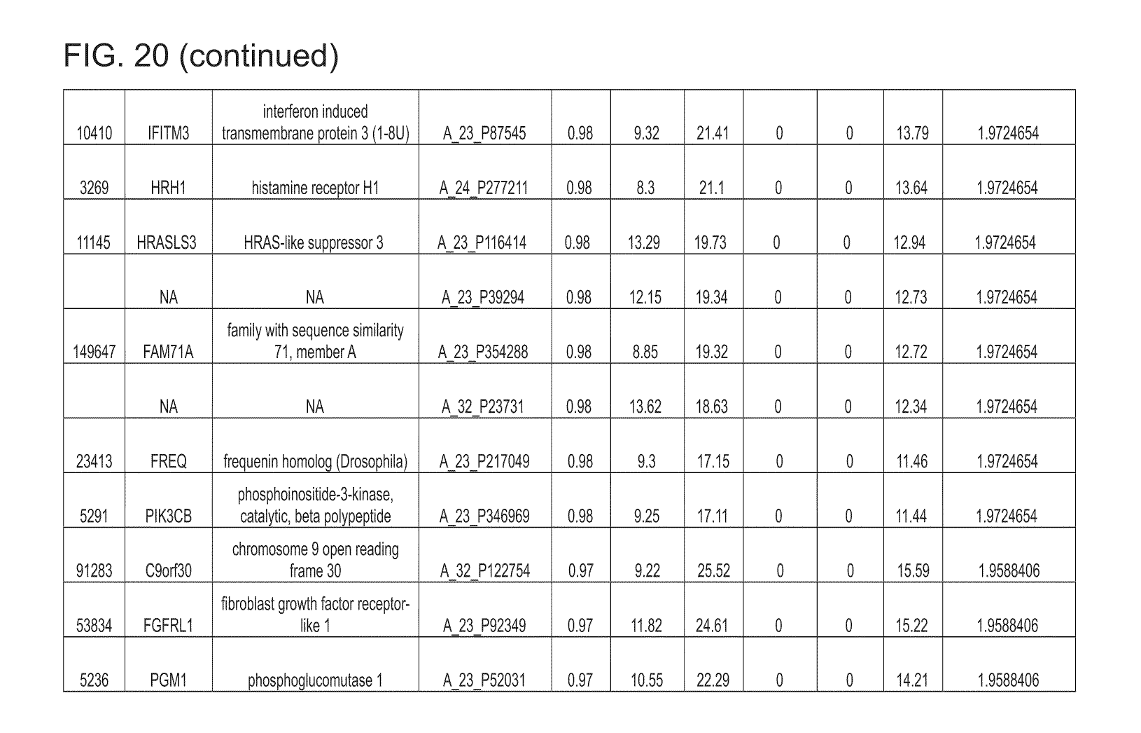

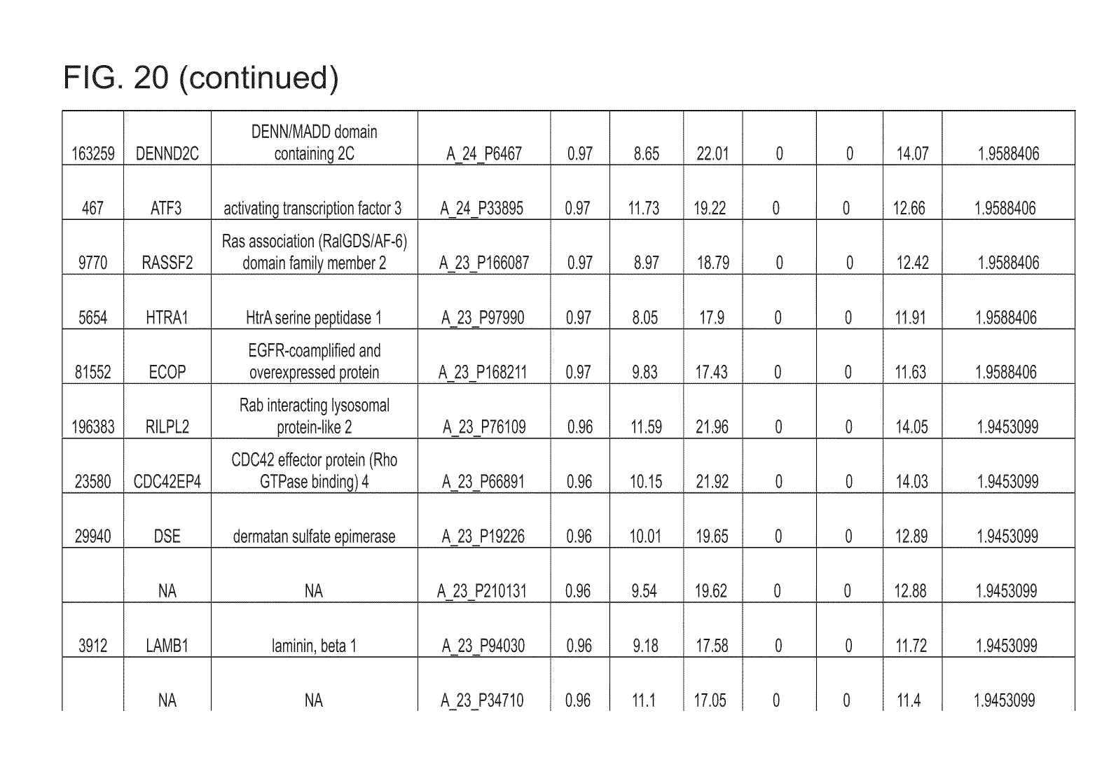

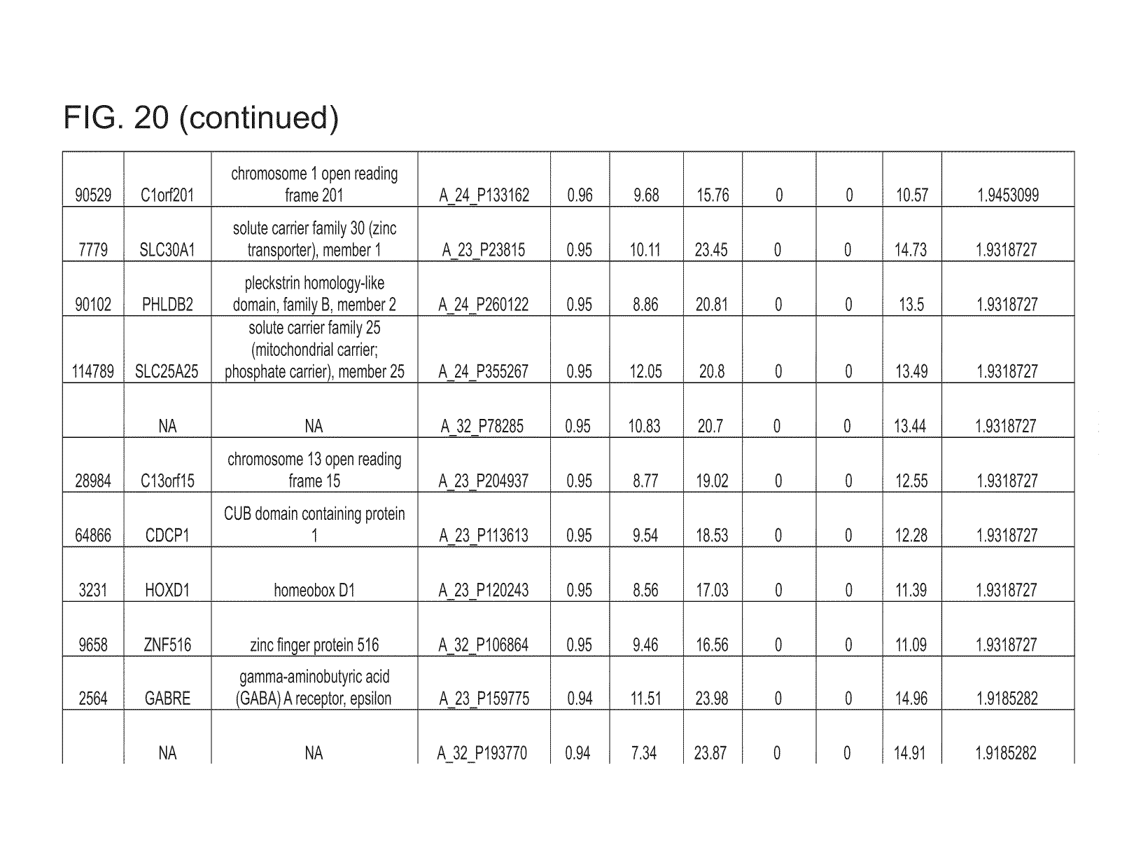

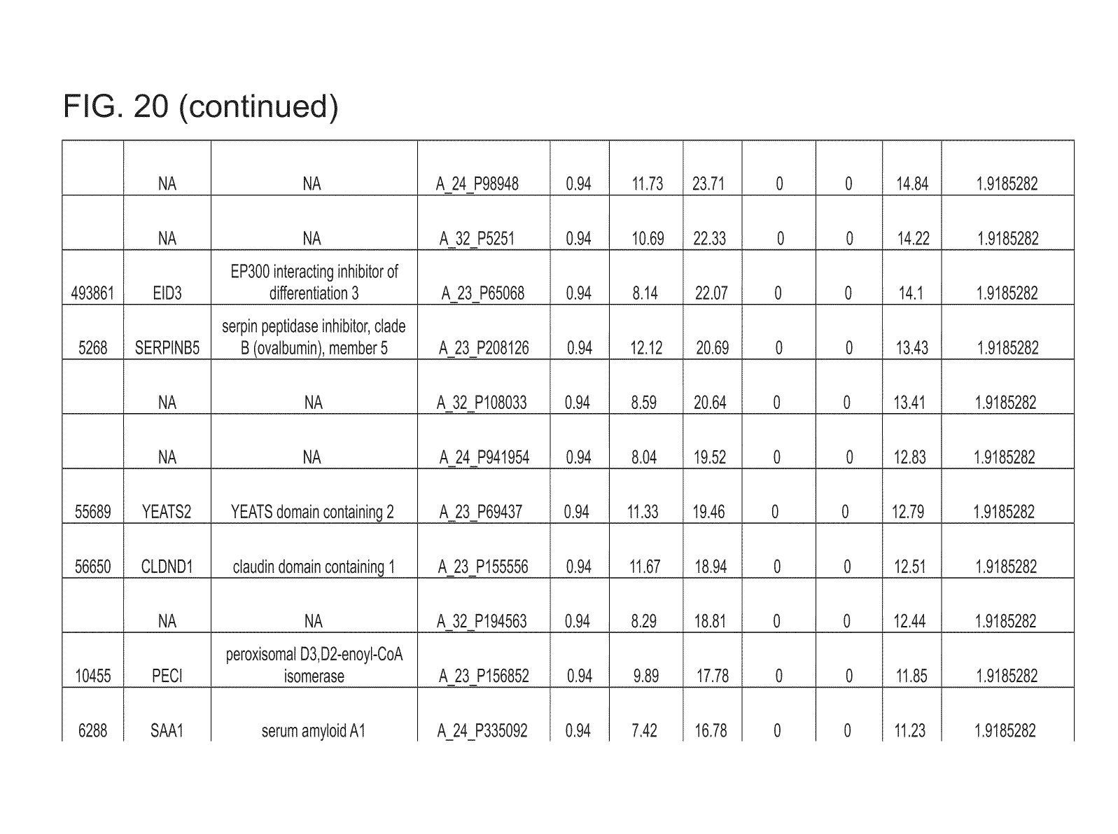

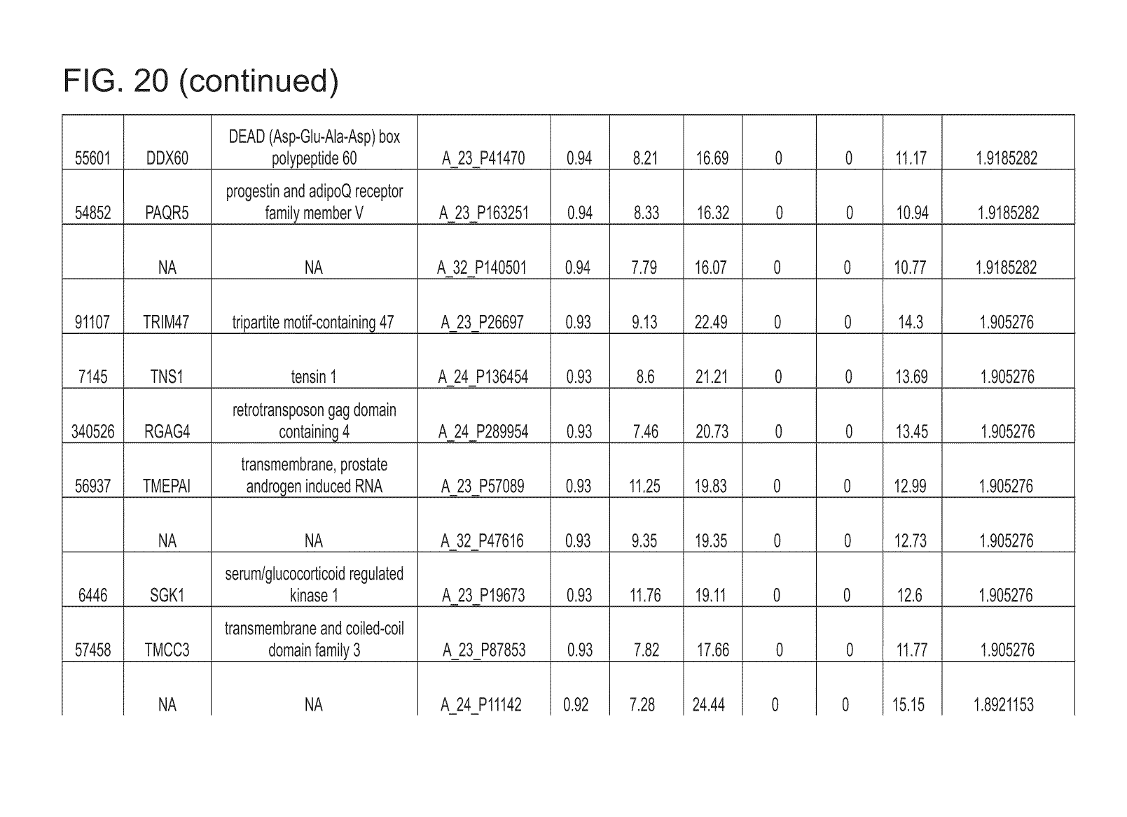

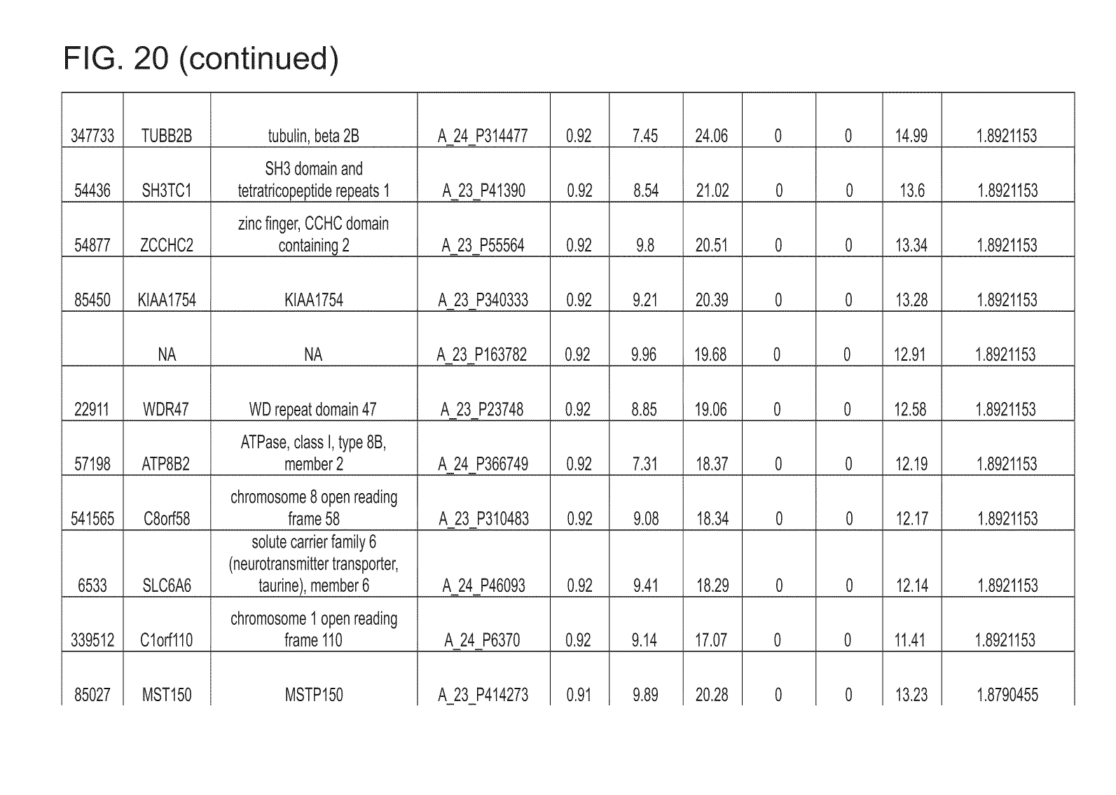

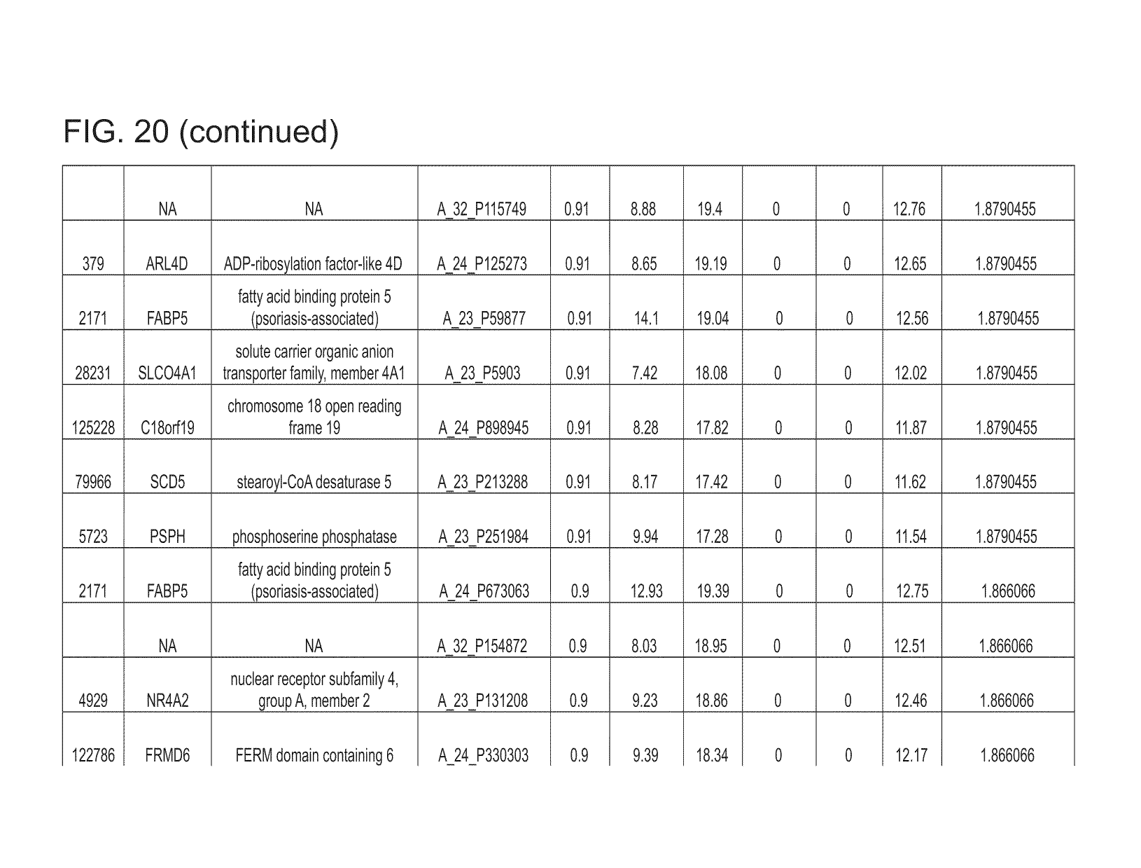

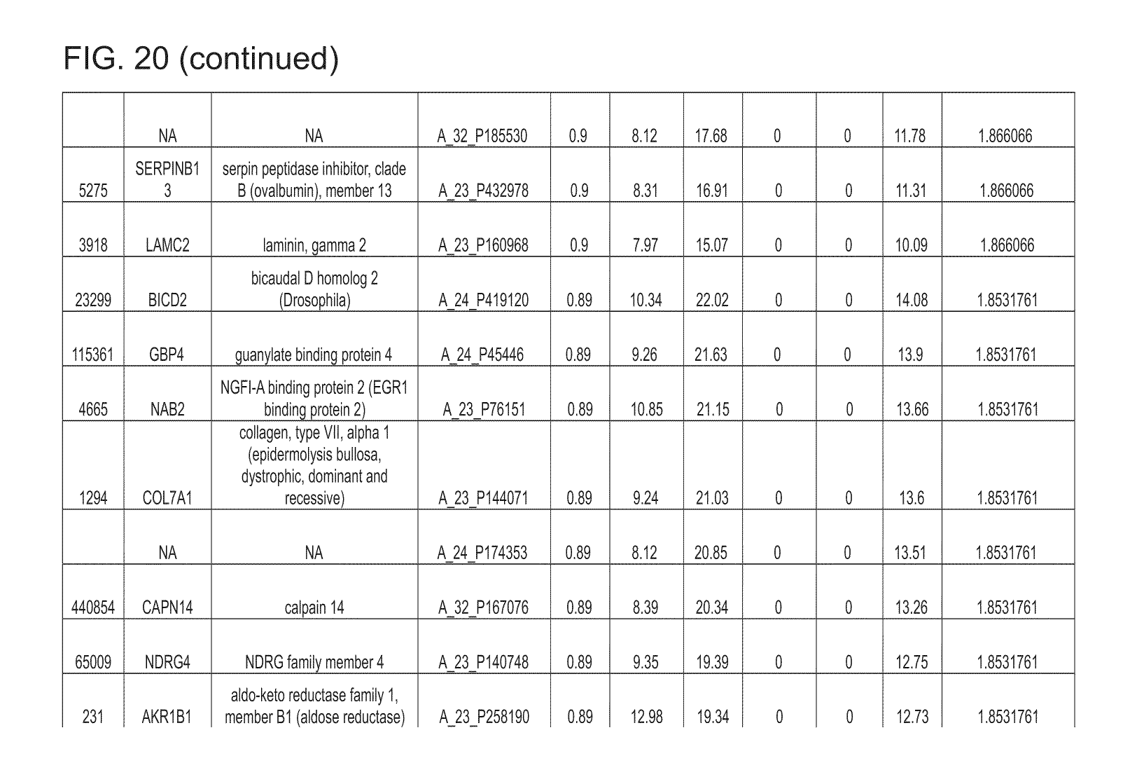

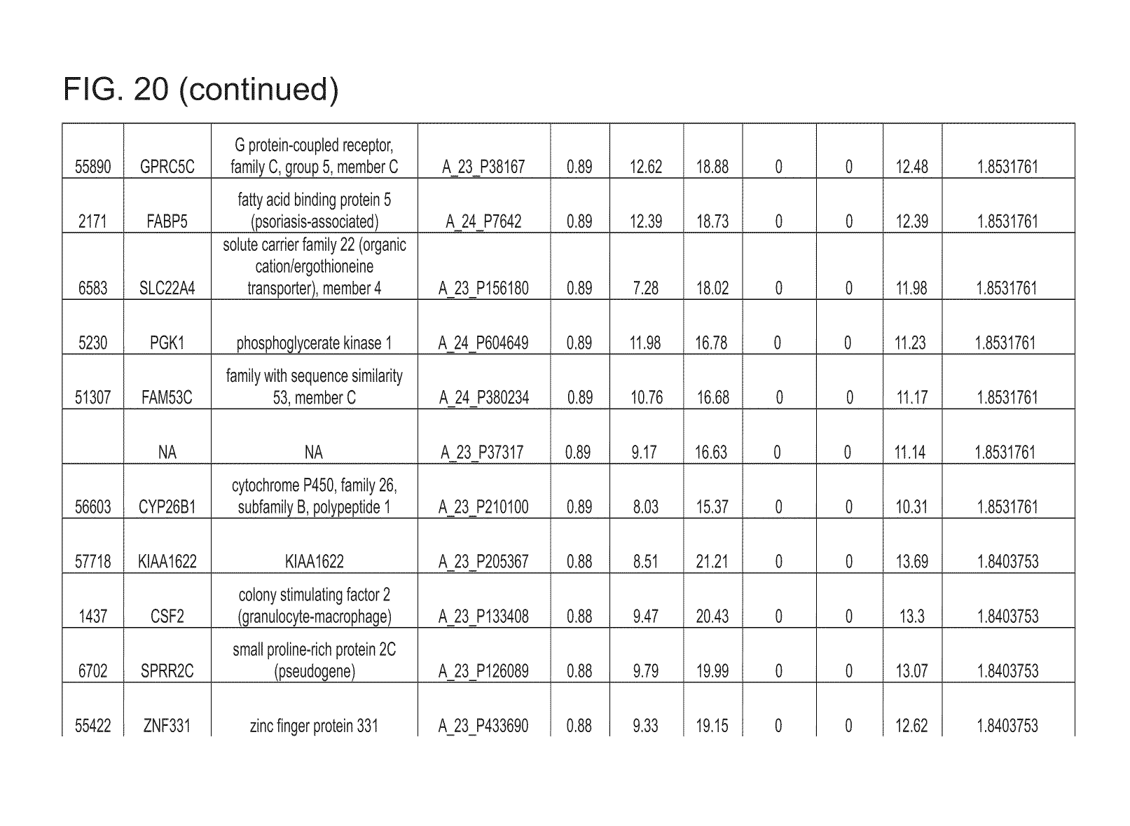

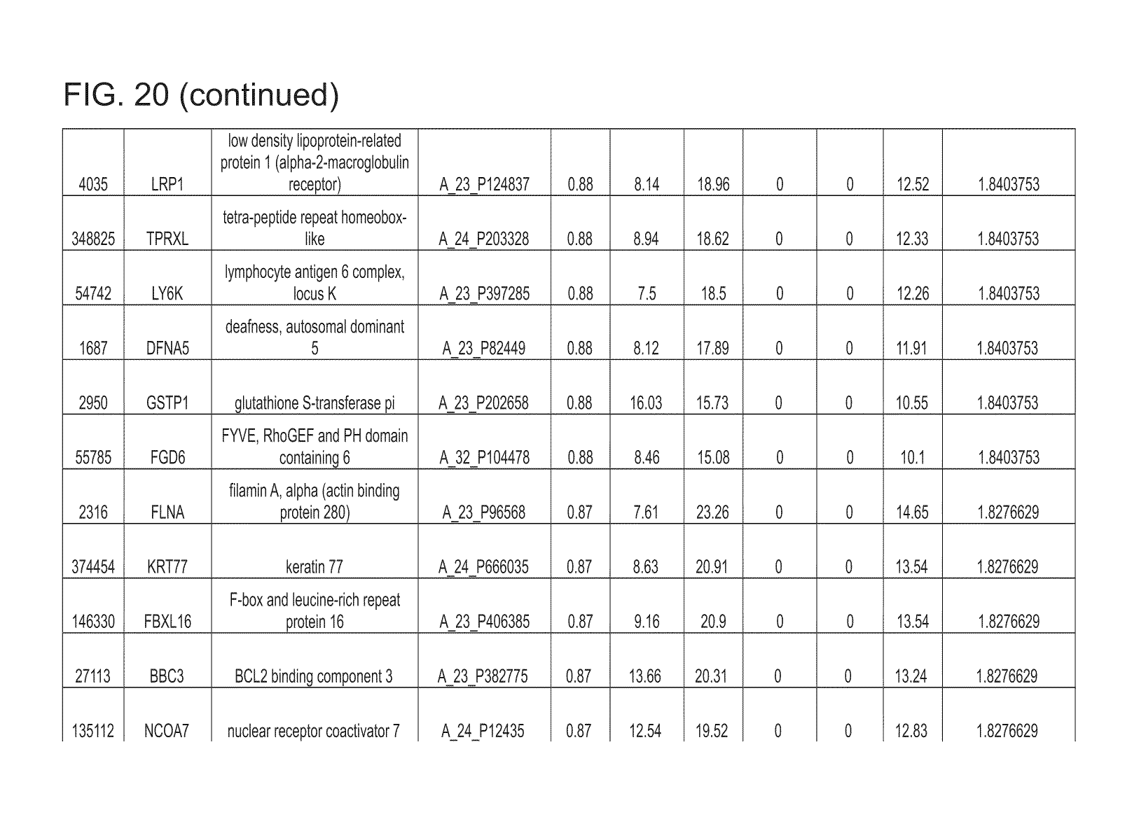

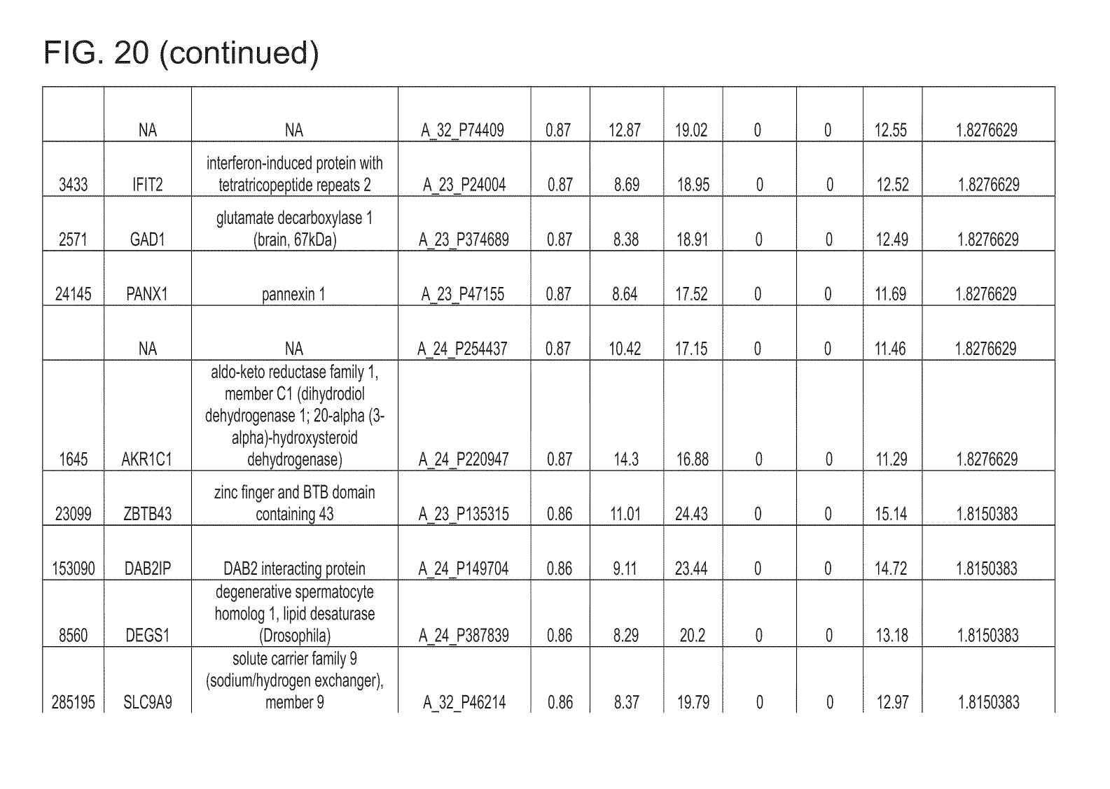

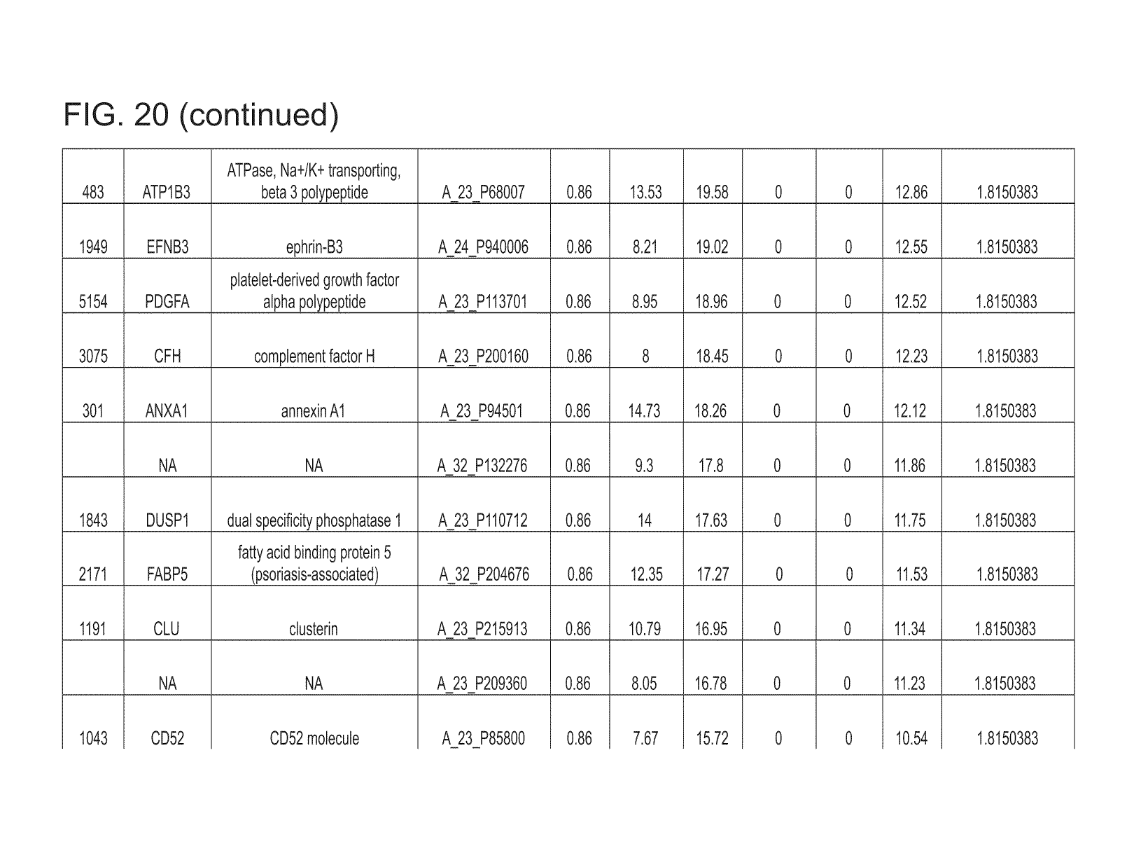

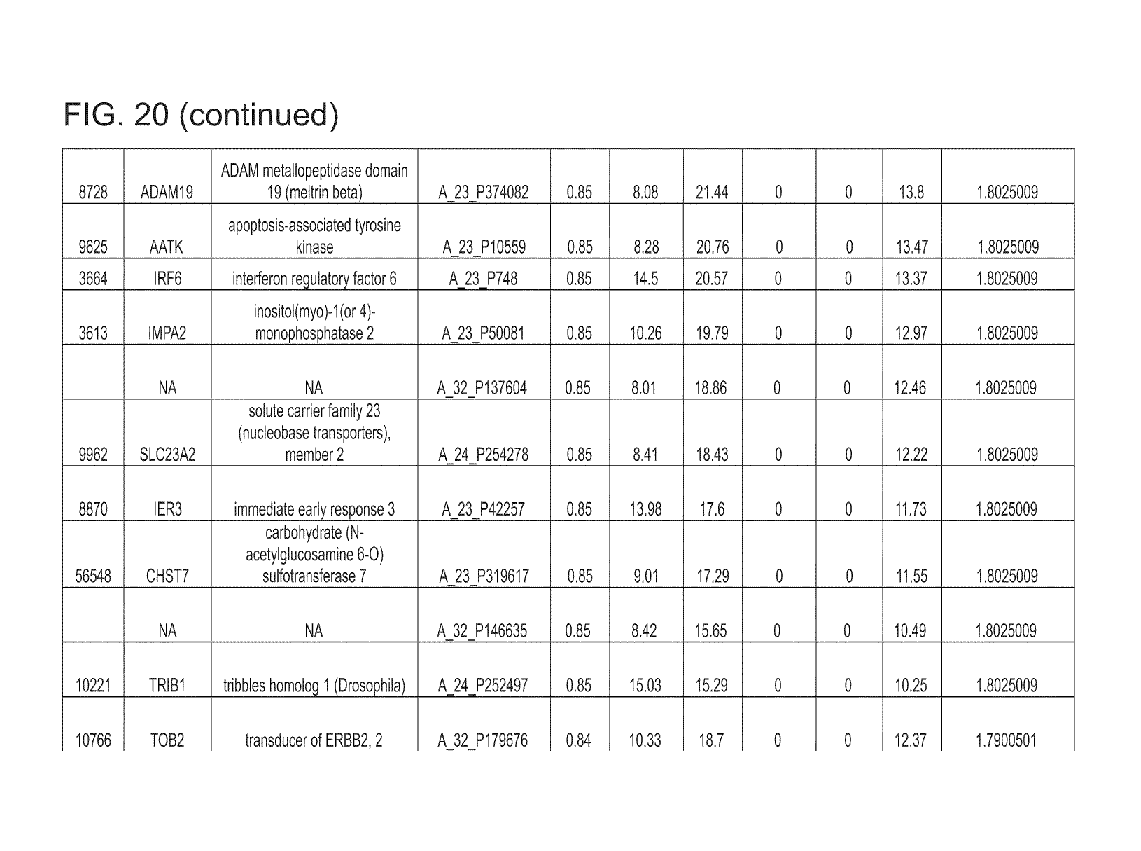

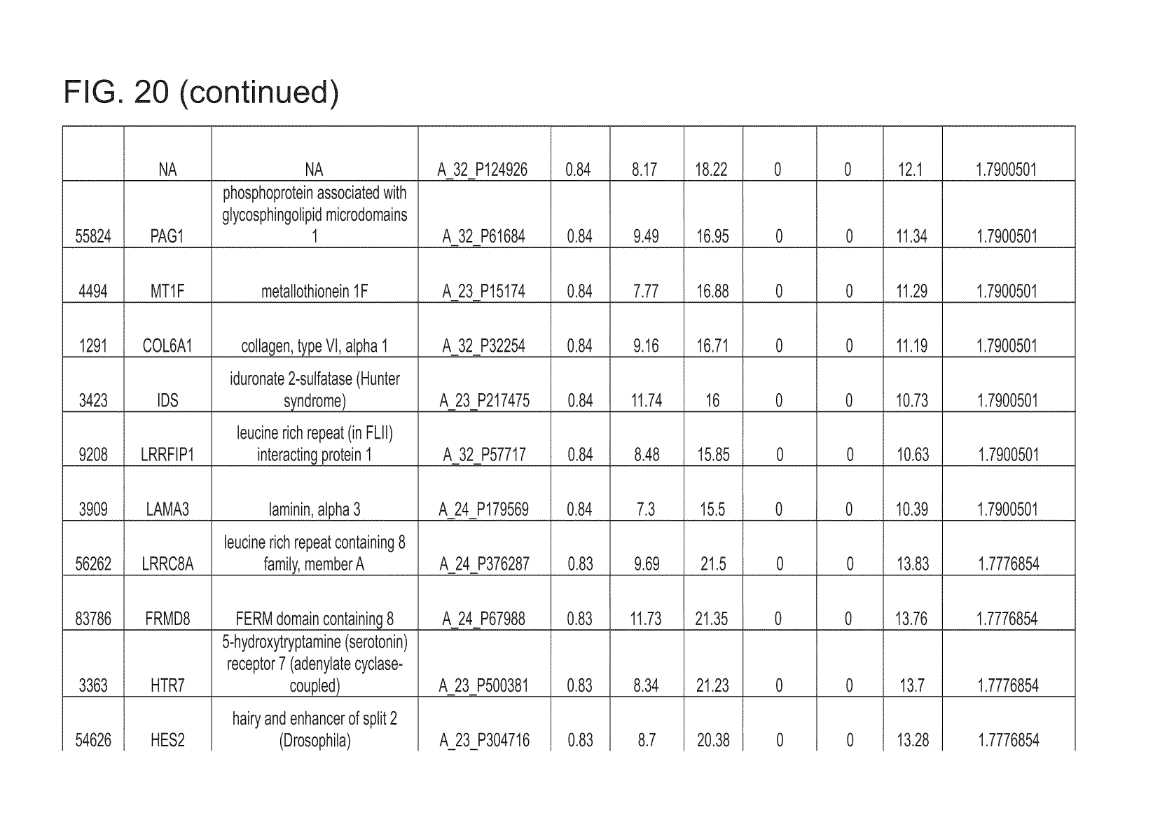

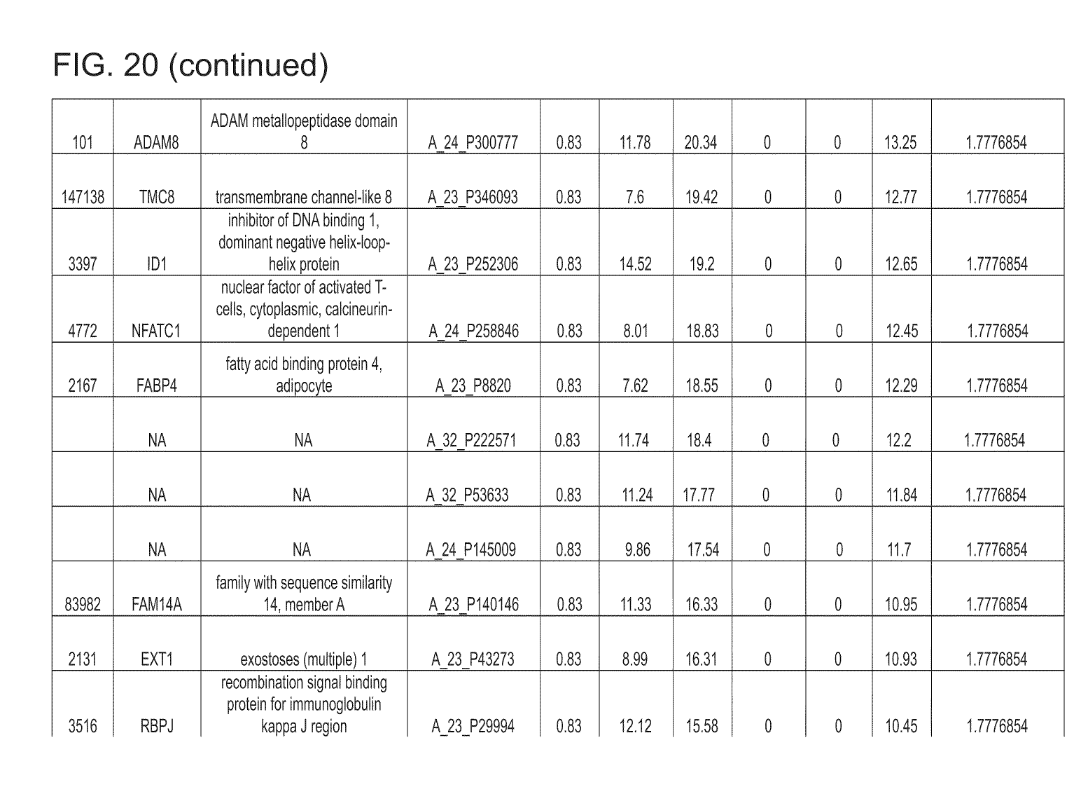

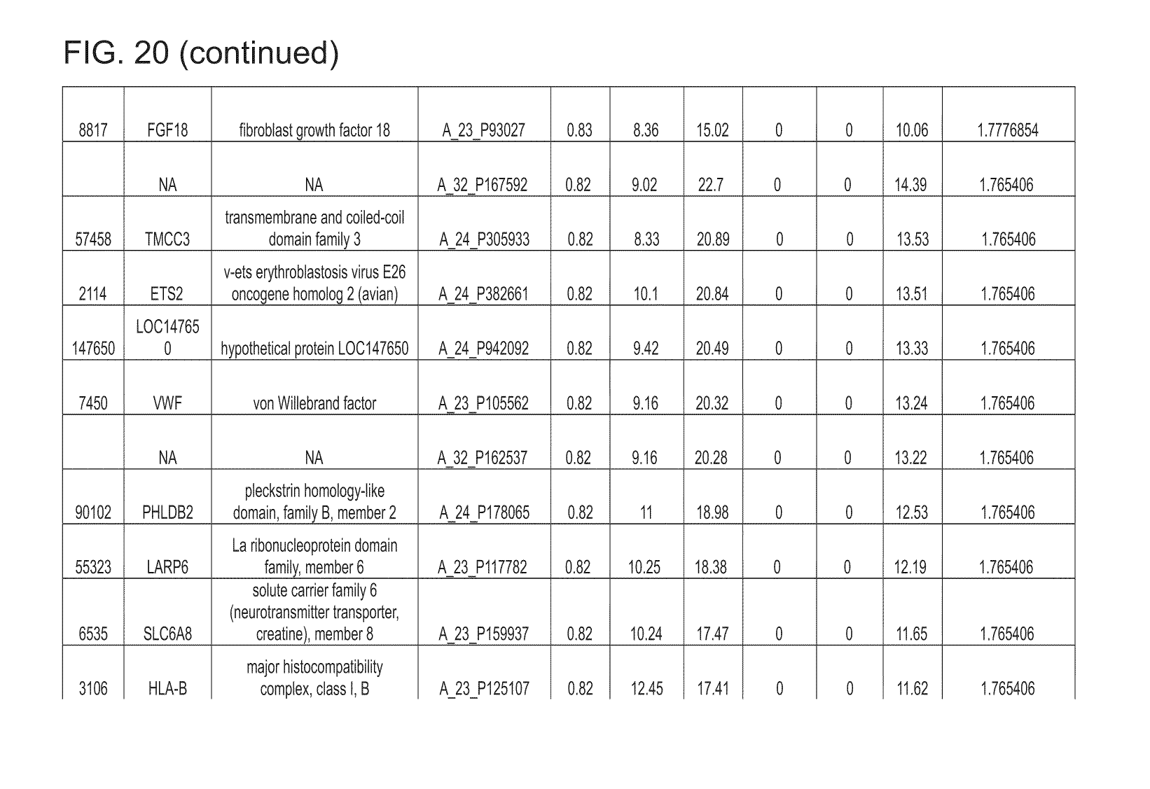

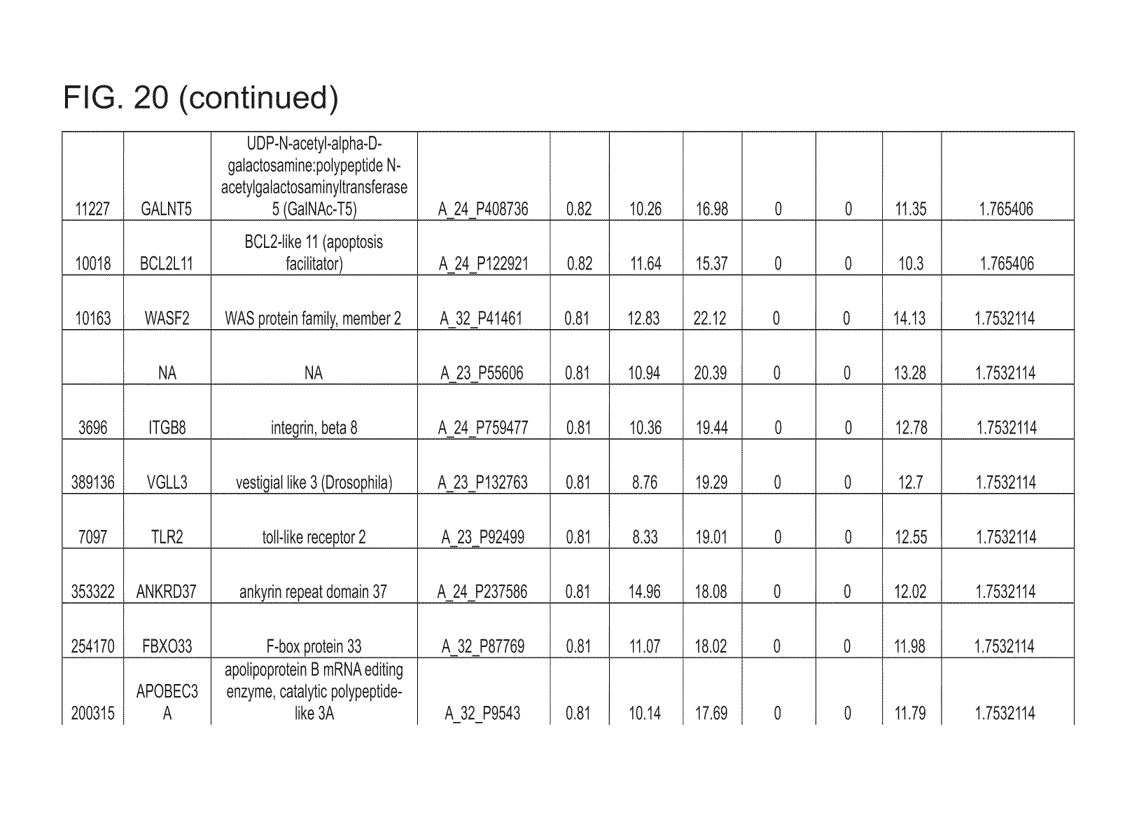

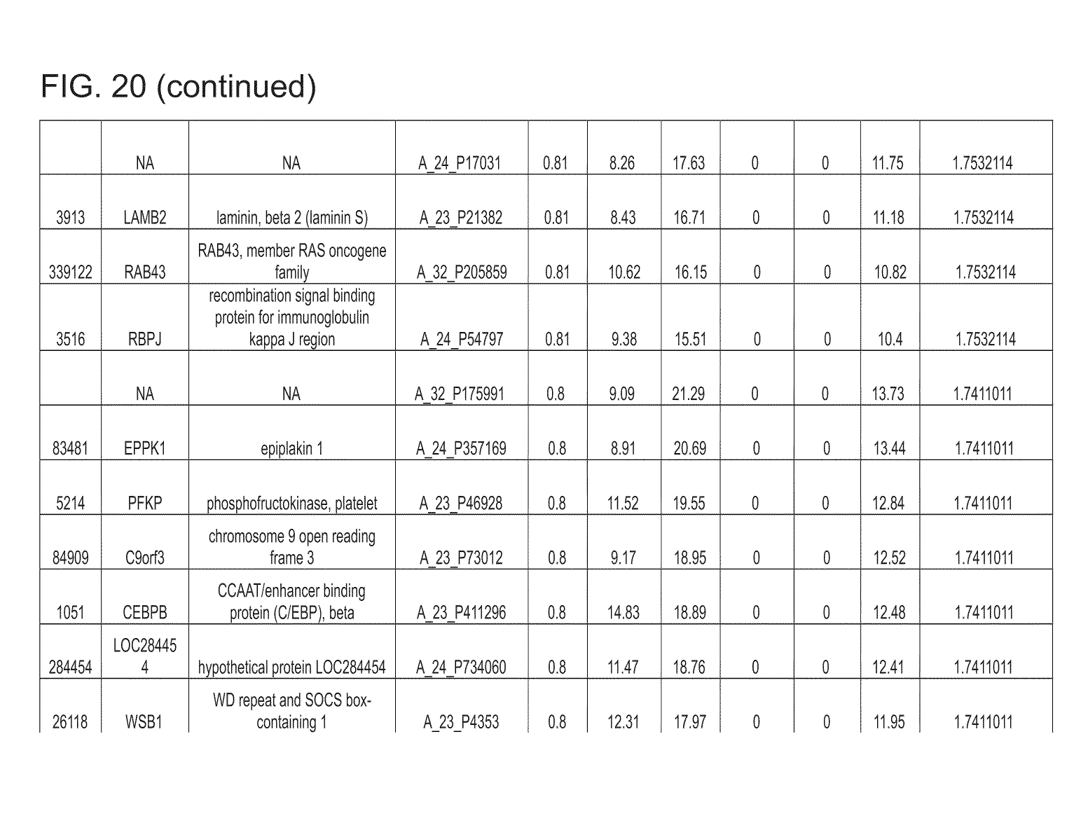

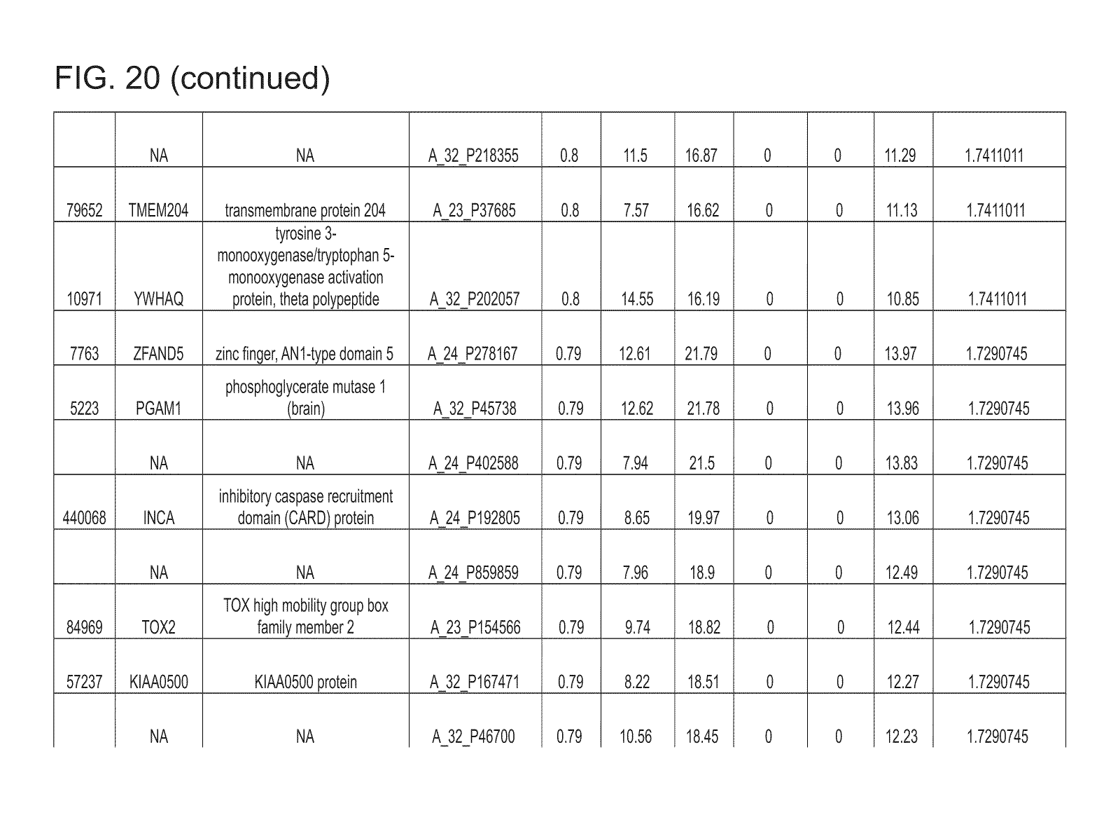

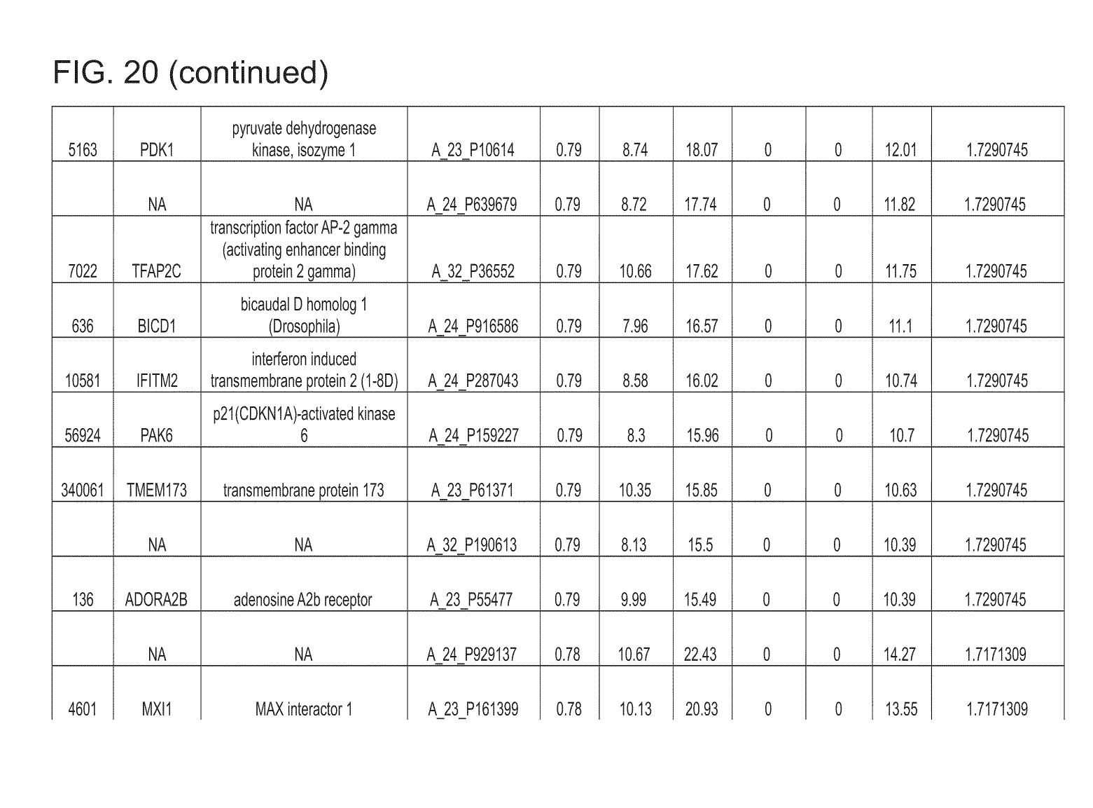

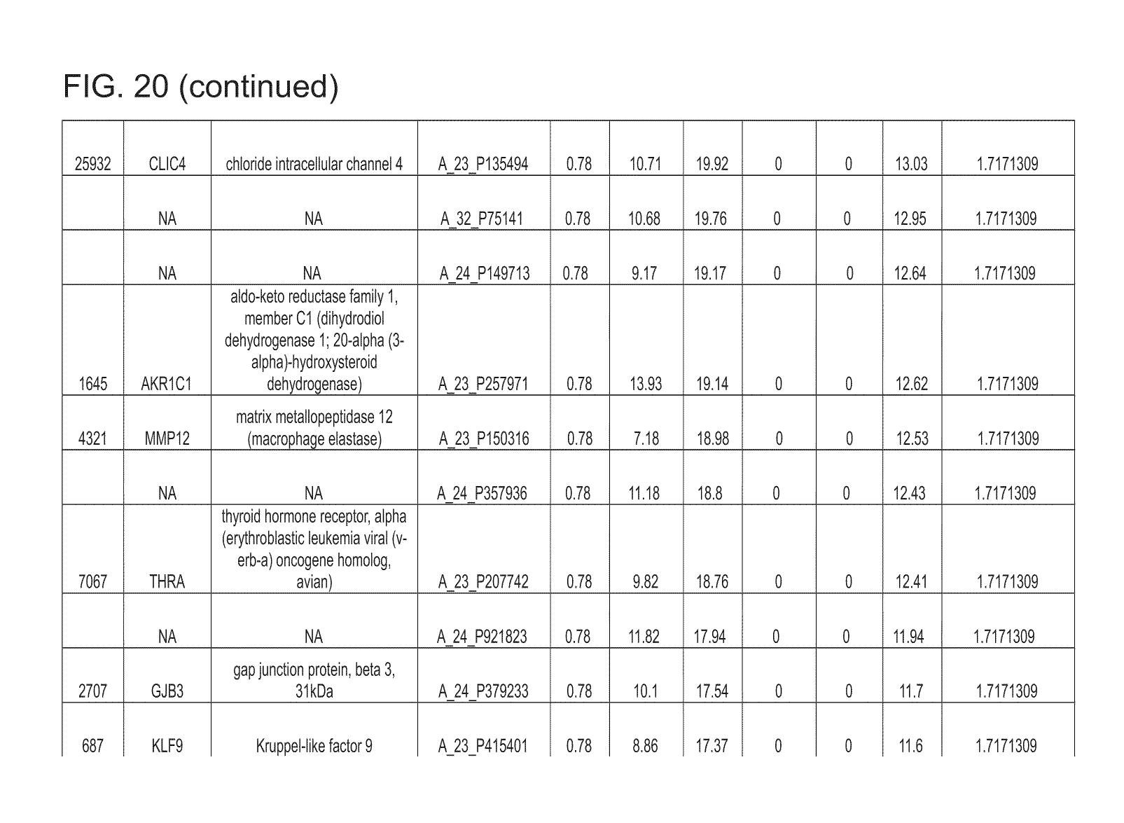

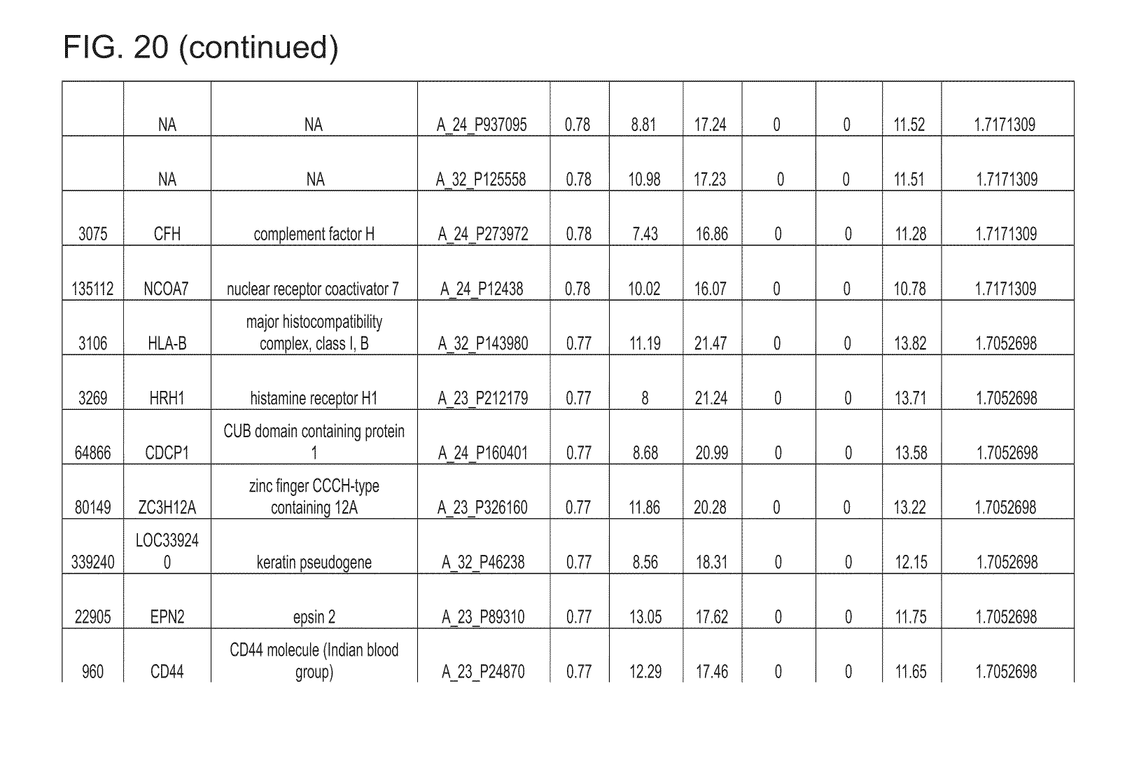

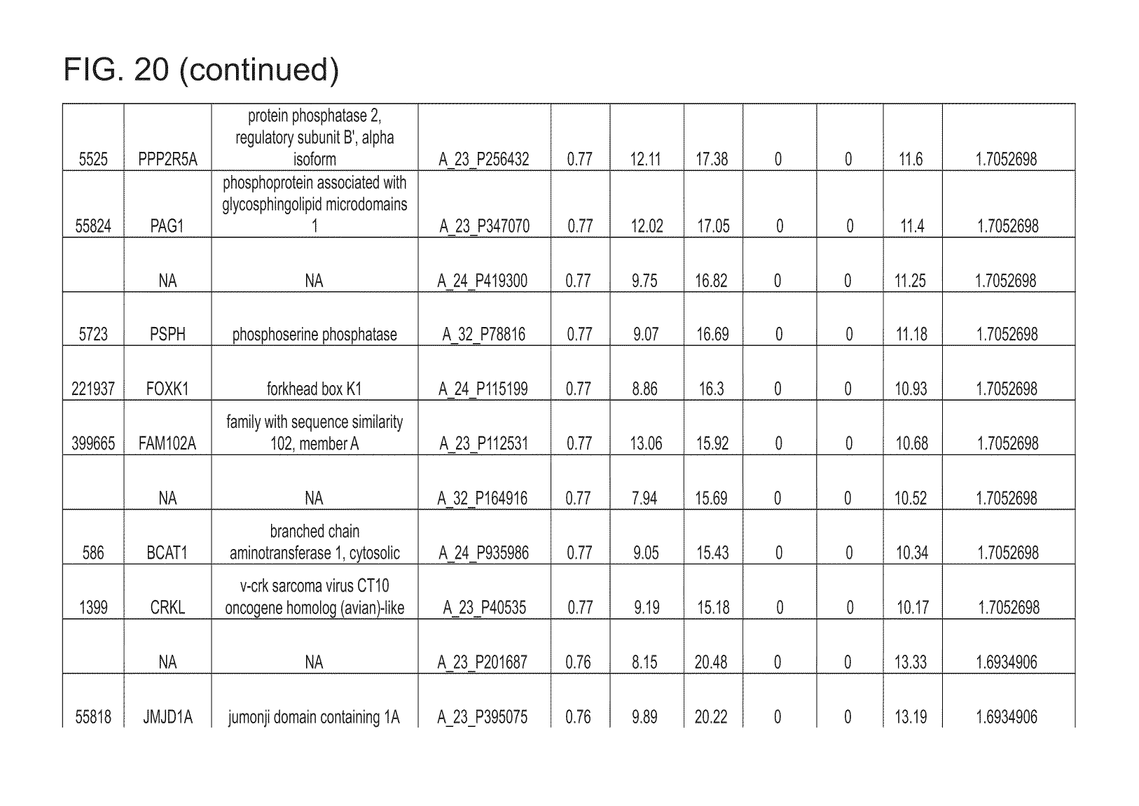

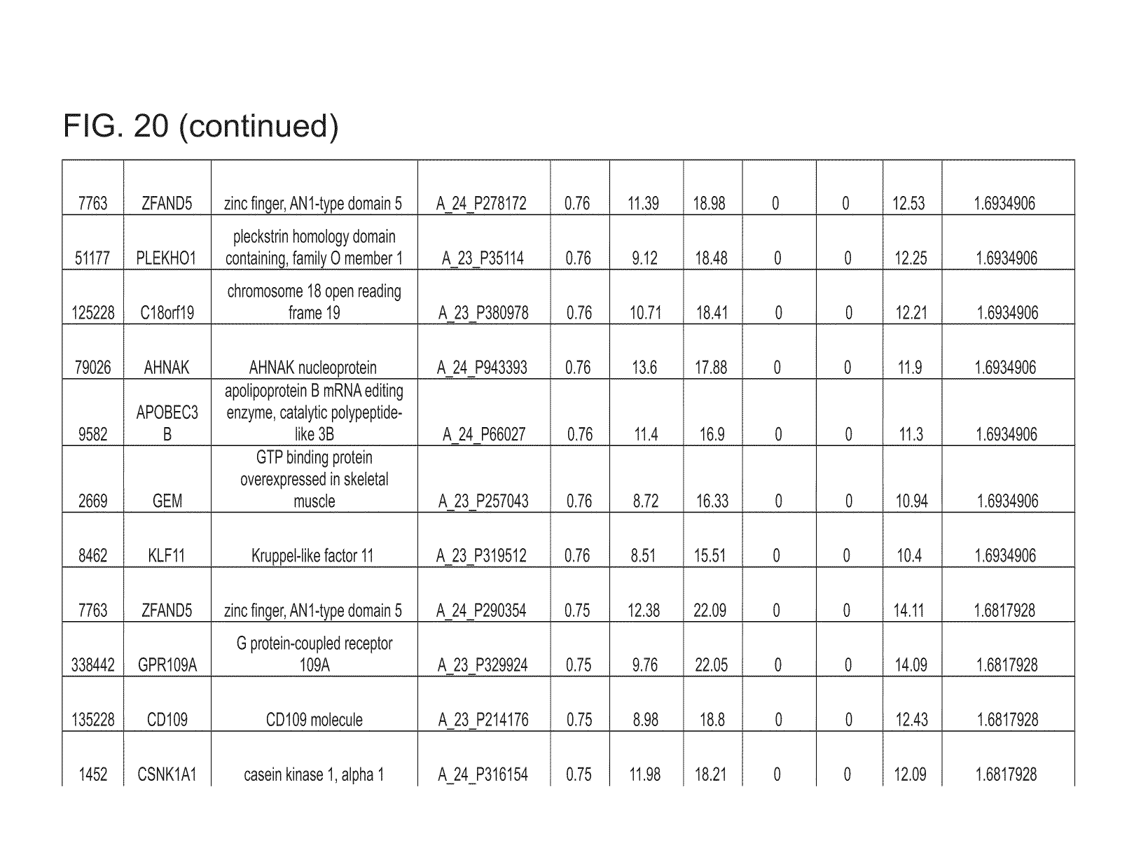

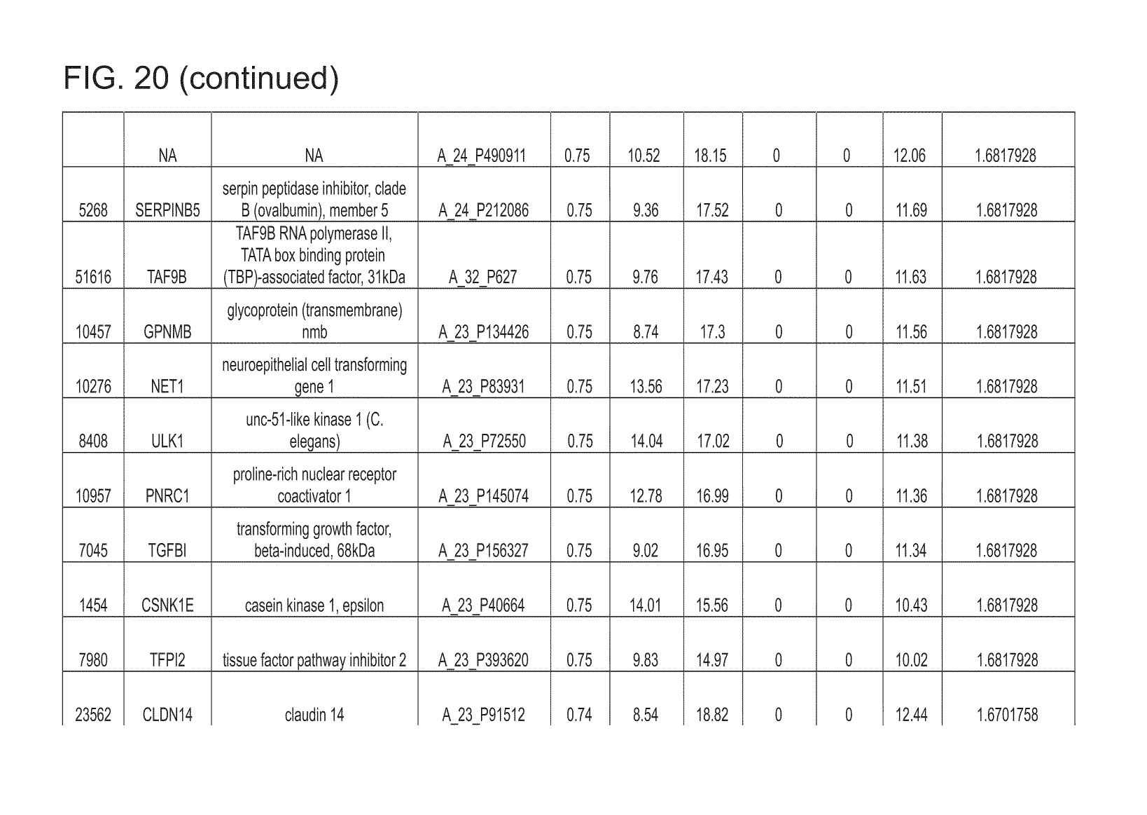

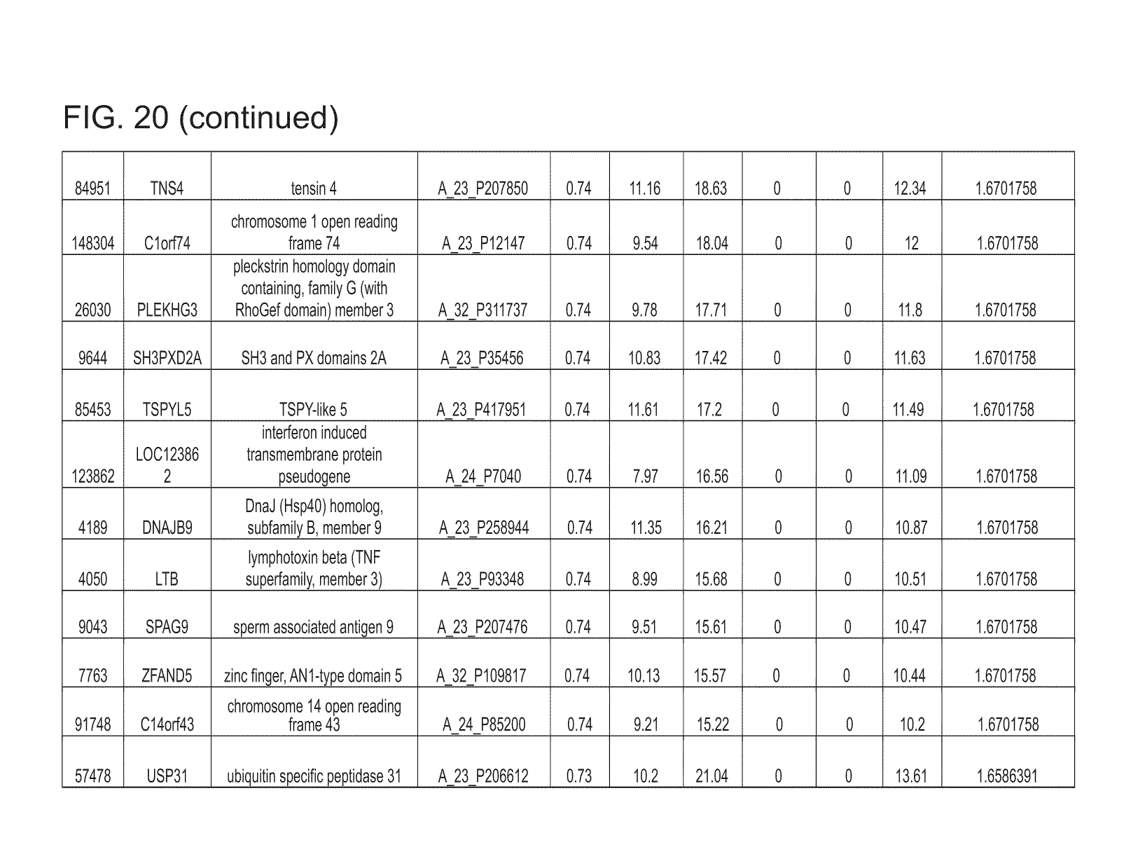

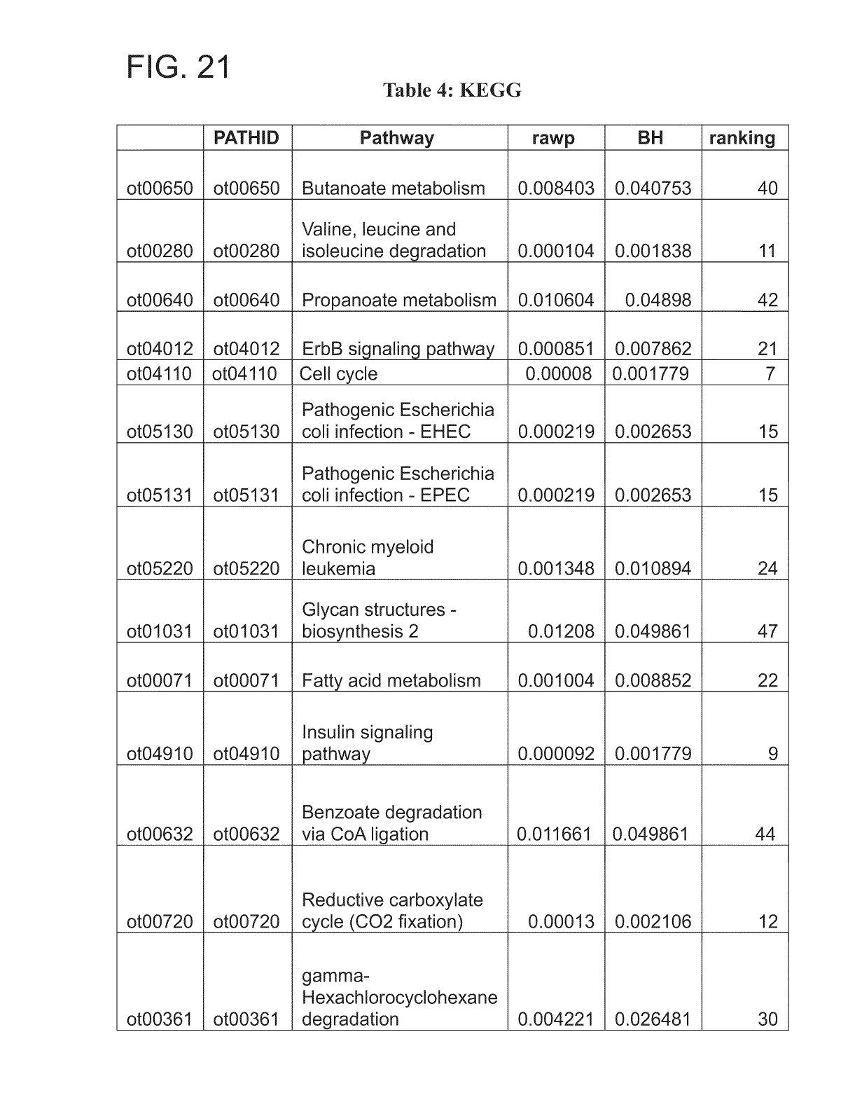

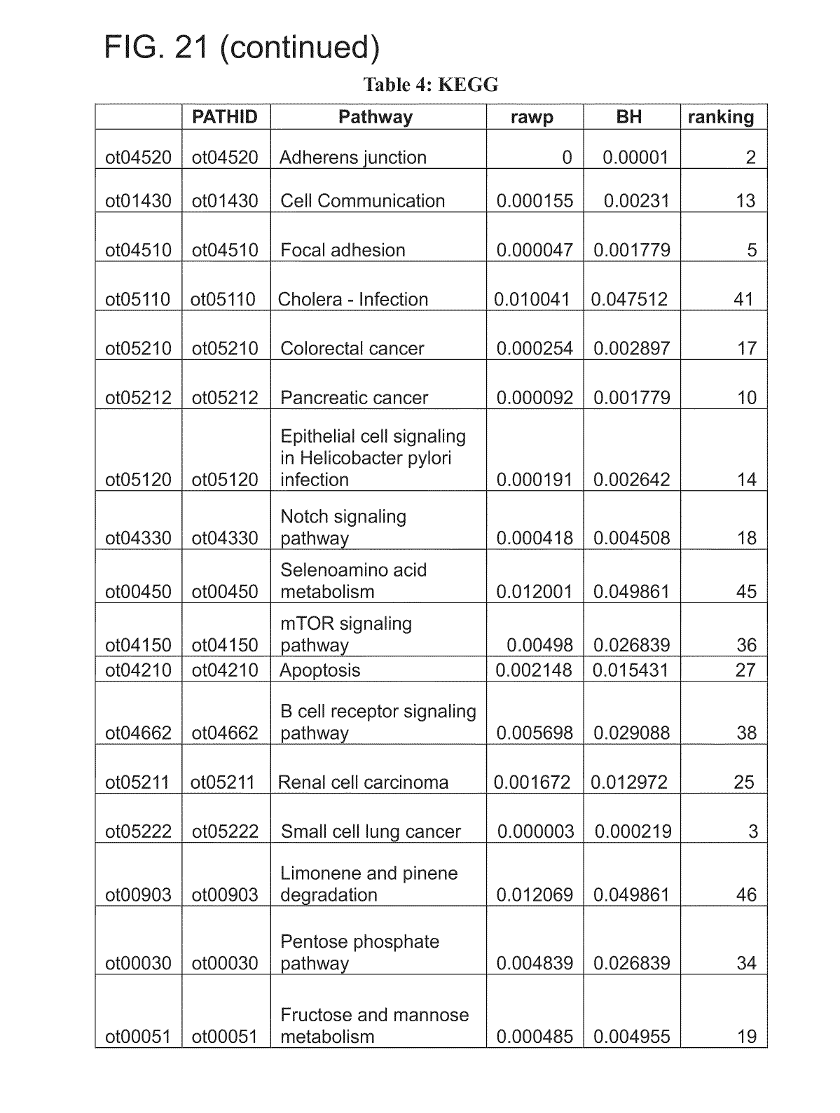

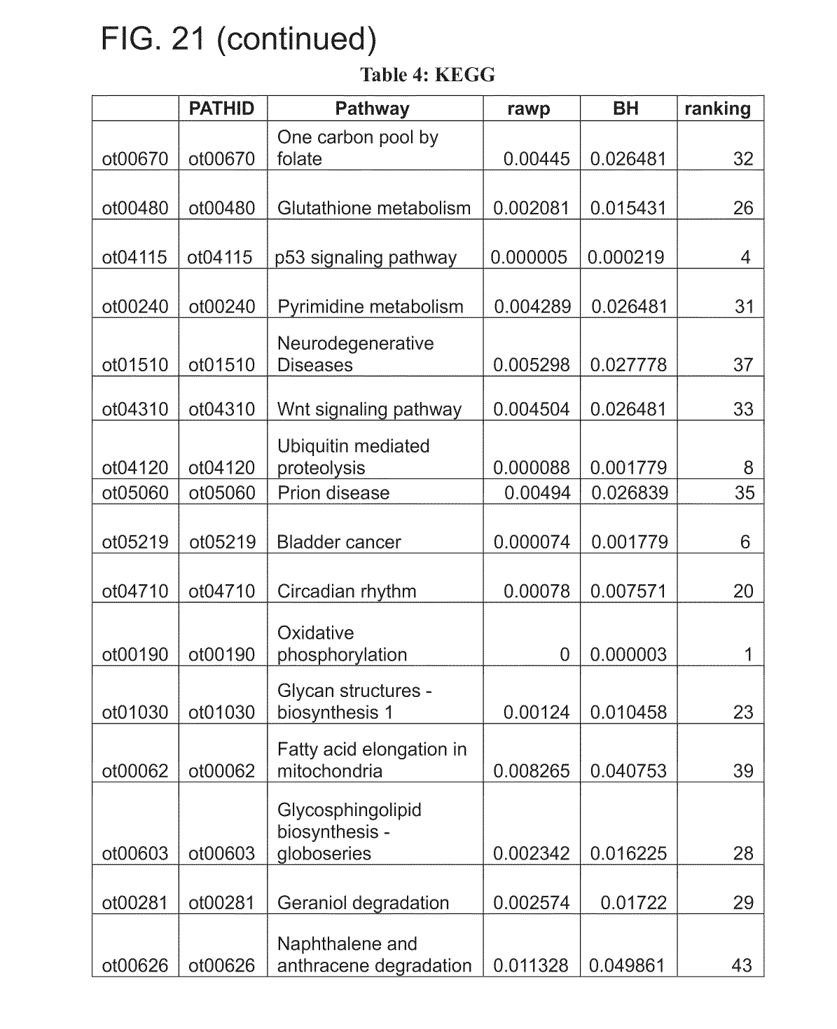

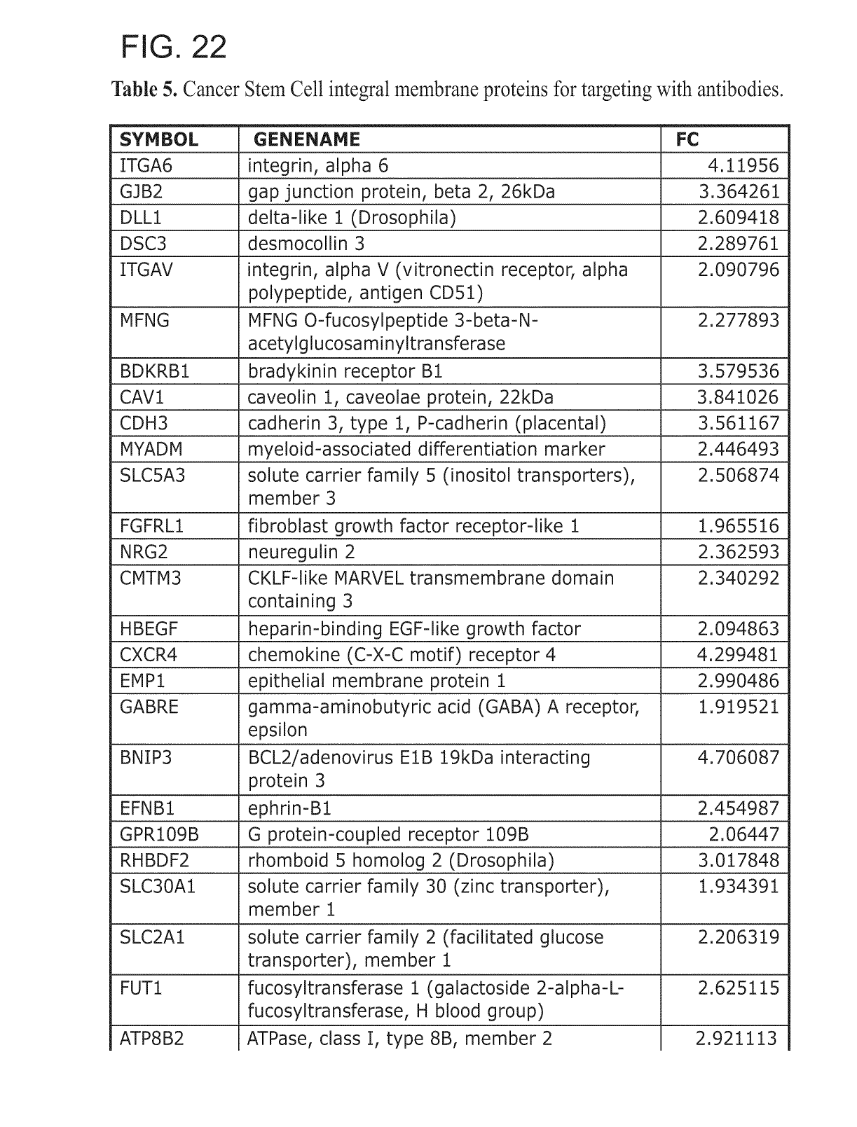

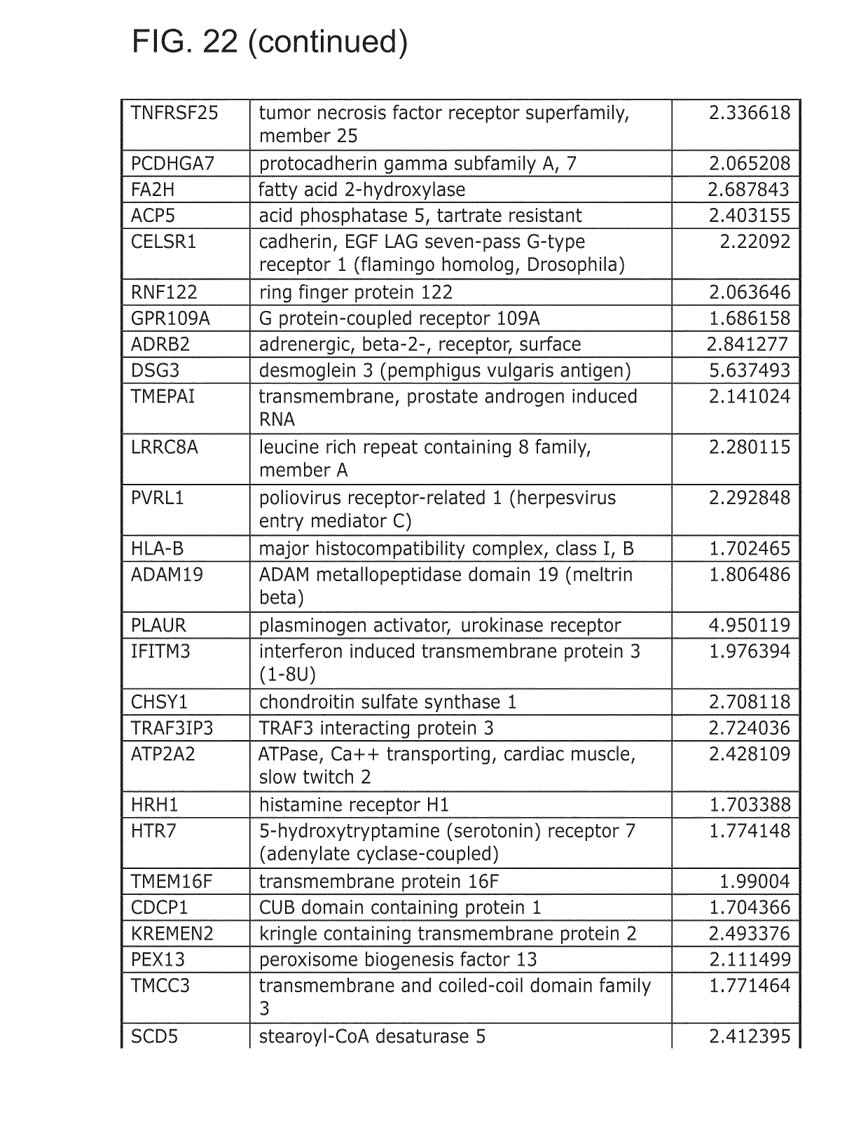

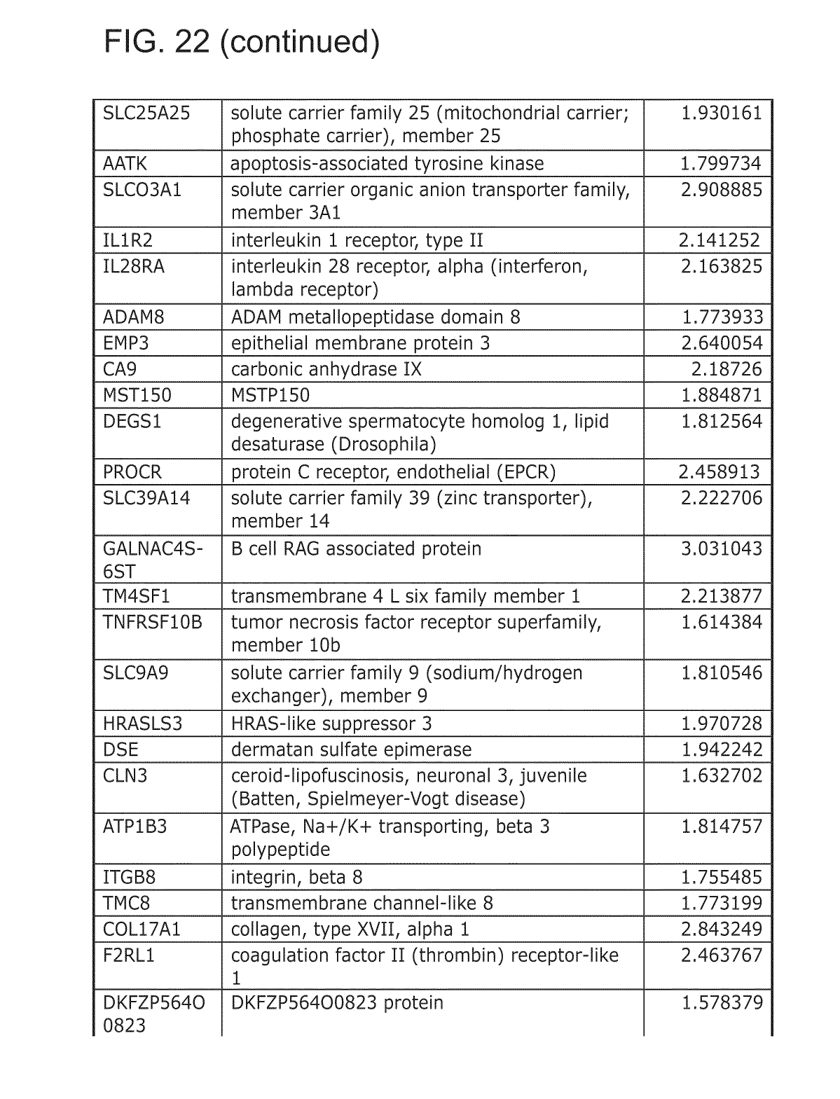

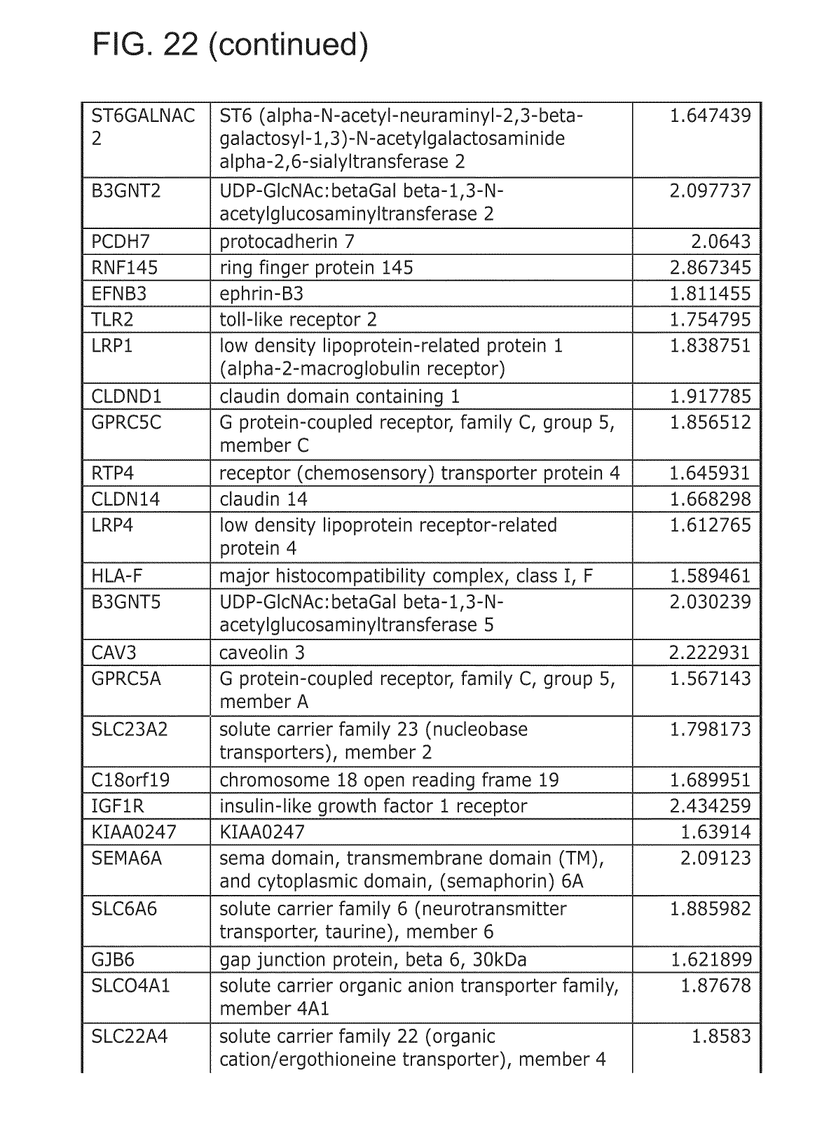

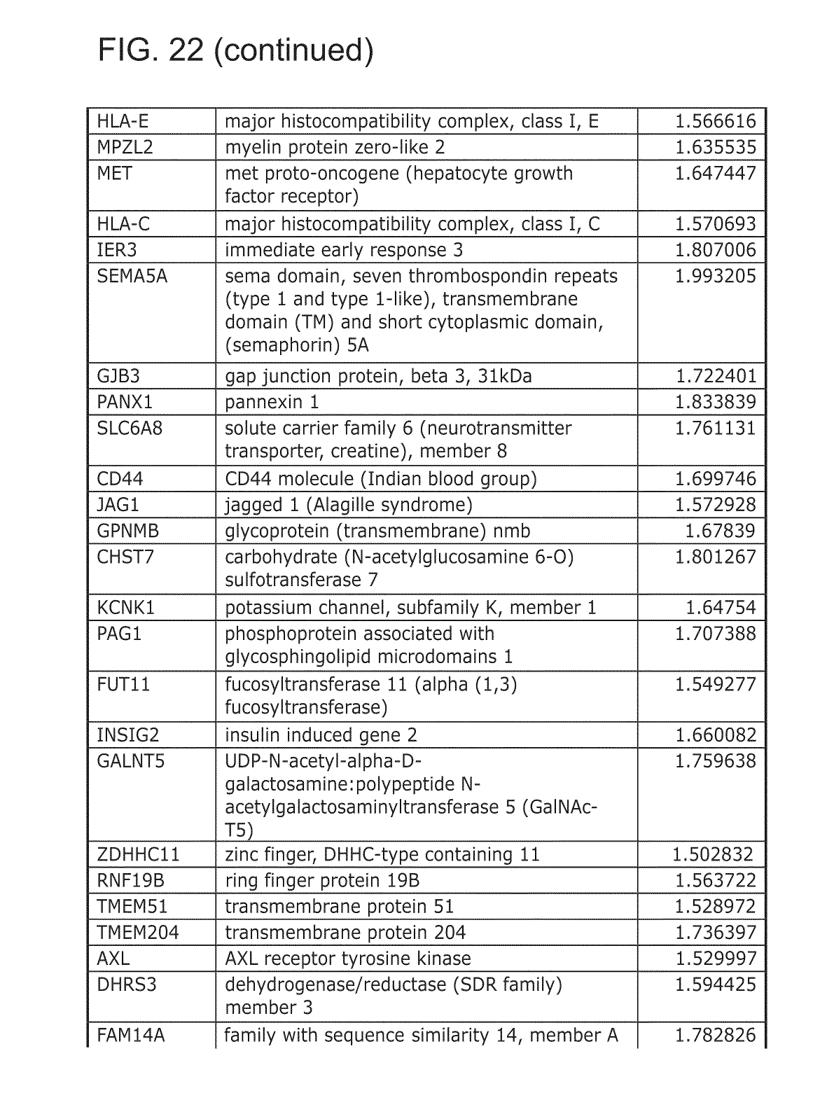

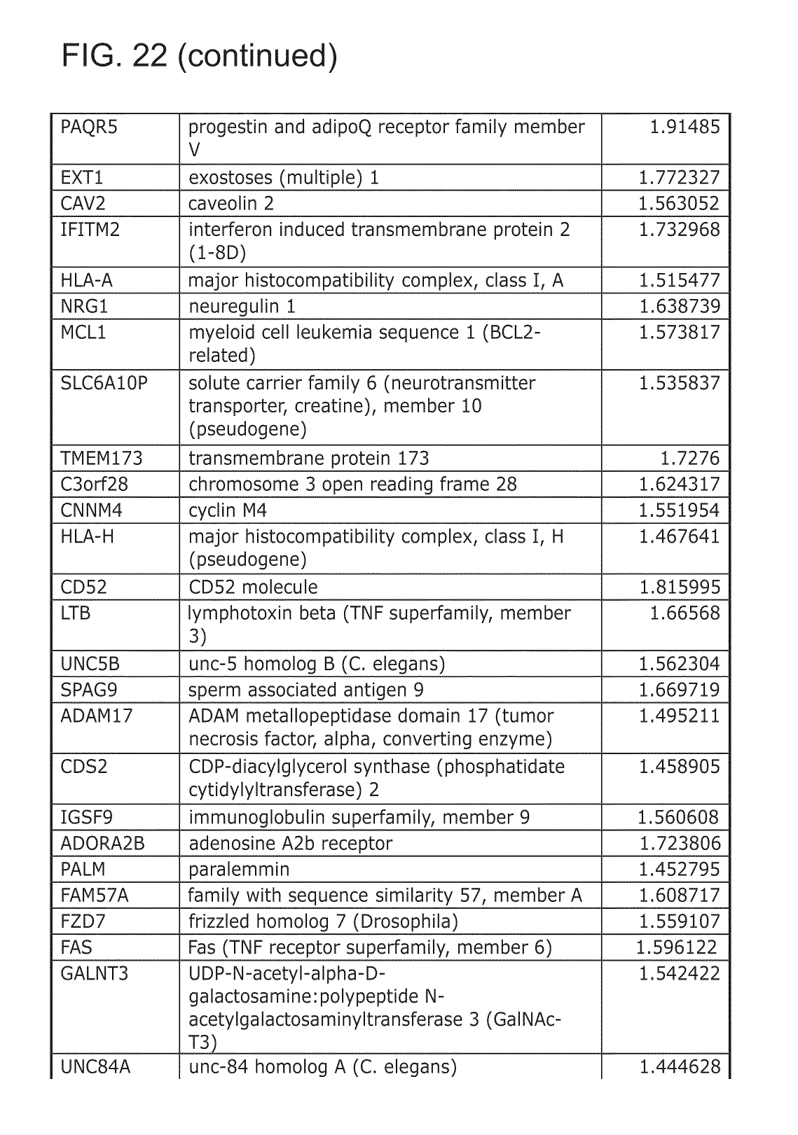

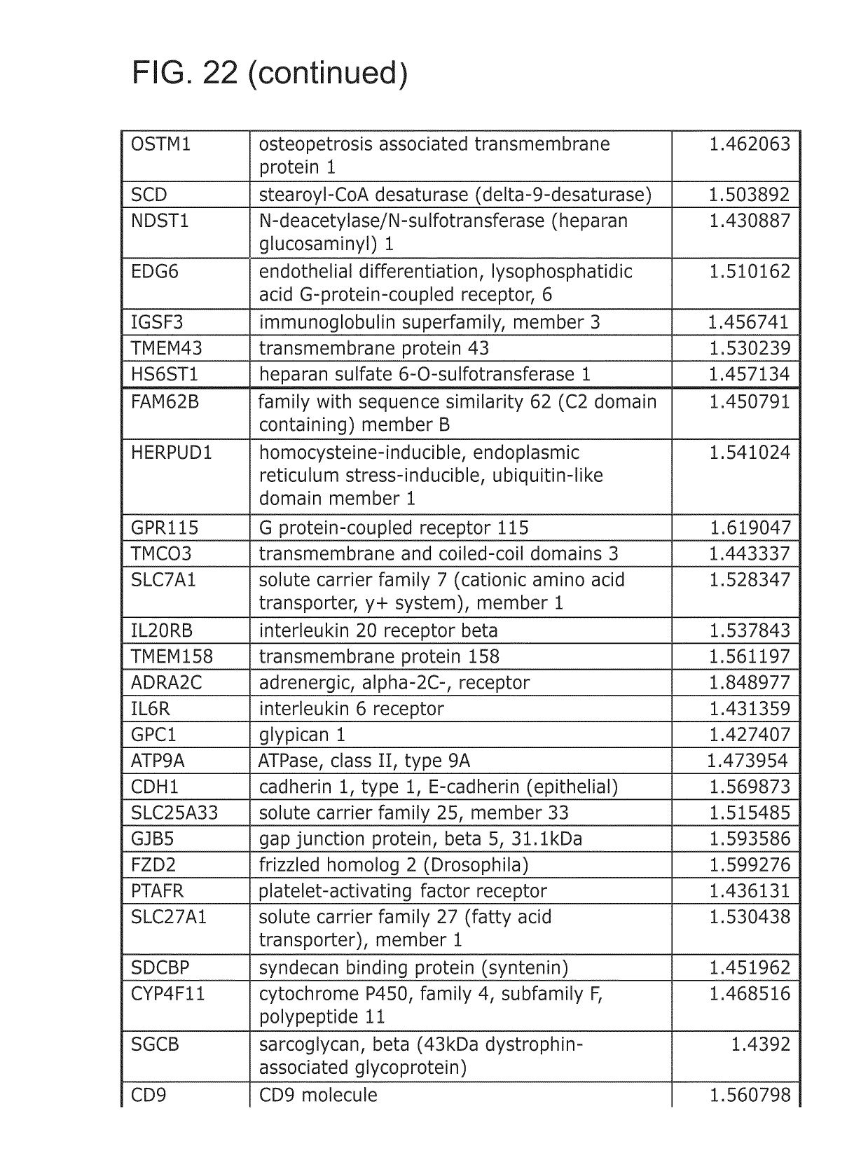

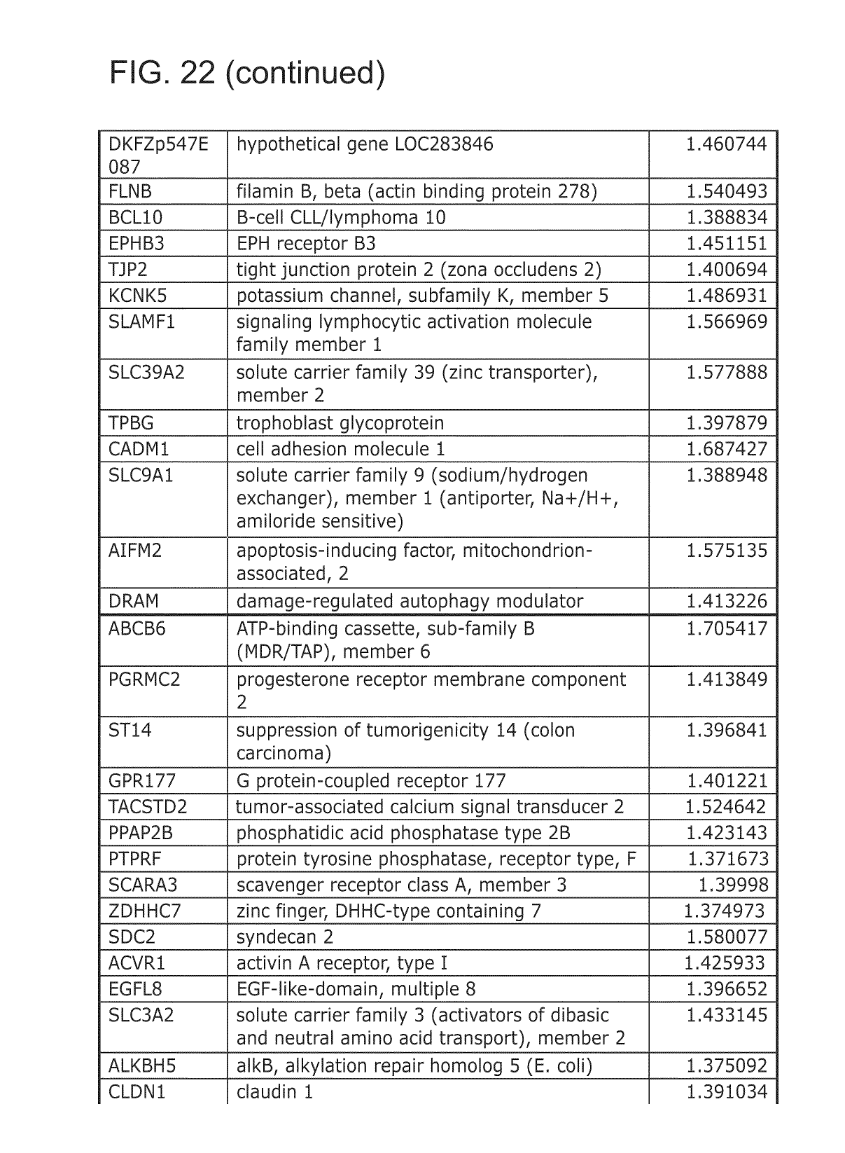

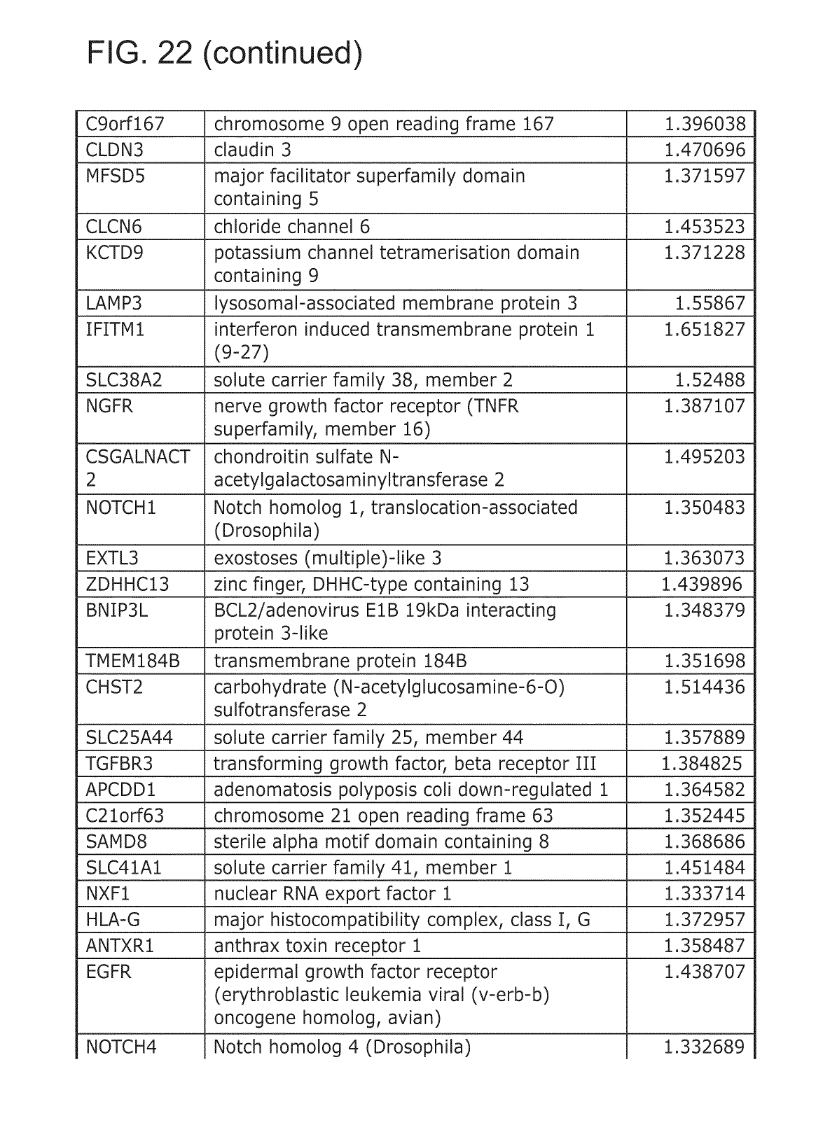

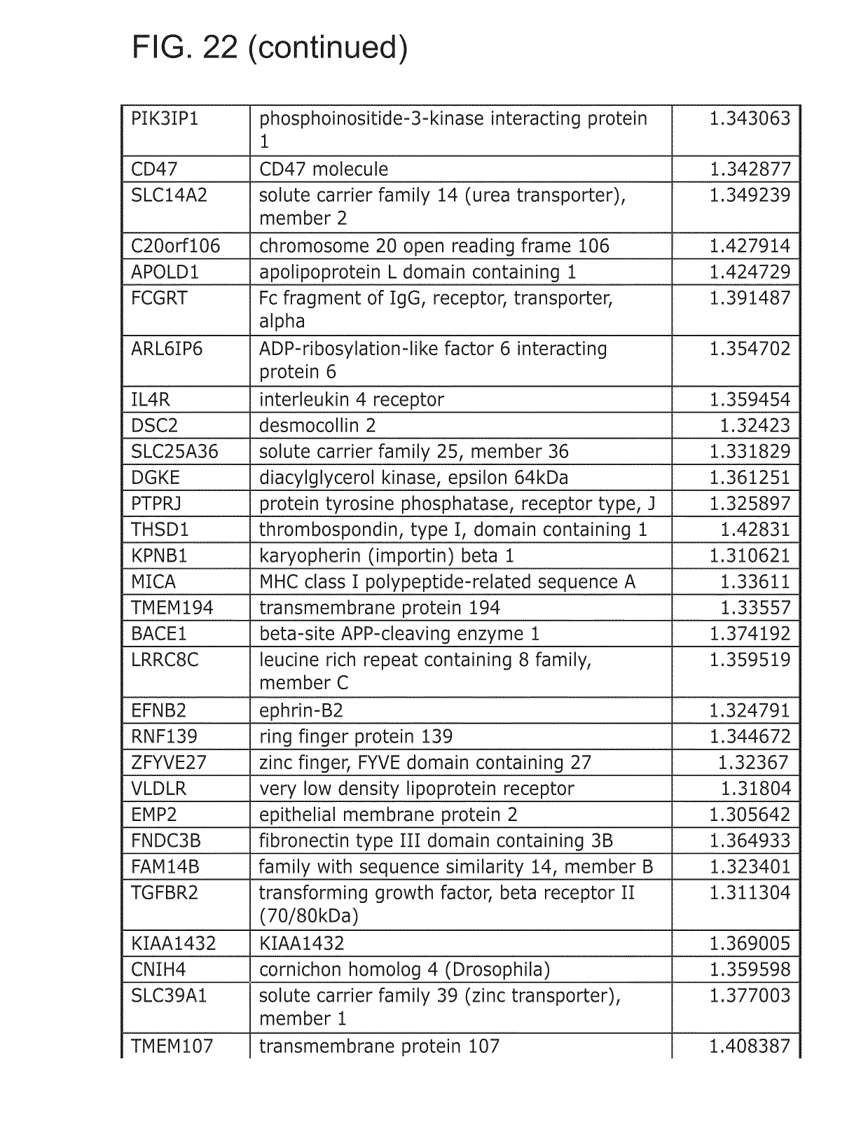

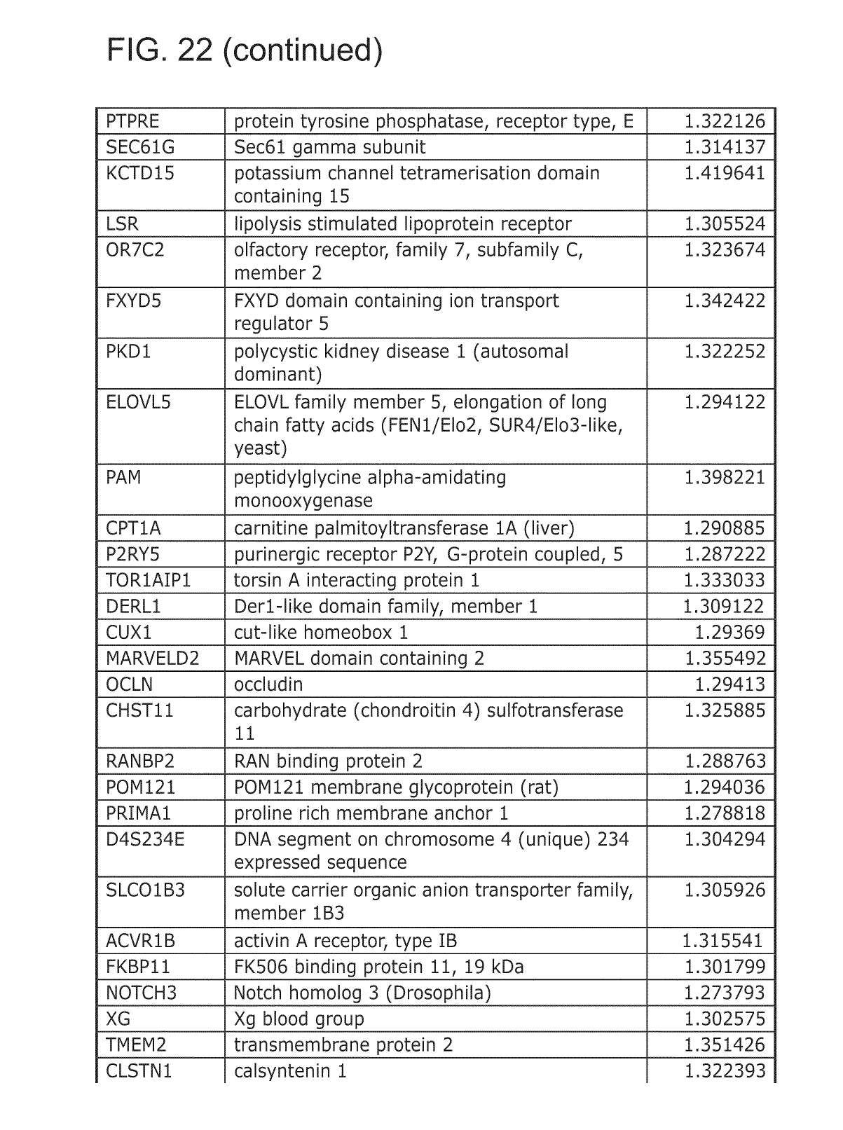

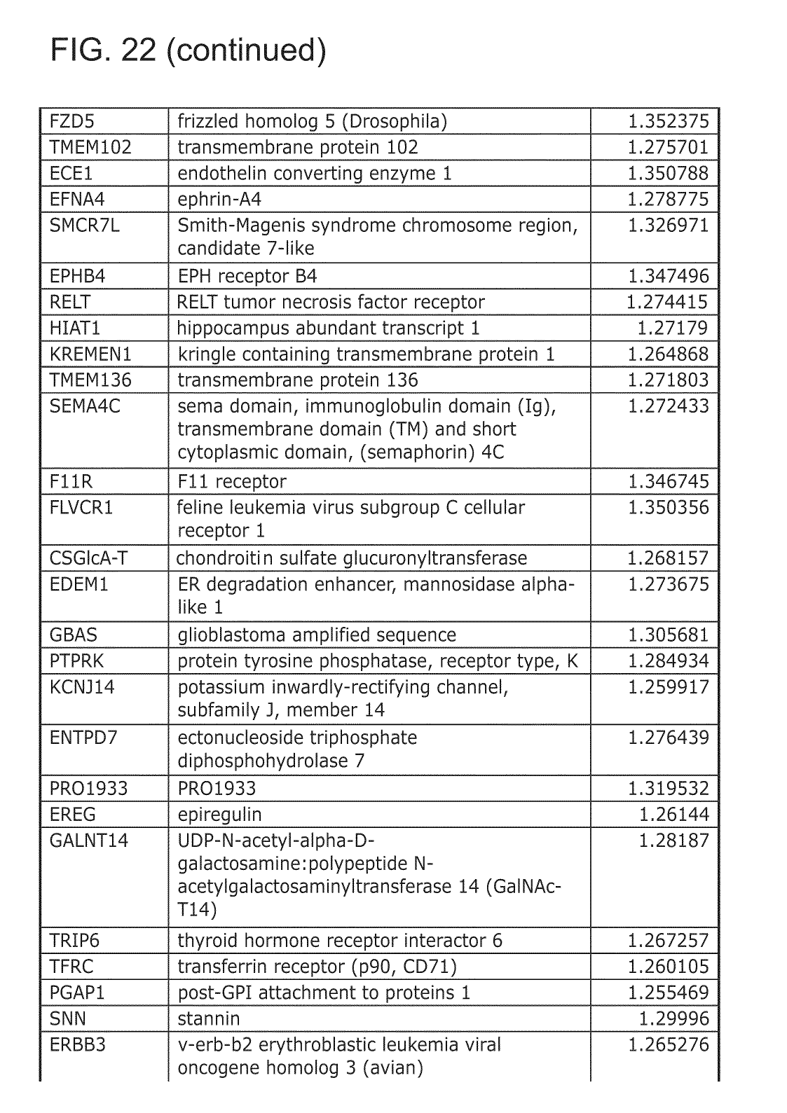

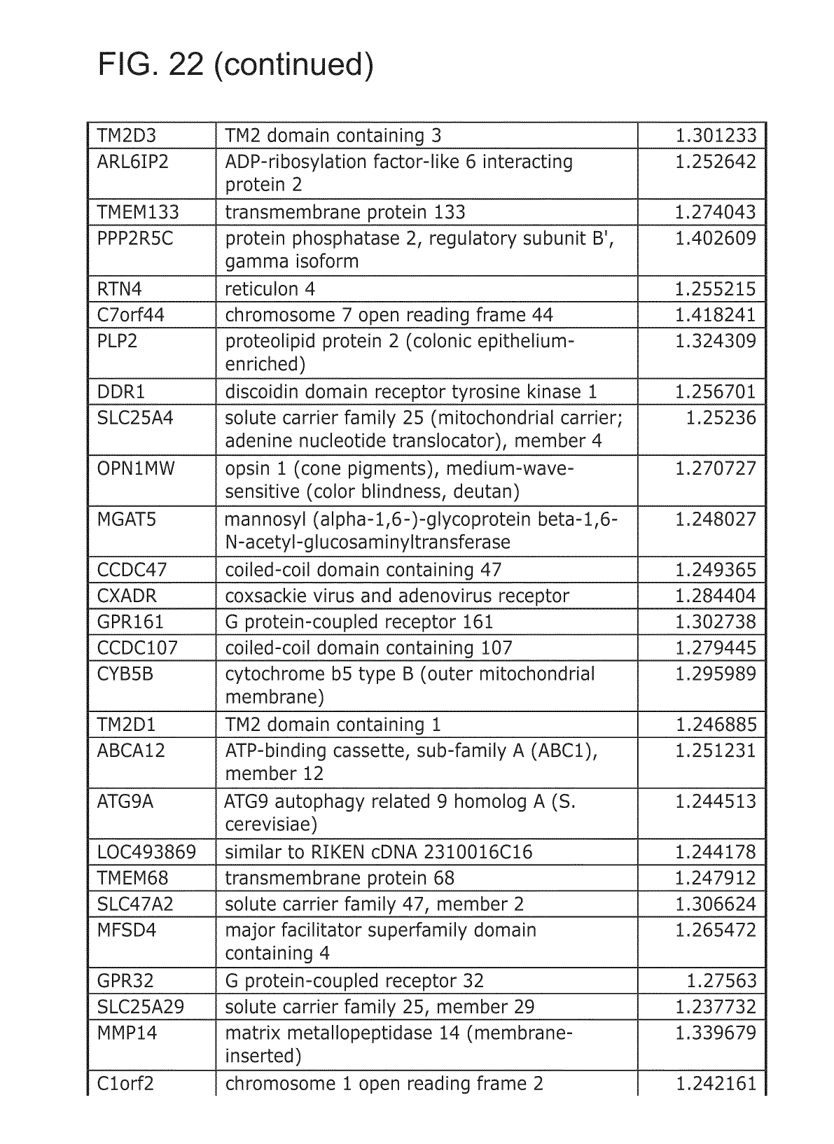

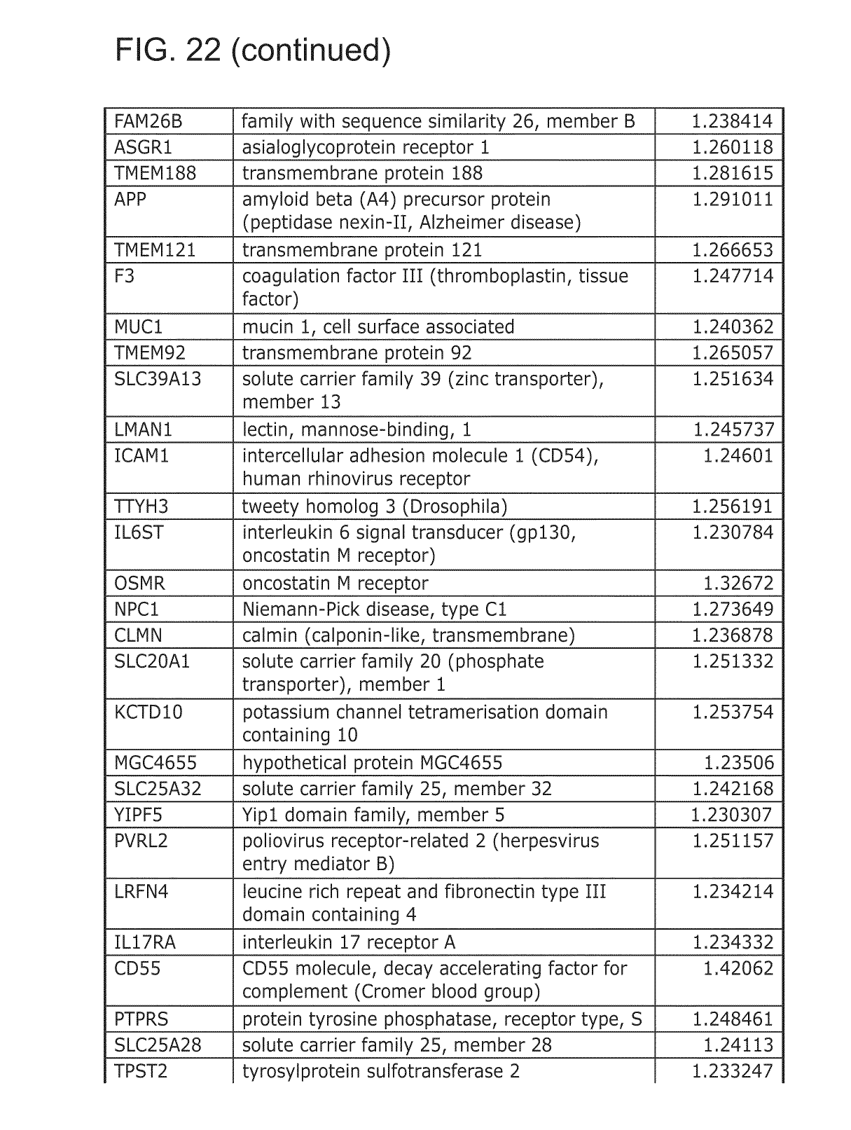

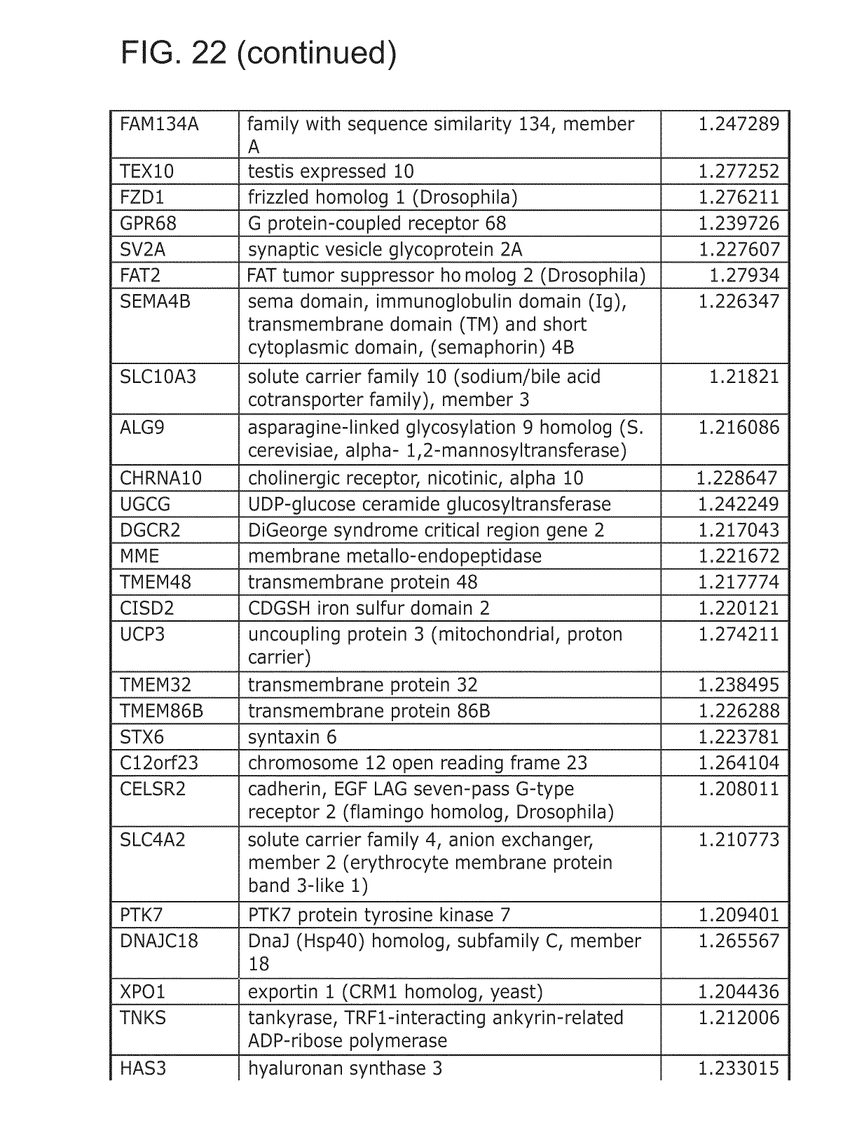

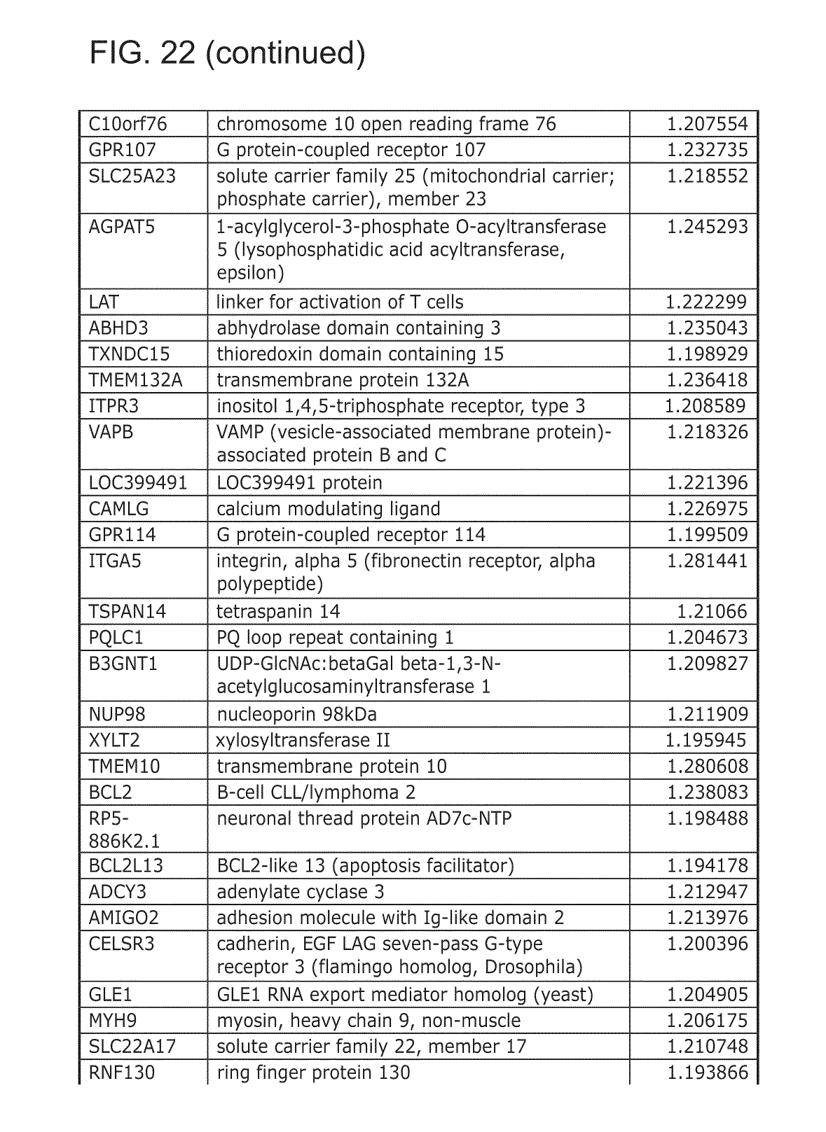

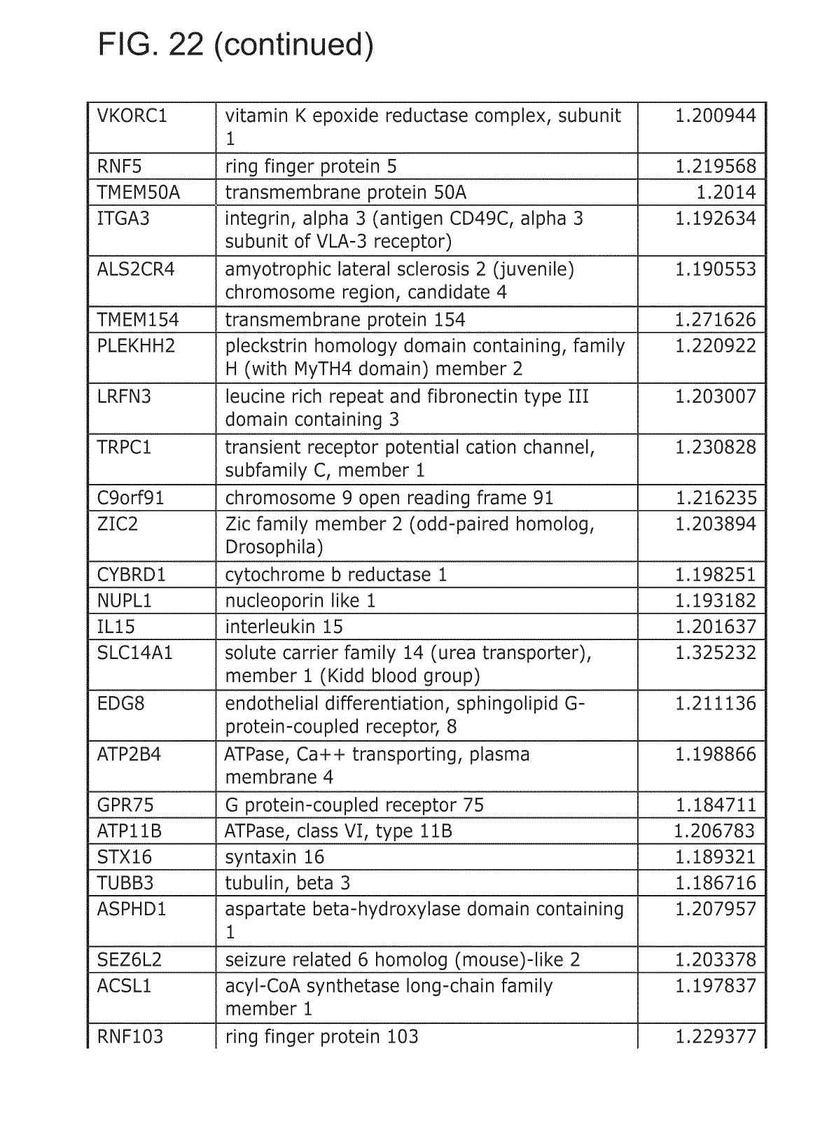

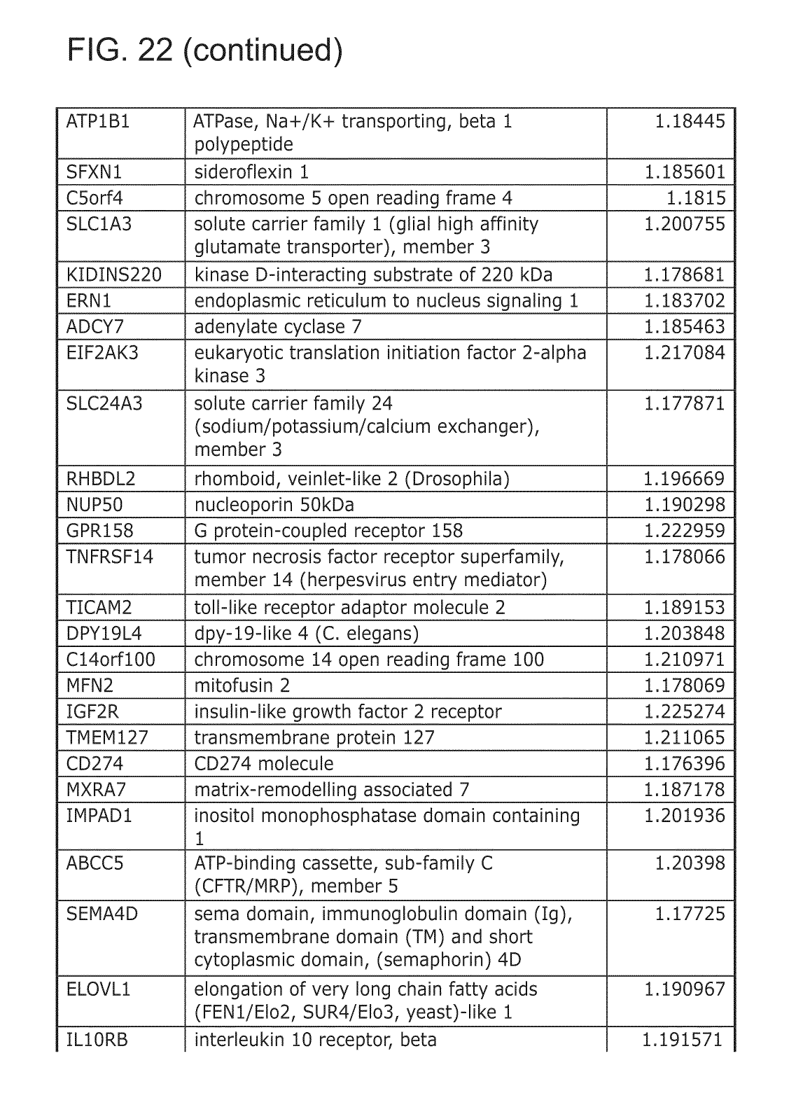

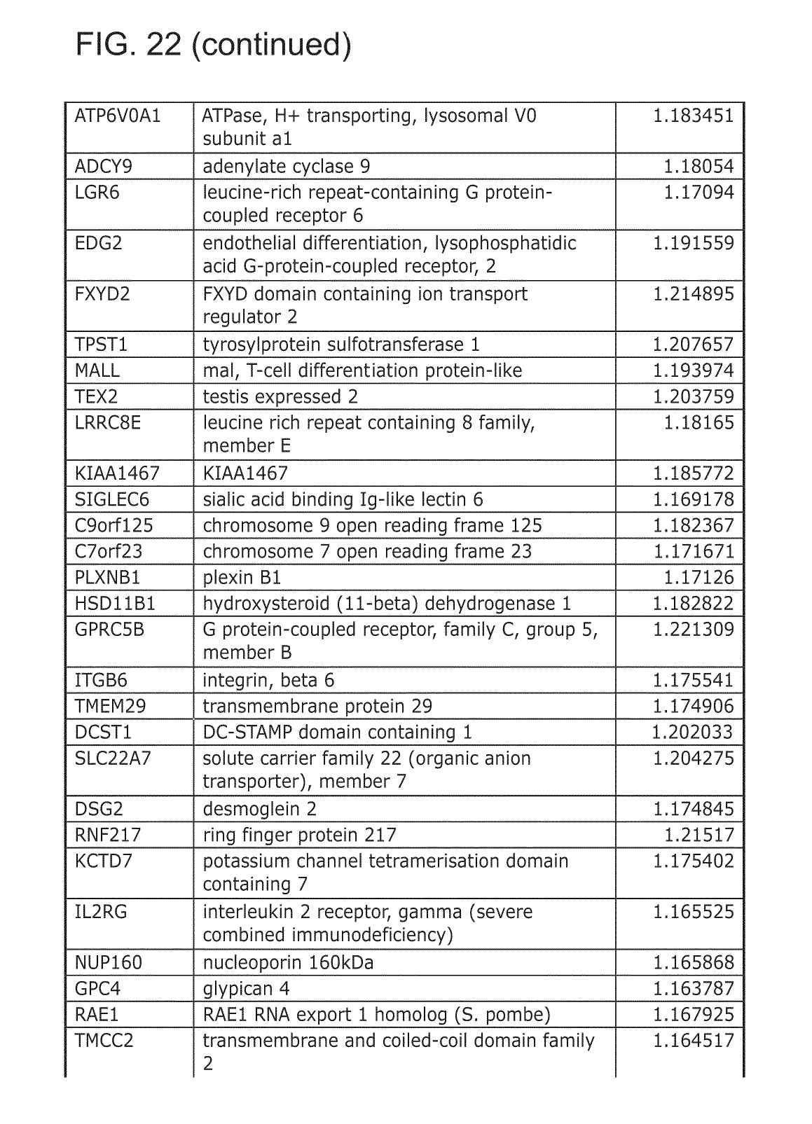

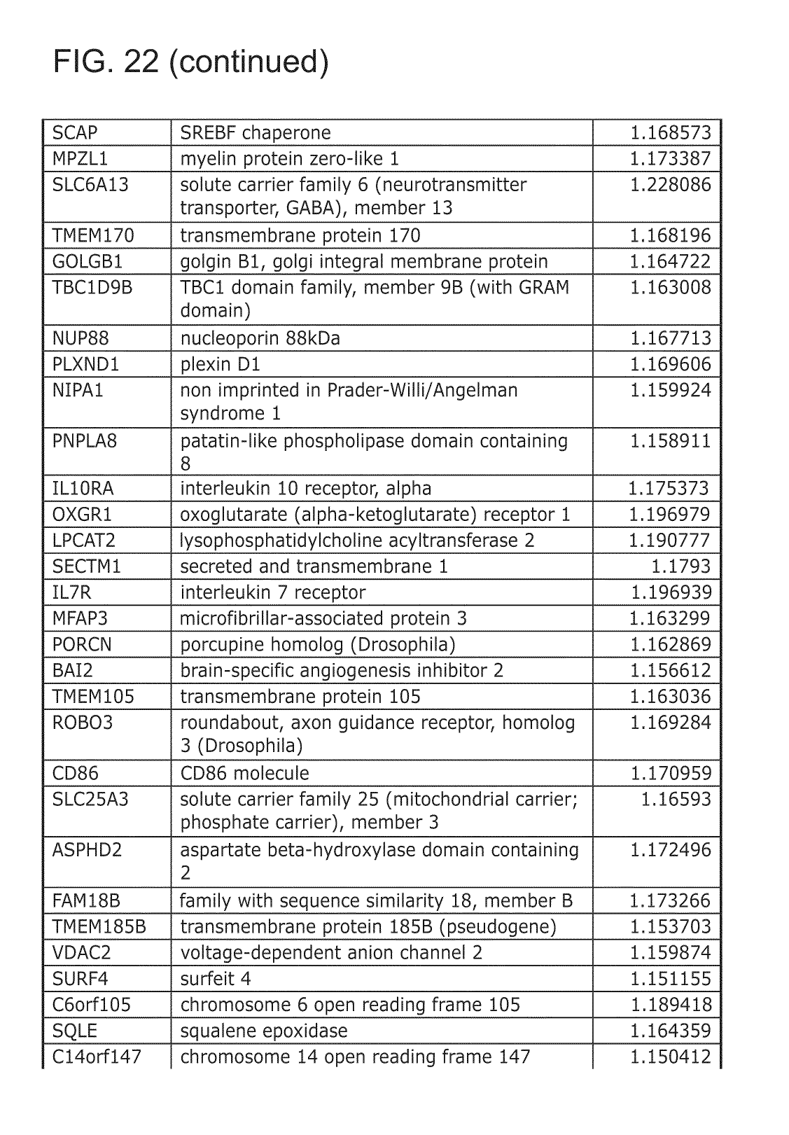

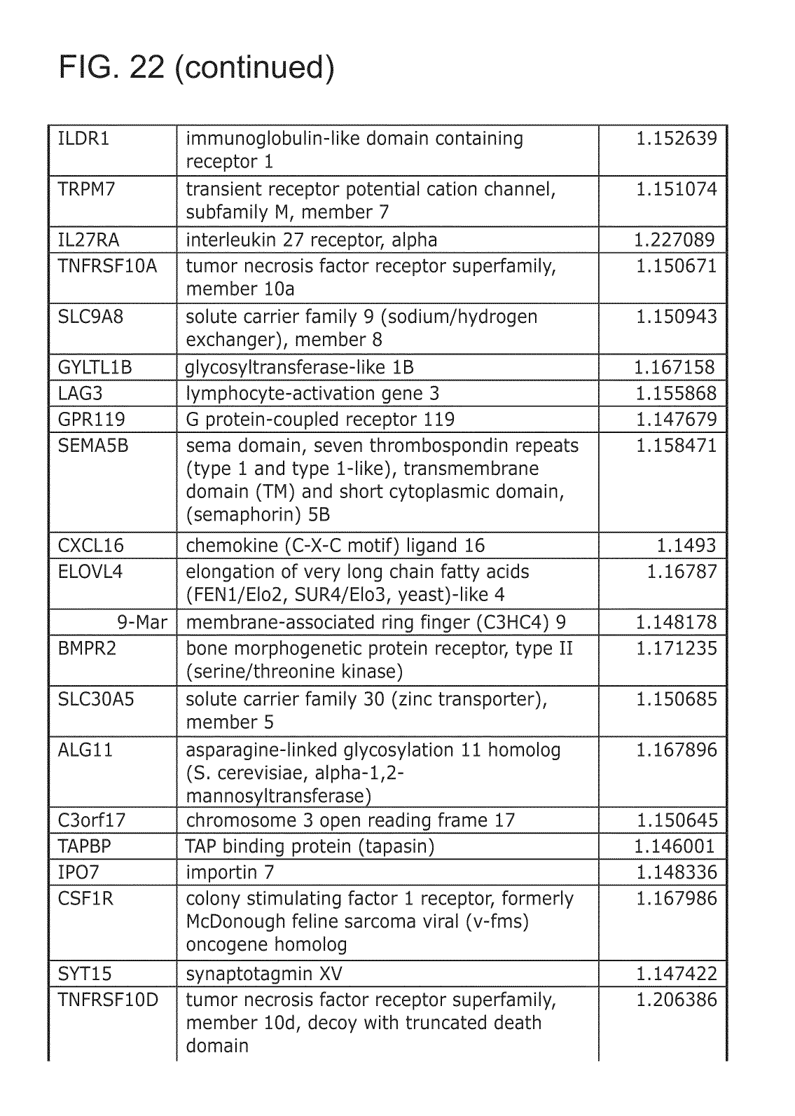

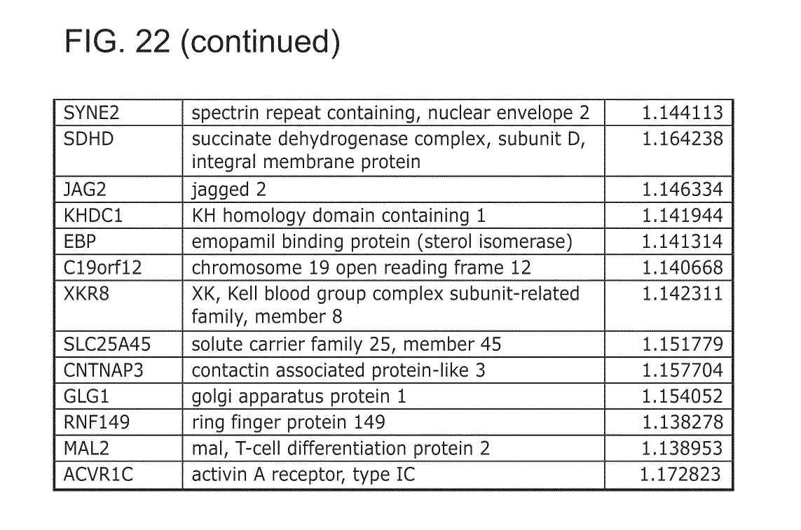

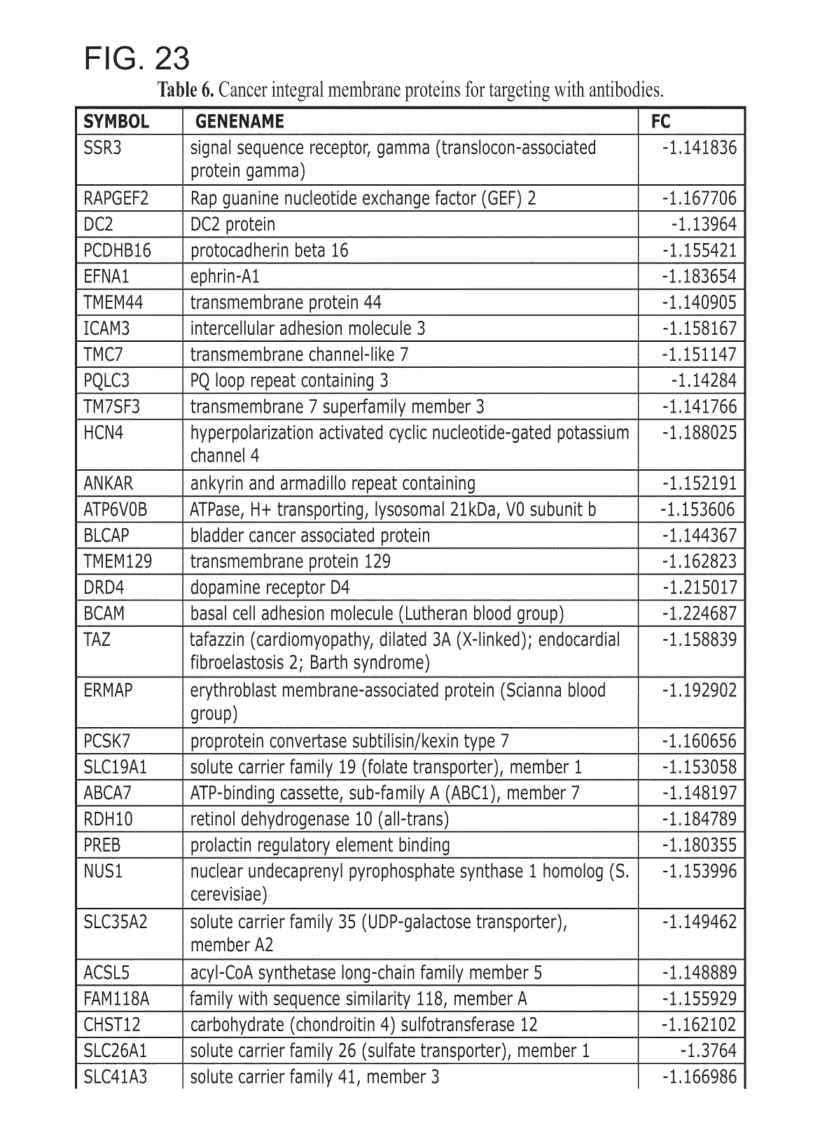

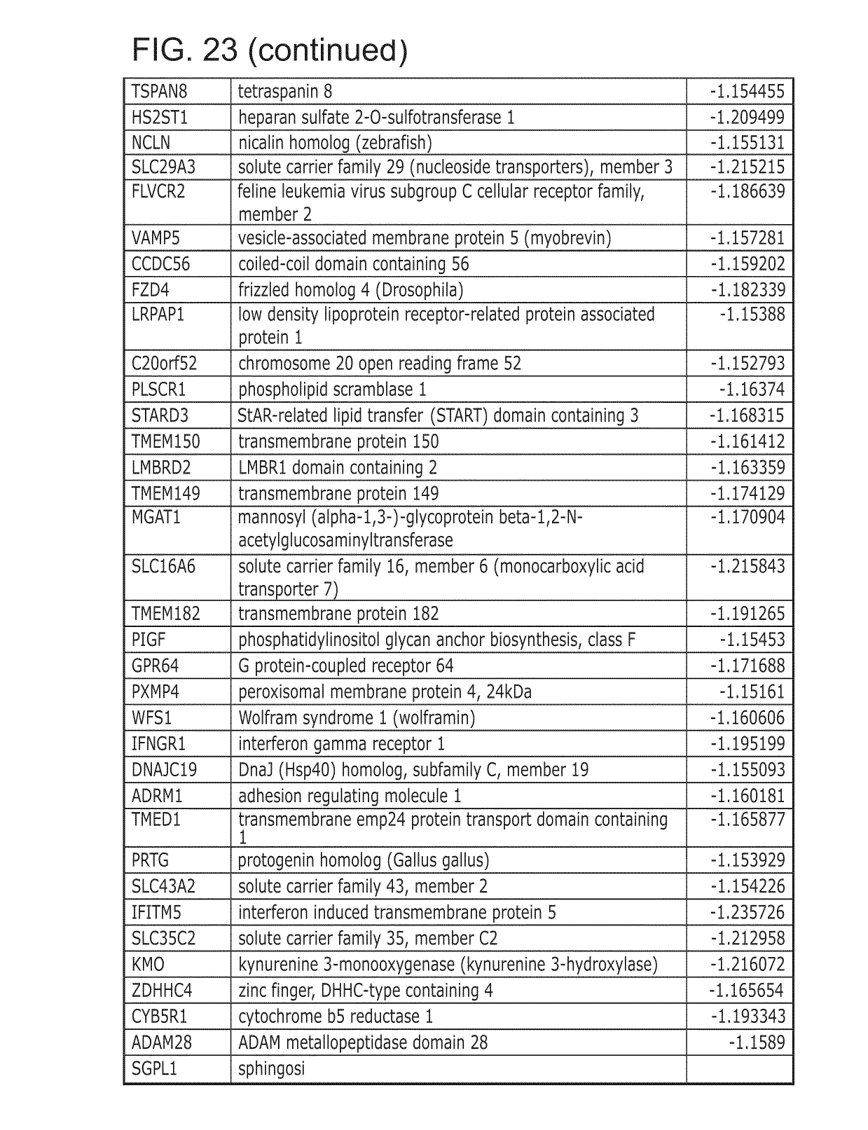

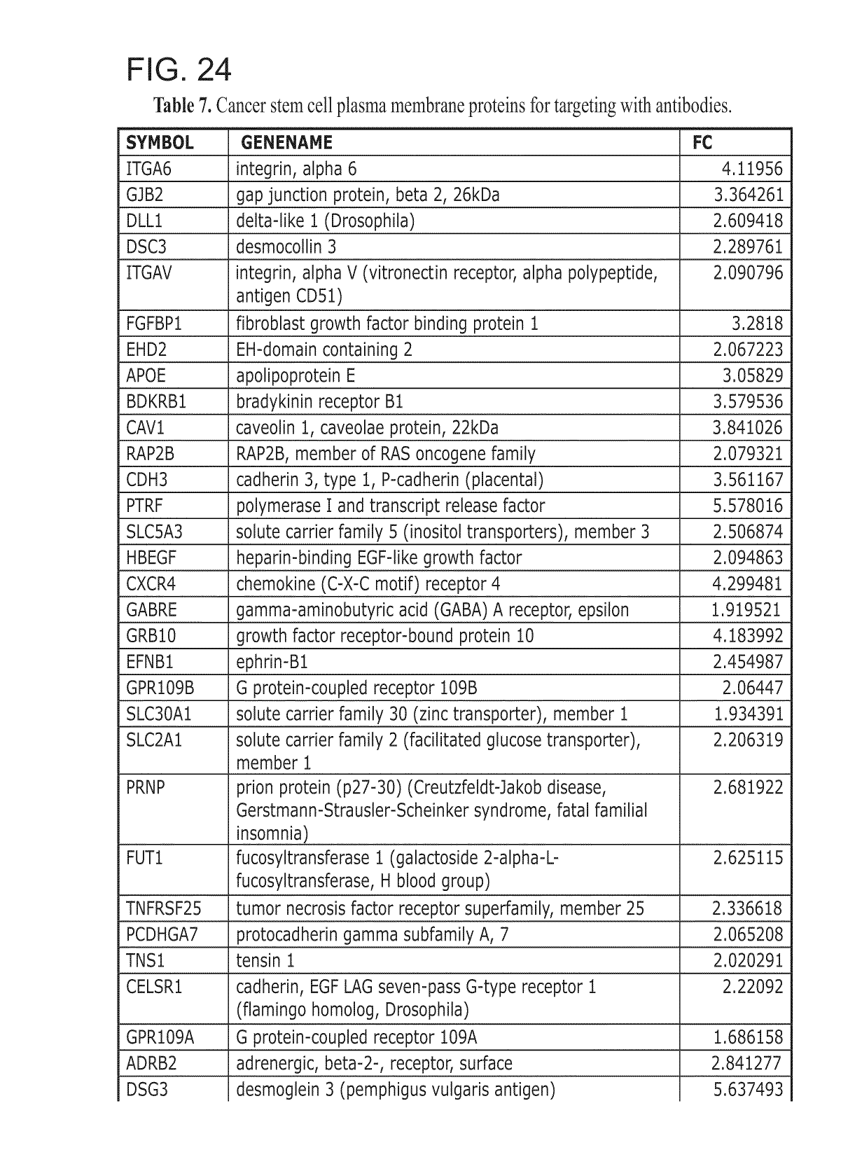

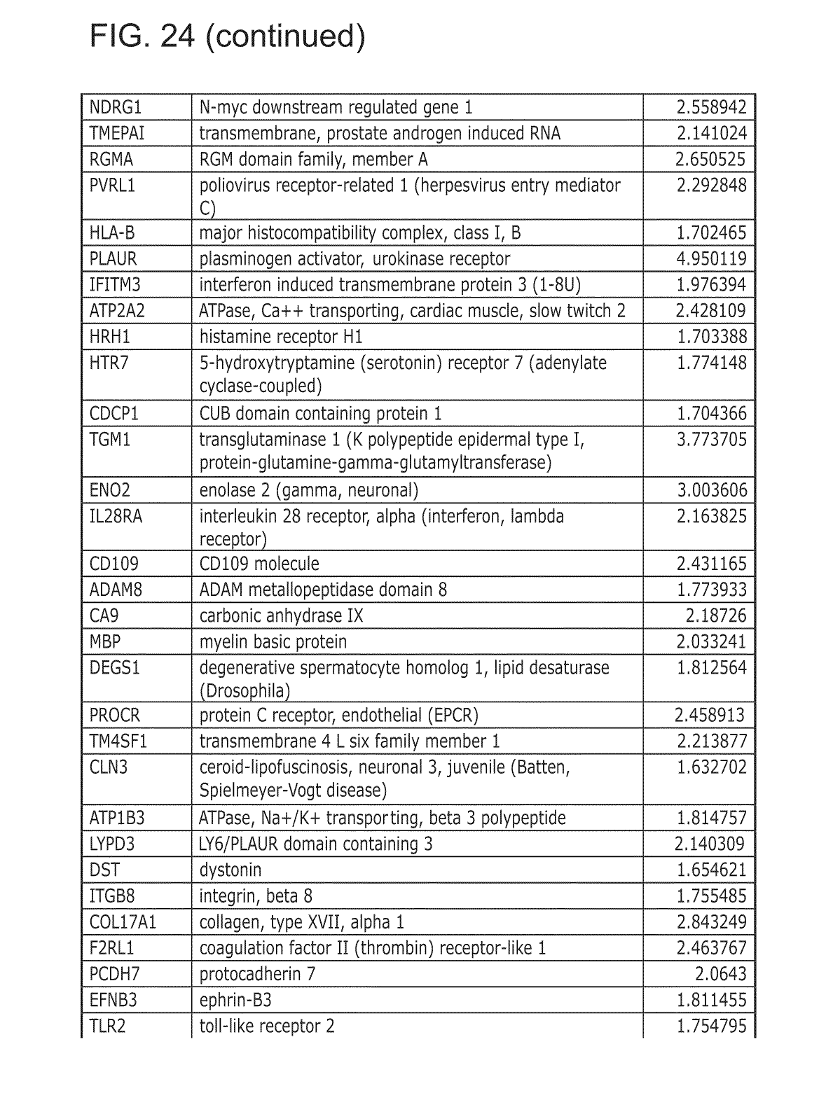

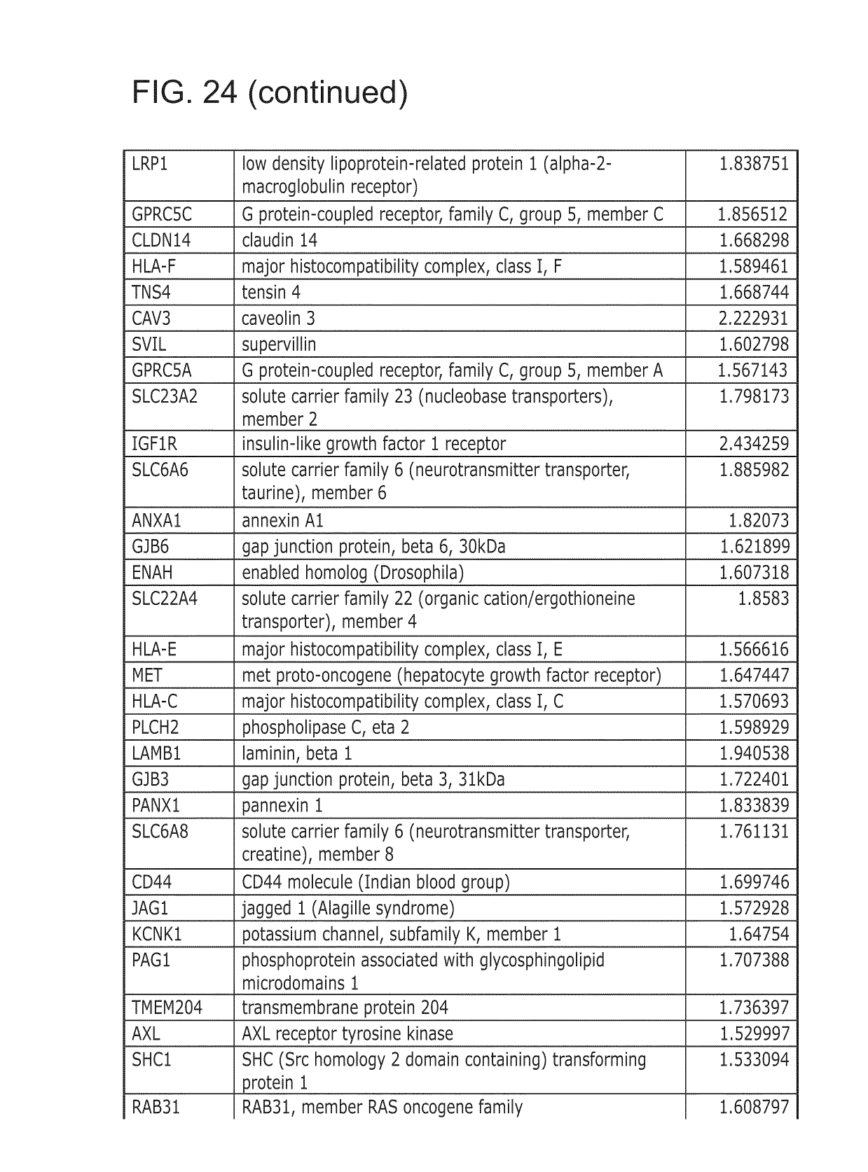

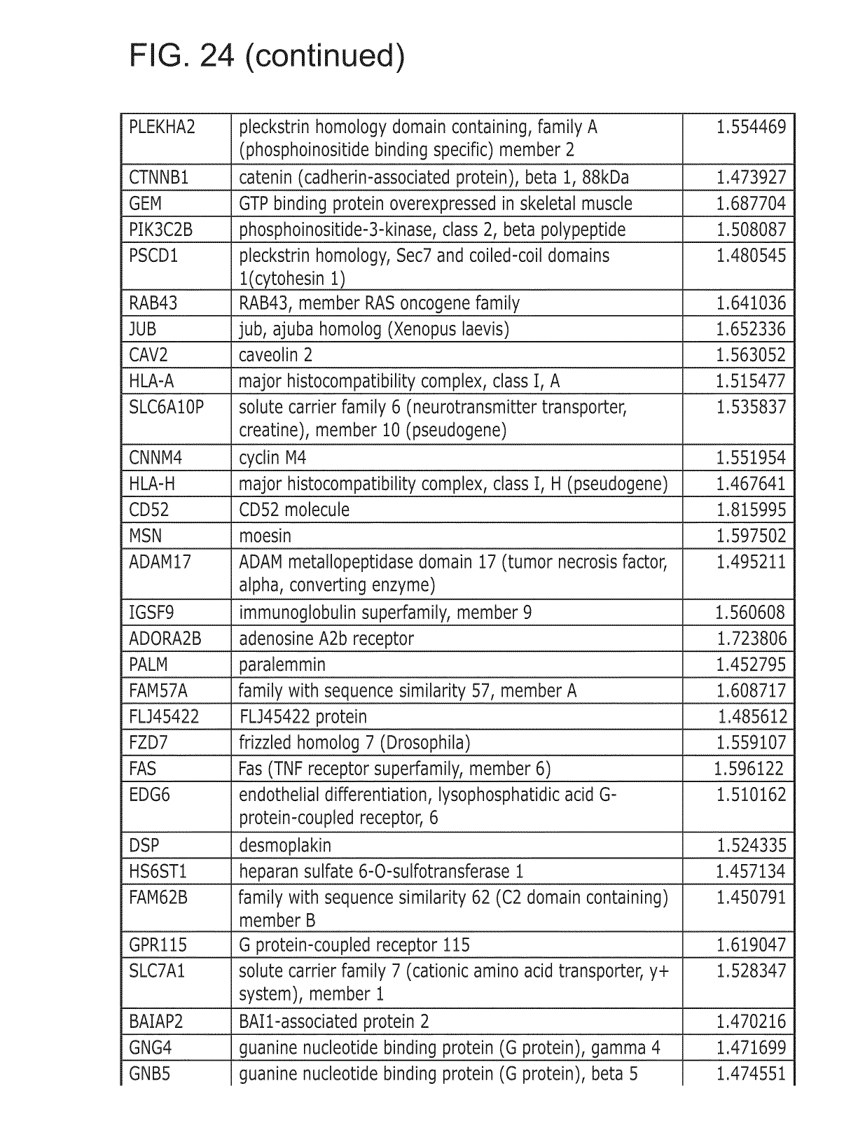

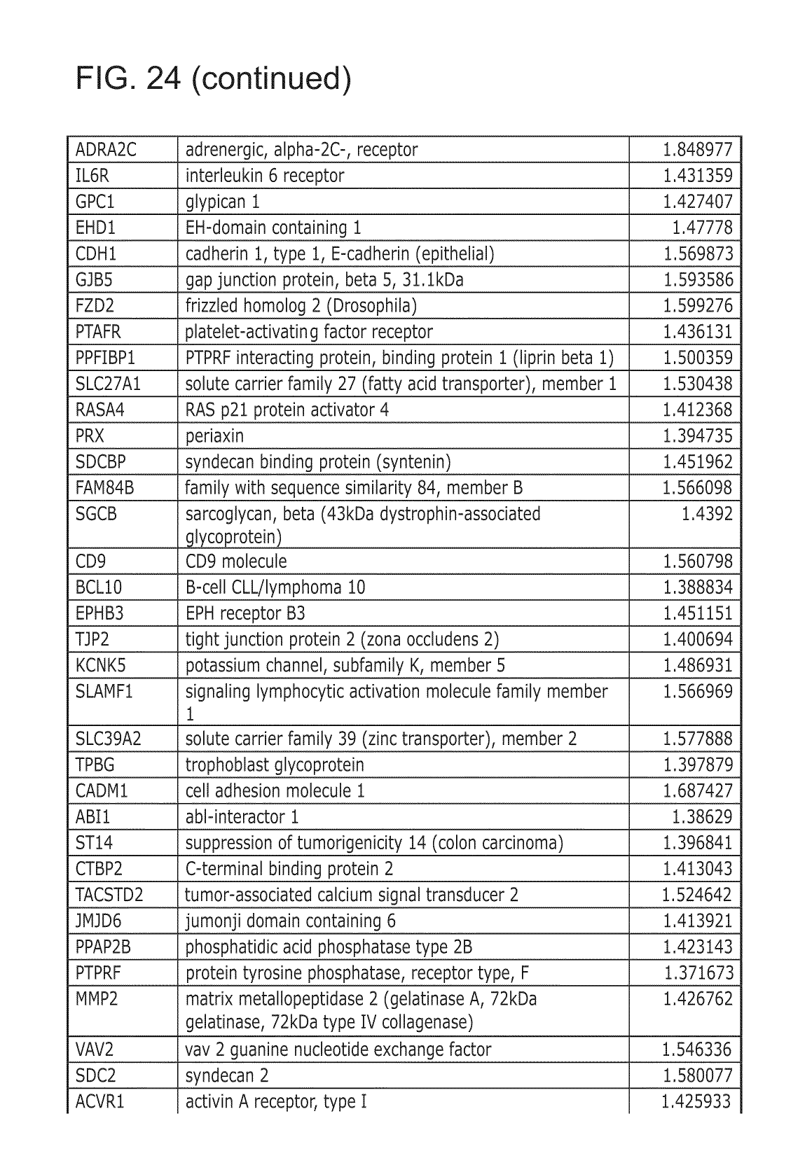

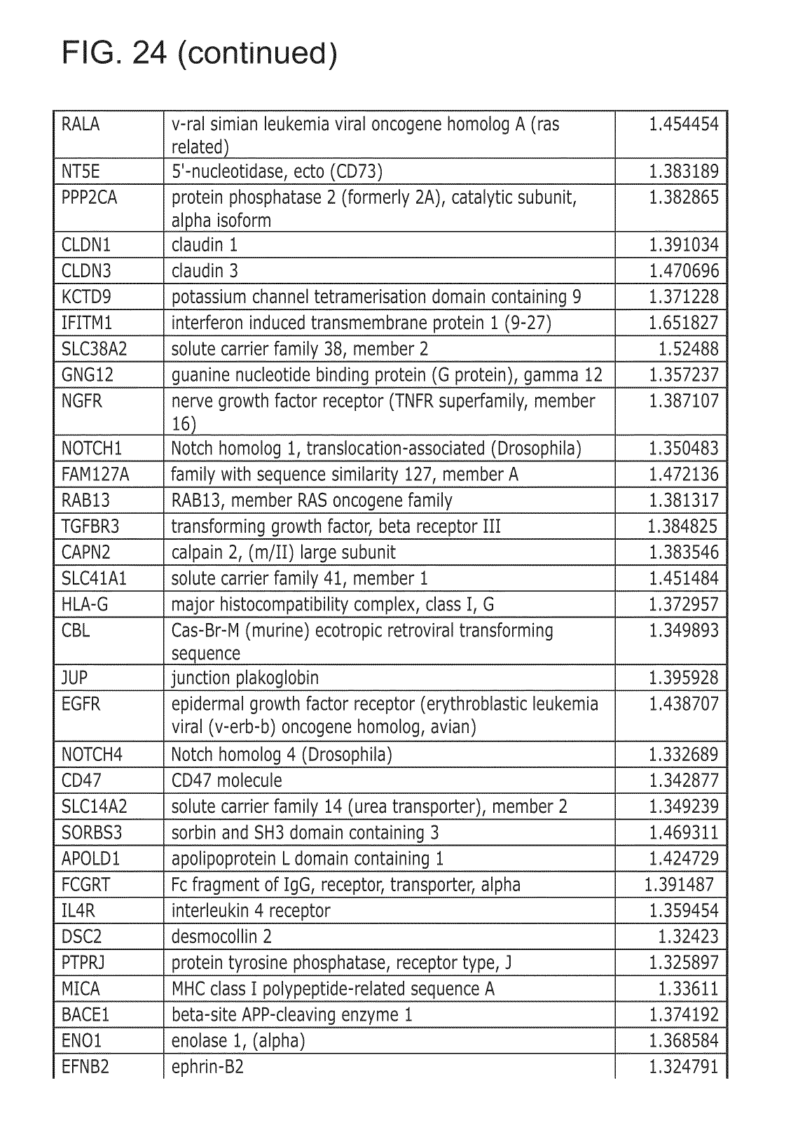

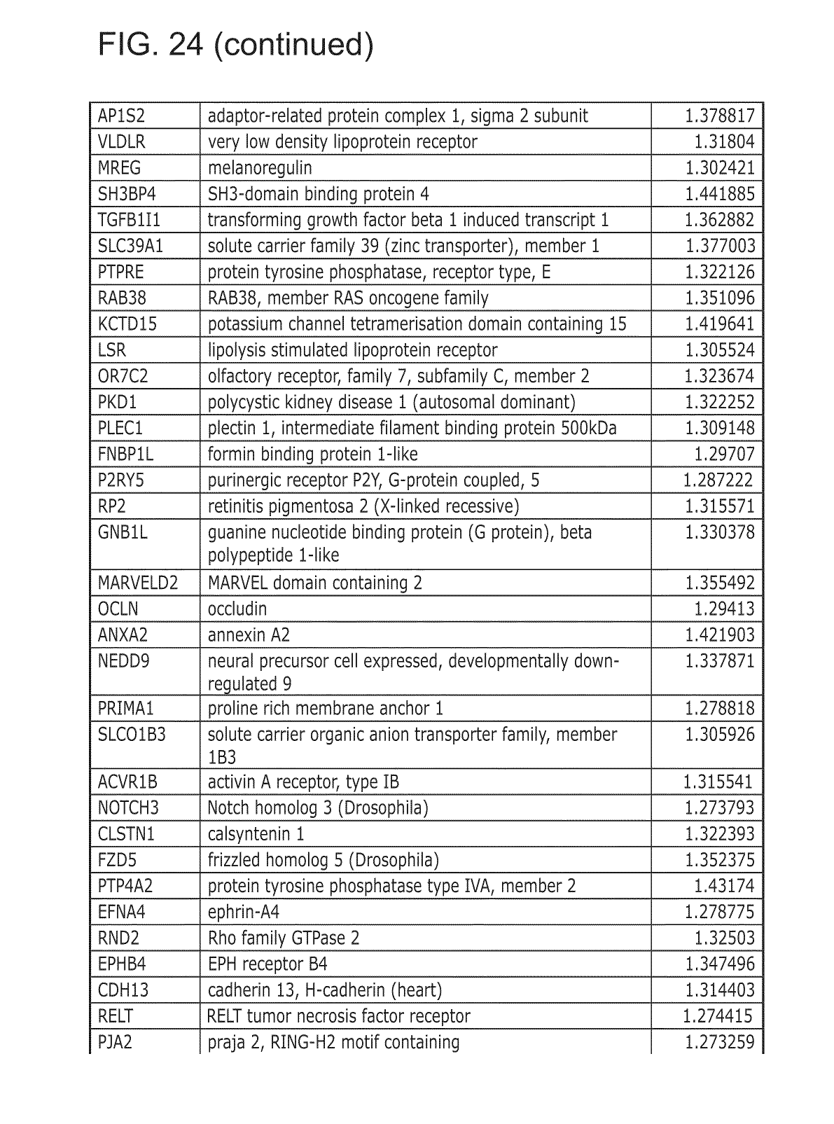

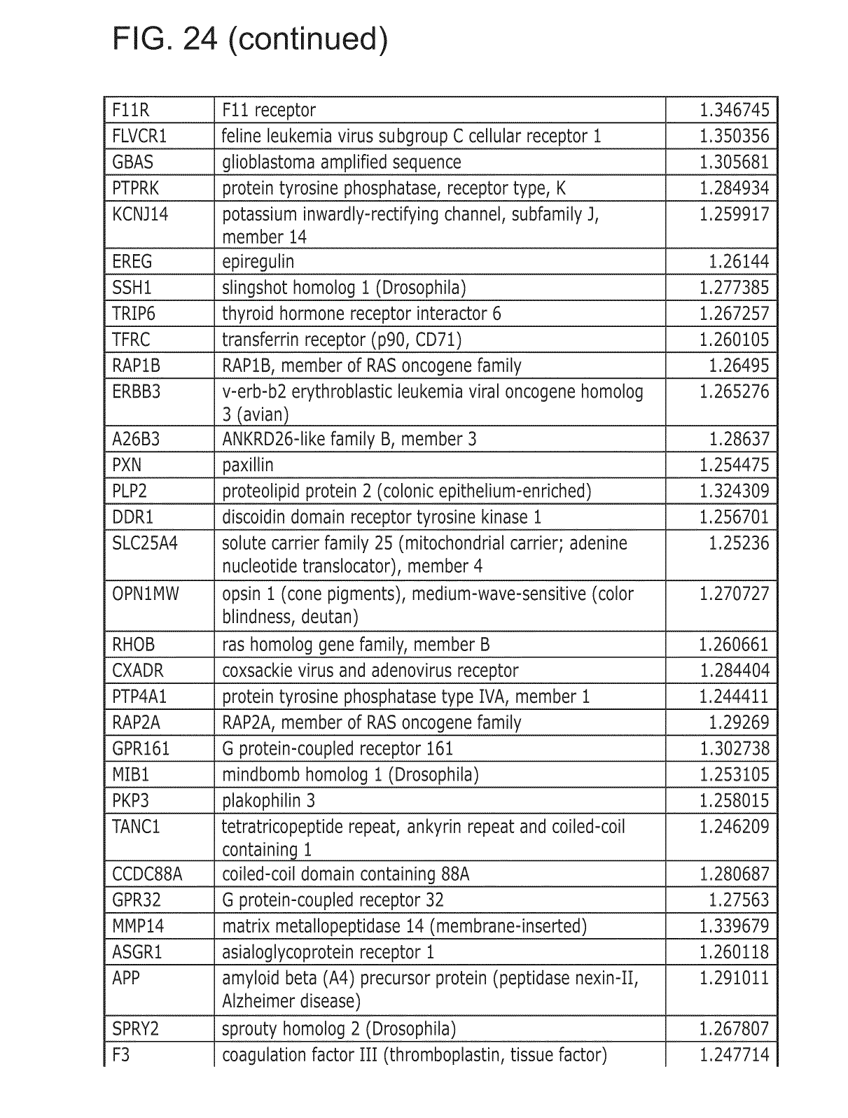

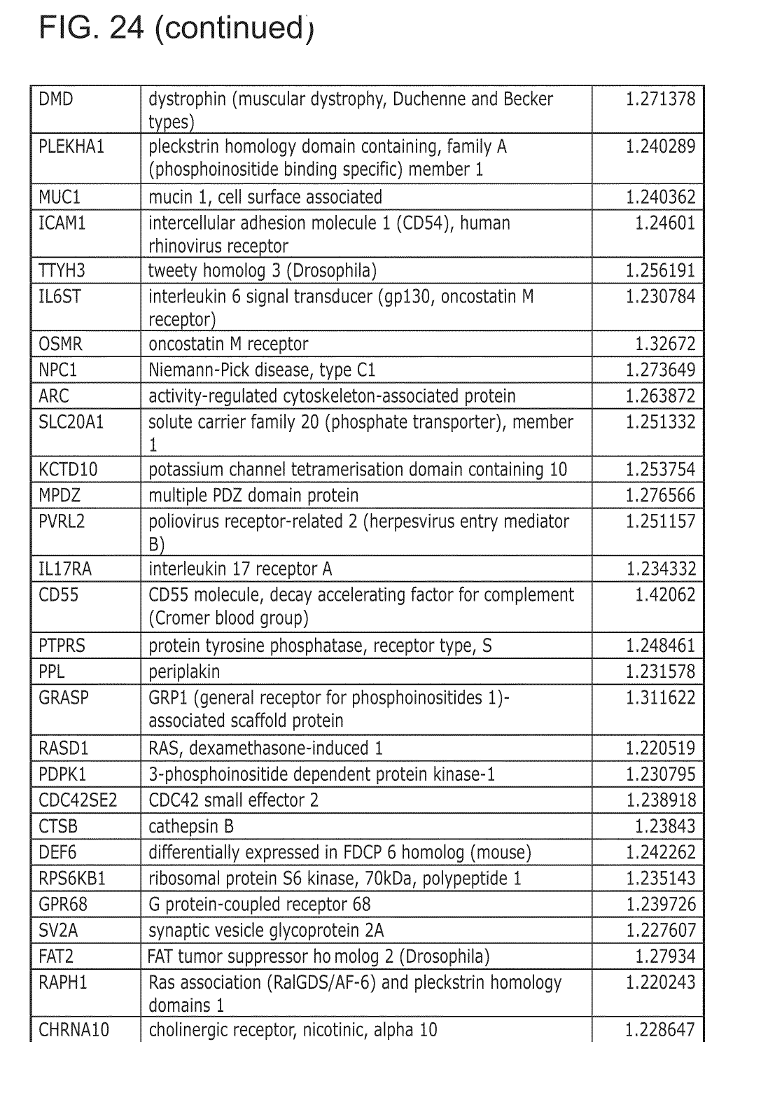

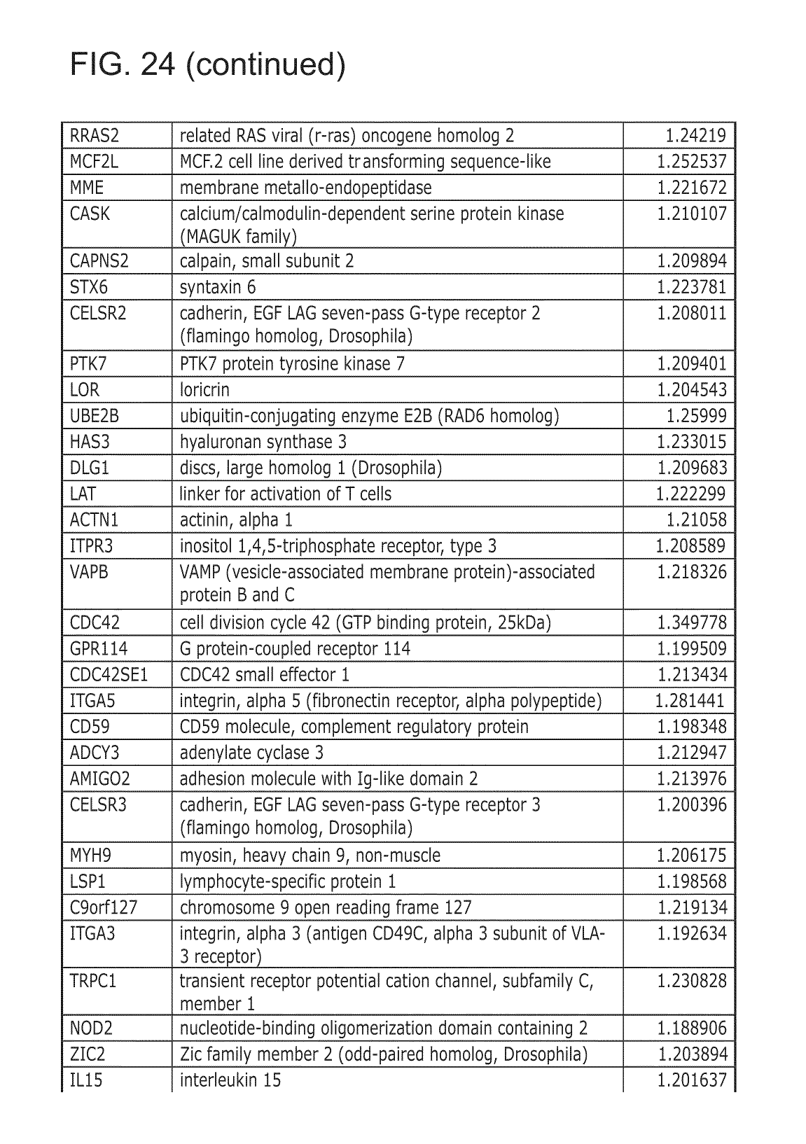

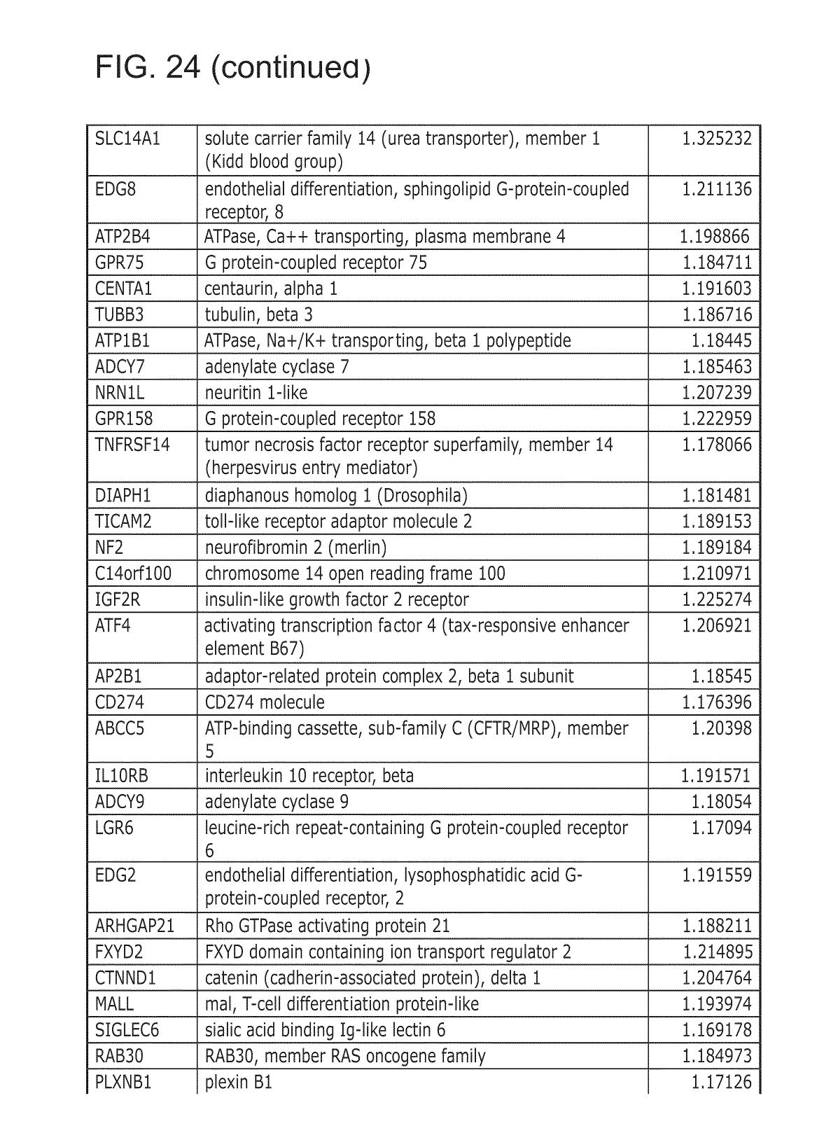

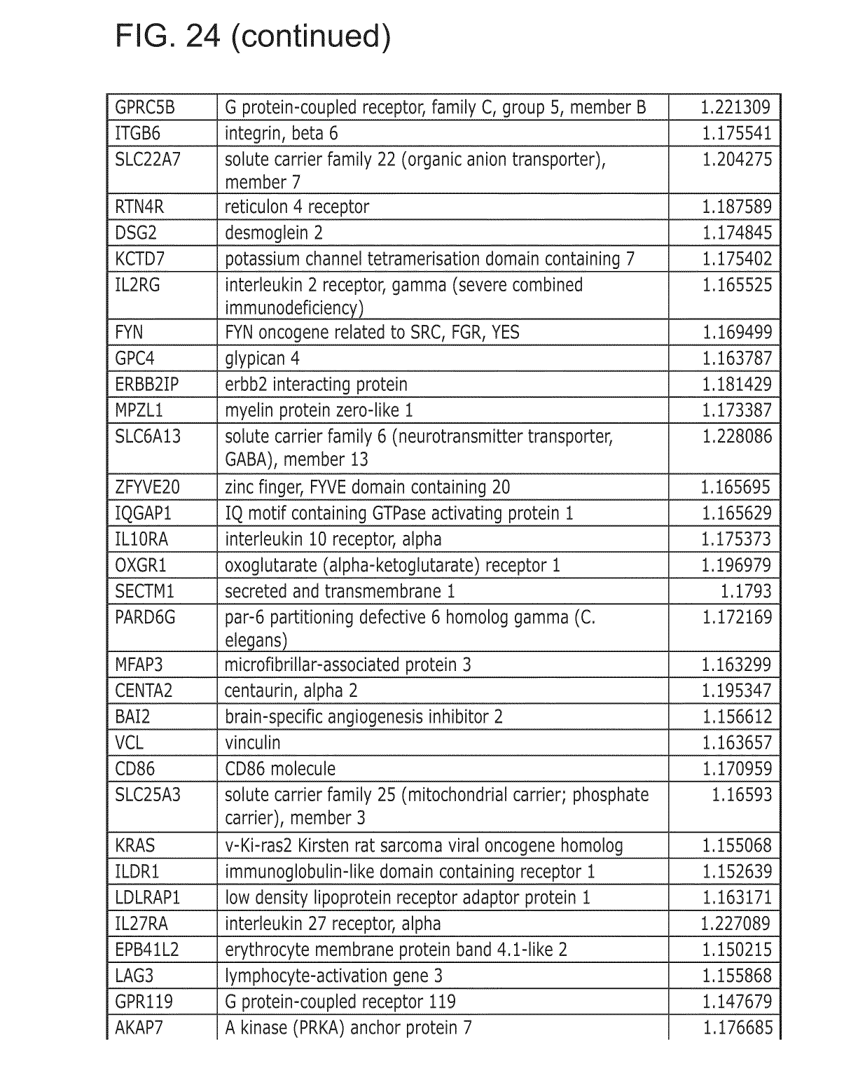

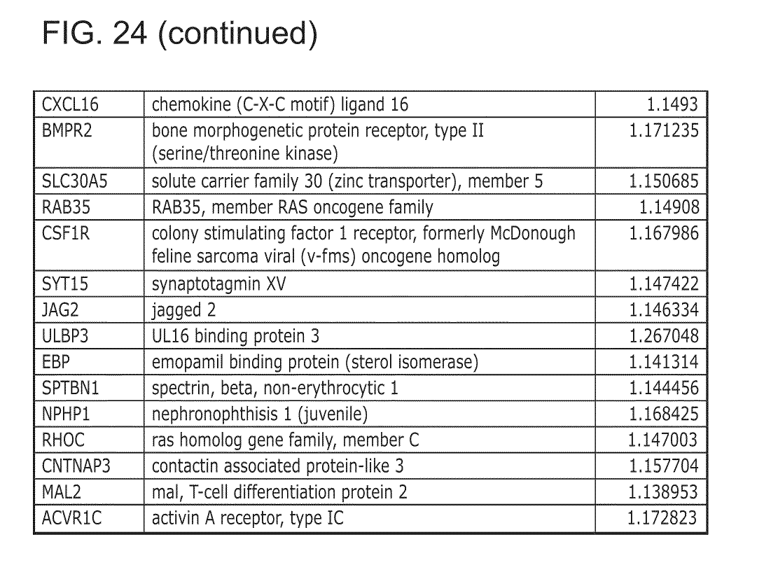

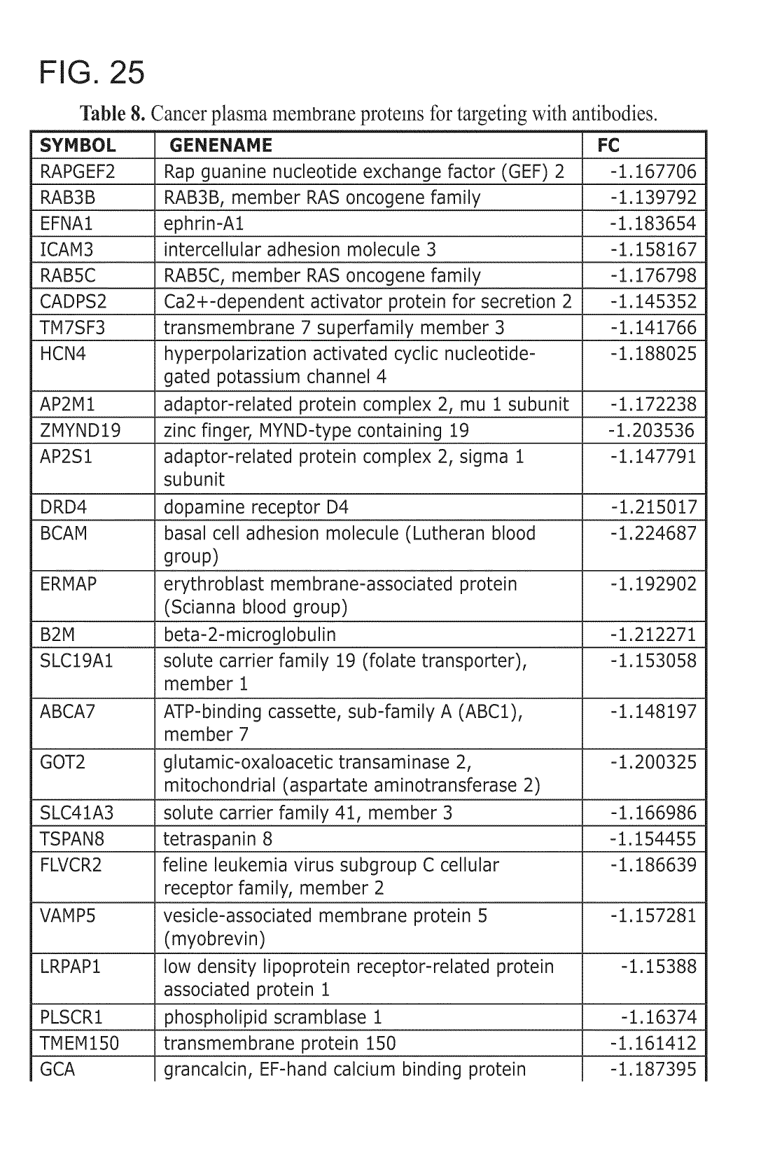

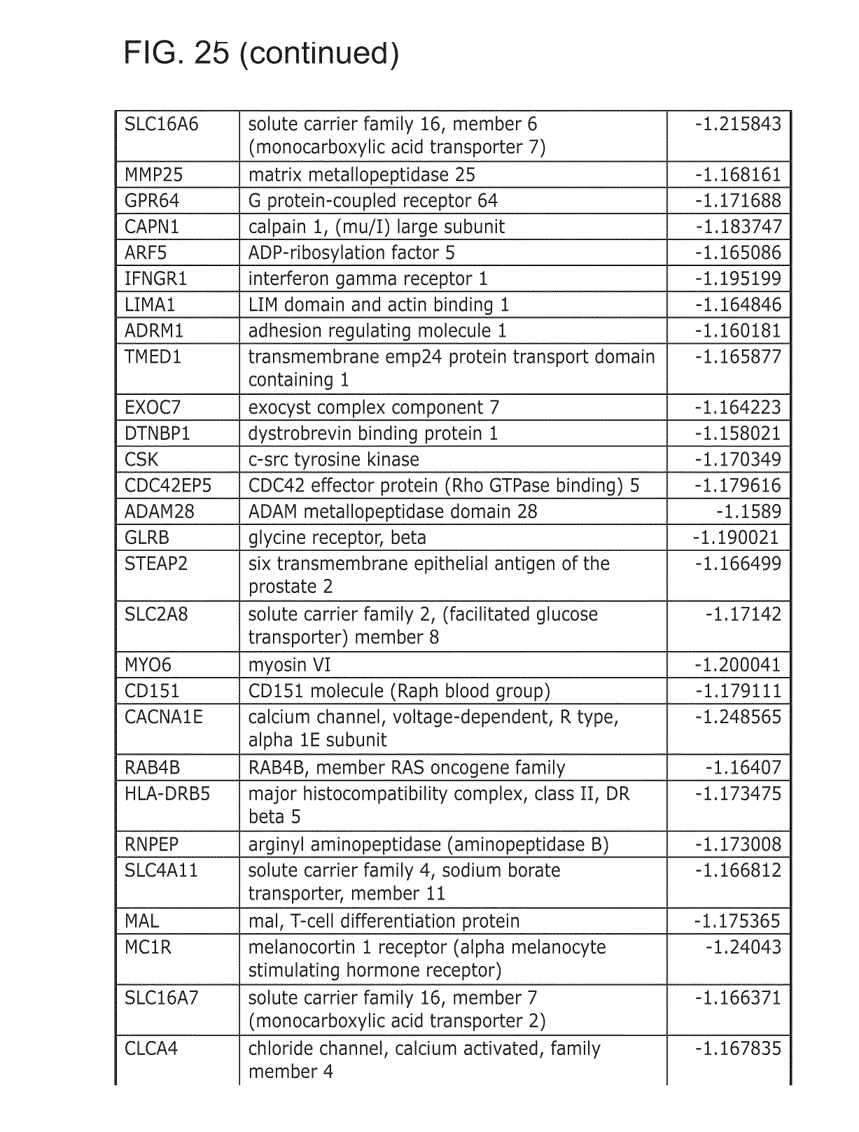

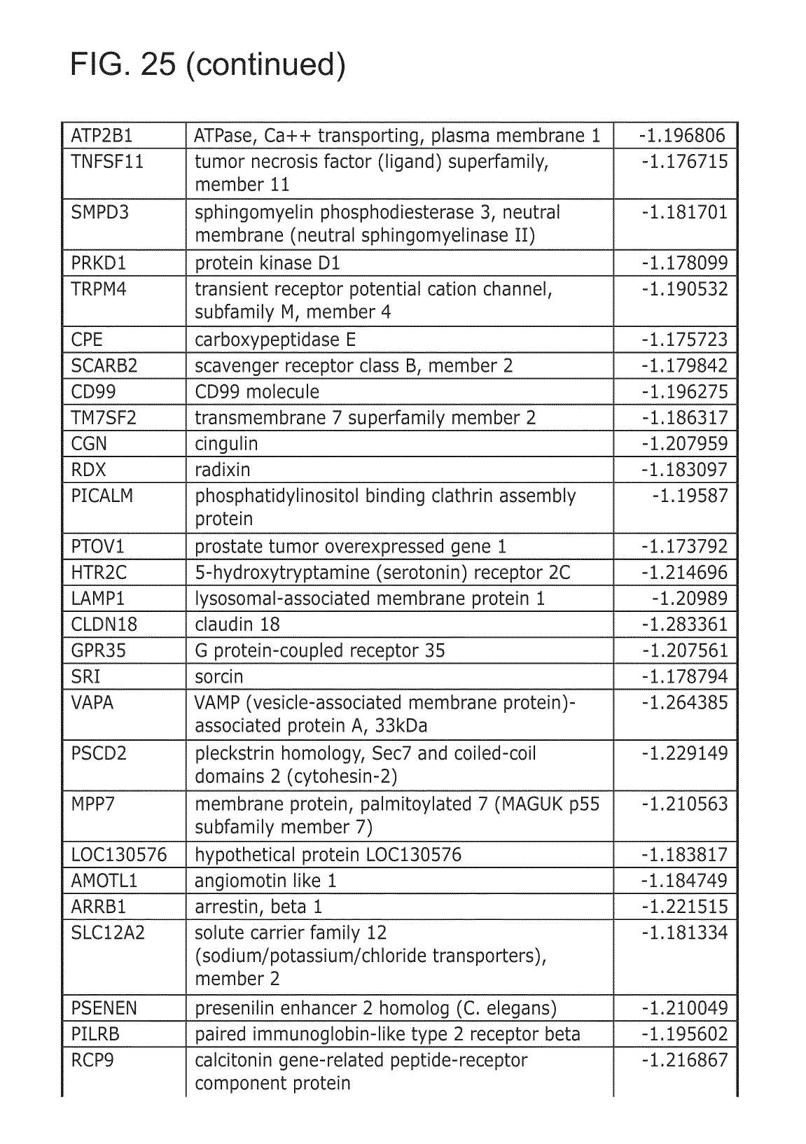

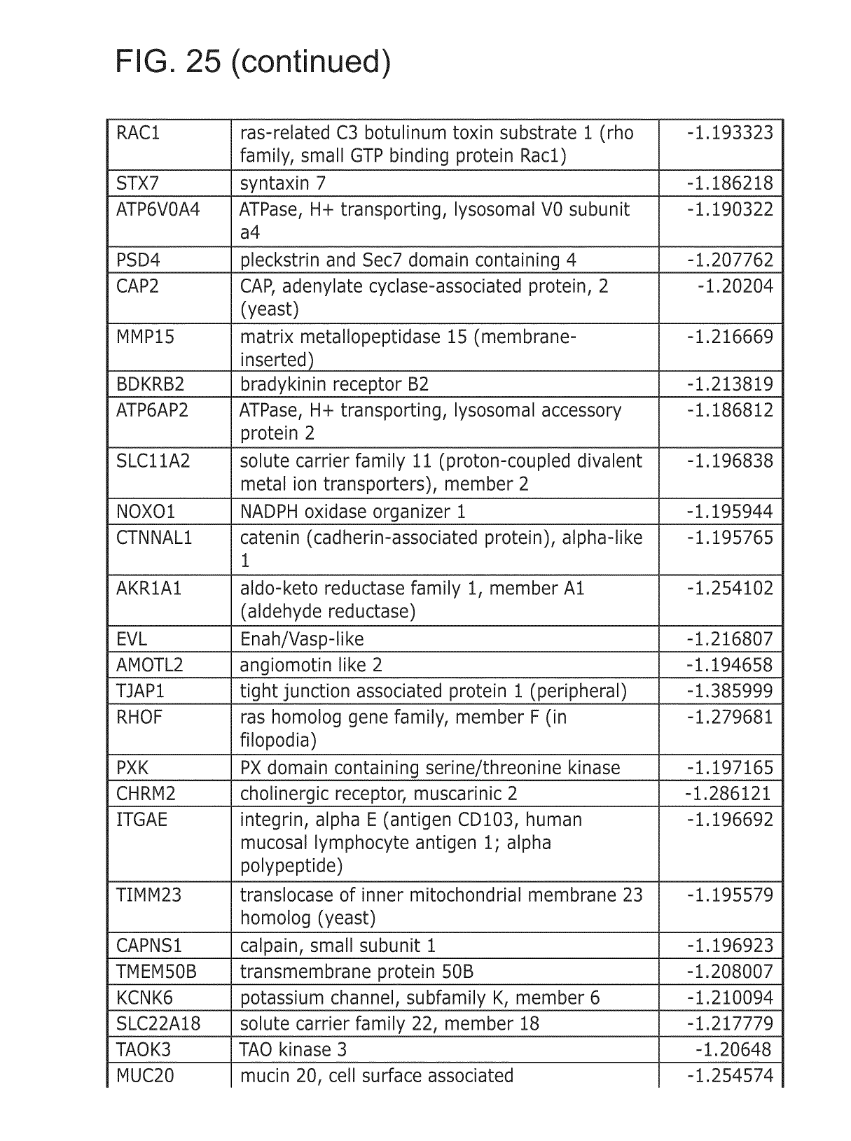

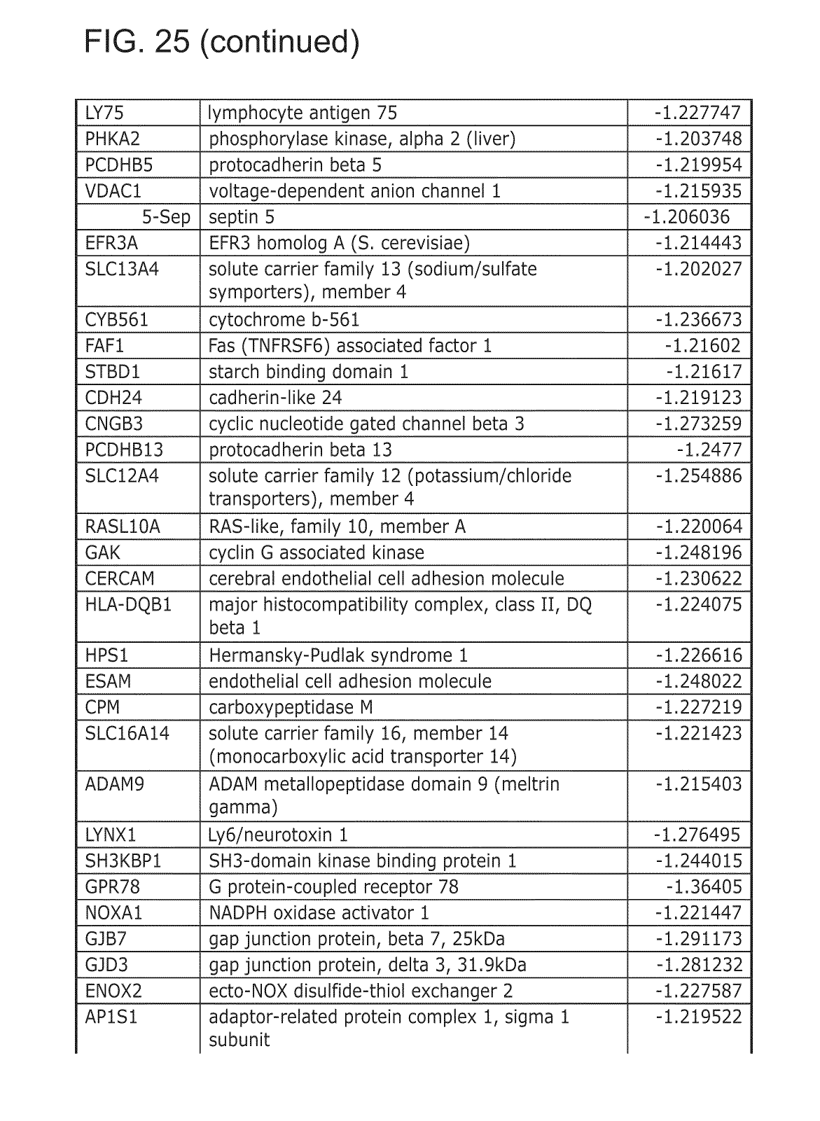

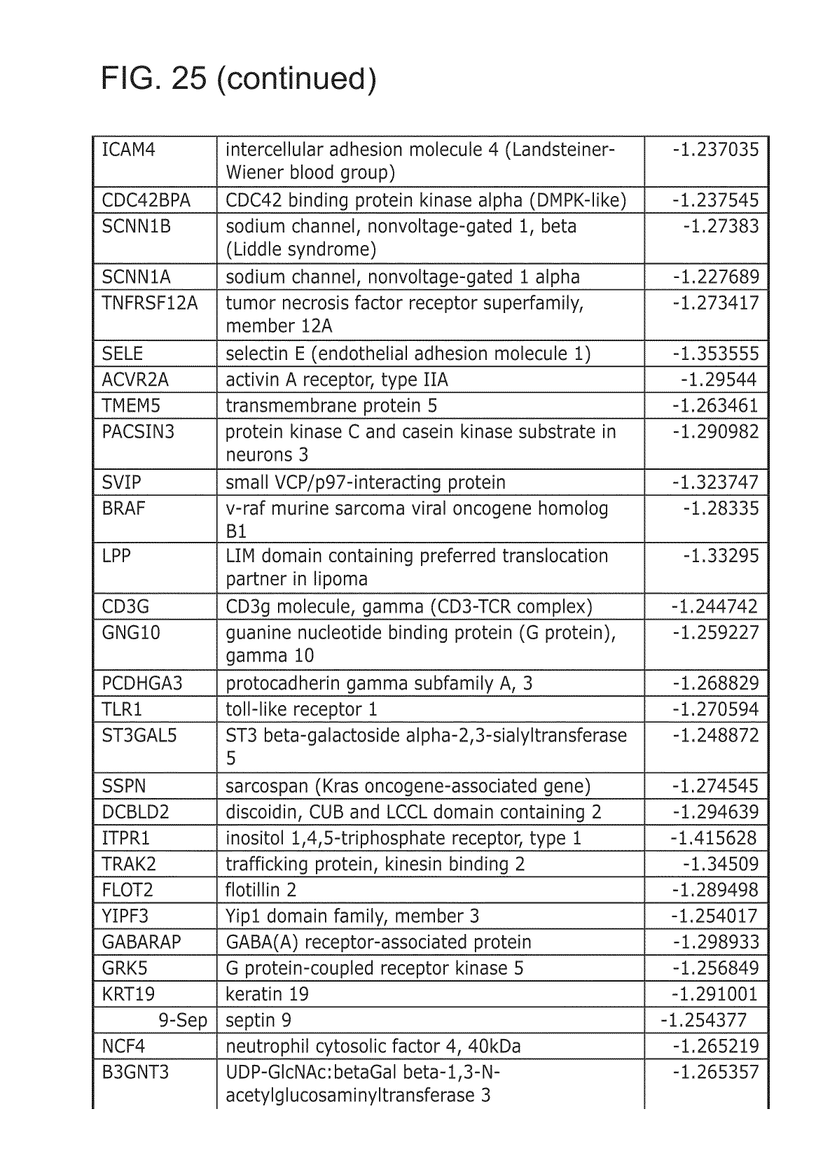

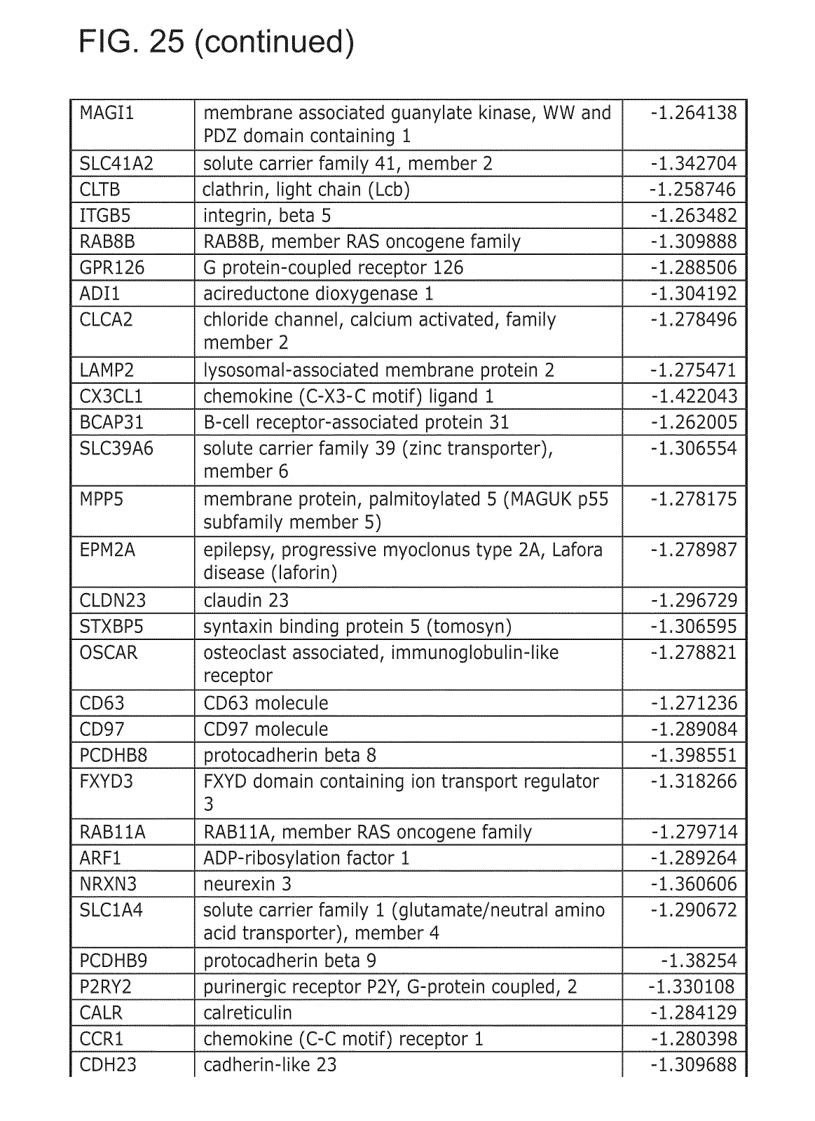

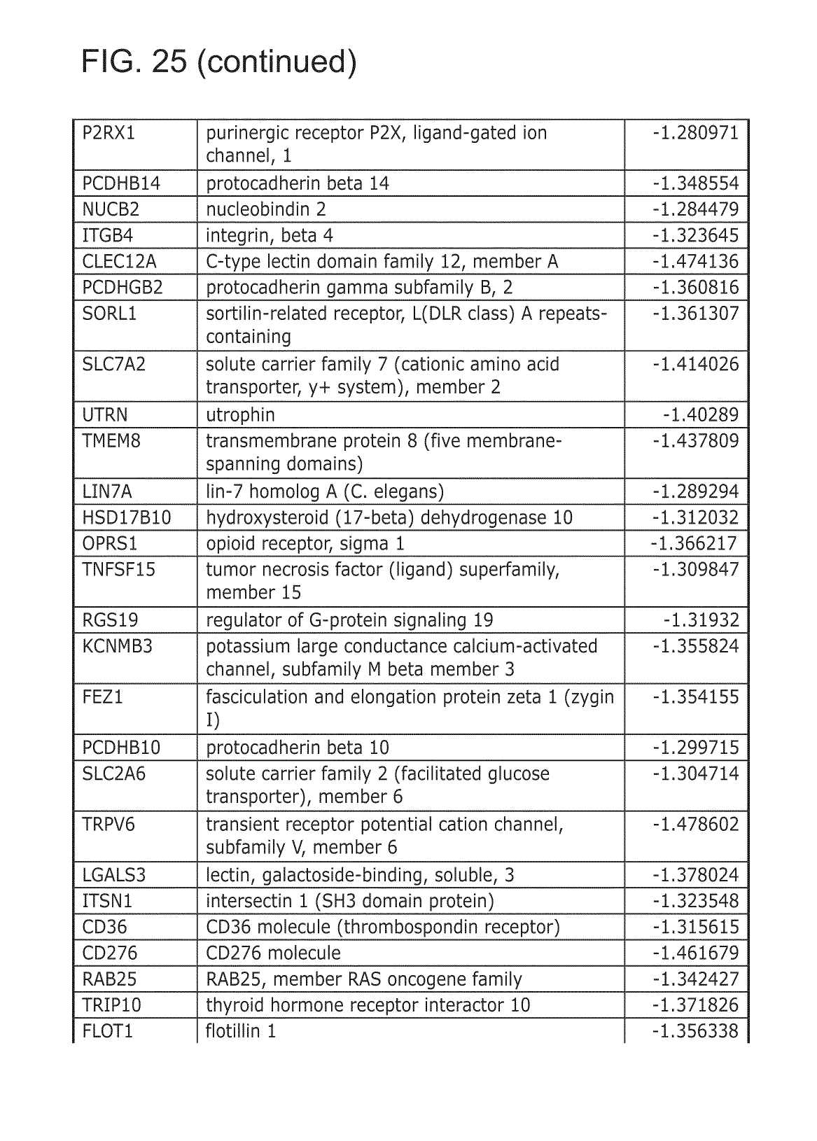

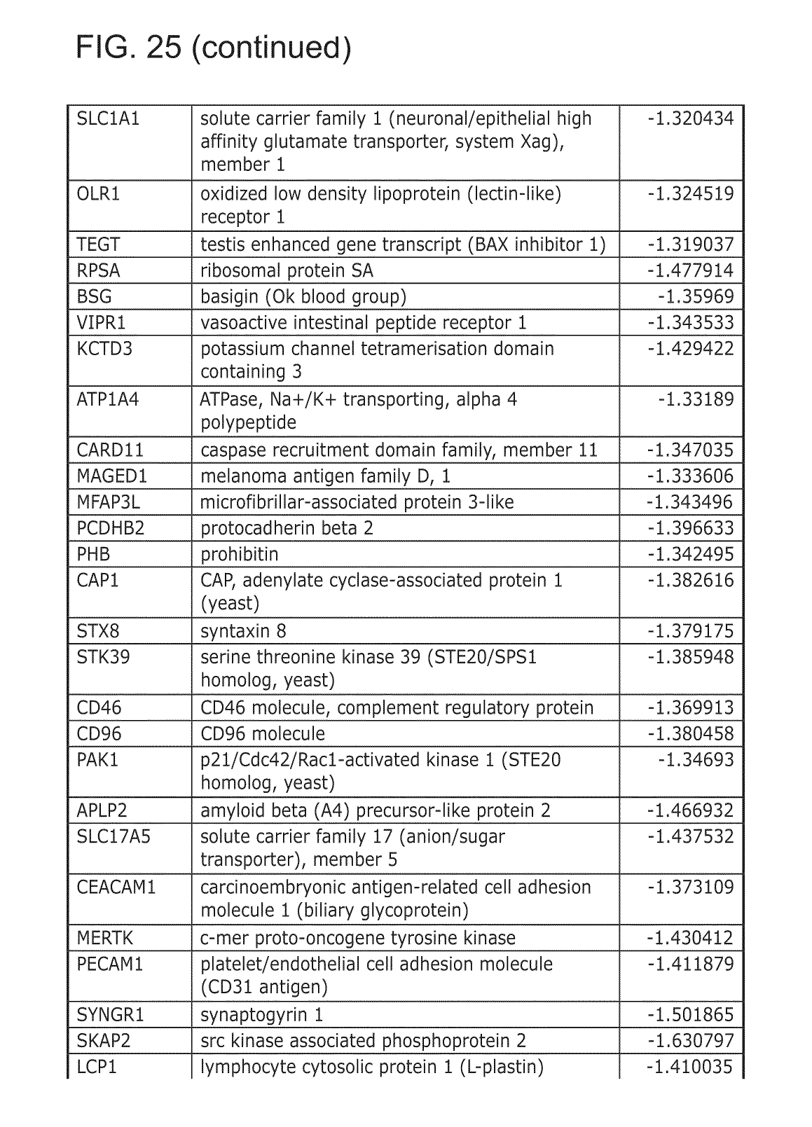

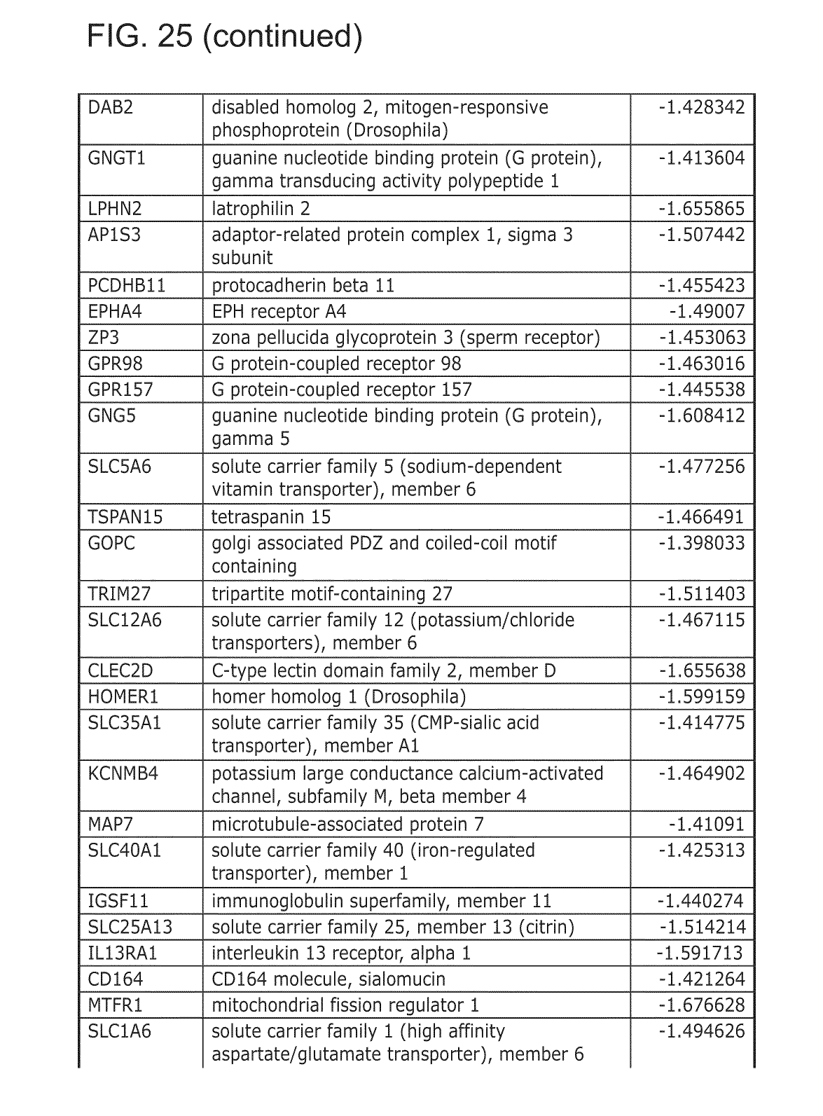

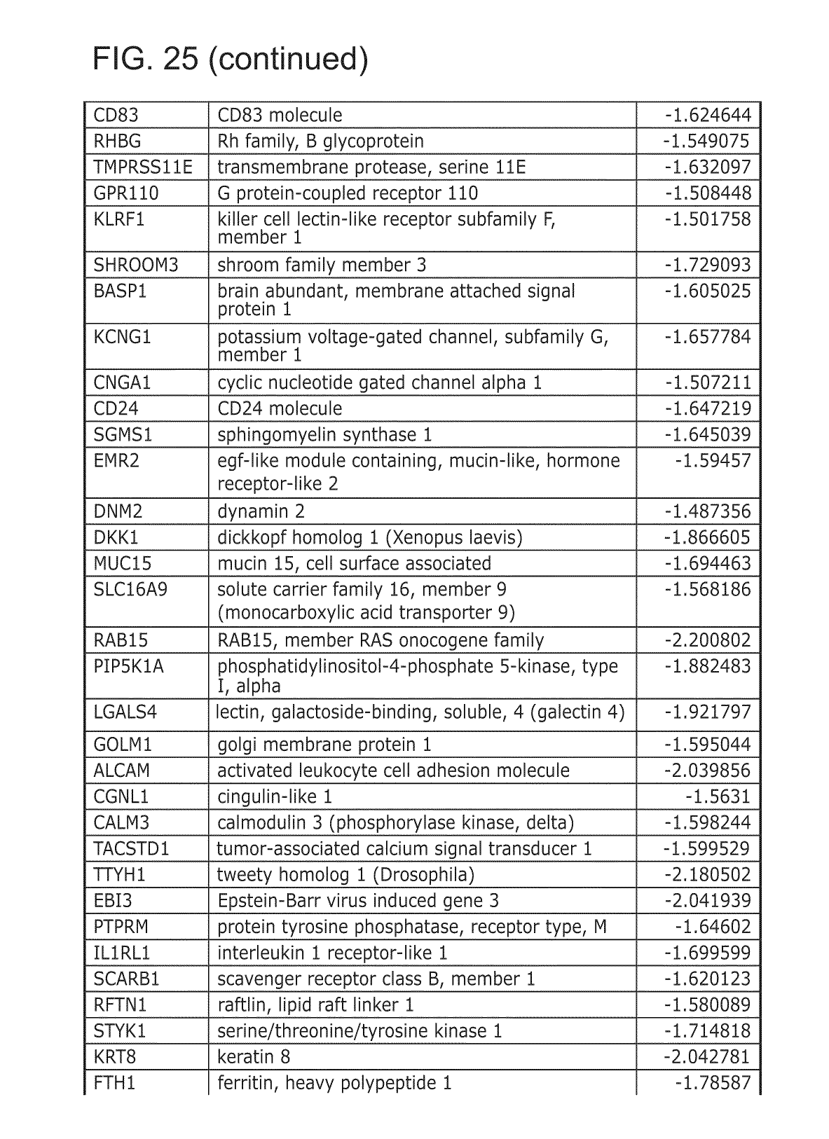

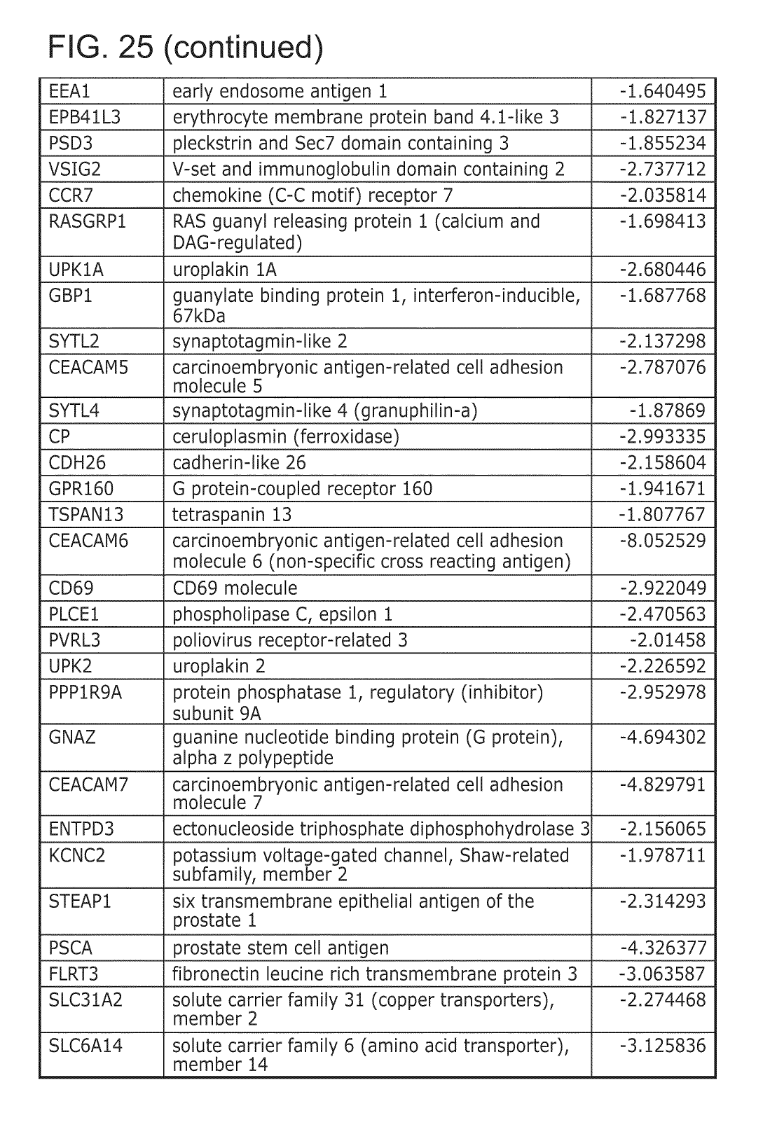

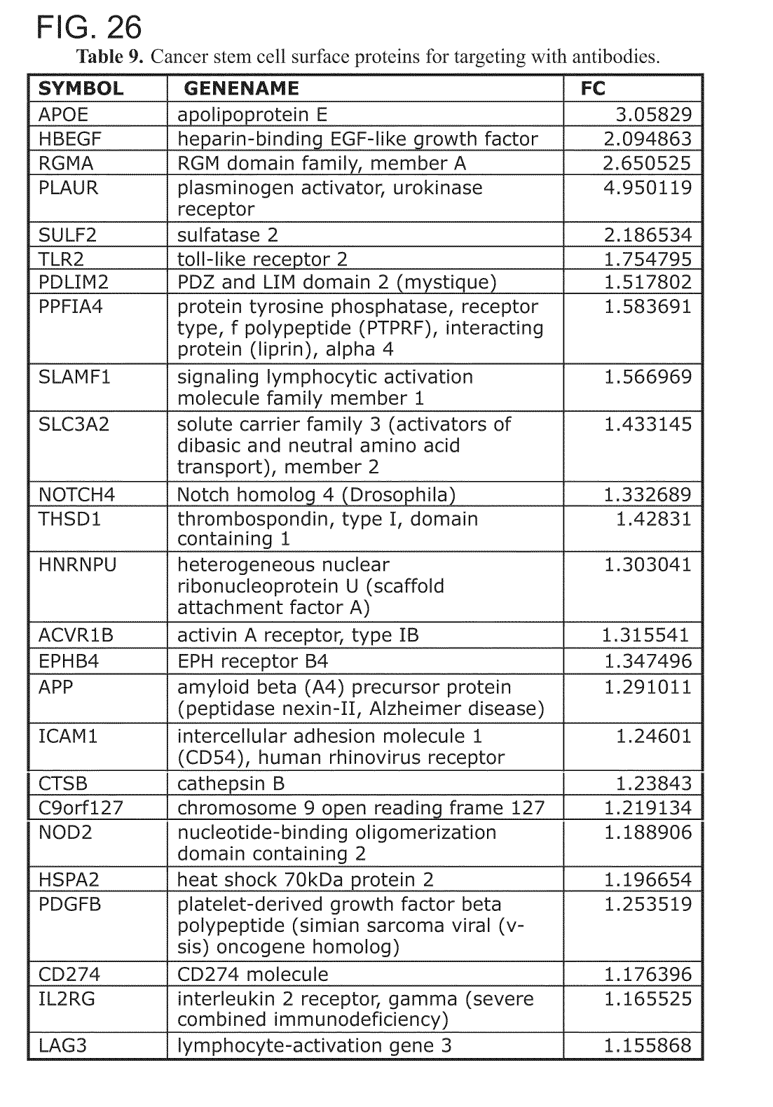

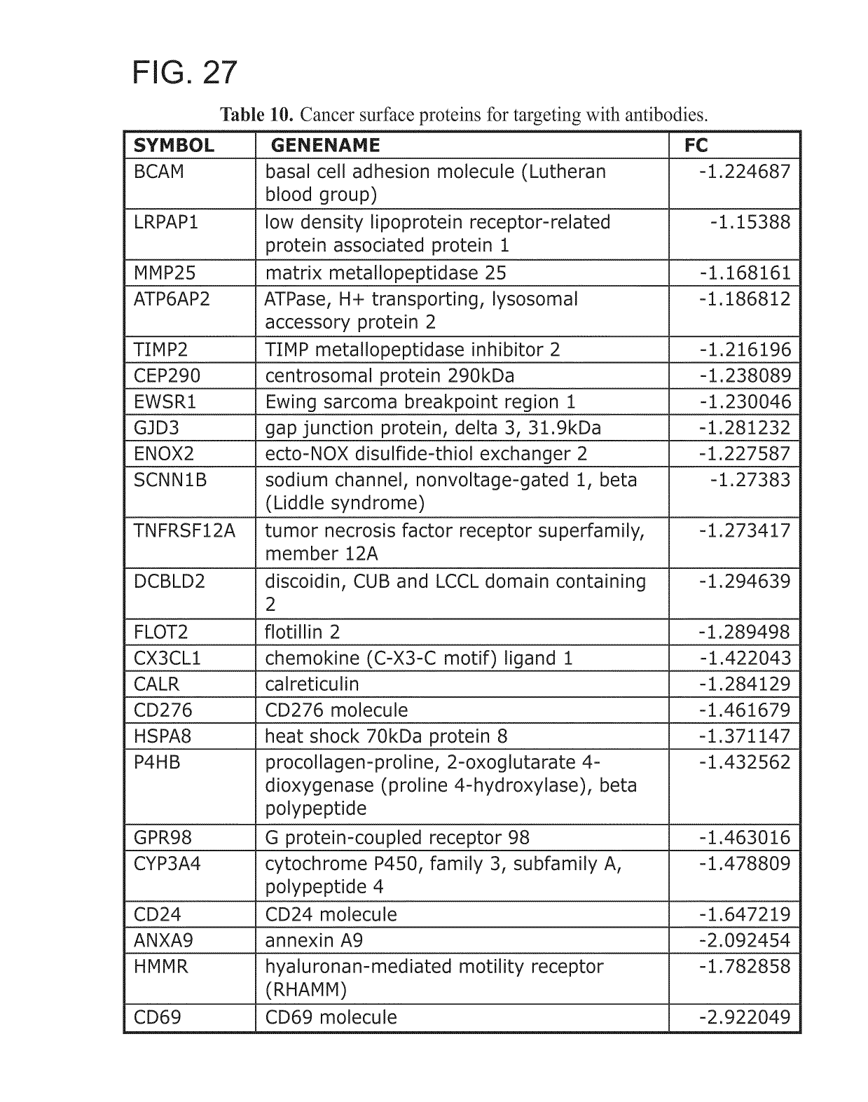

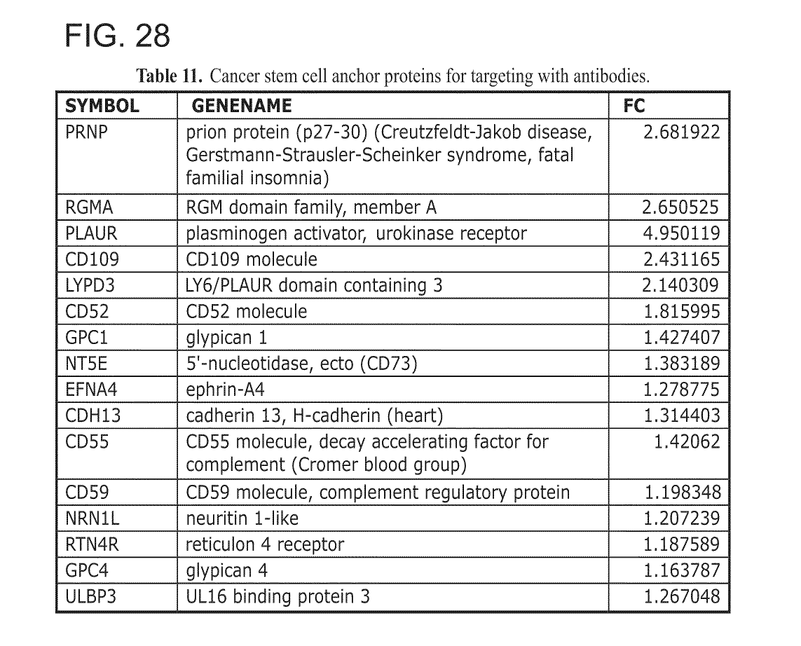

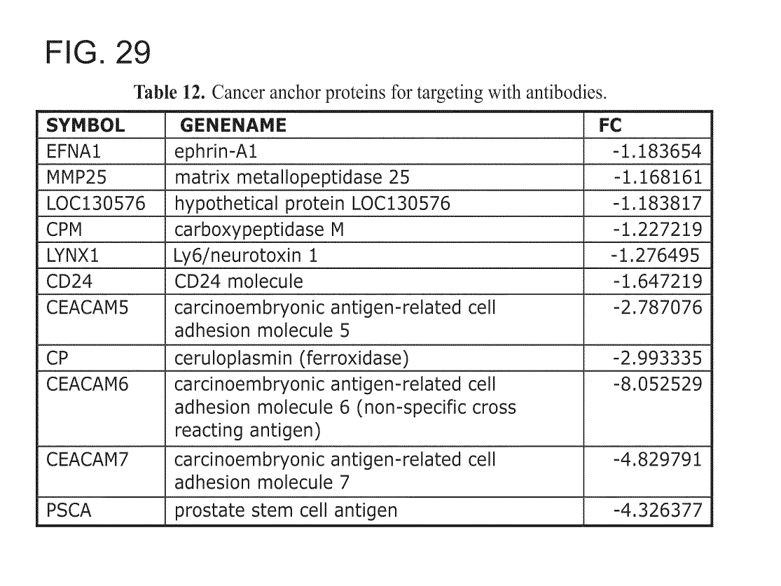

In certain embodiments, provided herein are methods of treating cancer comprising administering to a patient diagnosed with cancer an agent that modulates the activity of a cancer stem cell antigen identified herein, e.g., a cancer stem cell antigen described in FIG. 17 and the following tables: Table 5, Table 6, Table 7, Table 8, Table 9, Table 10, Table 11, Table 12. In a specific embodiment, the agent is an antibody fragment thereof. In another specific embodiment, the agent is an antibody fragment attached to a therapeutic moiety.

A patient can be diagnosed with cancer using any of a number of art recognized methods. For example, a patient can be diagnosed as having cancer by the methods set forth in U.S. Pat. No. 6,004,528 or U.S. Pat. No. 7,427,400.

In certain embodiments, the agent is administered to the patient in an amount sufficient to inhibit the proliferation of cancer cells in the patient. In certain embodiments, the method results in a reduction of cancer cells and/or a reduction of cancer stem cells in the patient. In certain embodiments, cancer cells and/or cancer stem cells do not increase in the patient. In a specific embodiment, the patient is in remission from cancer. In another specific embodiment, the patient has relapsed from cancer. In another specific embodiment, the patient has undergone cancer therapy. In another specific embodiment, the patient has failed one or more cancer treatments. In another specific embodiment, the method comprises the initial therapy for treating the patient.

In certain embodiments, the methods of treating cancer described herein further comprise detecting cancer cells and/or detecting cancer stem cells. In certain embodiments, the methods detect a reduction in tumor size. In certain embodiments, the method detects a failure of one or more tumors in the patient to increase in size. In certain embodiments, the detection methods utilize one or more specimens isolated from the patient including, but not limited to, a blood sample, a bone marrow sample or a tumor biopsy. In other embodiments, the detection methods utilize an imaging technique, e.g., radionuclide imaging, fluorescent imaging, CT scan, X-ray or MRI scan.

In certain embodiments, the agents used in the methods of treating cancer described herein result in one or more of the following: a decrease in viability of cancer stem cells; a decrease in growth of cancer stem cells; the modulation of cancer stem cells; the differentiation of cancer stem cells; the asymmetric cell division of cancer stem cells. In other embodiments, the agents used in the methods of treating cancer described herein kill cancer stem cells; inhibit the proliferation of cancer stem cells; decrease the viability of cancer stem cells; and/or decrease the growth of cancer stem cells. The ability of an agent to result in one or more of the foregoing can be assessed using the in vivo and in vitro assays described herein or using methods known to those of skill in the art. In certain embodiments, the cancer stem cells are slow growing, are mutationally spared relative to tumor bulk, and/or symmetrically divide [Include these

Tumor growth is conceptually analogous to organ formation in the embryo and to repair in adults, yet the precise relationships between these events are poorly understood. In particular, there is mounting evidence suggesting that tumors, like developing organs, comprise hierarchies of cells with phenotypically distinct subpopulations. Thus, cellular heterogeneity in tumors may represent the operation of differentiation programs rather than solely random events. Such heterogeneity is an important part of the functional organization of somatic tissues, reflecting a cell's differentiation state and governing its phenotypic repertoire, including its gene expression and its ability to produce daughter cells. In many stratified polarized epithelia, such as the urothelial lining of the bladder, differentiation proceeds from the basement membrane at the epithelial-stromal interface towards the luminal surface and is inversely correlated to replicative activity. See Kurzrock et al., “Label-retaining cells of the bladder: candidate urothelial stem cells.” Am J Physiol Renal Physiol. 294: F1415-1421 (2008); Farsund, “Cell kinetics of mouse urinary bladder epithelium. II. Changes in proliferation and nuclear DNA content during necrosis regeneration, and hyperplasia caused by a single dose of cyclophosphamide.” Virchows Arch B Cell Pathol. 21: 279-298 (1976). Accordingly, the basal layer is the proposed stem/progenitor compartment for urothelium responsible for generating enough cells to maintain homeostasis for the life of the animal, whereas the fully differentiated superficial cells have limited ability to replicate and function primarily to provide a distensible barrier that excludes excreted solutes in the urine, preventing them from re-entering the bloodstream. See Lewis, “Everything you wanted to know about the bladder epithelium but were afraid to ask.” Am J Physiol Renal Physiol. 278: F867-874 (2000). Aptly named intermediate cells separate basal and superficial cells and are intermediate in their differentiation and replicative ability. See Farsund, “Cell kinetics of mouse urinary bladder epithelium. II. Changes in proliferation and nuclear DNA content during necrosis regeneration, and hyperplasia caused by a single dose of cyclophosphamide.” Virchows Arch B Cell Pathol. 21: 279-298 (1976).

Carcinomas, cancers derived from epithelia, are responsible for vast the majority of cancer deaths. See Jemal et al., “Annual report to the nation on the status of cancer, 1975-2005, featuring trends in lung cancer, tobacco use, and tobacco control.” J Natl Cancer Inst. 100: 1672-1694 (2008). Cell populations within carcinomas are heterogeneous in their contributions to tumor growth and spread, complicating the ability of scientists to understand and treat cancer. See Lobo et al., “The biology of cancer stem cells.” Annu Rev Cell Dev Biol. 23: 675-699 (2007). Several investigators have used empirically derived surface markers to isolate phenotypically homogeneous and highly tumorigenic subpopulations of cells within solid cancers. See Lobo et al., “The biology of cancer stem cells.” Annu Rev Cell Dev Biol. 23: 675-699 (2007); Visvader and Lindeman “Cancer stem cells in solid tumours: accumulating evidence and unresolved questions.” Nat Rev Cancer. 8: 755-68 (2008). Upon transfer to a new host environment, these phenotypically homogeneous cancer cell populations have enhanced potency in forming a new tumor and are able to recreate the phenotypic diversity of the original tumor. See Lobo et al., “The biology of cancer stem cells.” Annu Rev Cell Dev Biol. 23: 675-699 (2007); Jordan, “Cancer stem cells: controversial or just misunderstood?” Cell Stem Cell. 4: 203-2055 (2009).; Reya et al., “Stem cells, cancer, and cancer stem cells.” Nature. 414: 105-111 (2001); and Wang and Dick, “Cancer stem cells: lessons from leukemia.” Trends Cell Biol. 15: 494-501 (2005). Through predominantly empiric approaches, HTCs have now been isolated from a handful of carcinomas, including those arising in breast, pancreas, colon, ovary, skin and the upper aerodigestive tract. See Visvader et al., “Cancer stem cells in solid tumours: accumulating evidence and unresolved questions.” Nat Rev Cancer. 8: 755-68 (2008). These studies indicate that cells within tumors are hierarchically organized with respect to their tumor forming potential, but yield limited insight into the basis of this hierarchy.

In normal epithelial tissues, cellular hierarchies are organized by differentiation programs, and differentiation has long been a recognized feature of cancer, including carcinomas. See Sun et al., “Keratin classes: molecular markers for different types of epithelial differentiation.” J Invest Dermatol. 81: 109s⁻¹¹⁵s (1983) and Grubb et al., “Squamous differentiation in carcinoma in situ of the cervix uteri. A cyto-histological correlation of malignant intraepithelial lesions with invasive carcinoma.” J Clin Pathol. 20: 7-14 (1967). As such, differentiation may represent a launching point from which to understand cancer cell heterogeneity. Although undifferentiated carcinomas are sometimes observed, most cases, even those with widespread metastases, express markers corresponding to the most differentiated cell types in the epithelia in which they arise. For example, two thirds of urothelial carcinomas express uroplakins, markers of differentiated superficial cells, and mRNA transcripts encoding these markers can be found in metastatic tumor cells isolated from blood or lymph nodes. See Huang et al., “Persistent uroplakin expression in advanced urothelial carcinomas: implications in urothelial tumor progression and clinical outcome.” Hum Pathol. 38: 1703-13 (2007); Li et al., “Detection of circulating uroplakin-positive cells in patients with transitional cell carcinoma of the bladder.” The Journal of Urology. 162: 931-5 (1999); Seraj et al., “Molecular determination of perivesical and lymph node metastasis after radical cystectomy for urothelial carcinoma of the bladder.” Clin Cancer Res. 7: 1516-22 (2001). From these results, one could suggest several possible relationships between cell differentiation and the ability to form new tumors at distant sites, including 1) carcinoma cells aberrantly express differentiation markers which have no relationship to their metastatic potential; 2) fully differentiated urothelial carcinoma cells have significant long term growth potential; and 3) relatively undifferentiated cells drive tumor formation and subsequently differentiate. See Brabletz et al., “Opinion: migrating cancer stem cells—an integrated concept of malignant tumour progression.” Nat Rev Cancer. 5: 744-9 (2005). The third possibility centers on differentiation as an organizing principle for the identification, isolation, and understanding of highly tumorigenic subpopulations of carcinoma cells. Example 1 provides evidence for differentiation of urothelial carcinomas that is analogous to normal urothelial differentiation. In particular, a basal-like urothelial cancer cell subpopulation was identified that 1) resides at and depends on the tumor-stroma interface and 2) possesses most, if not all of the tumor forming ability of the parental tumor. A novel and easily tractable model systems for analyzing differential expression of genes and pathways in basal-like vs. more differentiated cells were established. Using these systems, it was demonstrated that differential activity of pathways comprising the “Hallmarks” of cancer, and supported by several specific inter- and intracellular signaling cascades for which mechanism-based therapies are available or in development. See Hanahan and Weinberg, “The hallmarks of cancer.” Cell. 100: 57-70 (2000).

### EXAMPLES

The following examples are illustrative, and should not be viewed as limiting the scope of the present invention. Reasonable variations, such as those that occur to a reasonable artisan, can be made herein without departing from the scope of the present invention.

### Example 1

### Cancer Stem Cell Expression Patterns

### Study Method & Design

The following example describes the results of a study in which a screen was conducted to identify genes preferentially expressed in cancer stem cells.

A tissue array with 55 cases of invasive urothelial carcinomas, summary stage I-IV, was constructed as described in Fedor and De Marzo. See Fedor, De Marzo, “Practical methods for tissue microarray construction.” Methods Mol. Med. 103: 89-101 (2005). The presence of cancer in the array and immunohistochemical staining for CK17 and CK18 was scored as absent, present with peripheral pattern (strongest at the tumor-stroma interface) or present with equivalent pattern (intensity not increased at tumor-stromal interface). Cases were scored as positive if they fulfilled either of two criteria: intense staining in more than 5% of cells or moderate staining in more than 10% of cells. Two cases lacked cancer on the slide and were censored from the study. The SW780 (human bladder TCC) cell line was cultured in DMEM supplemented with 10% fetal bovine serum (FBS).

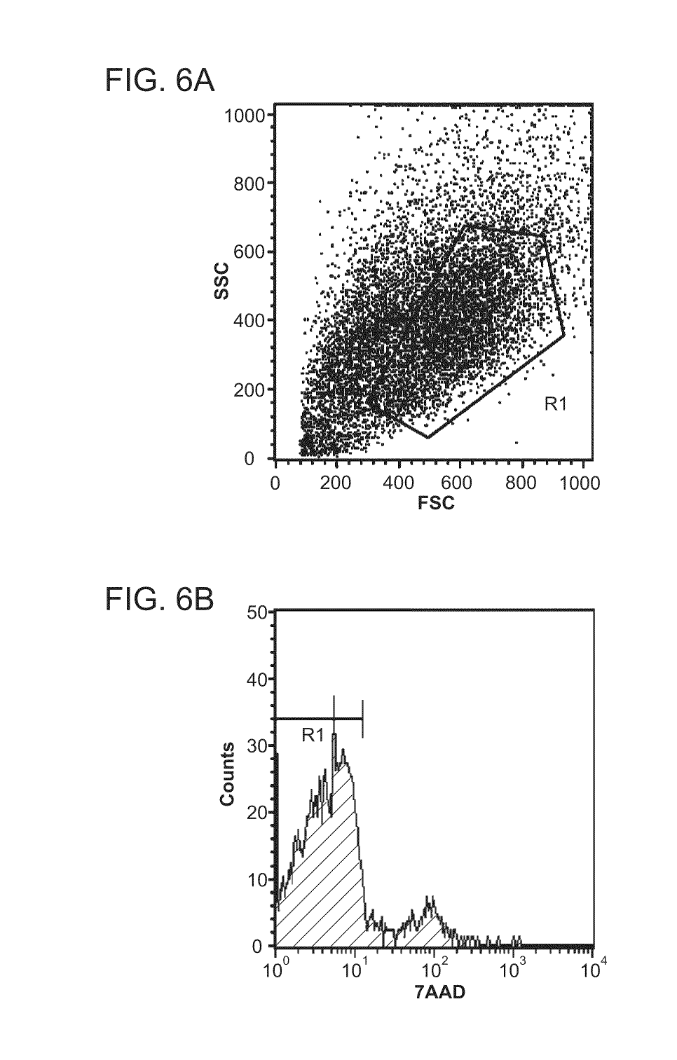

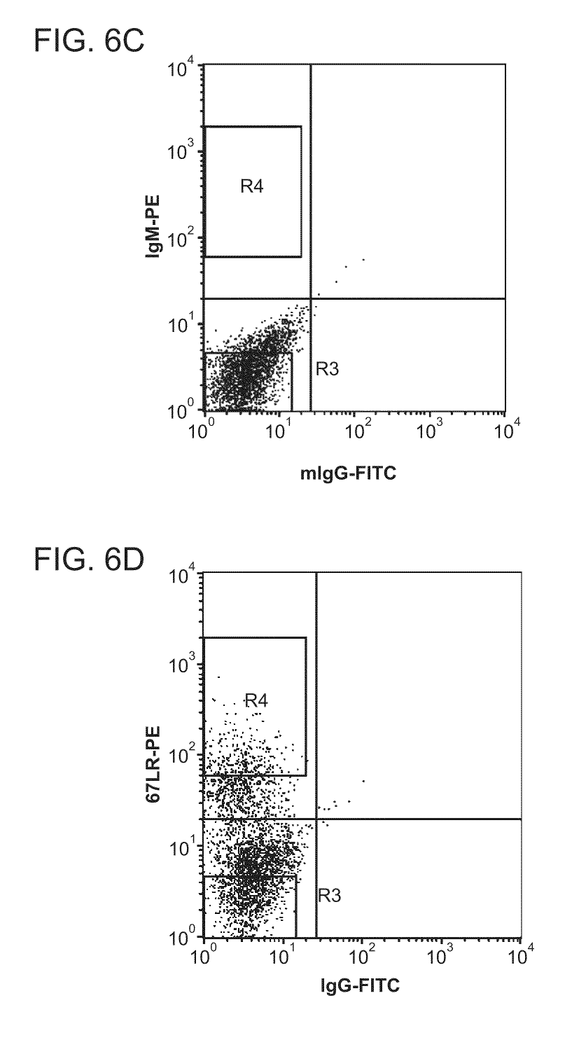

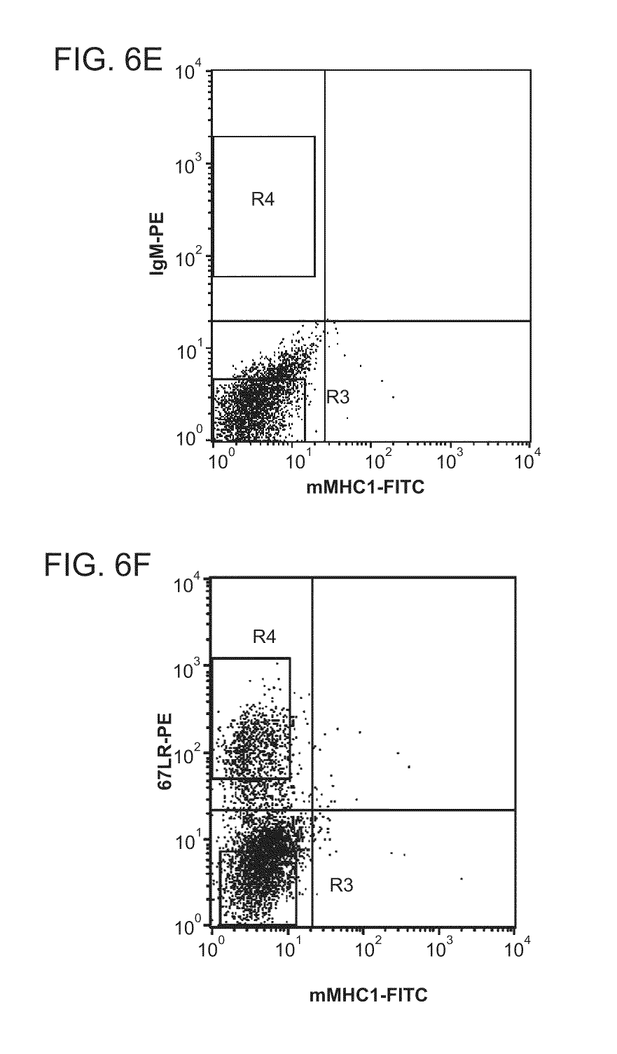

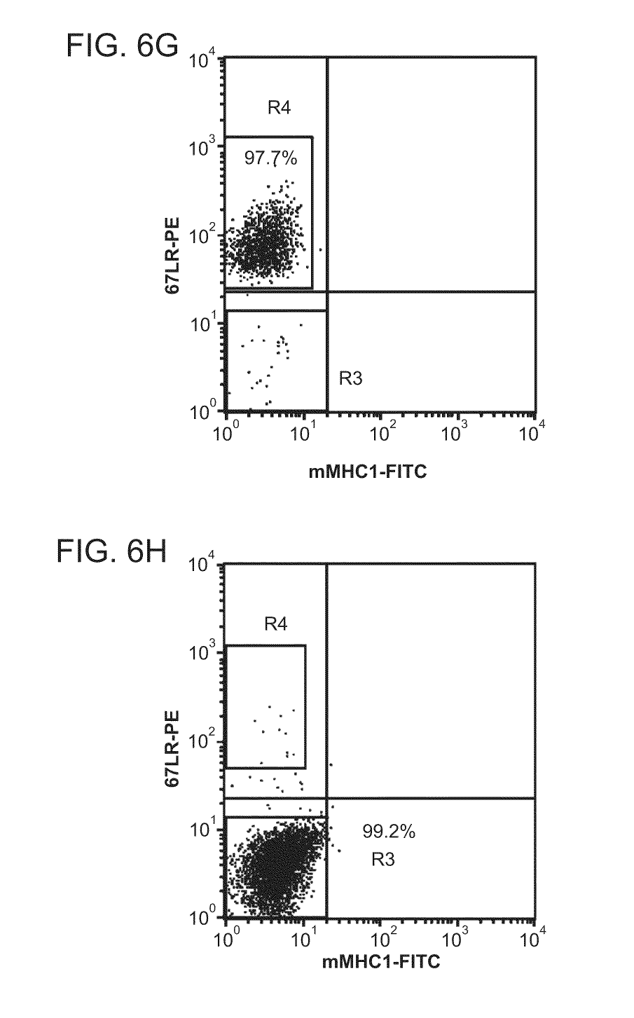

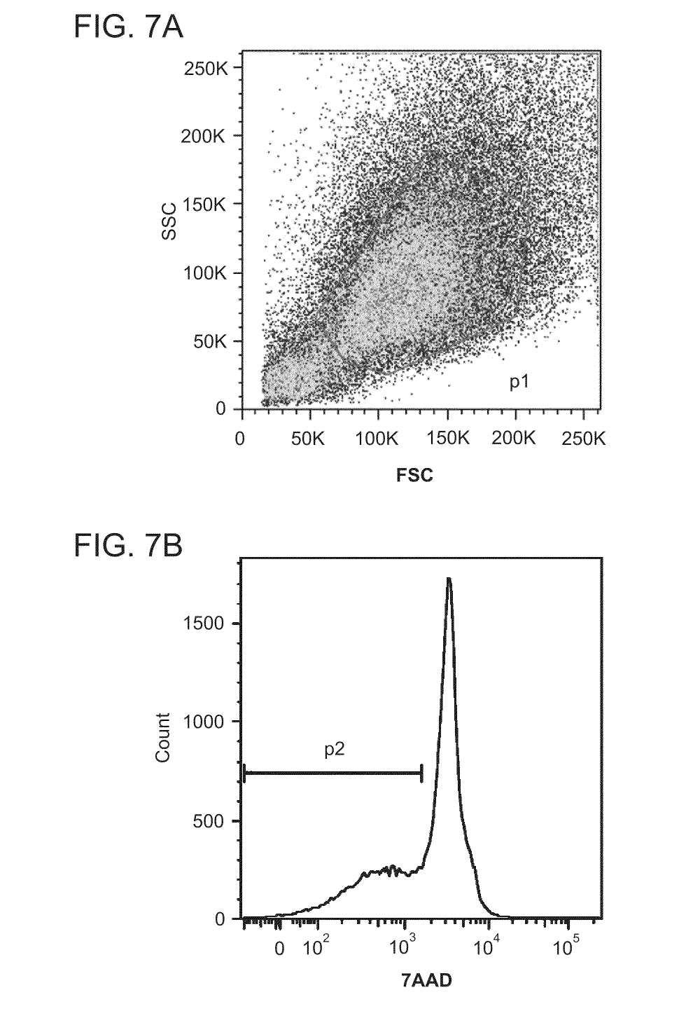

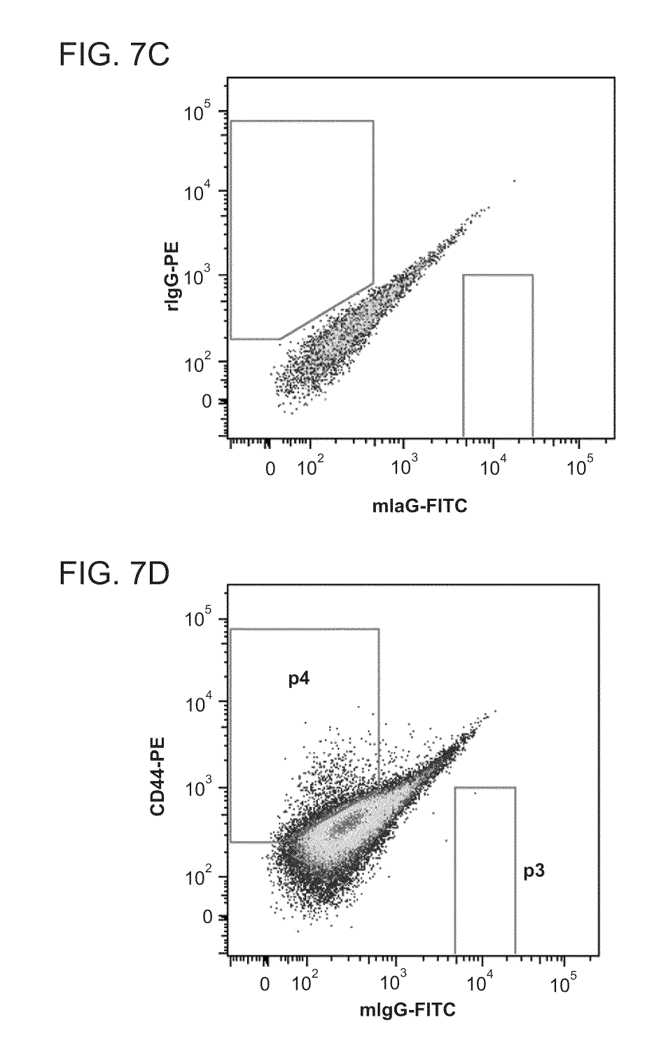

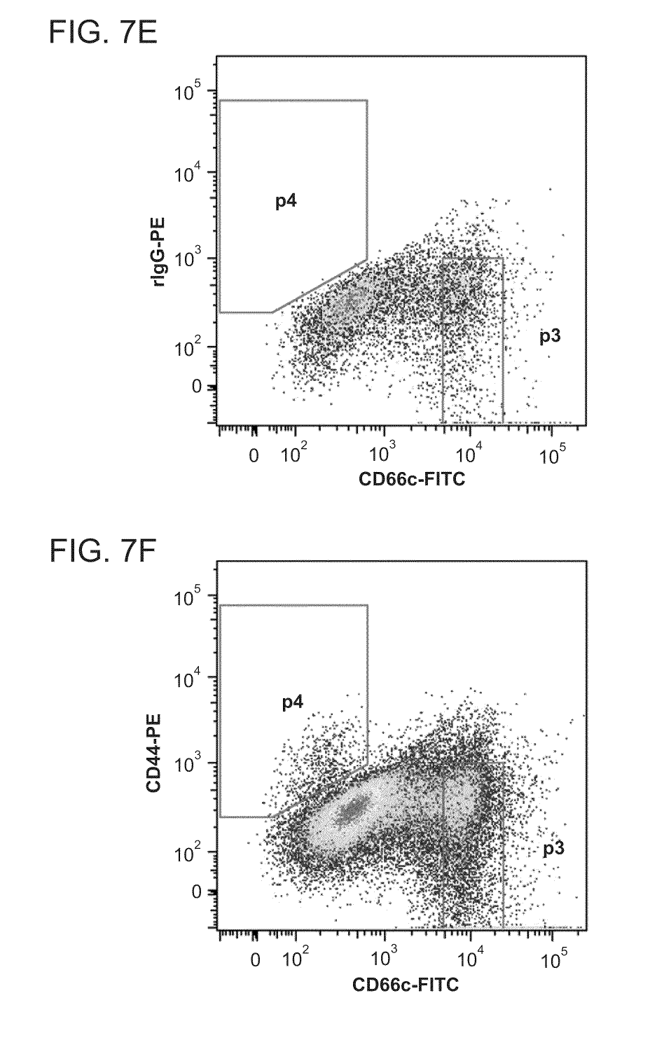

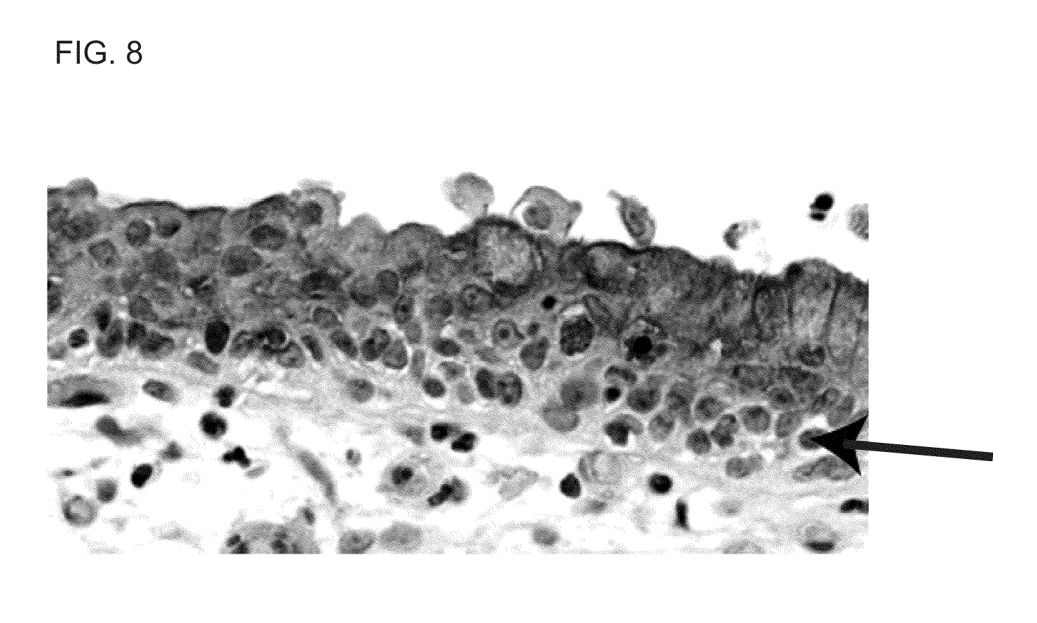

Bladder cancer xenografts were established and propagated from a sample of a stage pT3NOMx invasive urothelial carcinoma from a woman in her 7th decade and passaged once in athymic mice prior to use. The xenografts were further propagated by injecting 10⁶ cultured cells (SW780) or ˜100 mg of minced primary tumor fragments (XBL8 xenograft) subcutaneously into the flanks of six-week-old female athymic nude mice. The xenografts were then dissociated into single cell suspensions. Cells obtained from this procedure showed higher than 75% viability evaluated by Trypan blue exclusion assay. The SW780 cells were sorted into 67LR bright (67LR+mMHC1−) or dim (67LR−mMHC1−) populations based on 67LR staining profile, or bulk or control sorted (mMHC1−) cells. The purity of every population was 97-99% evaluated by post-sorting analysis (FIG. 6). The XBL8 tumor cells were sorted into CEACAM6-positive and CEACAM6-negative/CD44 positive populations (FIG. 7).

The tumorigenicity of the sorted cells XBL8 cells was assessed in an in vivo, Cell sorting. SW780/67LR: SW780 xenograft single cells were resuspended in PBS containing 2% heat-inactivated FBS (FACS buffer) at 1×10⁷ cells/ml. Nonspecific binding was blocked by incubation with 10% goat serum (Sigma) in FACS buffer for 30 min at 4° C. 67LR (M1u5, Alexis Biochemicals) monoclonal antibody was then added to 2.5 μg/ml and incubated for 40 min on ice. After 3×5 min washes with FACS buffer, cell suspensions were further incubated with phycoerythrin (PE)-conjugated goat anti-mouse IgM (Biolegend, 406507; 2.5 μg/ml), and fluorescein isothiocyanate (FITC) conjugated antibodies against mouse H-2Dk and H-2 Kb/H-2 Db major histocompatibility antigens (mMHC1, Biolegend, #110305 and #114605; 1:20 dilution) for 40 min on ice, protected from light. Cells were then washed three times with FACS buffer and resuspended in PBS containing 2% BSA and 1 μg/ml of 7-aminoactinomycin D (7-AAD). Isotype-matched negative controls and single-color controls were included with each experiment. Analysis and cell sorting was performed on a Vantage cell sorter (BD Biosciences) using Cellquest software. Side scatter (SSC) and forward scatter (FSC) profiles were used to eliminate cell doublets and debris. 7-AAD positive cells were gated out to eliminate dead cells. Mouse MHC1-FITC staining was used to eliminate contamination from cells of mouse origin. SW780 xenograft tumor cells were sorted into 67LR bright (67LR+mMHC1−) or dim (67LR−mMHC1 −) populations based on 67LR staining profile, or bulk or control sorted (mMHC1−) cells. The purity of every population was 97-99% evaluated by post-sorting analysis (FIG. 6). XBL8/CEACAM6: XBL8 xenograft single cells prepared as described herein were prepared and stained as described herein except that a CEACAM6 antibody (Neomarkers, Cat# MS-1731; 2.2 μg/ml) was used for the first incubation, followed by a cocktail of FITC conjugated goat anti-mouse IgG (Sigma, F2653; 1:100), and PE conjugated anti-human CD44 (Biolegend, #312306; 1:40). XBL8 tumor cells were sorted into CEACAM6-positive and CEACAM6-negative/CD44 positive populations (FIG. 7).

Tumorigenicity assays were performed. Different doses of sorted cell populations were injected as described. Tumor formation was monitored visually weekly for 4 months.

A microarray using total RNA isolated from the sorted cell samples was conducted to investigate the differential expression of genes across the cell types. The differential expression of 17 genes was confirmed by real time reverse transcription/polymerase chain reaction (ART-PCR) assays. Sample amplification and labeling procedures for microarray analysis were carried out by using the Low RNA Input Fluorescent Linear Amplification Kit (Agilent Technologies). 400 ng of total RNA was reverse transcribed into first strand and second strand cDNA by MMLV-RT using an oligo dT primer (System Biosciences) that incorporates a T7 promoter sequence. The cDNA was then used as a template for in vitro transcription in the presence of T7 RNA polymerase and Cyanine labeled CTPs. The labeled cRNA was purified using RNeasy mini kit (Qiagen), followed by quantification based on both concentrations of cRNA and dye. RNA spike-in controls (Agilent Technologies) were added to RNA samples before amplification and labeling according to manufacturer's protocol.

The following PCR primer sets were used in quantitative RT-PCR analysis (Primer pairs are herein listed in order of gene symbol, forward primer, reverse primer): FST, GATCTTGCAACTCCATTTCGG, GGCTATGTCAACACTGAACAC; TFF1, CCAGACAGAGACGTGTACAG, TCGATGGTATTAGGATAGAAGCAC; KRT5, GCAGTACATCAACAACCTCAG, CAGCATCTACATCCTTCTTCAG; KRT1, TCAAGAAGGATGTGGATGGT, TTGGTAGAGTGCTGTAAGGA; MMP1, GGACTTAGTCCAGAAATACCT, CTTTCAGCCCAAAGAATTCC; CEACAM6, TCTACAAAGAGGTGGACAGAG, GTTAGAAGTGAGGCTGTGAG; SPINK1, TTGAGTCTATCTGGTAACACTG, ACATAACACGCATTCATTGG; UPK1B, TCTTCTGGCGTATTTCATTCTG, CTGGTCATCATTGTTTGGAG; DS G3, AGACAAAGATGGAGAAGGAC, AATACGTGCTGAATACTGAGAG; MMP10, GCAGTTAAAGAACATGGAGAC, TCTGTGAATGAGTTGTAGAGTG; FABP4, AAAGTCAAGAGCACCATAACCT, CACCACCAGTTTATCATCCTC; HRPT, ATAAGCCAGACTTTGTTGGA, CAACTTGAACTCTCATCTTAGG; LAMB3, GACAGGATGAAAGACATGGAG, AAAGCATTCCAACCCAATCTG; AK3L1, TGAATTATACAAGAGCCGAGGA, GACACAGCACCAATACTACAC; HMOX1, ACAAAGTGCAAGATTCTGCC, ATTCACATGGCATAAAGCCC; ITGA6, AGACTCTTAACTGTAGCGTG, AATGTGCTGTTCCATAACCTC; CMAF, CAAGGAGAAATACGAGAAGTTGG, CTGGTAAGTACACGATGCTG; KRT18, GCGAGGACTTTAATCTTGGTG, CTCAGAACTTTGGTGTCATTGG.

1.5 μg of each sample labeled with Cy3 or Cy5 was mixed with control targets (Agilent Technologies). Fragmentation was carried out by incubating for 30 minutes at 60° C. and stopped by adding an equal volume of 2× hybridization buffer (Agilent Technologies).

Fragmented targets were added to the microarrays, assembled into a hybridization chamber (Agilent Technologies) and hybridized at 65° C. for 17 hours in a hybridization oven with rotation. Hybridized microarrays were washed and dried according to the Agilent microarray processing protocol.

Hybridized slides were scanned using a G2565BA Agilent scanner, with 100% PMT and 10 micrometer resolution according to the manufacturer's directions. The scanner was operated by the Agilent Scan Control 7.0 software. Data were extracted with the Agilent Feature Extraction 9.5.3.1 software. Images were subjected to quality checks recommended by the manufacturer. Images were visually inspected for artifacts and distributions of red and green channels, both for foreground and background, to identify anomalous arrays. No abnormalities were found and all arrays were retained for subsequent analyses.

For dual color experiments all pre-processing procedures described herein were performed using functions and methods implemented in the R/Bioconductor package limma. See Smyth. “Limma: linear models for microarray data.” In R. Gentleman, R. V. Carey, S. Dudoit, R. Irizarry, and W. Huber, editors, Bioinformatics and Computational Biology Solutions using R and Bioconductor. 397-420. Springer, N.Y., (2005); Smyth, “Linear models and empirical Bayes methods for assessing differential expression in microarray experiments.” Statistical Applications in Genetics and Molecular Biology. 3(Article 3) (2004). For Affymetrix experiments all pre-processing procedures described herein were performed using functions and methods implemented in the R/Bioconductor package affy. See Gautier et al., “affy-analysis of Affymetrix GeneChip data at the probe level.” Bioinformatics. 20: 307-315 (2004). Pre-processing and quality of the hybridizations was monitored performing standard diagnostic plots (MA-plots before and after normalization, 2D-image plots, boxplots of expression levels before and after normalization, and RNA degradation plots for the Affymetrix platform data sets).

Within-array dye effects were corrected by the “loess” normalization method. See Yang et al., “Normalization for cDNA microarray data: a robust composite method addressing single and multiple slide systematic variation.” Nucleic Acids Res. 30: 1362-4962 (2002). The “scale” normalization method was applied to standardize log2 Cy5/Cy3 ratio (the so called M-value) distributions across different arrays, by using the median-absolute-value as scale estimator. See Yang and Thorne “Normalization for two-color cDNA microarray data.” In D. R. Goldstein, editor, Science and Statistics: A Festschrift for Terry Speed, volume 40, pages 403-41 (2003); Smyth and Speed “Normalization of cDNA microarray data.” Methods, 31: 265-273, (2003). No background subtraction was performed prior to normalization, as previously described. See Scharpf et al., “When should one subtract background fluorescence in 2-color microarrays? Biostatistics. 8:695-707 (2007); Zahurak et al., “Pre-processing Agilent microarray data.” BMC Bioinformatics. 8: 1471-2105 (2007). Positive and negative microarray control features were not used to compute the “loess” smoothing and were further excluded from the subsequent analysis. For each individual array M-values corresponding to identical features were averaged after “scale” normalization between arrays and the resulting matrix was used in all subsequent steps of the analysis.

Affymetrix raw data were normalized at probe-level by fitting the empirical stochastic model de-scribed by Irizarry and colleagues. See Irizarry et al., “Exploration, normalization, and summaries of high density oligonucleotide array probe level data.” Biostatistics. 4: 249-264 (2003). Standardization across DNA-chips was attained by quantile normalization. See Yang and Thorne “Normalization for two-color cDNA microarray data.” In D. R. Goldstein, editor, Science and Statistics: A Festschrift for Terry Speed, volume 40, pages 403-41 (2003); Smyth and Speed “Normalization of cDNA microarray data.” Methods, 31: 265-273, (2003). For the E-TABM147 data set, since it accounted for hybridizations made on two different array version, (hgu95a and hgu95av2), the corresponding result files were pre-processed separately and then joined before analysis of differential gene expression.

In all data sets considered in the present study differential gene expression was investigated using functions and methods implemented in the R/Bioconductor package limma. See Ihaka and Gentleman. “R: A language for data analysis and graphics.” Journal of Computational and Graphical Statistics. 5: 299-314 (1996); Smyth. “Limma: linear models for microarray data.” In R. Gentleman, R. V. Carey, S. Dudoit, R. Irizarry, and W. Huber, editors, Bioinformatics and Computational Biology Solutions using R and Bioconductor. 397-420. Springer, N.Y., (2005); Smyth, “Linear models and empirical Bayes methods for assessing differential expression in microarray experiments.” Statistical Applications in Genetics and Molecular Biology. 3(Article 3) (2004); Gentleman et al., “Bioconductor: open software development for computational biology and bioinformatics.” Genome Biol. 5: R80 (2004). Briefly, a fixed effects linear model was fit for each individual feature to estimate expression differences between groups of samples to be compared. When technical replicates or matched samples from the same individual were available, correlation coefficients were computed between replicates and the associated consensus correlation was added to the model. See Smyth et al., “Use of within-array replicate spots for assessing differential expression in microarray experiments.” Bioinformatics. 21: 2067-75 (2005). For the data sets where a dye-swap dual-color design was applied, a coefficient for the dye-effect was further considered in the analysis. An empirical Bayes approach was applied to moderate standard errors of M-values. See Lonnstedt and Speed “Replicated microarray data.” Statistica Sinica. 12: 31-46 (2002); Smyth, “Linear models and empirical Bayes methods for assessing differential expression in microarray experiments.” Statistical Applications in Genetics and Molecular Biology. 3(Article 3) (2004). Finally, for each analyzed feature, t-statistics, log-odds ratios of differential expression (B-statistics), raw and adjusted p-values (FDR control by the Benjamini and Hochberg method) were obtained. See Benjamini and Hochberg “Controlling the false discovery rate: a practical and powerful approach to multiple testing.” Journal of the Royal Statistical Society Series B. 57: 289-300 (1995).

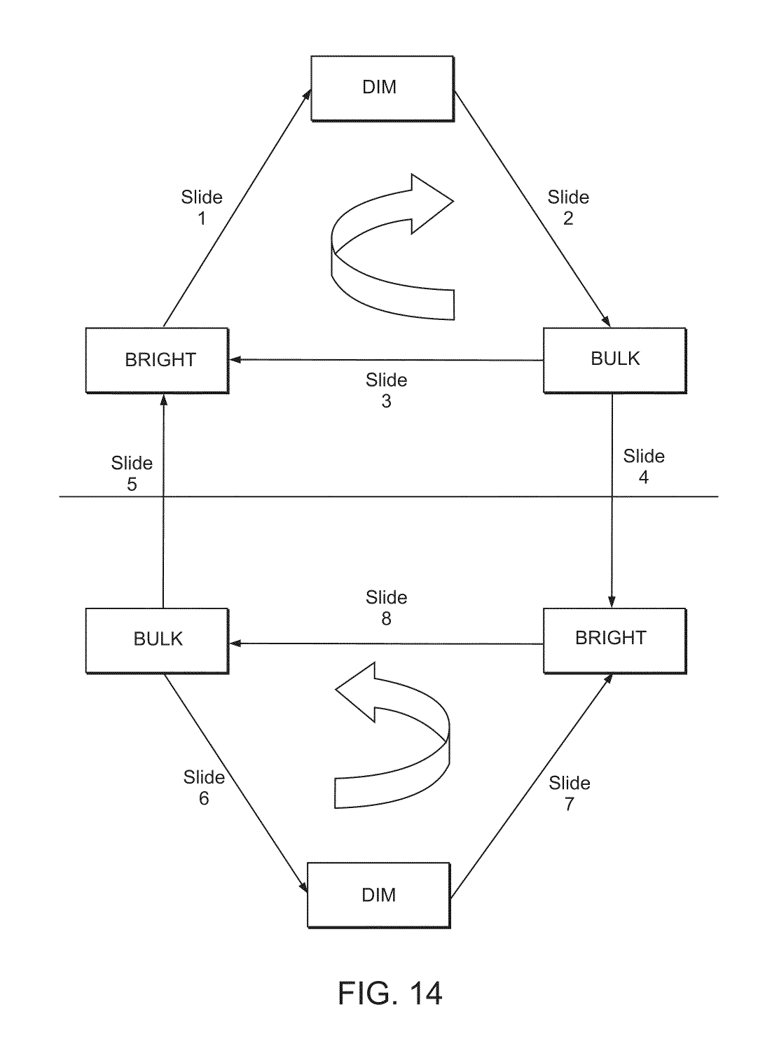

Two biological replicates were obtained for each of the three type of samples considered in the study (BRIGHT, DIM and BULK) analyzed, as shown in FIG. 14. A total number of 8 dual-color competitive hybridization was performed. Each RNA from BRIGHT and BULK was labeled three times, while RNA from DIM samples were labeled twice. The labeling scheme adopted allowed at least one set of measurements for each one of the two dyes used. A coefficient for the dye effect was added to the model.

In the analysis of the GSE3167 data set the following contrasts between distinct groups were performed: CIS versus normal bladder mucosa; Non invasive UC (Ta stage) versus normal bladder mucosa; Invasive UC (T1-T2 stage) versus normal bladder mucosa; Invasive UC (T1-T2 stage) versus non invasive UC (Ta stage); Cancer with adjacent CIS versus cancer without adjacent CIS. See Dyrskjøt et al., “Gene expression in the urinary bladder: a common carcinoma in situ gene expression signature exists disregarding histopathological classification.” Cancer Res 64:4040-4048 (2004).

In the analysis of the GSE88GSE89 data set the following contrasts between distinct groups were performed: Invasive UC (T1-T2 stage) versus non invasive UC (Ta stage); Cancer with adjacent CIS versus cancer without adjacent CIS. See Dyrskjøt et al., “Identifying distinct classes of bladder carcinoma using microarrays.” Nat. Genet. 33: 90-6 (2003).

In the analysis of the ETABM147 data set the following contrasts between distinct groups were performed: Non invasive UC (Ta stage) versus normal bladder mucosa; Invasive UC (T1-T4 stage) versus normal bladder mucosa; Invasive UC (T1-T4 stage) versus non invasive UC (Ta stage); In the mode, the consensus correlation obtained by comparing replicates between patients was added as a covariate. See Stransky et al., “Regional copy number-independent deregulation of transcription in cancer.” Nat. Genet. 38:1386-1396 (2006).

In the analysis of the GSE5287 data set, the overall survival time was used to fit a Cox proportional hazard model to identify associated genes. See Als et al., “Emmprin and survivin predict response and survival following cisplatin-containing chemotherapy in patients with advanced bladder cancer.” Clin Cancer Res. 13:4407-4414 (2007).

Results obtained from the different data sets and contrasts were compared at the individual gene level and at the Functional Gene Set (FGS) level, which is at the level of gene lists defined by biological knowledge. Findings were displayed using heatmaps, in which significance is represented by a suitable color scale, and by “correspondence at the top” plots. Functions and methods were developed for this purpose, and are implemented in a funcBox R-package. See Marchionni “funcbox, an r-package for comparative analysis of functional annotation.” Technical report, Johns Hopkins University, Manuscript, 2007.

Analysis of Functional Annotation (AFA) was performed to accomplish the following goals: capture relevant biological processes and pathways in the urothelial cancer xenograft model; test whether differential gene expression programs associated with UC 67LR bright and dim cells were enriched in any of the UC comparisons described herein. The Wilcoxon rank-sum test was used to perform the AFA in the UC xenograft model to search for and compare functional and biological concepts (Functional Gene Sets, FGS hereafter) associated with the gene expression program expressed by cells expressing high levels of 67LR (BRIGHT cells) when compared to those with low 67LR levels (DIM cells). The Wilcoxon rank-sum test computes a p-value to test the hypothesis that an FGS, defined by a functional annotation, tends to be more highly ranked in an ordered gene list. In the present study individual genes on the arrays were ranked by their absolute moderated t-statistics, as obtained from the linear model analysis. This analysis was done using two distinct reference populations: all the non-redundant genes (according to Entrez Gene identifiers) present on the microarray platform; all the non-redundant genes annotated to each specific functional theme (i.e., signaling path-ways);

After the statistical tests were performed, control of false discovery rate (correction for multiple hypothesis testing) was obtained by applying the Benjamini and Hochberg method, as implemented in the multtest R/Bioconductor package. See Benjamini and Hochberg “Controlling the false discovery rate: a practical and powerful approach to multiple testing.” Journal of the Royal Statistical Society Series B. 57: 289-300 (1995).

The following functional annotation themes were used to define gene sets fed into the analysis: Gene Ontology Terms (GO); KEGG Pathways gene sets. See Ashburner and Lewis “On ontologies for biologists: the Gene Ontology—untangling the web.” Novartis Found Symp. 247:66-80 (2002); Ashburner, et al., “Gene ontology: tool for the unification of biology. The Gene Ontology Consortium.” Nat. Genet. 25:25-29 (2000); Harris et al., “The Gene Ontology (GO) database and informatics resource.” Nucleic Acids Res. 32:D258-261 (2004); Kanehisa et al., “The KEGG resource for deciphering the genome.” Nucleic Acids Res. 32:D277-280 (2004) (Table 4).

AFA was performed to investigate whether the gene expression program of the 67LR bright cells was involved in the progression of human UC across the human UC contrasts described herein. The lists of genes differentially expressed between 67LR bright and dim cells as Functional Gene Sets (FGS) was used for the analysis, which was performed by applying different stringency cut-offs to select the genes differentially expressed between 67LR bright and dim cells, and similar results were obtained in all contrasts performed among the distinct groups of human UC specimens that were compared.

The up- or down-regulated genes in 67LR bright cells when compared to the 67LR dim cells were analyzed as separate sets. The enrichment analysis was performed at both tails of the moderated t-statistics distributions, as obtained from the linear models in the human UC data sets described (Table 2 and Table 3).

Mappings between individual features of each microarray platform used to the various functional themes considered were based on NCBI Entrez Gene, as obtained from the corresponding R— Bioconductor metadata packages.

AFA results as obtained from Wilcoxon tests were compared and displayed using heatmaps in which the level of significance (i.e. p-values from the Wilcoxon rank sum tests) was represented in a suitable color scale. Logarithmic transformation (base 10) was applied prior generation of the heatmaps,

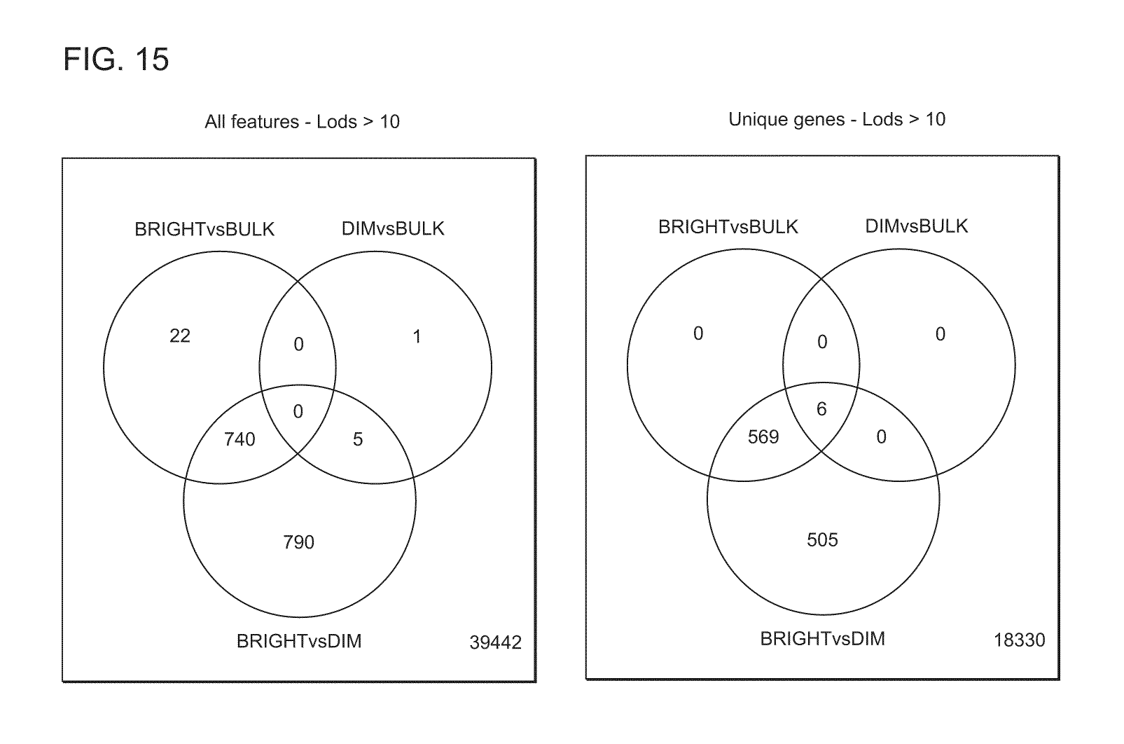

Venn diagrams were used to visualize the number of differentially expressed genes in common across the contrasts considered in the study. FIG. 15 shows the number of genes found to be differentially expressed in the three cell populations that were compared: DIM, BRIGHT and BULK. A large number of differentially expressed genes (Lods >10) was found only in the comparisons between BRIGHT and DIM, and between BRIGHT and BULK, while virtually no genes were found to be different when BULK and DIM were compared. Similar results were found also by using less stringent thresholds (lower Lods) to compute the intersections and make the Venn diagrams.

The “Concordance-at-the-top” plot (cat-plot) was developed to assess the agreement between microarray results from different platforms and laboratories. See Irizarry et al., “Multiple-laboratory comparison of microarray platforms.” Nat. Methods. 2:345-350 (2005). In particular, this technique enables comparing the correspondence of top ranking genes in two lists ranked by a predefined statistic. This was accomplished by: 1. Ordering the two lists according to a suitable statistic (i.e. differential gene expression, significance, probability); 2. Computing the proportion of elements in common for a given list size; 3. Reiterating the two steps above increasing the list size up to all common elements; 4. Plotting the proportion of common elements against the increasing size of the considered lists.

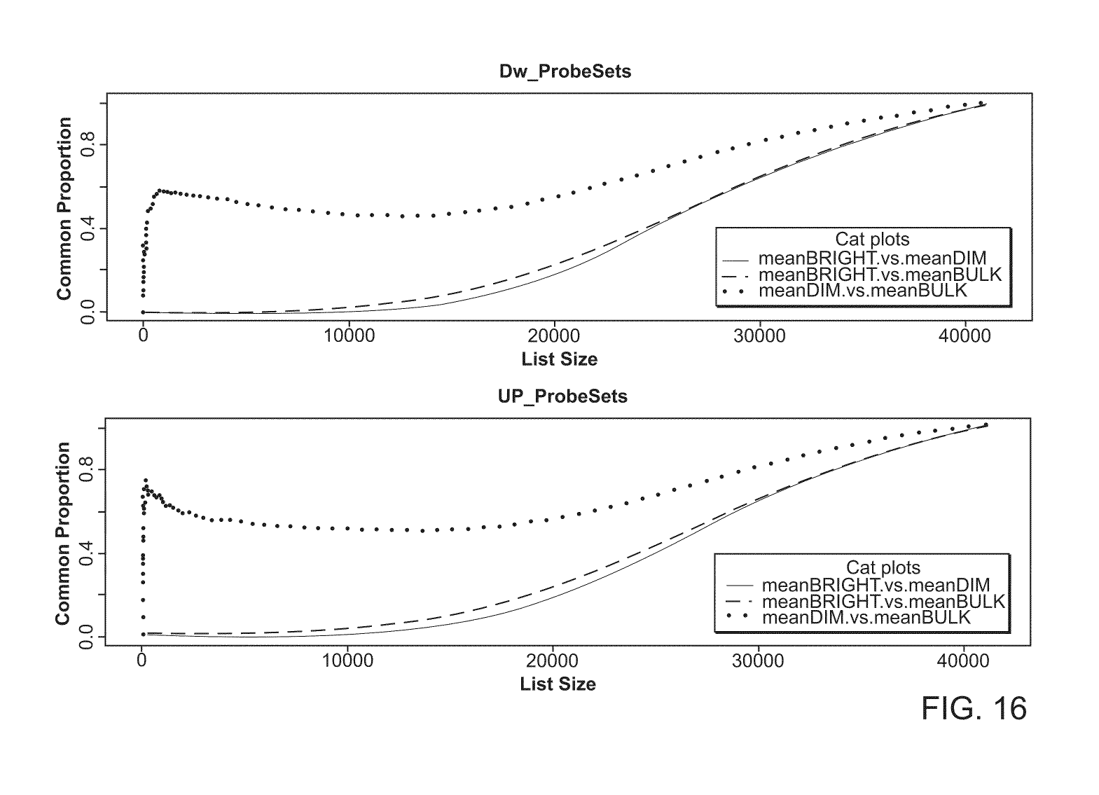

Cat-plots were used to evaluate the agreement of gene expression signatures across the comparisons considered in the present study (i.e. BRIGHT vs. DIM, DIM vs. BULK), by ranking the genes according to their fold-change to the median. This analysis, summarized in a CAT-plot in FIG. 16, revealed that the BRIGHT cells are distinct from BULK cells and DIM cells, since they showed virtually no overlap at the top of the ranked gene lists. On the contrary gene expression in the DIM cells is not distinct from control sorted cells (BULK), and about 60% of genes were in common at the top of the two gene lists (FIG. 16).

All analyses were performed using analytical packages from the R/Bioconductor project, including limma, affy, gcrma, multtest, and GEOquery. See Ihaka and Gentleman “A language for data analysis and graphics.” Journal of Computational and Graphical Statistics. 5:299-314 (1996); Gentleman et al., “Bioconductor: open software development for computational biology and bioinformatics.” Genome Biol. 5: R80 (2004); Smyth et al., “Use of within-array replicate spots for assessing differential expression in microarray experiments.” Bioinformatics. 21: 2067-75 (2005); Gautier et al., “affy-analysis of Affymetrix GeneChip data at the probe level.” Bioinformatics. 20: 307-315 (2004); Katz et al., “A summarization approach for Affymetrix GeneChip data using a reference training set from a large, biologically diverse database.” BMC Bioinformatics. 7:1471-2105 (2006).

Immunodetection of proteins was performed as described in Kleeberger et al., to visualize proteins, including those involved in cell differentiation and pathway activation. See Kleeberger et al., “Roles for the stem cell associated intermediate filament Nestin in prostate cancer migration and metastasis.” Cancer research. 67:9199-9206 (2007).

DNA microarrays were used to compare RNA transcript expression from distinct cell populations sorted by FACS with an antibody against the 67 Kd laminin receptor (67LR). Sorted cells were derived by digesting xenografted tumors into single cell suspensions. Differential gene expression was assessed by direct comparisons of labeled moieties, using a dye-swap design and biological replication. Hybridizations were performed on Agilent (Santa Clara, Calif.) Whole Human Genome DNA microarrays (hgug4112a). Three different samples were analyzed using two biological replicates: Urothelial cancer cells expressing 67LR (67LR+, BRIGHT); Urothelial cancer cells not expressing 67LR (67LR−, DIM); Control sorted urothelial cancer cells (BULK). The experimental design for the 8 hybridization is shown in FIG. 14.

Dyrskjøt and colleagues obtained gene expression in 60 specimens from UC patients of various stages, including invasive cancers with or without adjacent carcinoma in situ (CIS); CIS without adjacent invasive cancer; and normal bladder specimens. The authors used the gene expression profiles to build a gene classifier associated with the presence of CIS in the bladder, irrespective to UC stage. See Dyrskjøt et al., “Gene expression in the urinary bladder: a common carcinoma in situ gene expression signature exists disregarding histopathological classification.” Cancer Res 64:4040-4048 (2004). The hybridizations were performed using the Affymetrix GeneChip Human Genome U133 Array Set HG-U133A (hgul33a). Data were obtained from the NCBI Gene Expression Omnibus database (GEO, series GSE3167). See Wheeler et al., “Database resources of the National Center for Biotechnology Information.” Nucleic Acids Res. 33(Database issue):D39-45 (2005).

Study Results

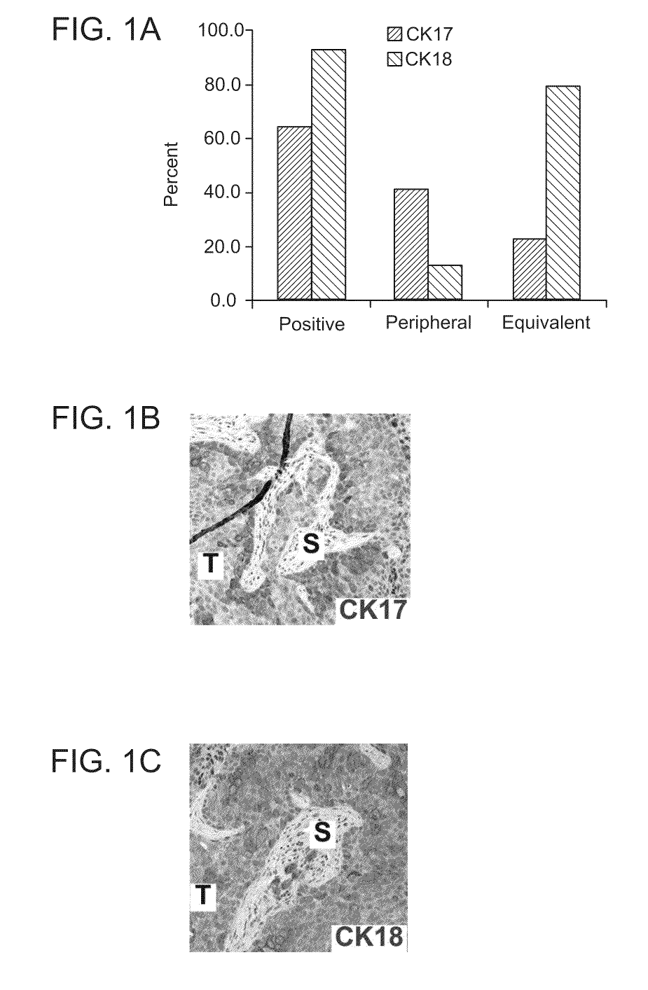

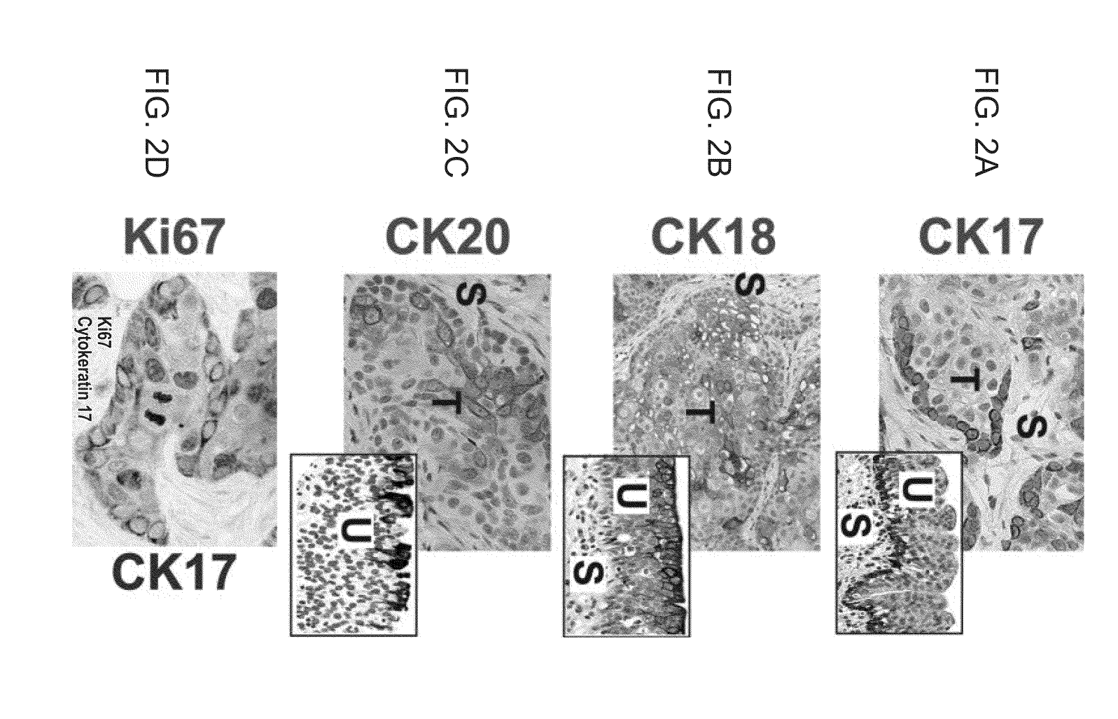

Immunohistochemical analysis revealed that in the majority of cases, the expression patterns of CK17 and CK18 in urothelial carcinomas mimic aspects of their physiologic expression in normal urothelium, with increasing differentiation as a function of distance from the basement membrane. The results from additional immunohistochemical analysis of CK17, CK18, and CK20 in SW780 UC xenografts indicate that UC nodules recapitulate normal basal-to-superficial urothelial differentiation, starting at the periphery and terminating in the interior. This differentiation can be supported, apparently, by either human bladder smooth muscle, as in the primary human tumors, or by mouse dermis, as in the xenografts (FIG. 1 and FIG. 2).

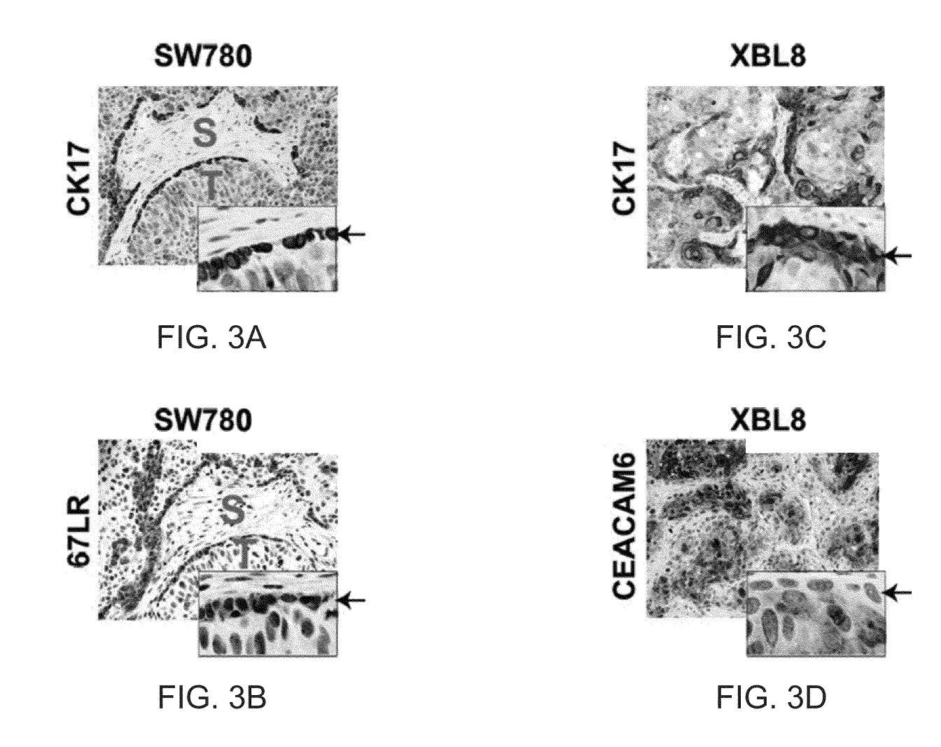

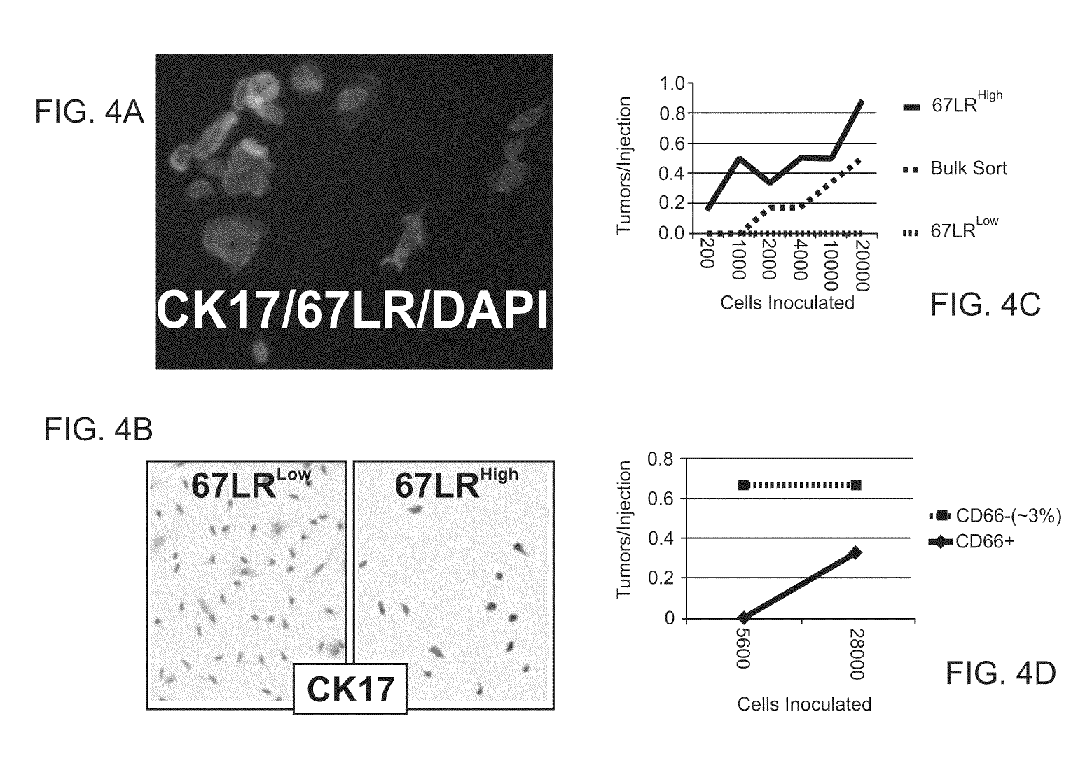

It was observed that the 67 kDa laminin receptor (67LR) was identified as a surface marker that preferentially decorated basal cells and colocalized with CK17 in SW780 xenografts in a stromal-dependent manner (FIGS. 3A,B). In contrast, SW780 cells grown in culture showed no obvious relationship between CK17 and 67LR expression (FIG. 4A). Together, these observations support the existence of a readily identifiable, spatially defined niche for carcinoma HTCs that is analogous to the normal urothelial stem cell niche.

Using a first-passage human UC xenograft, XBL8, it was confirmed that there is a readily identifiable, spatially defined niche for carcinoma HTCs that is analogous to the normal urothelial stem cell niche (FIGS. 3C,D).

The relationship between 67LR levels and tumorigenicity of SW780 UC cells grown alone in culture or purified from stroma-containing xenografted tumors was tested. No relationship between expression of 67LR and tumor forming potency upon subcutaneous inoculation into athymic (nude) mice.

In contrast to SW780 cells grown in vitro, 67LR expression in tumor cells grown as xenografts in vivo correlated dramatically with basal differentiation as determined by CK17 expression (FIG. 4B). 67LR bright cells constituted approximately 15% of the human cells in these analyses (FIG. 6), a fraction similar to that seen for HTCs in other carcinomas. See Visvader and Lindeman “Cancer stem cells in solid tumours: accumulating evidence and unresolved questions.” Nat Rev Cancer. 8: 755-68 (2008). The ability of these cells to initiate new tumors in immunodeficient mice was assayed by injecting 200-20,000 67LR bright and 67LR dim cells subcutaneously and waiting 4 months for discernable tumors to appear. Compared to bulk or the control-sorted SW780 cells, 67LR bright cells were ˜5-10-fold more potent at initiating new tumors in vivo (FIG. 4C). It was observed that the ability to form new tumors was an exclusive or nearly exclusive property of the basal cell compartment in SW780 xenografts (FIG. 4C).

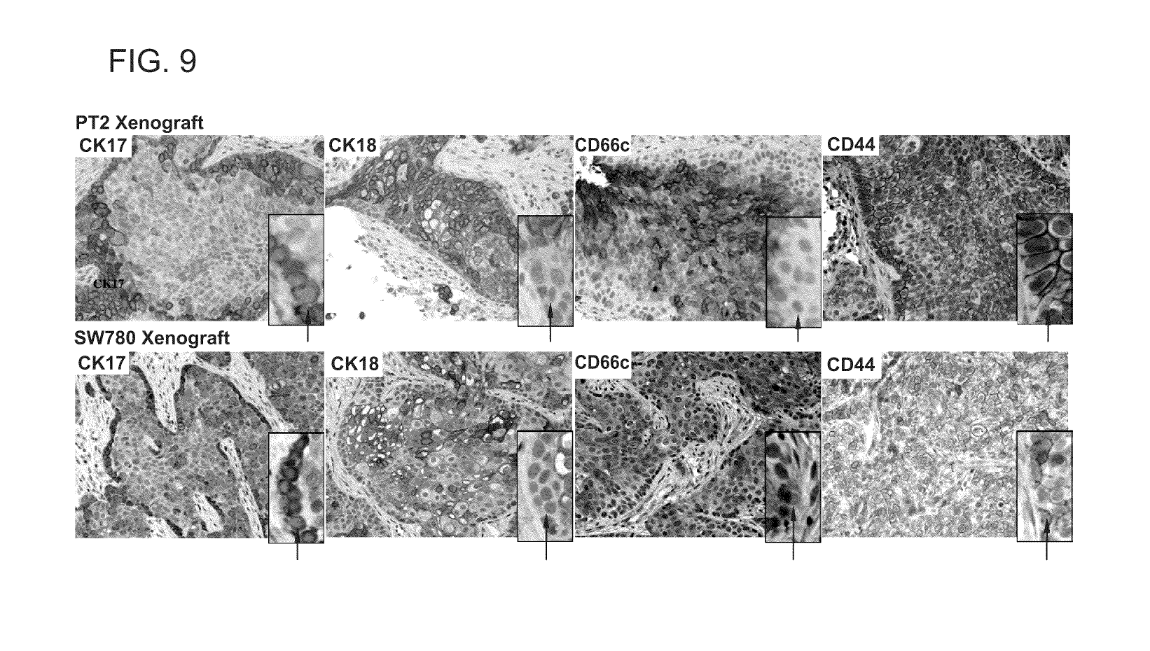

The enhanced tumorigenicity of basal-like UC cells was confirmed using XBL8 xenografts and fractionating them by expression of CEACAM6 and the hyaluronic acid receptor CD44. Contrary to studies by various groups, CD44 expression was not consistently found to be limited to the tumor-stroma interface (FIG. 9), and CD44 expression was substantial in cancer cells with and without basal cell differentiation (FIG. 9). CEACAM6 dim/CD44 bright basal-like XBL8 UC cells (FIG. 7) constituted approximately 3% of the parental tumor and were dramatically more potent than CEACAM6 bright non-basal cells in forming tumors (FIG. 4D). See Prince et al., “Identification of a subpopulation of cells with cancer stem cell properties in head and neck squamous cell carcinoma.” Proc Natl Acad Sci USA. 104: 973-8 (2007); Yang and Chang “Bladder cancer initiating cells (BCICs) are among EMA-CD44v6+ subset: novel methods for isolating undetermined cancer stem (initiating) cells.” Cancer Invest. 26:725-733 (2008).

These results, validated in two separate tumor models and using independent sorting strategies, further support the enhanced tumor-forming potential of basal-like UC cells.

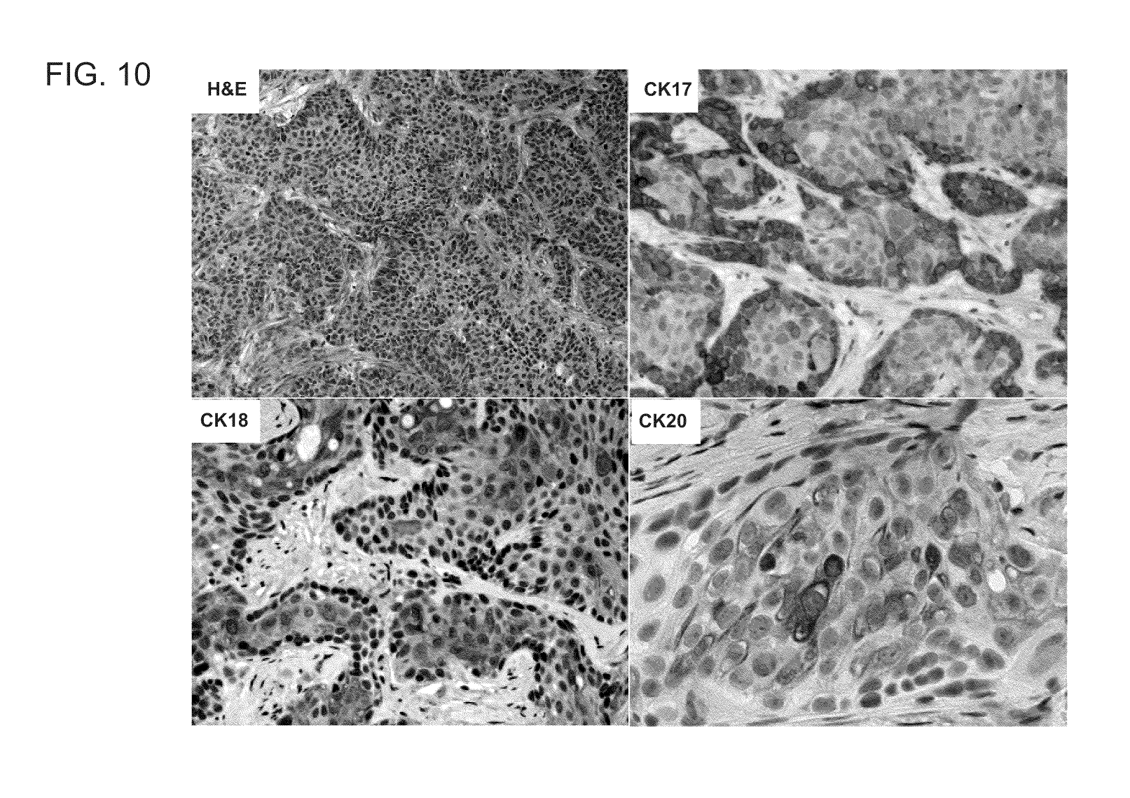

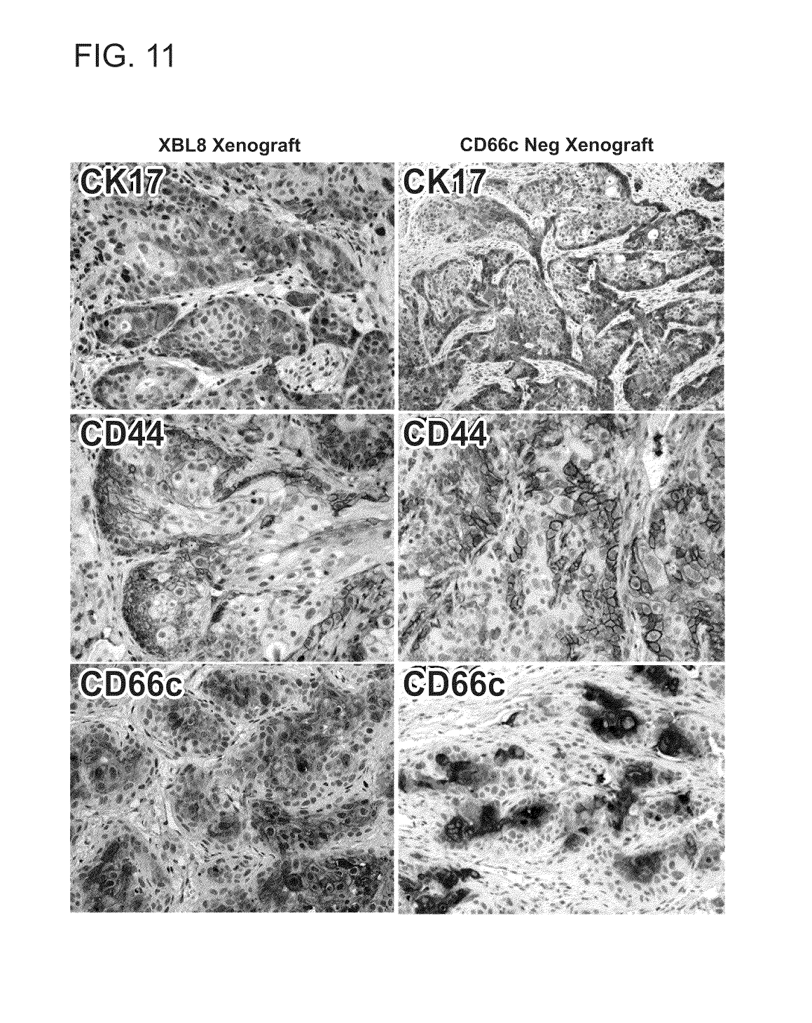

Histologic and immunohistochemical analysis of basal cell derived tumors demonstrated the presence of basal-like cells as well as more differentiated cells—recapitulating their arrangement in the parental tumor (FIG. 10 and FIG. 11). These results indicate that basal HTCs share functional properties normal urothelial basal stem cells, including enhanced growth potential and multipotency.

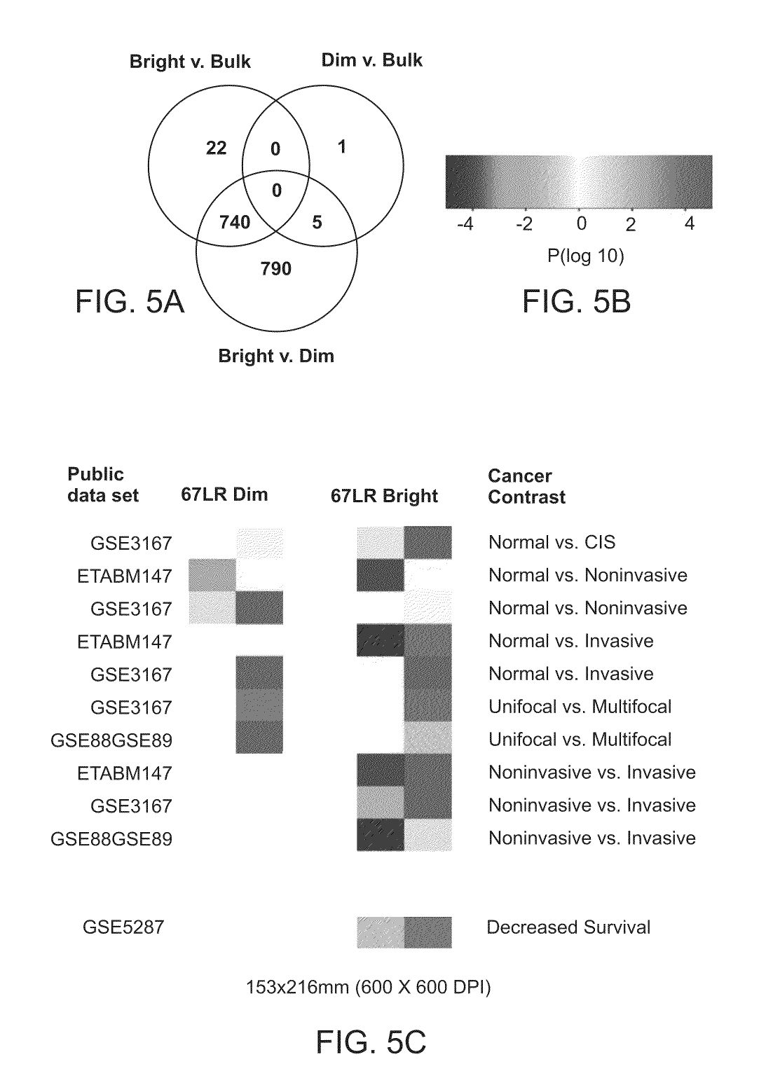

The cellular pathways underlying cancer progression and multipotency were investigated by mRNA expression analysis of sorted 67LR bright and 67LR dim fractions of human SW780 UC xenografts. A preliminary analysis of 44,000 feature whole genome array hybridizations identified robust differential expression of over 1,000 genes at the adj. P<0.05 level (corrected for multiple comparisons). Partial lists of differentially expressed genes are included in Tables 3-5. It was demonstrated that gene expression in 67LR dim and sham-sorted SW780 cells were essentially indistinguishable from each other and markedly different from 67LR bright basal-like cells (FIG. 5A). This result indicates that the basal-like cells are distinct from the tumor population as a whole, and that the UC basal HTC gene expression profile is completely hidden within the gene expression profile of the unfractionated tumor.

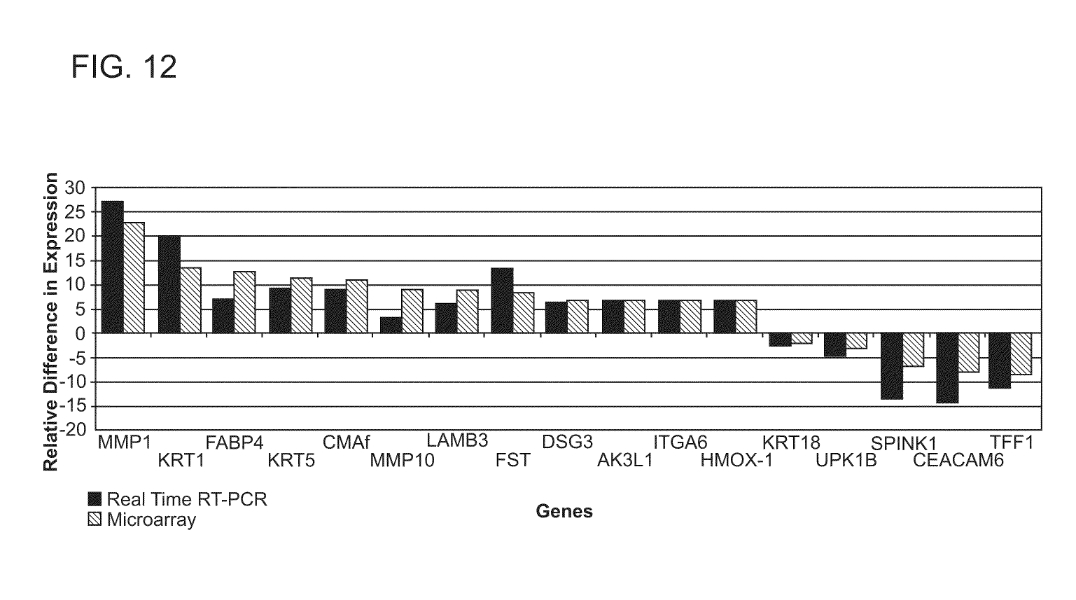

The differential expression of 17 of 17 genes was confirmed by real time RT-PCR (FIG. 12). Contrasts between 67LR bright and 67LR dim populations were focused on, by performing Analysis of Functional Annotation (AFA). See Schaeffer et al., “Androgen-induced programs for prostate epithelial growth and invasion arise in embryogenesis and are reactivated in cancer.” Oncogene. 27:7180-7191 (2008). This analysis revealed significant (adj. P<0.05) enrichment of genes associated with “Hallmark” characteristic activities of cancer including apoptosis, cell growth, and proliferation (Tables 2-5). See Hanahan and Weinberg, “The hallmarks of cancer.” Cell. 100: 57-70 (2000). There was also significant enrichment of genes comprising a variety of pharmacologically tractable signaling pathways, including Jak-STAT, Notch, Focal Adhesion, mTOR, ErbB, and Wnt. See Golubovskaya et al., “A Small Molecule Inhibitor, 1,2,4,5-Benzenetetraamine Tetrahydrochloride, Targeting the Y397 Site of Focal Adhesion Kinase Decreases Tumor Growth.” J Med. Chem. 51:7405-7416 (2008); Kim et al., “A small-molecule compound identified through a cell-based screening inhibits JAK/STAT pathway signaling in human cancer cells.” Mol Cancer Ther. 7:2672-80 (2008); Rizzo et al., “Rational targeting of Notch signaling in cancer.” Oncogene. 27:5124-31 (2008); Xue et al., “Palomid 529, a novel small-molecule drug, is a TORC1/TORC2 inhibitor that reduces tumor growth, tumor angiogenesis, and vascular permeability.” Cancer research. 68: 9551-9557 (2008); Wakeling “Inhibitors of growth factor signaling.” Endocr Relat Cancer. Suppl 1:S183-187 (2005); Chen et al., “Small molecule-mediated disruption of Wnt-dependent signaling in tissue regeneration and cancer.” Nat Chem. Biol. 5:100-107 (2009).

The Wnt signaling Pathway was specifically explored by gene expression analysis and WNT signaling was found to be highly significant (p<0.03). 67LR bright HTCs showed highly significant upregulation (adj. p<10⁻⁶ in comparison to levels in 67LR dim cells) of several activating pathway components, including WNT4, 6, and 10 A ligands, their FZD6 receptor, and the Wnt effector/transcriptional co-activator β-catenin. These cells also showed significant downregulation (adj. p<10⁻⁶) of the Wnt ligand antagonist DKK1. KREMEN2 levels were markedly elevated in 67LR bright HTCs, which, in the setting of low DKK levels, can convert KREMEN proteins from Wnt pathway antagonists to pathway agonists by stabilizing the Wnt ligand coreceptors LRP5 and LRP6. See Stoehr et al., “No evidence for involvement of beta-catenin and APC in urothelial carcinomas.” Int J Oncol. 20: 905-911 (2002); Cselenyi and Lee “Context-dependent activation or inhibition of Wnt-beta-catenin signaling by Kremen.” Sci Signal. 1:pe10 (2008).

Wnt ligand binding leads to stabilization of β-catenin and allows it to activate Wnt pathway target genes. Although such targets vary across tissue types, several common Wnt targets, including MYC and MMP7 (43, 44), were significantly (adj p<10⁻⁵) upregulated in 67LR bright cells. See Crawford et al., “The metalloproteinase matrilysin is a target of beta-catenin transactivation in intestinal tumors.” Oncogene. 18: 2883-2891 (1999); He et al., “Identification of c-MYC as a target of the APC pathway.” Science. 281:1509-1512 (1998).

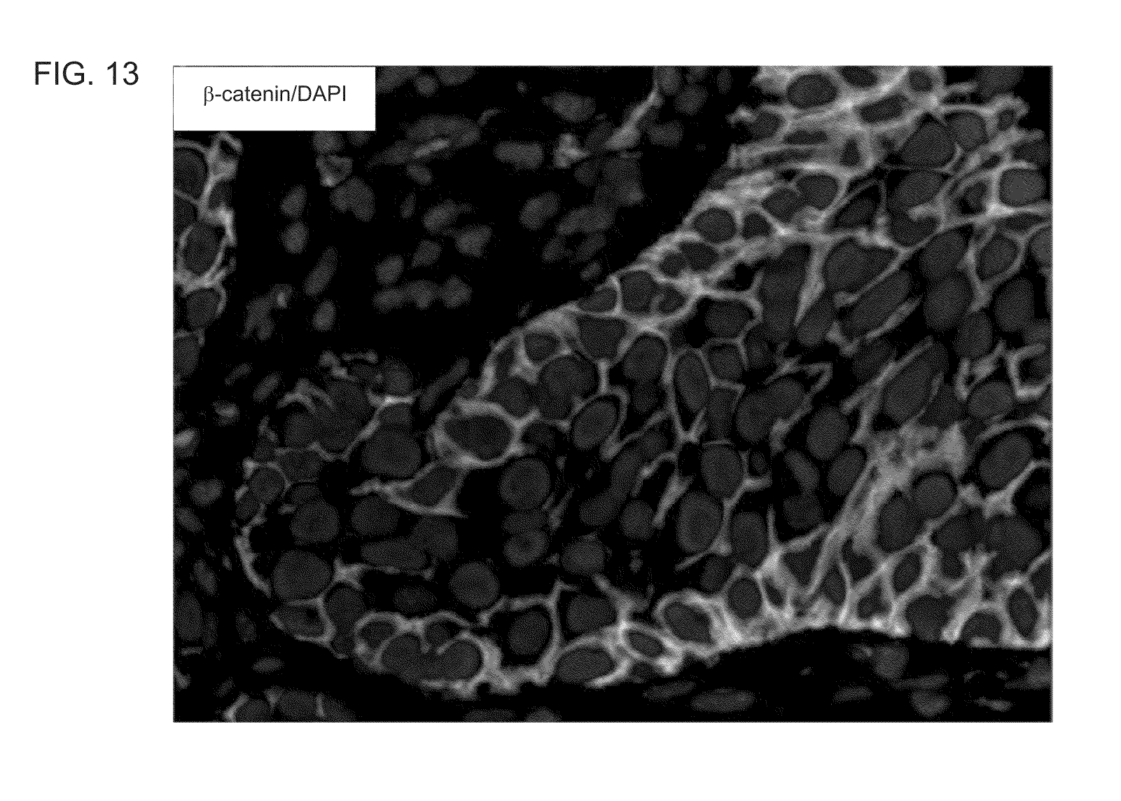

These mRNA expression profiling results demonstrate selective expression of WNT activating components and target genes in 67LR-bright basal/HTCs. In addition, immunofluorescent staining of SW780 human UC xenografts with β-catenin specific antibodies demonstrated basal-specific cytoplasmic accumulation of the Wnt pathway effector β-catenin (FIG. 13), consistent with moderate levels of pathway activity. See Gao et al., “Cytoplasmic expression of E-cadherin and beta-Catenin correlated with LOH and hypermethylation of the APC gene in oral squamous cell carcinomas.” J Oral Pathol Med. 34: 116-9 (2005). Little staining was observed in the interior non-tumorigenic regions of tumor nodules. Taken together, these results show regulation of WNT pathway components in a pattern expected to stimulate WNT signaling activity and expression of target genes in basal/HTCs.

Expression analysis indicated that 67LR bright HTCs were significantly enriched for expression of potential chemoresistance pathways, including genes with oxidoreductase activity (p<10⁻⁶), and >2-fold upregulation of several individual genes with established protective roles in stem cells and/or cancer. These included antioxidant and detoxification enzymes such as aldehyde dehydrogenase which mediates resistance to cyclophosphamide; heme oxygenase, which mediates resistance to gemcitabine and radiation therapy, and superoxide dismutase, which mediates resistance to both daunorubicin and radiotherapy. See Kastan et al., “Direct demonstration of elevated aldehyde dehydrogenase in human hematopoietic progenitor cells.” Blood. 75: 1947-1950 (1990); Berberat et al., “Inhibition of heme oxygenase-1 increases responsiveness of pancreatic cancer cells to anticancer treatment.” Clin Cancer Res. 11: 3790-3798 (2005); Eisele et al., “Differential expression of drug-resistance-related genes between sensitive and resistant blasts in acute myeloid leukemia.” Acta Haematol. 117: 8-15 (2007); Josson et al., “RelB regulates manganese superoxide dismutase gene and resistance to ionizing radiation of prostate cancer cells.” Oncogene. 25: 1554-9 (2006).

These results indicate the potential utility of studying chemo- and radio-responsiveness in SW780 xenografts, particularly since this model is easily monitored by histology and immunohistochemistry for effects on numbers of basal-like vs. differentiated cells.

The 67LR bright HTC gene expression signature is associated with UC progression and death. As a further indication of a role for basal-like cells in human UC biology, highly significant (in many cases well below p=0.01) identity was found in genes that were concordantly (up and/or down) regulated in both 67LR bright cells and in increasingly aggressive forms of UC (FIG. 5). There was the significant enrichment (p<0.001) of genes predicting poor survival in UC. See Als et al., “Emmprin and survivin predict response and survival following cisplatin-containing chemotherapy in patients with advanced bladder cancer.” Clin Cancer Res. 13: 4407-14 (2007). Since the basal HTC gene signature should be masked by more differentiated cells, as indicated herein (FIG. 5A), these results suggest that a particularly strong relationship between HTCs and aggressive behavior in UCs. Compared to gene expression in non-tumorigenic cancer cells, basal HTC gene expression was far more significantly related to gene expression in UC progression (FIGS. 5B and C). These results support a recent report relating HTC gene expression and poor prognosis in breast cancer and further validate the biologic and clinical relevance of the experimental xenograft system. See Liu et al., “The prognostic role of a gene signature from tumorigenic breast-cancer cells.” The New England Journal of Medicine. 356:217-26 (2007). Thus basal-like cells, a minor fraction of UC tumors, may play a disproportionately important role in cancer progression.

A differentiation program in the majority of urothelial carcinomas that mirrors normal urothelial differentiation was identified. The experimental data support the hypothesis that carcinomas have a basal population at the tumor-stroma interface that resembles benign urothelial stem cells, differentiates as it moves away from the basal compartment, and loses tumor-forming potential upon differentiation. The finding that urothelial HTCs localize to the basal compartment and likely interact with adjacent stroma provides empirical support for proposed links between stem cell differentiation and the “invasive front” of carcinomas (15). See Brabletz et al., “Opinion: migrating cancer stem cells—an integrated concept of malignant tumour progression.” Nat Rev Cancer. 5: 744-9 (2005).

The idea of a stromal niche, was supported by the observation that mouse subcutaneous stroma was able to direct adjacent UC cells to differentiate in a way that is very much analogous to the differentiation of normal basal urothelial cells by bladder stroma. This differentiation was absent in vitro, but apparent in xenografts, whether inoculated from either low passage xenografts or from long established cultured cells. These results demonstrate a potentially convenient system for assaying HTC and stem cell related properties using cells readily available from public repositories and easily manipulated for engineered expression of genes of interest. These observations point to the potential utility of studying epithelial-stromal interactions and their malignant (carcinoma-stroma) counterparts in stem cell and HTC biology.

The enhanced tumor forming potential of the basal compartment is accompanied by distinctive gene expression programs associated with fundamental (“Hallmark”) properties of cancer as well as pharmacologically tractable signaling pathways (Tables 2-5). See Hanahan and Weinberg, “The hallmarks of cancer.” Cell. 100: 57-70 (2000). Basal HTC gene expression is masked by other cells in the tumor, but is clinically relevant as demonstrated by its strong overlap with genes driving neoplastic transformation, invasion and death in UC (FIG. 5).

In addition to xenografting efficiency, several other observations support the hypothesis that HTCs are disproportionately important in promoting human UC progression including 1) the similarity of basal-like HTCs to normal multipotent, self renewing urothelial stem cells; 2) the mulitpotency of HTCs in xenografting assays (FIGS. 10 and 11); and 3) the concordant expression by HTCs of genes driving UC progression in humans (FIGS. 5B,C).

Incorporation by Reference

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating cancer comprising administering to a patient diagnosed with cancer an antibody or an antibody fragment thereof that binds to a protein that is expressed by a gene listed in Table 3 or Table 6, in an amount sufficient to inhibit the proliferation of cancer cells in the patient.

2. (canceled)

3. A method of treating cancer comprising administering to a patient diagnosed with cancer antibody or an antibody fragment thereof attached to a therapeutic moiety, wherein said antibody fragment binds to a protein that is expressed by a gene listed in Table 3 or Table 6, in an amount sufficient to inhibit the proliferation of cancer cells in the patient.

4. (canceled)

5. The method of claim 1, wherein the cancer cells are reduced in the patient.

6. The method of claim 5, wherein the method further comprises detecting cancer cells in the patient.

7. The method of claim 6, wherein the method detects a reduction in tumor size.

8. The method of claim 6, wherein the detection utilizes a specimen from the patient.

9. The method of claim 8, wherein the specimen is from a blood sample, a bone marrow sample or a tumor biopsy.

10. (canceled)

11. (canceled)

12. The method of claim 1, wherein cancer cells do not increase in the patient.

13. The method of claim 12, wherein the method further comprises detecting cancer cells in the patient.

14. The method of claim 13, wherein the method detects a lack of increase in tumor size.

15. The method of claim 13, wherein the detection utilizes a specimen from the patient.

16. (canceled)

17. The method of claim 13, wherein the detection utilizes an imaging technique.

18. (canceled)

19. The method of claim 1, wherein the method results in a reduction in cancer stem cells in the patient.

20. The method of claim 1, wherein the method further comprises detecting cancer stem cells in the patient.

21. The method of claim 20, wherein the detection utilizes a specimen from the patient, wherein the specimen is selected from the group consisting of a blood sample, a bone marrow sample, or a tumor biopsy.

22. (canceled)

23. (canceled)

24. (canceled)

25. (canceled)

26. (canceled)

27. The method of claim 3, wherein the therapeutic moiety is selected from alkylating agents, anti-metabolites, plant alkaloids, chemotherapeutic agents, radionuclides, therapeutic enzymes, cytokines, cytotoxins, or growth modulators.

28. The method of claim 1, wherein the antibody or fragment thereof results in a decrease in viability of cancer stem cells.

29. (canceled)

30. The method of claim 1, wherein the antibody or fragment thereof modulates cancer stem cells.

31. The method of claim 1, wherein the patient has been diagnosed with a solid tumor or with a hematologic cancer and has undergone cancer therapy.

32. (canceled)

34. (canceled)

35. (canceled)

36. (canceled)

37. The method of claim 1, wherein the cancer is brain cancer, neural cancer, breast cancer, leukemia, kidney cancer, colorectal cancer, prostate cancer, rhabdomyosarcoma, retinoblastoma, hepatic cancer, epidermal cancer, gastrointestinal cancer, pancreatic cancer, hematopoietic cancer, cervical cancer, or lung cancer.

38. (canceled)

39. (canceled)

40. (canceled)

41. (canceled)
